(19) United States
(12) Patent Application Publication
(10) Pub. No.: US 2023/0331825 A1
(43) Pub. Date: Oct. 19, 2023

(54) SARS-COV-2 ASSOCIATED ANTIBODY COMPOSITIONS AND METHODS OF USE

(71) Applicant: Target Discovery Merger Sub II, LLC, Vancouver, WA (US)

(72) Inventors: Goran Rakocevic, Belgrade (RS); Daniele Biasci, Poole, Dorest (GB)

(21) Appl. No.: 18/042,639

(22) PCT Filed: Aug. 25, 2021
(86) PCT No.: PCT/US21/47526

§ 371 Date: Feb. 23, 2023

Related U.S. Application Data
(60) Provisional application No. 63/069,971, filed on Aug. 25, 2020.

(51) Int. Cl. C07K 16/10; G01N 33/531; G01N 33/569; A61P 31/14; A61K 45/06; A61K 39/42

(52) U.S. Cl. CPC C07K 16/1003; G01N 33/531; G01N 33/56983; A61P 31/14; A61K 45/06; A61K 39/42; G01N 2333/165; G01N 2469/10; C07K 2317/565; C07K 2317/34; C07K 2317/76

(57) ABSTRACT

The disclosure herein relates to novel antibodies and antigen binding fragments that are used in the treatment, prevention and diagnosis of COVID-19, the disease caused by SARS-CoV-2. The complete polypeptide and nucleic acid consensus sequences of the antibodies and antigen binding fragments disclosed herein are reconstructed in silico.

### CROSS REFERENCE

This application claims the benefit of U.S. Provisional Patent Application Ser. No. 63/069,971, filed on Aug. 25, 2020, titled “SARS-CoV-2 Associated Antibody Compositions and Methods of Use”, the contents of which are incorporated by reference in their entirety.

### SEQUENCE LISTING

The present application includes a Sequence Listing which has been submitted electronically in ASCII format and is hereby incorporated by reference in its entirety. Said ASCII copy, created on Aug. 20, 2021, is named “61631730601_sequencelisting.txt” and is 10,856,000 bytes in size.

### BACKGROUND OF THE INVENTION

COVID-19 is a human disease caused by a recently emerged strain of coronavirus, SARS-CoV-2. Antibodies with suitable paratope to bind to a viral epitope, packaged into an appropriate pharmaceutical delivery mechanism, may be effective at neutralizing the virus thereby slowing the spread of disease or reducing its burden.

### SUMMARY OF THE INVENTION

In one aspect, provided herein is an antibody or antigen-binding fragment thereof comprising at least one of: (a) a variable heavy chain complementarity-determining region CDR-H1, CDR-H2 and CDR-H3, wherein: (i) CDR-H1 comprises a reconstructed polypeptide consensus sequence selected from any one of SEQ ID NOs: 10001-11250, (ii) CDR-H2 comprises a reconstructed polypeptide consensus sequence selected from any one of SEQ ID NOs: 12501-13750, and (iii) CDR-H3 comprises a reconstructed polypeptide consensus sequence selected from any one of SEQ ID NOs: 15001-16250; and (b) a variable light chain complementarity-determining region CDR-L1, CDR-L2 and CDR-L3, wherein: (i) CDR-L1 comprises a reconstructed polypeptide consensus sequence selected from any one of SEQ ID NOs: 11251-12500, (ii) CDR-L2 comprises a reconstructed polypeptide consensus sequence selected from any one of SEQ ID NOs: 13751-15000, and (iii) CDR-L3 comprises a reconstructed polypeptide consensus sequence selected from any one of SEQ ID NOs: 16251-17500.

In one aspect of the present disclosure, provided herein is an antibody or antigen-binding fragment thereof comprising at least one of: (a) a variable heavy chain, wherein the variable heavy chain comprises a reconstructed polypeptide consensus sequence having at least 90% sequence identity to an amino acid sequence selected from any one of SEQ ID NOs: 17501-18750; and (b) a variable light chain, wherein the variable light chain comprises a reconstructed polypeptide consensus sequence having at least 90% sequence identity to an amino acid sequence selected from any one of SEQ ID NOs: 18751-20000.

In one aspect, provided herein is an antibody or antigen-binding fragment thereof that comprises: (a) a variable heavy chain complementarity-determining region CDR-H1, CDR-H2, and CDR-H3, wherein CDR-H1 comprises a reconstructed polypeptide consensus sequence selected from any one of SEQ ID NOs: 10001-11250, CDR-H2 comprises a reconstructed polypeptide consensus sequence selected from any one of SEQ ID NOs: 12501-13750, and CDR-H3 comprises a reconstructed polypeptide sequence selected from any one of SEQ ID NOs: 15001-16250; and (b) a variable light chain complementarity-determining region CDR-L1, CDR-L2, and CDR-L3, wherein CDR-L1 comprises a reconstructed polypeptide consensus sequence selected from any one of SEQ ID NOs: 11251-12500, CDR-L2 comprises a reconstructed polypeptide consensus sequence selected from any one of SEQ ID NOs: 13751-15000, and CDR-L3 comprises a reconstructed polypeptide sequence selected from any one of SEQ ID NOs: 16251-17500.

In one aspect, provided herein is an antibody or antigen-binding fragment thereof that comprises a variable heavy chain, wherein the variable heavy chain comprises a reconstructed polypeptide consensus sequence selected from any one of SEQ ID NOs: 17501-18750; and a variable light chain, wherein the variable light chain comprises a reconstructed polypeptide consensus sequence selected from any one of SEQ ID NOs: 18751-20000.

In one aspect, provided herein is an antibody or antigen-binding fragment thereof that comprises a variable heavy chain complementarity-determining region CDR-H1, CDR-H2, and CDR-H3, wherein CDR-H1 comprises a reconstructed polypeptide consensus sequence selected from any one of SEQ ID NOs: 10001-11250, CDR-H2 comprises a reconstructed polypeptide consensus sequence selected from any one of SEQ ID NOs: 12501-13750, and CDR-H3 comprises a reconstructed polypeptide sequence selected from any one of SEQ ID NOs: 15001-16250.

In one aspect, provided herein is an antibody or antigen-binding fragment thereof that comprises a variable light chain complementarity-determining region CDR-L1, CDR-L2, and CDR-L3, wherein CDR-L1 comprises a reconstructed polypeptide consensus sequence selected from any one of SEQ ID NOs: 11251-12500, CDR-L2 comprises a reconstructed polypeptide consensus sequence selected from any one of SEQ ID NOs: 13751-15000, and CDR-L3 comprises a reconstructed polypeptide sequence selected from any one of SEQ ID NOs: 16251-17500.

In one aspect, provided herein is an antibody or antigen-binding fragment thereof that comprises: a variable heavy chain complementarity-determining region CDR-H1, CDR-H2, and CDR-H3, wherein CDR-H1 comprises a reconstructed polypeptide consensus sequence selected from any one of SEQ ID NOs: 10001-11250, CDR-H2 comprises a reconstructed polypeptide consensus sequence selected from any one of SEQ ID NOs: 12501-13750, and CDR-H3 comprises a reconstructed polypeptide sequence selected from any one of SEQ ID NOs: 15001-16250; and a variable light chain complementarity-determining region CDR-L1, CDR-L2, and CDR-L3, wherein CDR-L1 comprises a reconstructed polypeptide consensus sequence selected from any one of SEQ ID NOs: 11251-12500, CDR-L2 comprises a reconstructed polypeptide consensus sequence selected from any one of SEQ ID NOs: 13751-15000, and CDR-L3 comprises a reconstructed polypeptide sequence selected from any one of SEQ ID NOs: 16251-17500.

In one aspect, provided herein is an antibody or antigen-binding fragment thereof that comprises a variable heavy chain, wherein the variable heavy chain comprises a reconstructed polypeptide consensus sequence selected from any one of SEQ ID NOS: 17501-18750.

In one aspect, provided herein is an antibody or antigen-binding fragment thereof that comprises a variable light chain, wherein the variable light chain comprises a reconstructed polypeptide consensus sequence selected from any of SEQ ID NOS: 18751-20000.

In one aspect, provided herein is an antibody or antigen-binding fragment thereof that comprises: (a) a variable heavy chain, wherein the variable heavy chain comprises a reconstructed polypeptide consensus sequence selected from any one of SEQ ID NOS: 17501-18750; and (b) an antibody or antigen-binding fragment thereof that comprises a variable light chain, wherein the variable light chain comprises a reconstructed polypeptide consensus sequence selected from any of SEQ ID NOS: 18751-20000.

In one aspect, provided herein is an antibody or antigen-binding fragment thereof that comprises: (i) a variable heavy chain complementarity-determining region CDR-H1, CDR-H2 and CDR-H3, wherein (a) the CDR-H1 comprises the amino acid sequence of SEQ ID NO: 10369, (b) the CDR-H2 comprises the amino acid sequence of SEQ ID NO: 12869, and (c) the CDR-H3 comprises the amino acid sequence of SEQ ID NO: 15369; (ii) a variable light chain complementarity-determining region CDR-L1, CDR-L2 and CDR-L3, wherein: (a) CDR-L1 comprises the amino acid sequence of SEQ ID NO: 11619, (b) CDR-L2 comprises the amino acid sequence of SEQ ID NO: 14119, and (c) CDR-L3 comprises the amino acid sequence of SEQ ID NO: 16619; or (iii) the variable heavy chain complementarity-determining region CDR-H1, CDR-H2 and CDR-H3 of (i), and the variable light chain complementarity-determining region CDR-L1, CDR-L2 and CDR-L3 of (ii).

In one aspect, provided herein is an antibody or antigen binding fragment thereof that comprises: (a) a variable heavy chain that comprises a polypeptide sequence having at least 90% sequence identity to an amino acid sequence of SEQ ID NO: 17869; (b) a variable light chain that comprises a polypeptide sequence having at least 90% sequence identity to an amino acid sequence of SEQ ID NO: 19119; or (c) the variable heavy chain of (a), and the variable light chain of (b).

In one aspect, provided herein is an antibody or antigen-binding fragment thereof that comprises: (i) a variable heavy chain complementarity-determining region CDR-H1, CDR-H2 and CDR-H3, wherein (a) the CDR-H1 comprises the amino acid sequence of SEQ ID NO: 10260, (b) the CDR-H2 comprises the amino acid sequence of SEQ ID NO: 12760, and (c) the CDR-H3 comprises the amino acid sequence of SEQ ID NO: 15260; (ii) a variable light chain complementarity-determining region CDR-L1, CDR-L2 and CDR-L3, wherein: (a) the CDR-L1 comprises the amino acid sequence of SEQ ID NO: 11510, (b) the CDR-L2 comprises the amino acid sequence of SEQ ID NO: 14010, and (c) the CDR-L3 comprises the amino acid sequence of SEQ ID NO: 16510; or the variable heavy chain complementarity-determining region CDR-H1, CDR-H2 and CDR-H3 of (i), and the variable light chain complementarity-determining region CDR-L1, CDR-L2 and CDR-L3 of (ii).

In one aspect, provided herein is an antibody or antigen binding fragment thereof that comprises: (a) a variable heavy chain that comprises a polypeptide sequence having at least 90% sequence identity to an amino acid sequence of SEQ ID NO: 17760; (b) a variable light chain that comprises a polypeptide sequence having at least 90% sequence identity to an amino acid sequence of SEQ ID NO: 19010; or (c) the variable heavy chain of (a), and the variable light chain of (b).

In one aspect, provided herein is an antibody or antigen-binding fragment thereof that comprises: (i) a variable heavy chain complementarity-determining region CDR-H1, CDR-H2 and CDR-H3, wherein (a) the CDR-H1 comprises the amino acid sequence of SEQ ID NO: 10705, (b) the CDR-H2 comprises the amino acid sequence of SEQ ID NO: 13205, and (c) the CDR-H3 comprises the amino acid sequence of SEQ ID NO: 15705; (ii) a variable light chain complementarity-determining region CDR-L1, CDR-L2 and CDR-L3, wherein: (a) the CDR-L1 comprises the amino acid sequence of SEQ ID NO: 11955, (b) the CDR-L2 comprises the amino acid sequence of SEQ ID NO: 14455, and (c) the CDR-L3 comprises the amino acid sequence of SEQ ID NO: 16955; or (iii) the variable heavy chain complementarity-determining region CDR-H1, CDR-H2 and CDR-H3 of (i), and the variable light chain complementarity-determining region CDR-L1, CDR-L2 and CDR-L3 of (ii).

In one aspect, provided herein is an antibody or antigen binding fragment thereof that comprises: (a) a variable heavy chain that comprises a polypeptide sequence having at least 90% sequence identity to an amino acid sequence of SEQ ID NO: 18205; (b) a variable light chain that comprises a polypeptide sequence having at least 90% sequence identity to an amino acid sequence of SEQ ID NO: 19455; or (c) the variable heavy chain of (a), and the variable light chain of (b).

In one aspect, provided herein is an antibody or antigen-binding fragment thereof that comprises: (i) a variable heavy chain complementarity-determining region CDR-H1, CDR-H2 and CDR-H3, wherein (a) the CDR-H1 comprises the amino acid sequence of SEQ ID NO: 10484, (b) the CDR-H2 comprises the amino acid sequence of SEQ ID NO: 12984, and (c) the CDR-H3 comprises the amino acid sequence of SEQ ID NO: 15484; (ii) a variable light chain complementarity-determining region CDR-L1, CDR-L2 and CDR-L3, wherein: (a) the CDR-L1 comprises the amino acid sequence of SEQ ID NO: 11734, (b) the CDR-L2 comprises the amino acid sequence of SEQ ID NO: 14234, and (c) the CDR-L3 comprises the amino acid sequence of SEQ ID NO: 16734; or (iii) the variable heavy chain complementarity-determining region CDR-H1, CDR-H2 and CDR-H3 of (i), and the variable light chain complementarity-determining region CDR-L1, CDR-L2 and CDR-L3 of (ii).

In one aspect, provided herein is an antibody or antigen binding fragment thereof that comprises: (a) a variable heavy chain that comprises a polypeptide sequence having at least 90% sequence identity to an amino acid sequence of SEQ ID NO: 17984; (b) a variable light chain that comprises a polypeptide sequence having at least 90% sequence identity to an amino acid sequence of SEQ ID NO: 19234; or (c) the variable heavy chain of (a), and the variable light chain of (b).

In one aspect, provided herein is an antibody or antigen-binding fragment thereof that comprises: (i) a variable heavy chain complementarity-determining region CDR-H1, CDR-H2 and CDR-H3, wherein (a) the CDR-H1 comprises the amino acid sequence of SEQ ID NO: 10291, (b) the CDR-H2 comprises the amino acid sequence of SEQ ID NO: 12791, and (c) the CDR-H3 comprises the amino acid sequence of SEQ ID NO: 15291; (ii) a variable light chain complementarity-determining region CDR-L1, CDR-L2 and CDR-L3, wherein: (a) the CDR-L1 comprises the amino acid sequence of SEQ ID NO: 11541, (b) the CDR-L2 comprises the amino acid sequence of SEQ ID NO: 14041, and (c) the CDR-L3 comprises the amino acid sequence of SEQ ID NO: 16541; or (iii) the variable heavy chain complementarity-determining region CDR-H1, CDR-H2 and CDR-H3 of (i), and the variable light chain complementarity-determining region CDR-L1, CDR-L2 and CDR-L3 of (ii).

In one aspect, provided herein is an antibody or antigen binding fragment thereof that comprises: (a) a variable heavy chain that comprises a polypeptide sequence having at least 90% sequence identity to an amino acid sequence of SEQ ID NO: 17791; (b) a variable light chain that comprises a polypeptide sequence having at least 90% sequence identity to an amino acid sequence of SEQ ID NO: 19041; or (c) the variable heavy chain of (a), and the variable light chain of (b).

In one aspect, provided herein is an antibody or antigen-binding fragment thereof that comprises: (i) a variable heavy chain complementarity-determining region CDR-H1, CDR-H2 and CDR-H3, wherein (a) the CDR-H1 comprises the amino acid sequence of SEQ ID NO: 10114, (b) the CDR-H2 comprises the amino acid sequence of SEQ ID NO: 12614, and (c) the CDR-H3 comprises the amino acid sequence of SEQ ID NO: 15114; (ii) a variable light chain complementarity-determining region CDR-L1, CDR-L2 and CDR-L3, wherein: (a) the CDR-L1 comprises the amino acid sequence of SEQ ID NO: 11364, (b) the CDR-L2 comprises the amino acid sequence of SEQ ID NO: 13864, and (c) the CDR-L3 comprises the amino acid sequence of SEQ ID NO: 16364; or (iii) the variable heavy chain complementarity-determining region CDR-H1, CDR-H2 and CDR-H3 of (i), and the variable light chain complementarity-determining region CDR-L1, CDR-L2 and CDR-L3 of (ii).

In one aspect, provided herein is an antibody or antigen binding fragment thereof that comprises: (a) a variable heavy chain that comprises a polypeptide sequence having at least 90% sequence identity to an amino acid sequence of SEQ ID NO: 17614; (b) a variable light chain that comprises a polypeptide sequence having at least 90% sequence identity to an amino acid sequence of SEQ ID NO: 18864; or (c) the variable heavy chain of (a), and the variable light chain of (b).

In one aspect, provided herein is an antibody or antigen-binding fragment thereof that comprises: (i) a variable heavy chain complementarity-determining region CDR-H1, CDR-H2 and CDR-H3, wherein (a) the CDR-H1 comprises the amino acid sequence of SEQ ID NO: 10394, (b) the CDR-H2 comprises the amino acid sequence of SEQ ID NO: 12894, and (c) the CDR-H3 comprises the amino acid sequence of SEQ ID NO: 15394; (ii) a variable light chain complementarity-determining region CDR-L1, CDR-L2 and CDR-L3, wherein: (a) the CDR-L1 comprises the amino acid sequence of SEQ ID NO: 11644, (b) the CDR-L2 comprises the amino acid sequence of SEQ ID NO: 14144, and (c) the CDR-L3 comprises the amino acid sequence of SEQ ID NO: 16644; or (iii) the variable heavy chain complementarity-determining region CDR-H1, CDR-H2 and CDR-H3 of (i), and the variable light chain complementarity-determining region CDR-L1, CDR-L2 and CDR-L3 of (ii).

In one aspect, provided herein is an antibody or antigen binding fragment thereof that comprises: (a) a variable heavy chain that comprises a polypeptide sequence having at least 90% sequence identity to an amino acid sequence of SEQ ID NO: 17894; (b) a variable light chain that comprises a polypeptide sequence having at least 90% sequence identity to an amino acid sequence of SEQ ID NO: 19144; or (c) the variable heavy chain of (a), and the variable light chain of (b).

In some embodiments of the aspects described above, the antibody comprises an IgG, IgA, IgM, or IgE antibody. In some embodiments, the IgG comprises IgG1, IgG2, IgG3, IgG4, IgGA1, or IgGA2. In some embodiments, the antibody comprises a bispecific antibody, a multispecific antibody, a multivalent antibody, a chimeric antibody, a human antibody, humanized antibody, a monoclonal antibody, a deimmunized antibody, or a combination thereof. In some embodiments, the antigen-binding fragment comprises a Fab, Fab′, Fab′-SH, Fv, scFv, F(ab′)2, a diabody, a linear antibody, a single domain antibody (sdAb), a camelid V_{H}H domain, or a multi-specific antibody formed from antibody fragments.

In some embodiments, the antibody or antigen-binding fragment thereof is recombinant or synthetic. In some embodiments, the antibody or antigen-binding fragment thereof further comprise an enzyme, a substrate, cofactor, a fluorescent marker, a chemiluminescent marker, a peptide tag, a magnetic particle, a drug, a toxin, or a combination thereof. In some embodiments, the antibody or antigen-binding fragment thereof binds to a SARS-CoV-2. In some embodiments, the antibody or antigen binding fragment thereof binds a SARS-Cov-2 spike (S) protein, or a homolog thereof, or a variant thereof. In some embodiments, the antibody or antigen binding fragment thereof binds subunit 51, or a subunit S2 of the SARS-Cov-2 spike (S) protein. In some embodiments, the antibody or antigen binding fragment thereof binds a receptor binding domain of the subunit 51. In some embodiments, the antibody or antigen-binding fragment thereof inhibits infection from SARS-CoV-2. In some embodiments, the antibody or antigen-binding fragment thereof inhibits binding of a receptor binding domain of a subunit 51 of a SARS-CoV-2 with a receptor on a host cell. In some embodiments, the antibody or antigen-binding fragment thereof inhibits entry of a SARS-CoV-2 in a host cell. In some embodiments, the antibody or antigen-binding fragment is useful for treating COVID-19.

In one aspect, provided herein is a pharmaceutical composition or a medicament that comprises the antibody or antigen-binding fragment thereof of any one of aspects described above, and a pharmaceutically acceptable carrier, excipient or diluent.

In some embodiments, the pharmaceutical composition or medicament is formulated for administration via a subcutaneous, intravenous, intradermal, intraperitoneal, intramuscular, intracerebroventricular, intracranial, intracelial, or intracerebellar administration route. In some embodiments, the pharmaceutical composition or medicament is in an aqueous or in a lyophilized form. In some embodiments, the pharmaceutical composition or medicament is contained in a delivery device selected from the group consisting of a syringe, a blunt tip syringe, a catheter, and an implantable pump. In some embodiments, the pharmaceutical composition or medicament comprises an additional therapeutic agent. In some embodiments, the additional therapeutic agent is a nonsteroidal anti-inflammatory drug, a corticosteroid, a dietary supplement such as an antioxidant, a small molecule, a therapeutic vaccine, an immunomodulator, an angiotensin-converting enzyme [ACE] inhibitor, an angiotensin receptor blockers [ARBs], a HMG-CoA Reductase Inhibitors (Statins), an anti-viral agent, acetaminophen, or an additional anti-SARS-CoV-2 antibody.

In one aspect, provided herein is a method for preventing a SARS-CoV-2 infection or COVID-19 in a subject in need thereof, the method comprising administering to the subject a therapeutically effective amount of either the antibody or antigen binding fragment of any one of aspects described above, or the pharmaceutical composition of any one of aspects described above.

In one aspect, provided herein is a method for treating a SARS-CoV-2 infection or COVID-19 in a subject in need thereof, the method comprising administering to the subject, (a) the antibody or antigen-binding fragment thereof of any one of aspects described above; or (b) the pharmaceutical composition or medicament of any one of aspects described above.

In some embodiments, the antibody or antigen-binding fragment thereof binds to the SARS-CoV-2. In some embodiments, the antibody or antigen binding fragment thereof binds a SARS-Cov-2 spike (S) protein, or a homolog thereof, or a variant thereof. In some embodiments, the antibody or antigen binding fragment thereof binds subunit S1, or a subunit S2 of the SARS-Cov-2 spike (S) protein. In some embodiments, the antibody or antigen binding fragment thereof binds a receptor binding domain of the subunit S1. In some embodiments, the antibody or antigen-binding fragment thereof inhibits binding of a receptor binding domain of a subunit S1 of the SARS-CoV-2 with a receptor on a host cell. In some embodiments, the antibody or antigen-binding fragment thereof inhibits entry of the SARS-CoV-2 in a host cell. In some embodiments, the antibody or antigen-binding fragment thereof inhibits fusion of the SARS-CoV-2 membrane with a host cell membrane. In some embodiments, the antibody or antigen binding fragment thereof neutralizes the SARS-CoV-2.

In some embodiments, of the methods described herein the administering reduces one or more symptoms associated with a SARS-CoV-2 infection. In some embodiments, the administering reduces viral load in the subject. In some embodiments, the antibody or antigen binding fragment thereof is administered to the subject with an additional therapeutic agent. In some embodiments, the additional therapeutic agent is a nonsteroidal anti-inflammatory drug, a corticosteroid, a dietary supplement such as an antioxidant, a small molecule, a therapeutic vaccine, an immunomodulator, an angiotensin-converting enzyme [ACE] inhibitor, an angiotensin receptor blockers [ARBs], a HMG-CoA Reductase Inhibitors (Statins), an anti-viral agent, acetaminophen, or an additional anti-SARS-CoV-2 antibody.

Provided herein is a hybridoma that produces the antibody or antigen-binding fragment thereof of any one of aspects described above.

Provided herein is a fusion protein that comprises the antibody or antigen-binding fragment thereof of any one of aspects above.

Provided herein is an immunoconjugate comprising the antibody or the antigen binding fragment thereof of any one of aspects above, and a therapeutic agent.

In one aspect, provided herein is an isolated nucleic acid comprising at least one of: (a) a nucleic acid sequence encoding CDR-H1, wherein the nucleic acid sequence is selected from SEQ ID NOs: 1-1250; (b) a nucleic acid sequence encoding CDR-L1, wherein the nucleic acid sequence is selected from SEQ ID NOs: 1251-2500; (c) a nucleic acid sequence encoding CDR-H2, wherein the nucleic acid sequence is selected from SEQ ID NOs: 2501-3750; (d) a nucleic acid sequence encoding CDR-L2, wherein the nucleic acid sequence is selected from SEQ ID NOs: 3751-5000; (e) a nucleic acid sequence encoding CDR-H3, wherein the nucleic acid sequence is selected from SEQ ID NOs: 5001-6250; or (f) a nucleic acid sequence encoding CDR-L3, wherein the nucleic acid sequence is selected from SEQ ID NOs: 6251-7500.

In one aspect, provided herein is an isolated nucleic acid comprising at least one of: (a) a nucleic acid sequence encoding a heavy chain polypeptide of an antibody, wherein the nucleic acid sequence is selected from any one of SEQ ID NOs: 7501-8750, and (b) a nucleic acid sequence encoding a light chain polypeptide of an antibody, wherein the nucleic acid sequence is selected from any one of SEQ ID NOs: 8751-10000.

In one aspect, provided herein is an isolated nucleic acid that comprises a reconstructed nucleic acid consensus sequence encoding a heavy chain polypeptide of an antibody, wherein the nucleic acid consensus sequence is selected from any of SEQ ID NOS: 7501-8750.

In one aspect, provided herein is an isolated nucleic acid that comprises a reconstructed nucleic acid consensus sequence encoding a light chain polypeptide of an antibody, wherein the nucleic acid consensus sequence is selected from any of SEQ ID NOS: 8751-10000.

In one aspect, provided herein is an expression vector comprising the isolated nucleic acid molecule of any one of aspects above. In some embodiments, the isolated nucleic acid is operably linked to a regulatory control sequence.

Provided herein is a host cell comprising the expression vector of any one of aspects above, or the isolated nucleic acid of any one of aspects above. In some embodiments, said host cell is a mammalian cell, or a bacterial cell. In some embodiments, said bacterial cell is an Escherichia. coli cell. In some embodiments, the expression of the nucleic acid is under control of one or more inducible promoters.

In one aspect, provided herein is a method of diagnosing a subject as being infected with a SARS-Cov-2 virus or suspected of being infected with a SARS-Cov-2 virus, the method comprising contacting a sample obtained from the subject with the antibody or the antigen-binding fragment of any one of aspects above; detecting the presence or absence of the antibody or the antigen-binding fragment; and diagnosing the subject as being infected with a SARS-CoV-2 virus when the presence of the antibody or the antigen-binding fragment is detected. In some embodiments, the sample comprises a nasal swab, a tissue sample, saliva, or blood. In some embodiments, detecting the presence or absence of the antibody or the antigen-binding fragment comprises an enzyme linked immunosorbent assay (ELISA), an immunospot assay, a lateral flow assay, flow cytometry, immunohistochemistry, or a western blot.

In one aspect, provided herein is an immunohistochemical assay comprising; (a) contacting a sample obtained from a subject with the antibody or antigen binding fragment thereof of any one of aspects above under conditions permitting selective binding of the antibody or antigen binding fragment thereof with a SARS-CoV-2, to form an antibody-antigen complex; and (b) detecting the presence or absence of the antibody-antigen complex by an immunodetection method. In some embodiments, the sample is a nasal swab, a tissue sample, saliva, or blood. In some embodiments, the sample is obtained from a subject suspected to be suffering from a SARS-CoV-2 infection or COVID-19.

Provided herein is a method of inhibiting binding of a SARS-CoV-2 with a host cell, or inhibiting entry of a SARS-CoV2 in a host cell, the method comprising contacting the SARS-CoV-2 with the antibody or antigen binding fragment thereof of any one of aspects described above. In some embodiments, the antibody or antigen binding fragment thereof binds a SARS-Cov-2 spike (S) protein, or a homolog thereof, or a variant thereof. In some embodiments, the antibody or antigen binding fragment thereof binds subunit S1, or a subunit S2 of the SARS-Cov-2 spike (S) protein. In some embodiments, the antibody or antigen binding fragment thereof binds a receptor binding domain of the subunit S1. In some embodiments, the antibody or antigen-binding fragment thereof inhibits binding of a receptor binding domain of a subunit S1 of the SARS-CoV-2 with a receptor on the host cell. In some embodiments, the antibody or antigen-binding fragment thereof inhibits fusion of the SARS-CoV-2 membrane with the host cell membrane. In some embodiments, the antibody or antigen binding fragment thereof neutralizes the SARS-CoV-2.

Provided herein is a method of producing an antibody or an antigen binding fragment thereof, the method comprising: (a) culturing the host cell of any one of aspects above, in a medium under conditions permitting expression of a polypeptide encoded by the isolated nucleic acid, and assembling of the antibody or an antigen binding fragment thereof; and (b) purifying the antibody or antigen binding fragment thereof from the cultured cell or the cell culturing medium.

In one aspect, provided herein is an antibody or antigen-binding fragment thereof comprising at least one of: (a) a variable heavy chain complementarity-determining region CDR-H1, CDR-H2 and CDR-H3, wherein: (i) CDR-H1 comprises a polypeptide sequence selected from any one of SEQ ID NOs: 10001-11250, (ii) CDR-H2 comprises a polypeptide sequence selected from any one of SEQ ID NOs: 12501-13750, and (iii) CDR-H3 comprises a polypeptide sequence selected from any one of SEQ ID NOs: 15001-16250; and (b) a variable light chain complementarity-determining region CDR-L1, CDR-L2 and CDR-L3, wherein: (i) CDR-L1 comprises a polypeptide sequence selected from any one of SEQ ID NOs: 11251-12500, (ii) CDR-L2 comprises a polypeptide sequence selected from any one of SEQ ID NOs: 13751-15000, and (iii) CDR-L3 comprises a polypeptide sequence selected from any one of SEQ ID NOs: 16251-17500.

In one aspect, provided herein is an antibody or antigen-binding fragment thereof comprising at least one of: (a) a variable heavy chain, wherein the variable heavy chain comprises a polypeptide sequence having at least 90% sequence identity to an amino acid sequence selected from any one of SEQ ID NOs: 17501-18750; and (b) a variable light chain, wherein the variable light chain comprises a polypeptide sequence having at least 90% sequence identity to an amino acid sequence selected from any one of SEQ ID NOs: 18751-20000.

In some embodiments, the antibody comprises an IgG, IgA, IgM, or IgE antibody. In some embodiments, the IgG comprises IgG1, IgG2, IgG3, IgG4, IgGA1, or IgGA2. In some embodiments, the antibody comprises a bispecific antibody, a multispecific antibody, a multivalent antibody, a chimeric antibody, a human antibody, humanized antibody, a monoclonal antibody, a deimmunized antibody, or a combination thereof. In some embodiments, the antigen-binding fragment comprises a Fab, Fab′, Fab′-SH, Fv, scFv, F(ab′)2, a diabody, a linear antibody, a single domain antibody (sdAb), a camelid V_{H}H domain, or a multi-specific antibody formed from antibody fragments. In some embodiments, the antibody or antigen-binding fragment thereof is recombinant or synthetic. In some embodiments, the antibody or antigen-binding fragment binds SARS-CoV-2, the virus that causes COVID-19.

Provided herein is a hybridoma that produces the antibody or antigen-binding fragment thereof of any one of aspects described above.

Provided herein is a pharmaceutical composition or a medicament that comprises the antibody or antigen-binding fragment thereof of any one of aspects above, and a pharmaceutically acceptable carrier, excipient or diluent. In some embodiments, the pharmaceutical composition or medicament is formulated for administration via a subcutaneous, intravenous, intradermal, intraperitoneal, intramuscular, intracerebroventricular, intracranial, intracelial, or intracerebellar administration route. In some embodiments, the pharmaceutical composition or medicament is in an aqueous or in a lyophilized form. In some embodiments, the pharmaceutical composition or medicament is contained in a delivery device selected from the group consisting of a syringe, a blunt tip syringe, a catheter, and an implantable pump.

In one aspect, provided herein is a method for treating or preventing a SARS-CoV2 infection or a COVID-19 in a subject tin need thereof, the method comprising administering to the subject the antibody or antigen-binding fragment thereof of any one of aspects above, or the pharmaceutical composition or medicament of any one of aspects above.

Provided herein is use of the antibody or antigen binding fragment of any one of aspects above for treatment or prevention of a SARS-CoV-2 infection or COVID-19.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a dose response curve for antibody of the disclosure TOTCOVID00425 generated from a bioluminescence assay testing neutralization activity against SARS-CoV-2, including a calculated half maximal inhibitory concentration (IC₅₀) value.

FIG. 2 depicts a dose response curve for positive control REGN-10933, including a calculated IC₅₀ value.

FIGS. 3A-3B depict dose response curves for antibody of the disclosure TOTCOVID00425 across two independent assays, including calculated IC₅₀ values.

FIGS. 4A-4B depict dose response curves for antibody of the disclosure TOTCOVID00316 across two independent assays, including calculated IC₅₀ values.

FIGS. 5A-5C depict dose response curves for antibody of the disclosure TOTCOVID00761 across two independent assays and using an additional replicate sample, including calculated IC₅₀ values.

FIGS. 6A-6C depict does response curves for antibody of the disclosure TOTCOVID00540 across two independent assays and using an additional replicate sample, including calculated IC₅₀ values.

FIGS. 7A-7C depict dose response curves for antibody of the disclosure TOTCOVID00347 across two independent assays and using an additional replicate sample, including calculated IC₅₀ values.

FIG. 8 depicts a dose response curve for antibody of the disclosure TOTCOVID00124, including a calculated IC₅₀ value.

### DETAILED DESCRIPTION OF THE INVENTION

It is to be understood that this application is not limited to particular formulations or process parameters, as these may, of course, vary. It is also to be understood that the terminology used herein is for the purpose of describing particular embodiments only, and is not intended to be limiting. Further, it is understood that a number of methods and materials similar or equivalent to those described herein can be used in the practice of the present inventions.

In accordance with the present application, there may be employed conventional molecular biology, microbiology, and recombinant DNA techniques as explained fully in the art. The definitions contained herein supplement those in the art and are directed to the current application and are not to be imputed to any related or unrelated case, e.g., to any commonly owned patent or application. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise. Furthermore, use of the term “including” as well as other forms, such as “include”, “includes,” and “included,” is not limiting.

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “or” can be used conjunctively or disjunctively, unless the context specifically refers to a disjunctive use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the invention, and vice versa. Furthermore, compositions of the invention can be used to achieve methods of the invention.

As used herein the term “consisting essentially of” refers to those elements required for a given embodiment. The term permits the presence of elements that do not materially affect the basic and novel or functional characteristic(s) of that embodiment of the invention.

As used herein the term “consisting of” refers to compositions, methods, and respective components thereof as described herein, which are exclusive of any element not recited in that description of the embodiment.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the inventions.

### SARS-CoV-2

Provided herein are antibodies and antigen binding fragment thereof that bind a SARS-CoV-2, and are useful in treatment, and prevention of a SARS-CoV-2 infection and COVID-19. The antibodies of the present disclosure can be useful for detection of SARS-CoV-2 virus, and diagnosis of a SARS-CoV-2 infection and COVID-19. As used herein, the term “severe acute respiratory syndrome coronavirus 2 (SARS-CoV-2)” (also referred to as a “Wuhan coronavirus,” a “2019 novel coronavirus,” or a “2019-nCoV”) refers to a newly emergent coronavirus that was initially identified from the Chinese city Wuhan in December 2019. SARS-Cov-2 belongs to the broad family of viruses known as coronaviruses. It is a positive-sense single stranded RNA virus, with a single linear RNA segment. The genomes of multiple strains of SARS-CoV-2 have been sequenced, and the homology between the nucleic acid sequences of these strains has been measured at typically greater than 99.99% (see, e.g., Wang, C. et al. J. Med. Virol. 92(6):667-674 (2020), hereby incorporated by reference). Thus, “SARS-CoV-2” means any virus with a high level of nucleic acid or amino acid sequence homology: e.g., having at least 90% sequence identity with a reference nucleic acid sequence of a viral genome identified as SARS-CoV-2 in a commonly used genomic research database, such as those maintained by the National Center for Biotechnology Information or GISAID. Non-limiting exemplary reference nucleic acid sequence of the SARS-Cov-2 genome is available at RefSeq reference number: NC 045512.2, which is incorporated herein in its entirety. The term “SARS-CoV-2” also includes any known variant thereof, for example, including but not limited to the Alpha (B.1.1.7) variant, Beta (B.1.351) variant, Gamma (P.1) variant, Delta (B.1.617.2) variant, Epsilon (B.1.429/B.1.427/CAL.20C) variant, Iota (B.1.526) variant, Eta (B.1.525) variant, Kappa (B.1.617.1) variant, or Lambda (C.37) variant, Zeta (P.2) variant, and Theta (P.3) variant. The term “SARS-CoV-2” also includes a variant comprising at least 1, 2, 3, 4, 5, 6, 7, 8, 9, 10 or more mutations relative to a reference nucleic acid sequence. In some embodiments, a mutation is in the nucleic acid sequence that encodes subunit S1 relative to that of the reference nucleic acid sequence.

As used herein, the term “coronavirus disease of 2019” or “COVID-19” refers to the disease caused by SARS-CoV-2. A patient suffering from COVID-19 would generally display clinical symptoms associated with this disease (see, e.g., Guan, W. et al. N Engl. J. Med. 382(18):1708-1720 (2020)) and would also test positive for presence of the SARS-CoV-2 viral genome using a real-time RT-PCR diagnostic assay (see, e.g., Pefiarrubia, L. et al. Int. J. Infect. Dis. 97:225-229 (2020)).

### Reconstructed Nucleic Acid Sequences

Provided herein are reconstructed nucleic acid and polypeptide consensus sequences for SARS-CoV-2 associated antibodies. The consensus sequences were reconstructed in silico from RNA-Seq data. Non-limiting examples of computational tools known in the art for reconstructing full-length antibody repertoires including MIGEC (Shugay, M. et al., Nat. Methods 11(6):653-655 (2014)), pRESTO (Vander Heiden, J. A. et al., Bioinformatics 30(13):1930-1932 (2014)), MiXCR (Bolotin, D. A. et al., Nat. Methods 12(5):380-381 (2015)), and IgRepertoireConstructor (Safonova, Y. et al., Bioinformatics 31(12):i53-i61 (2015)). Further examples are provided in the Examples section below.

The term, “nucleic acid consensus sequence” as used herein refers to a nucleic acid sequence, which comprises the most frequently occurring nucleotide residues at each location in all immunoglobulin nucleic acid sequence of any particular subclass or subunit structure. The nucleic acid consensus sequence may be based on immunoglobulins of a particular species or of many species. A nucleic acid “consensus” sequence, or “consensus” structure, is understood to encompass a human nucleic acid consensus sequence as described in certain embodiments of this invention, and to refer to a nucleic acid sequence which comprises the most frequently occurring nucleotide residues at each location in all human immunoglobulins nucleic acid of any particular subclass or subunit structure.

The term “polypeptide consensus sequence” as used herein refers to an amino acid sequence which comprises the most frequently occurring amino acid residues at each location in all immunoglobulins of any particular subclass or subunit structure. The polypeptide consensus sequence may be based on immunoglobulins of a particular species or of many species. A polypeptide “consensus” sequence, “consensus” structure, or “consensus” antibody is understood to encompass a human polypeptide consensus sequence as described in certain embodiments provided herein, and to refer to an amino acid sequence which comprises the most frequently occurring amino acid residues at each location in all human immunoglobulins of any particular subclass or subunit structure. The embodiments herein provide consensus human structures and consensus structures, which consider other species in addition to human.

As used herein, the terms “protein”, “peptide” and “polypeptide” are used interchangeably to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof. The term “fusion protein” as used herein refers to a polypeptide that comprises an amino acid sequence of an antibody or fragment thereof and an amino acid sequence of a heterologous polypeptide (i.e., an unrelated polypeptide).

As used herein, an “isolated” nucleic acid molecule or “isolated” nucleic acid sequence is a nucleic acid molecule that is either: (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined, is considered isolated. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The terms “synthetic polynucleotide,” “synthetic gene” or “synthetic polypeptide,” as used herein, mean that the corresponding polynucleotide sequence or portion thereof, or amino acid sequence or portion thereof, is derived, from a sequence that has been designed, or synthesized de novo, or modified, compared to an equivalent naturally-occurring sequence. Synthetic polynucleotides (antibodies or antigen-binding fragments) or synthetic genes can be prepared by methods known in the art, including but not limited to, the chemical synthesis of nucleic acid or amino acid sequences. Synthetic genes are typically different from naturally occurring genes, either at the amino acid, or polynucleotide level, (or both) and are typically located within the context of synthetic expression control sequences. Synthetic gene polynucleotide sequences, may not necessarily encode proteins with different amino acids, compared to the natural gene; for example, they can also encompass synthetic polynucleotide sequences that incorporate different codons but which encode the same amino acid (i.e., the nucleotide changes represent silent mutations at the amino acid level).

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR®) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Nucleic Acid Sequences

One aspect of the present disclosure pertains to reconstructed nucleic acid consensus sequences that encode an antibody polypeptide, described herein or antigen-binding fragment thereof. In some embodiments, the nucleic acid sequence encoding a heavy chain polypeptide is selected from any one of SEQ ID NOs: 7501-8750. In some embodiments, the nucleic acid sequence encoding a light chain polypeptide is selected from any one of SEQ ID NOs: 8751-10000. In some embodiments, the reconstructed nucleic acid sequence encodes a CDR1, CDR2, or CDR3 polypeptide of a variable heavy chain, such that:
(a) the nucleic acid sequence encoding the CDR1 polypeptide of a variable heavy chain is selected from any one of SEQ ID NOS: 1-1250,
(b) the nucleic acid sequence encoding the CDR2 polypeptide of a variable heavy chain is selected from any one of SEQ ID NOS: 2501-3750, or
(c) the nucleic acid sequence encoding the CDR3 polypeptide of a variable heavy chain is selected from any one of SEQ ID NOS: 5001-6250.
   In some embodiments, the reconstructed nucleic acid sequence encodes a CDR1, CDR2, or CDR3 polypeptide of a variable light chain, such that:
(a) the nucleic acid sequence encoding the CDR1 region of a variable light chain polypeptide is selected from any one of SEQ ID NOS: 1251-2500,
(b) the nucleic acid sequence encoding the CDR2 region of a variable light chain polypeptide is selected from any one of SEQ ID NOS: 3751-5000, or
(c) the nucleic acid sequence encoding the CDR3 region of a variable light chain polypeptide is selected from any one of SEQ ID NOS: 6251-7500.

### Antibody Synthesis and Purification

Starting from in silico reconstructed nucleic acid consensus sequences, antibody polypeptides may be synthesized and purified using conventional procedures. In one embodiment, an artificial gene construct encoding an antibody or antibody fragment thereof is synthesized (see, e.g., Khorana, H. G. et al., J. Mol. Biol. 72(2):209-217 (1972); Itakura, K. et al., Science 198(4321):1056-1063 (1977); and Edge, M. D. et al. Nature 292(5825):756-762 (1981)). The DNA template for the synthetic gene construct may then be cloned into a suitable expression vector and operably linked to a regulatory control sequence, transformed into an appropriate host for amplification, and the resulting amplified quantities of expression vector purified and transfected into an appropriate host for transient expression of the final resulting polypeptide encoding an antibody or antibody fragment thereof (see, e.g., Vazquez-Lombardi, R. et al., Nat. Protoc. 13(1):99-117 (2018)).

Using the information provided herein, for example, the reconstructed nucleic acid and amino acid sequences of the antibodies; a nucleic acid encoding the antibodies or antigen-binding fragment thereof can be obtained. Such a nucleic acid can be obtained, for example, using conventional methods disclosed in the art. Nucleic acids of the present disclosure may be in the form of RNA, such as mRNA, hnRNA, tRNA or any other form, or in the form of DNA, including but not limited to, cDNA and genomic DNA obtained by cloning or produced synthetically, or any combinations thereof. The DNA may be triplex, duplex or single-stranded, or any combination thereof. Any portion of at least one strand of the DNA or RNA may be the coding strand, also known as the sense strand, or it can be the antisense strand, also known as the antisense strand.

“Polynucleotide,” or “nucleic acid as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A nucleic acid can comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including isolated nucleic acid, RNA and DNA.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine. In some embodiments, the nucleic acid molecule comprises an isolated nucleic acid.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including, but not limited to alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid according to at least some embodiments of the disclosure can be, for example, DNA or RNA and may or may not contain intronic sequences. In a preferred embodiment, the nucleic acid is a cDNA molecule.

Another aspect of the present disclosure pertains to nucleic acid comprising reconstructed consensus nucleic acid sequences that encode the antibody polypeptide, described herein or antigen-binding fragment thereof. In some embodiments, the isolated nucleic acid comprises a nucleic acid sequence encoding a heavy chain polypeptide of an antibody. In some embodiments, the nucleic acid sequence encoding a heavy chain polypeptide is selected from SEQ ID NOs: 7501-8750. In some embodiments, the isolated nucleic acid comprises a nucleic acid sequence encoding a light chain polypeptide of an antibody. In some embodiments, the nucleic acid sequence encoding a light chain polypeptide is selected from SEQ ID NOs: 8751-10000.

In some embodiments, the isolated nucleic acid comprises a nucleic acid sequence encoding a CDR1 polypeptide of a variable heavy chain. In some embodiments, the isolated nucleic acid molecule comprises a nucleic acid sequence encoding a CDR2 polypeptide of a variable heavy chain. In some embodiments, the isolated nucleic molecule comprises a nucleic acid sequence encoding a CDR3 polypeptide of a variable heavy chain. In some embodiments, the nucleic acid sequence encoding the CDR1 polypeptide of a variable heavy chain (CDR-H1) comprises a sequence selected from SEQ ID NOS: 1-1250. In some embodiments, the nucleic acid sequence encoding the CDR2 polypeptide of a variable heavy chain (CDR-H2) comprises a sequence selected from SEQ ID NOS: 2501-3750. In some embodiments, the nucleic acid sequence encoding the CDR3 polypeptide of a variable heavy chain (CDR-H3) comprises a sequence selected from SEQ ID NOS: 5001-6250. In some embodiments, the isolated nucleic acid comprises a nucleic acid sequence encoding a CDR1 polypeptide of a variable light chain. In some embodiments, the isolated nucleic acid molecule comprises a nucleic acid sequence encoding a CDR2 polypeptide of a variable light chain. In some embodiments, the isolated nucleic acid comprises a nucleic acid sequence encoding a CDR3 polypeptide of a variable light chain. In some, embodiments, the nucleic acid sequence encoding the CDR1 region of a variable light chain polypeptide (CDR-L1) comprises a sequence selected from SEQ ID NOS: 1251-2500. In some embodiments, the nucleic acid sequence encoding the CDR2 region of a variable light chain polypeptide (CDR-L2) comprises a sequence selected from SEQ ID NOS: 3751-5000. In some, embodiments, the nucleic acid sequence encoding the CDR3 region of a variable light chain polypeptide (CDR-L3) comprises a sequence selected from SEQ ID NOS: 6251-7500. Nucleic acids according to at least some embodiments of the present disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library. Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame. The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region. To create a scFv gene, the VH- and VL-encoding DNA fragments are operatively linked to another fragment encoding a flexible linker, e.g., encoding the amino acid sequence (Gly-4-Ser)3, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

Nucleic acids comprising a nucleotide sequence substantially different from those described above but which, due to the degeneracy of the genetic code, still encode at least antibody or antigen binding fragment thereof as described herein and/or as it is known in the art are also contemplated. Of course, the genetic code is well known in the art. Therefore, it would be routine for one skilled in the art to generate such degenerate nucleic acid variants encoding specific antibodies of the present disclosure. See for example, Ausubel et al., Supra, and such nucleic acid variants are included in the present invention.

In some embodiments, the nucleic acid is one that encodes for any of the amino acid sequences for the antibodies in the Table 1 herein. In some embodiments, the nucleic acid sequence is one that is at least 80% identical to a nucleic acid encoding any of the amino acid sequences for the antibodies in the in the Table 1 herein, for example, at least 80, 85, 90, 91, 92, 93, 94, 95, 96, 97, 98, or 99% identical. In some embodiments, the nucleic acid is one that hybridizes to any one or more of the nucleic acid sequences provided herein. In some of the embodiments, the hybridization is under moderate conditions. In some embodiments, the hybridization is under highly stringent conditions, such as: at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.

Nucleic acids can be constructed using recombinant DNA techniques conventional in the art. In some embodiments, a nucleic acid disclosed herein is placed in an expression vector that is suitable for expression in a selected host cell. Vectors comprising nucleic acids that encode the antibodies or antigen binding fragment herein are provided. Vectors comprising nucleic acids that encode a heavy chains and/or a light chains are also provided. Such vectors include, but are not limited to, DNA vectors, phage vectors, viral vectors, retroviral vectors, etc. In one embodiment, the nucleic acid coding for the light chain and that coding for the heavy chain are isolated separately by the procedures outlined above. In one embodiment, the isolated nucleic acid encoding the light chain and that coding for the heavy chain may be inserted into separate expression plasmids, or together in the same plasmid, so long as each is under suitable promoter and translation control. In some embodiments, the suitable promoter is an inducible promoter. In some embodiments a suitable promoter is a constitutive promoter. In some embodiments, the heavy chain and light chain are expressed as part of a single polypeptide, such as, for example, when the antibody is an scFv.

In some embodiments, a first vector comprises a nucleic acid that encodes a heavy chain and a second vector comprises a nucleic acid that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used. In some embodiments, a vector is selected that is optimized for expression of polypeptides in CHO or CHO-derived cells, or in NSO cells. Exemplary such vectors are described, for example, in Running Deer et al., Biotechnol. Prog. 20:880-889 (2004).

In one aspect, the present disclosure provides methods for treatment or prevention of cancer comprising administering a nucleic acid, wherein the nucleic acid encode for a VH, VL, CDR3 region of VH or CDR 3 region of VL or antigen binding fragment thereof, wherein the nucleic acid comprises a sequence disclosed herein by way of gene therapy. Gene therapy refers to therapy performed by the administration to a subject of an expressed or expressible nucleic acid. In this embodiment of the disclosure, the nucleic acids produce their encoded protein that mediates a prophylactic or therapeutic effect. Any of the methods for gene therapy available in the art can be used according to the embodiments herein.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215 Methods. commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY. Delivery of a therapeutic antibody to appropriate cells can be effected via gene therapy ex vivo, in situ, or in vivo by use of any suitable approach known in the art, including by use of physical DNA transfer methods (e.g., liposomes or chemical treatments) or by use of viral vectors (e.g., adenovirus, adeno-associated virus, or a retrovirus). For example, for in vivo therapy, a nucleic acid encoding the desired antibody, either alone or in conjunction with a vector, liposome, or precipitate may be injected directly into the subject, and in some embodiments, may be injected at the site where the expression of the antibody compound is desired. For ex vivo treatment, the subject's cells are removed, the nucleic acid is introduced into these cells, and the modified cells are returned to the subject either directly or, for example, encapsulated within porous membranes which are implanted into the patient. See, e.g. U.S. Pat. Nos. 4,892,538 and 5,283,187. There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, and calcium phosphate precipitation. A commonly used vector for ex vivo delivery of a nucleic acid is a retrovirus.

The term “host cell” as used herein refers to the particular subject cell, for example cell of a subject suffering from an infection of a SARS-CoV-2 or COVID-19, or at a risk of, or suspected of suffering from an infection of a SARS-CoV-2 or COVID-19. In some embodiments, the host cell can be transfected with a nucleic acid disclosed herein. In some embodiments, the host cell is in the subject. In some embodiments, the host cell is an ex vivo cell obtained from the subject.

Other in vivo nucleic acid transfer techniques include transfection with viral vectors (such as adenovirus, Herpes simplex I virus, or adeno-associated virus) and lipid-based systems. The nucleic acid and transfection agent are optionally associated with a microparticle. Exemplary transfection agents include calcium phosphate or calcium chloride co-precipitation, DEAE-dextran-mediated transfection, quaternary ammonium amphiphile DOTMA ((dioleoyloxypropyl) trimethylammonium bromide, commercialized as Lipofectin by GIBCO-BRL))(Felgner et al, (1987) Proc. Natl. Acad. Sci. USA 84, 7413-7417; Malone et al. (1989) Proc. Natl Acad. Sci. USA 86 6077-6081); lipophilic glutamate diesters with pendent trimethylammonium heads (Ito et al. (1990) Biochem. Biophys. Acta 1023, 124-132); the metabolizable parent lipids such as the cationic lipid dioctadecylamido glycylspermine (DOGS, Transfectam, Promega) and dipalmitoylphosphatidyl ethanolamylspermine (DPPES)(J. P. Behr (1986) Tetrahedron Lett. 27, 5861-5864; J. P. Behr et al. (1989) Proc. Natl. Acad. Sci. USA 86, 6982-6986); metabolizable quaternary ammonium salts (DOTB, N-(1-[2,3-dioleoyloxylpropyl)-N,N,N-trimethylammonium methylsulfate (DOTAP)(Boehringer Mannheim), polyethyleneimine (PEI), dioleoyl esters, ChoTB, ChoSC, DOSC)(Leventis et al. (1990) Biochim. Inter. 22, 235-241); 3be ta[N—(N′,N′-dimethylaminoethane)-carbamoyl]cholesterol (DC-Chol), dioleoylphosphatidyl ethanolamine (DOPE)/3beta[N—(N′,N′ dimethylaminoethane)-carbamoyl] cholesterolDC-Chol in one to one mixtures (Gao et al., (1991) Biochim. Biophys. Acta 1065, 8-14), spermine, spermidine, lipopolyamines (Behr et al., Bioconjugate Chem, 1994, 5: 382-389), lipophilic polylysines (LPLL) (Zhou et al., (1991) Biochim. Biophys. Acta 939, 8-18), [[(1,1,3,3 tetramethylbutyl)cresoxy]ethoxylethylldimethylbnzylammonium hydroxide (DEBDA hydroxide) with excess phosphatidylcholine/cholesterol (Ballas et al., (1988) Biochim. Biophys. Acta 939, 8-18), cetyltrimethylammonium bromide (CTAB)/DOPE mixtures (Pinnaduwage et al, (1989) Biochim. Biophys. Acta 985, 33-37), lipophilic diester of glutamic acid (TMAG) with DOPE, CTAB, DEBDA, didodecylammonium bromide (DDAB), and stearylamine in admixture with phosphatidylethanolamine (Rose et al., (1991) Biotechnique 10, 520-525), DDAB/DOPE (TransfectACE, GIBCO BRL), and oligogalactose bearing lipids. Exemplary transfection enhancer agents that increase the efficiency of transfer include, for example, DEAE-dextran, polybrene, lysosome-disruptive peptide (Ohmori N I et al, Biochem Biophys Res Commun Jun. 27, 1997; 235(3):726-9), chondroitan-based proteoglycans, sulfated proteoglycans, polyethylenimine, polylysine (Pollard H et al. J Biol Chem, 1998 273 (13):7507-11), integrin-binding peptide CYGGRGDTP, linear dextran nonasaccharide, glycerol, cholesteryl groups tethered at the 3′-terminal internucleoside link of an oligonucleotide (Letsinger, R. L. 1989 Proc Natl Acad Sci USA 86: (17):6553-6), lysophosphatide, lysophosphatidylcholine, lysophosphatidylethanolamine, and 1-oleoyl lysophosphatidylcholine.

In some situations, it may be desirable to deliver the nucleic acid with an agent that directs the nucleic acid containing vector to host cells. Such “targeting” molecules include antibodies specific for a cell-surface membrane protein on the target cell, or a ligand for a receptor on the target cell. Where liposomes are employed, proteins which bind to a cell-surface membrane protein associated with endocytosis may be used for targeting and/or to facilitate uptake. Examples of such proteins include capsid proteins and fragments thereof tropic for a particular cell type, antibodies for proteins which undergo internalization in cycling, and proteins that target intracellular localization and enhance intracellular half-life. In other embodiments, receptor-mediated endocytosis can be used. Such methods are described, for example, in Wu et al., 1987 or Wagner et al., 1990. For review of the currently known gene marking and gene therapy protocols, see Anderson 1992. See also WO 93/25673 and the references cited therein.

### Artificial Gene Synthesis

A variety of standard recombinant DNA techniques may be used for manipulating domains or functional segments within an antibody nucleic acid sequence. Once DNA fragments encoding V_{H} and V_{L} segments are obtained, these DNA fragments can be further manipulated, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame. The isolated DNA encoding the V_{H} region can be converted to a full-length heavy chain gene by operatively linking the V_{H}-encoding DNA to another DNA molecule encoding heavy chain constant regions (C_{H}1, C_{H}2 and C_{H}3). The sequences of human heavy chain constant region genes are known in the art (see e.g., Kabat, E. A. et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H}1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat, E. A. et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the V_{H}- and V_{L}-encoding DNA fragments are operatively linked to another fragment encoding a flexible linker, e.g., encoding the amino acid sequence (Gly-Gly-Gly-Gly-Ser)3, such that the V_{H} and V_{L} sequences can be expressed as a contiguous single-chain protein, with the V_{L} and V_{H} regions joined by the flexible linker (see, e.g., Bird et al. (1988) Science 242:423-426; Huston et al. (1988) Proc. Natl. Acad. Sci. USA 85:5879-5883; McCafferty et al., (1990) Nature 348:552-554).

### Selection and Transformation of Host Cells

In one aspect, provided herein is a host cell that comprises the isolated nucleic acids described above or a vector comprising said isolated nucleic acids described above. The vector can be a cloning vector or an expression vector. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescens, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1 776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NP\7, and such viruses may be used as the virus herein according to the present invention, particularly for transfection of Spodoptera frugiperda cells.

Plant cell cultures of cotton, corn, potato, soybean, petunia, tomato, tobacco, lemna, and other plant cells can also be utilized as hosts. However, interest has been greatest in vertebrate cells, and propagation of vertebrate cells in culture (tissue culture) has become routine procedure. Examples of useful mammalian host cell lines are Chinese hamster ovary cells, including CHOK1 cells (ATCC CCL61), DXB-11, DG-44, and Chinese hamster ovary cells/-DHFR (CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Viral. 36: 59 (1977)]; baby hamster kidney cells (BHK, ATCC CCL 10); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals NY Acad. Sci. 383: 44-68 (1982)); MRC 5 cells; FS4 cells; and a human hepatoma line (Hep G2).

Host cells are transformed or transfected with the above-described expression or cloning vectors for antibody production and cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful and preferred for the expression of antibodies, described herein.

For transfection of the expression vectors and production of the chimeric, humanized, or composite human antibodies described herein, the recipient cell line can be a myeloma cell. Myeloma cells can synthesize, assemble and secrete immunoglobulins encoded by transfected immunoglobulin nucleic acid sequences and possess the mechanism for glycosylation of the immunoglobulin. For example, in some embodiments, the recipient cell is the recombinant Ig-producing myeloma cell SP2/0 (ATCC #CRL 8287). SP2/0 cells produce only immunoglobulin encoded by the transfected genes. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells. An expression vector carrying a chimeric, humanized, or composite human antibody construct or antibody polypeptide described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et al., 240 Science 1538 (1988), as known to one of ordinary skill in the art.

Yeast provides certain advantages over bacteria for the production of immunoglobulin H and L chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes peptides bearing leader sequences (i.e., pre-peptides). Hitzman et al., 11th Intl. Conf. Yeast, Genetics & Molec. Biol. (Montpelier, France, 1982). Yeast gene expression systems can be routinely evaluated for the levels of production, secretion and the stability of antibody polypeptide or antigen-binding fragment peptide thereof, and assembled chimeric, humanized, or composite human antibodies, fragments and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. A number of approaches can be taken for evaluating optimal expression plasmids for the expression of cloned immunoglobulin cDNAs in yeast.

Bacterial strains can also be utilized as hosts for the production of the antibody molecules or fragments thereof described herein, E. coli K12 strains such as E. coli W3110 (ATCC 27325), Bacillus species, enterobacteria such as Salmonella typhimurium or Serratia marcescens, and various Pseudomonas species can be used. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of chimeric, humanized, or composite humanized antibodies and fragments thereof encoded by the cloned immunoglobulin cDNAs or CDRs in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin protein molecules including leader peptide removal, folding and assembly of H and L chains, glycosylation of the antibody molecules, and secretion of functional antibody protein. Mammalian cells which can be useful as hosts for the production of antibody proteins, in addition to the cells of lymphoid origin described above, include cells of fibroblast origin, such as Vero (ATCC CRL 81) or CHO-K1 (ATCC CRL 61) cells. Exemplary eukaryotic cells that can be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, a particular eukaryotic host cell is selected based on its ability to make desired post-translational modifications to the variable heavy chains and/or variable light chains. For example, in some embodiments, CHO cells produce polypeptides that have a higher level of sialylation than the same polypeptide produced in 293 cells.

In some embodiments, polypeptides of the antibodies or antigen-binding fragment thereof, disclosed herein can be produced in vivo in an animal that has been engineered or transfected with one or more nucleic acid molecules encoding the polypeptides, according to any suitable method.

In some embodiments, an antibody or antigen-binding fragment thereof is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695-713 (2003).

Many vector systems are available for the expression of H and L chain nucleic acid sequence in mammalian cells (see Glover, 1985). Different approaches can be followed to obtain complete H2L2 antibodies. As discussed above, it is possible to co-express H and L chains in the same cells to achieve intracellular association and linkage of H and L chains into complete tetrameric H2L2 antibodies and/or antigen-binding fragment peptides. The co-expression can occur by using either the same or different plasmids in the same host. Genes for both H and L chains and/or CDR3 regions peptides can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. Cell lines producing antigen-binding peptide fragments and/or H₂L₂ molecules via either route could be transfected with plasmids encoding additional copies of peptides, H, L, or H plus L chains in conjunction with additional selectable markers to generate cell lines with enhanced properties, such as higher production of assembled H2L2 antibody molecules or enhanced stability of the transfected cell lines.

Additionally, plants have emerged as a convenient, safe and economical alternative main-stream expression systems for recombinant antibody production, which are based on large scale culture of microbes or animal cells. Antibodies can be expressed in plant cell culture, or plants grown conventionally. The expression in plants may be systemic, limited to sub-cellular plastids, or limited to seeds (endosperms). Several plant-derived antibodies have reached advanced stages of development (see, e.g., Biolex, NC).

In some aspects, provided herein are methods and systems for the production of a humanized antibody, which is prepared by a process which comprises maintaining a host transformed with a first expression vector which encodes the light chain of the humanized antibody and with a second expression vector which encodes the heavy chain of the humanized antibody under such conditions that each chain is expressed and isolating the humanized antibody formed by assembly of the thus-expressed chains. The first and second expression vectors can be the same vector. Also provided herein are DNA sequences encoding the light chain or the heavy chain of the humanized antibody; an expression vector which incorporates a said DNA sequence; and a host transformed with a said expression vector. Generating a humanized antibody from the sequences and information provided herein can be practiced by those of ordinary skill in the art without undue experimentation. In one approach, there are four general steps employed to humanize a monoclonal antibody. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

### Purification

In one aspect, disclosed herein is a purified antibody or antigen-binding fragment as provided herein. Once expressed, the whole antibodies, their dimers, individual light and heavy chains, or other immunoglobulin forms of the present invention can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, NY, 1982).

Substantially pure immunoglobulins of at least about 90% to 95% homogeneity are advantageous, as are those with 98% to 99% or more homogeneity, particularly for pharmaceutical uses. When using recombinant techniques, the antibody can be produced intracellularly, in the periplasmic space, or directly secreted into the medium, including from microbial cultures. If the antibody is produced intracellularly, as a first step, the particulate debris, either host cells or lysed fragments, is removed, for example, by centrifugation or ultrafiltration. Better et al. Science 240: 1041-1043 (1988); ICSU Short Reports 10: 105 (1990); and Proc. Natl. Acad. Sci. USA 90: 457-461 (1993) describe a procedure for isolating antibodies which are secreted to the periplasmic space of E. coli. (See also, [Carter et al., Bio/Technology 10: 163-167 (1992)].

The antibody composition prepared from microbial or mammalian cells can be purified using, for example, hydroxylapatite chromatography cation or avian exchange chromatography, and affinity chromatography, with affinity chromatography being the preferred purification technique. The suitability of protein A as an affinity ligand depends on the species and isotype of any immunoglobulin Fe domain that is present in the antibody. Protein A can be used to purify antibodies that are based on human γ1, γ2, or γ4 heavy chains (Lindmark et al., J. Immunol. Meth. 62: 1-13 (1983)). Protein G is recommended for all mouse isotypes and for human y3 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered. Once purified, partially or to homogeneity as desired, a humanized or composite human antibody can then be used therapeutically or in developing and performing assay procedures, immunofluorescent staining, and the like. See generally, Vols. I & II Immunol. Meth. (Lefkovits & Pernis, eds., Acad. Press, N Y, 1979 and 1981).

### Antibodies

### Antibody Terminology

As used herein, the term “antibody” refers to an immunoglobulin (Ig) whether natural or partly or wholly synthetically produced. The term also covers any polypeptide or protein having a binding domain which is, or is homologous to, an antigen-binding domain. The term further includes “antigen-binding fragments” and other interchangeable terms for similar binding fragments such as described below.

An antibody includes, but is not be limited to, any specific binding member, immunoglobulin class and/or isotype (e.g., IgG1, IgG2, IgG3, IgG4, IgM, IgA, IgD, IgE and IgM); and biologically relevant fragment or specific binding member thereof. Thus, an antibody includes, for example, monoclonal antibodies, chimeric antibodies, humanized antibodies, human antibodies, recombinant antibodies, chemically engineered antibodies, deimmunized antibodies, affinity-matured antibodies, multispecific antibodies (for example, bispecific antibodies and polyreactive antibodies), heteroconjugate antibodies, antibody fragments, and combinations thereof (e.g., a monoclonal antibody that is also deimmunized, a humanized antibody that is also deimmunized, etc.).

The present disclosure provides antibodies that find use in treatment and/or prevention of infection with SARS-CoV-2. The term “SARS-CoV-2 associated antibody” as used herein refers to an antibody specific for a SARS-CoV-2 associated antigen. In some embodiments, the SARS-CoV-2 associated antibody comprises at least one antigen-binding region specific for a SARS-CoV-2 associated antigen. Disclosed herein are the complete reconstructed nucleic acid consensus sequences and complete reconstructed polypeptide consensus sequences of the variable heavy chain (V_{H}) and variable light chain (V_{L}) of the antibodies. The nucleic acid and polypeptide sequences of the three complementarity-determining regions (CDRs) of the V_{H} and the V_{L} are also provided.

Native antibodies and native immunoglobulins are usually heterotetrameric glycoproteins of about 150,000 Daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The antibodies or antigen-binding fragment thereof of the present disclosure can comprise a deletion at an end of a light chain. The antibodies or antigen-binding fragment thereof of the invention can comprise a deletion of 3 or more amino acids at an end of the light chain. The antibodies or antigen-binding fragment thereof of the invention can comprise a deletion of 7 or less amino acids at an end of the light chain. The antibodies or antigen-binding fragment thereof of the invention can comprise a deletion of 3, 4, 5, 6, or 7 amino acids at an end of the light chain.

The antibodies or antigen-binding fragment thereof of the present disclosure can comprise an insertion in a light chain. The antibodies or antigen-binding fragment thereof of the invention can comprise an insertion of 1, 2, 3, 4, 5, 6, 7, 8, 9, or 10, or more amino acids in the light chain. The antibodies or antigen-binding fragment thereof of the invention can comprise an insertion of 3 amino acids in the light chain.

A “variable region” of an antibody refers to the variable region of the antibody light chain or the variable region of the antibody heavy chain, either alone or in combination. The variable regions of the heavy and light chain each consist of four framework regions (FR) connected by three complementarity-determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (e.g., Kabat et al. Sequences of Proteins of Immunological Interest, (5th ed., 1991, National Institutes of Health, Bethesda Md.)); and (2) an approach based on crystallographic studies of antigen-antibody complexes (Allazikani et al. (1997) J. Molec. Biol. 273:927-948)). A CDR may refer to CDRs defined by either approach or by a combination of both approaches.

A “constant region” of an antibody refers to the constant region of the antibody light chain or the constant region of the antibody heavy chain, either alone or in combination. The constant region does not vary with respect to antigen specificity.

As used herein, the term “heavy chain region” includes amino acid sequences derived from the constant domains of an immunoglobulin heavy chain. A polypeptide comprising a heavy chain region comprises at least one of: a C_{H}1 domain, a hinge (e.g., upper, middle, and/or lower hinge region) domain, a C_{H}2 domain, a C_{H}3 domain, or a variant or fragment thereof. In an embodiment, an antibody or an antigen-binding fragment thereof may comprise the Fc region of an immunoglobulin heavy chain (e.g., a hinge portion, a C_{H}2 domain, and a C_{H}3 domain). In another embodiment, an antibody or an antigen-binding fragment thereof lacks at least a region of a constant domain (e.g., all or part of a C_{H}2 domain). In certain embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, C_{H}2 and C_{H}3 domain sequences from a human immunoglobulin). In certain embodiments, the constituent constant domains of the heavy chain region are from different immunoglobulin molecules. For example, a heavy chain region of a polypeptide may comprise a domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule. That is, the polypeptides of the invention disclosed herein may comprise alterations or modifications to one or more of the heavy chain constant domains (C_{H}1, hinge, C_{H}2 or C_{H}3) and/or to the light chain constant domain (C_{L}). Exemplary modifications include additions, deletions or substitutions of one or more amino acids in one or more domains.

The antibodies or antigen-binding fragment thereof of the present disclosure can comprise a CDR3 region that is a length of at least about 10, 11, 12, 13, 14, 15, 16, 17, 18, 19, 20, 21, 22, 23, 24, 25, 26, 27, 28, 29, or 30 amino acids in length. The antibodies or antigen-binding fragment thereof of the present disclosure can comprise a CDR3 region that is at least about 18 amino acids in length.

As used herein, the term “hinge region” includes the region of a heavy chain molecule that joins the C_{H}1 domain to the C_{H}2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al. J. Immunol. 1998 161:4083).

As used herein, the term “Fv” is the minimum antibody fragment that contains a complete antigen-recognition and -binding site. This fragment consists of a dimer of one heavy- and one light-chain variable region domain in tight, non-covalent association.

“Heavy chain variable region” or “V_{H}” with regard to an antibody refers to the fragment of the heavy chain that contains three CDRs interposed between flanking stretches known as framework regions, these framework regions are generally more highly conserved than the CDRs and form a scaffold to support the CDRs.

Six hypervariable loops (three loops each from the H and L chain) contribute the amino acid residues for antigen-binding and confer antigen-binding specificity to the antibody. However, even a single variable domain (or half of an Fv comprising only three CDRs specific for an antigen) has the ability to recognize and bind antigen, although at a lower affinity than the entire binding site.

“Framework” or FR residues are those variable domain residues other than the hypervariable region residues.

It is understood in the art that an antibody is a glycoprotein having at least two heavy (H) chains and two light (L) chains interconnected by disulfide bonds, or an antigen-binding portion thereof. A heavy chain is comprised of a heavy chain variable region (V_{H}) and a heavy chain constant region (C_{H}1, C_{H}2 and C_{H}3). A light chain is comprised of a light chain variable region (VL) and a light chain constant region (C_{L}). The variable regions of both the heavy and light chains comprise framework regions (FRs or FWRs) and hypervariable regions (HVRs). The HVRs are the amino acid residues of an antibody that are responsible for antigen binding. The hypervariable region generally comprises amino acid residues from a complementarity determining region (CDR), which have the highest sequence variability and/or involved in antigen recognition. With the exception of CDR1 in V_{H}, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See, e.g., Fransson, Front. Biosci. 13:1619-1633 (2008).)

Unless otherwise indicated, HVR residues and other residues in the variable domain (e.g., FR residues) are numbered herein according to Kabat et al., supra. A variable region is a domain of an antibody heavy or light chain that is involved in binding the antibody to antigen. (See, e.g., Kindt et al. Kuby Immunology, 6th ed., W.H. Freeman and Co., p. 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. (See, e.g., Portolano et al., J. Immunol. 150:880-887 (1993); Clarkson et al., Nature 352:624-628 (1991)). The four FWR regions are typically more conserved while CDR regions (CDR1, CDR2 and CDR3) represent hypervariable regions and are arranged from NH2 terminus to the COOH terminus as follows: FWR1, CDR1, FWR2, CDR2, FWR3, CDR3, and FWR4. The variable regions of the heavy and light chains contain a binding domain that interacts with an antigen while, depending of the isotype, the constant region(s) may mediate the binding of the immunoglobulin to host tissues or factors. An antibody also includes chimeric antibodies, humanized antibodies, and recombinant antibodies, human antibodies generated from a transgenic non-human animal, as well as antibodies selected from libraries using enrichment technologies available to the artisan.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (“κ”) and lambda (“λ”) light chains refer to the two major antibody light chain isotypes.

An antibody or antigen-binding fragment thereof “specifically binds” or “preferentially binds” to a target antigen if it binds with greater affinity and/or avidity than it binds to epitopes on unrelated polypeptides. The specificity of an antibody or antigen-binding fragment or portion thereof can be determined based on affinity and/or avidity. Methods to determine such specific binding are also well known in the art. According to certain embodiments of the present disclosure, the antibodies or antigen-binding fragment thereof can bind to a SARS-CoV-2 antigen but not to antigens from other viruses. In some embodiments, an antibody or antigen binding fragment thereof disclosed herein specifically bind to a target antigen disclosed herein.

The affinity, represented by the equilibrium constant for the dissociation (K_{D}) of an antigen with an antigen-binding protein, is a measure for the binding strength between an antigenic determinant and an antigen-binding site on the antigen-binding protein: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific antigen of interest. Accordingly, an antibody or antigen-binding fragment thereof as defined herein is said to be “specific for” a first target or antigen compared to a second target or antigen when it binds to the first antigen with an affinity (as described above, and suitably expressed, for example as a K_{D} value) that is at least 50 times, such as at least 100 times, and preferably at least 1000 times, and up to 10,000 times or more better than the affinity with which said amino acid sequence or polypeptide binds to another target or polypeptide. Preferably, when an antibody or antigen-binding fragment thereof is “specific for” a target or antigen, compared to another target or antigen, it can bind the target or antigen, but does not bind the other target or antigen. However, as understood by one of ordinary skill in the art, in some embodiments, where a binding site on a target is shared or partially shared by multiple, different ligands, an antibody or antigen-binding fragment thereof can specifically bind to a target, such as a SARS-CoV-2 antigen, and have the functional effect of, for example, inhibiting/preventing the spread of SARS-CoV-2 infection.

In some embodiments, an antibody provided herein has a dissociation constant (K_{D}) of about 1 μM, 100 nM, 10 nM, 5 nM, 2 nM, 1 nM, 0.5 nM, 0.1 nM, 0.05 nM, 0.01 nM, or 0.001 nM or less (e.g., 10⁻⁸ M or less, e.g., from 10⁻⁸ M to 10⁻¹³ M, e.g., from 10⁻⁹ M to 10⁻¹³ M). Another aspect of the invention provides for an antibody or antigen-binding fragment thereof with an increased affinity for its target, for example, an affinity matured antibody. An affinity matured antibody is an antibody with one or more alterations in one or more hypervariable regions (HVRs), compared to a parent antibody which does not possess such alterations, such alterations resulting in an improvement in the affinity of the antibody for antigen. These antibodies can bind to antigen with a K_{D} of about 5×10⁻⁹ M, 2×10⁻⁹ M, 1×10⁻⁹ M, 5×10⁻¹⁰ M, 2×10⁻⁹ M, 1×10⁻¹⁰ M, 5×10⁻¹¹ M, 1×10⁻¹¹ M, 5×10⁻¹² M, 1×10⁻¹² M, or less. In some embodiments, the present disclosure provides an antibody or antigen-binding fragment thereof which has an increased affinity of at least 1.5 fold, 2 fold, 2.5 fold, 3 fold, 4 fold, 5 fold, 10 fold, 20 fold or greater as compared to a germline antibody containing the heavy chain sequence and light chain sequence, or both. In other embodiments, an antibody is provided that competes for binding to the same epitope as an antibody as described herein. In some embodiments, the antibody or antigen-binding fragment thereof that binds to the same epitope, and/or competes for binding to the same epitope as an antibody exhibits effector function activities, such as, for example, Fc-mediated cellular cytotoxicity, including ADCC activity.

K_{D} can be measured by any suitable assay. For example, K_{D} can be measured by a radiolabeled antigen-binding assay (RIA) (see, e.g., Chen et al., J. Mol. Biol. 293:865-881 (1999); Presta et al., Cancer Res. 57:4593-4599 (1997)). For example, K_{D} can be measured using a surface plasmon resonance assay (e.g., using a BIACORE®-2000 or a BIACORE®-3000). For example, K_{D} can be measured using a competitive ELISA.

Avidity is the measure of the strength of binding between an antigen-binding molecule and the pertinent antigen. Avidity is related to both the affinity between an antigenic determinant and its antigen-binding site on the antigen-binding molecule, and the number of pertinent binding sites present on the antigen-binding molecule. Typically, antigen-binding proteins will bind to their cognate or specific antigen with a dissociation constant (K_{D} of 10⁻⁵ to 10⁻¹² M or less, and preferably 10⁻⁷ to 10⁻¹² M or less and more preferably 10⁻⁸ to 10⁻¹² M (i.e. with an association constant (K_{A}) of 10⁵ to 10¹² M⁻¹ or more, and preferably 10⁷ to 10¹² M⁻¹ or more and more preferably 10⁸ to 10¹² M⁻¹). Any K_{D} value greater than 10⁻⁴ M (or any K_{A} value lower than 10⁴ M⁻¹) is generally considered to indicate non-specific binding. The K_{D} for biological interactions which are considered meaningful (e.g., specific) are typically in the range of 10⁻¹⁰ M (0.1 nM) to 10⁻⁵ M (10000 nM). The stronger an interaction is, the lower is its K_{D}. Preferably, a binding site on an anti-LAP antibody or antigen-binding fragment thereof described herein will bind with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

The term “kₒₙ”, as used herein, is intended to refer to the rate constant for association of an antibody or antigen-binding fragment thereof to an antigen.

The term “k_{off}”, as used herein, is intended to refer to the rate constant for dissociation of an antibody or antigen-binding fragment thereof from the antibody/antigen complex.

### Computationally Reconstructed Antibodies

Provided herein are reconstructed polypeptide and nucleic acid consensus sequences for SARS-CoV-2 associated antibodies. The consensus sequences are reconstructed in silico. The term “polypeptide consensus sequence” as used herein refers to an amino acid sequence which comprises the most frequently occurring amino acid residues at each location in all immunoglobulins of any particular subclass or subunit structure. The polypeptide consensus sequence may be based on immunoglobulins of a particular species or of many species. A polypeptide “consensus” sequence, “consensus” structure, or “consensus” antibody is understood to encompass a human polypeptide consensus sequence as described in certain embodiments provided herein, and to refer to an amino acid sequence which comprises the most frequently occurring amino acid residues at each location in all human immunoglobulins of any particular subclass or subunit structure. The embodiments herein provide consensus human structures and consensus structures, which consider other species in addition to human.

The term, “nucleic acid consensus sequence” as used herein refers to a nucleic acid sequence, which comprises the most frequently occurring nucleotide residues at each location in all immunoglobulin nucleic acid sequence of any particular subclass or subunit structure. The nucleic acid consensus sequence may be based on immunoglobulins of a particular species or of many species. A nucleic acid “consensus” sequence, or “consensus” structure, is understood to encompass a human nucleic acid consensus sequence as described in certain embodiments of this invention, and to refer to a nucleic acid sequence which comprises the most frequently occurring nucleotide residues at each location in all human immunoglobulins nucleic acid of any particular subclass or subunit structure.

Provided herein are consensus human structures. Methods to computationally reconstruct the consensus sequences from RNA seq data are described in the examples herein. Non limiting examples of computational tools known in the art for reconstructing full-length antibody repertoires including MIGEC (Shugay et al. 2014), PRESTO (Vander Heiden et al. 2014), MiXCR (Bolotin et al. 2015), and IGREPERTOIRECONSTRUCTOR (Safonova et al. 2015). In some embodiments, the TraCeR pipeline by Stubbington and Teichmann is implemented, which uses de novo assembly after a prefiltering step against a custom database containing in silico combinations for all known human V and J gene segments/alleles in the International Immunogenetics Information System (IMGT) repository. In some embodiments, another pipeline, VDJPuzzle, is implemented which filters in reads by mapping to TCR genes followed by a Trinity-based assembly; whereby the total reads are then mapped back to the assemblies in order to retrieve reads missed in the initial mapping step, followed by another round of assembly with Trinity. An exemplary method for computationally reconstructing consensus sequences can comprise somatic sequence identification, manual IGV investigation and (if necessary) correction of somatic vdj sequence and identification of germline sequence and CDR regions.

In some embodiments, RNA-seq FASTQ files retrieved for patients e.g., a COVID-19 patient are recorded and analyzed. Kallisto, BWA, MiXCR or other known tools can be used, in some embodiments, to perform a first alignment of RNA-seq samples to reference V, D and J genes of immunoglobulins in order to identify the repertoire present in the samples. In further embodiments, identical CDR3 sequences are identified and grouped in clonotypes (Bolotin D A et al., Nature Methods, 2015; Bolotin D A et al. Nature Biotechnology, 2017). VDJ tools are used, in some embodiments, (Shugay M. et al. PLoS Computational Biology, 2015) to filter out nonfunctional (non-coding) clonotypes and to compute basic diversity statistics. In further embodiments, non-functional clonotypes are identified as those containing a stop codon or frameshift in their receptor sequence. In some embodiments, the diversity of the Ig repertoire is obtained based on the effective number of species which is calculated as the exponent of the Shannon-Wiener Entropy index (MacArthur RH. Biological reviews. 1965).

In some embodiments, further alignments against the immunoglobulin segments present in the samples are performed for viewing the results to explore the frequency distribution of sequence mismatches along the V, D, J gene segments and, in particular in the CDR3 region length statistics. This alignment step can be useful, for example, for summarizing repertoires, as well as offering a detailed view of rearrangements and region alignments for individual query sequences. Exemplary methodology for alignment and assembly is described in the examples herein.

In some embodiments, the immunoglobulin segments present in the samples are identified using IMGT reference files or equivalent. In some instances, the heavy D segment and light V-J junction sequences can be assembled using an assembler. Non limiting examples of assembler known in the art include Trinity and V'DJer. A FASTA file with corrected heavy D and light V-J junction sequences can be generated for each sample in some embodiments. In addition to the assembled FASTA files, germline FASTA files can be generated, for example, by using IgBLAST v1.9.0 [Ye J, et al Nucleic Acids Research, 2013] and the IMGT database. In further embodiments, the somatic FASTA sequence can be input to IgBLAST to obtain the closest segment ids for the heavy and light chain. The germline FASTA can be generated by merging corresponding segment sequences from the IMGT database. The final assembled FASTA sequences can serve as ‘reference’ sequences for the alignment and visualization steps.

In further embodiments, using the reference files generated from the assembly step, the FASTQs can be aligned in BowTie2 default mode. Other alignment tools, known in the art, for example STAR or TopHat2 can also be used. The output BAM file can be used for IGV visualization and mutations in the patient can be observed.

In further embodiments, the identification of the CDR3 region and corresponding V, D, and J chains from the final assembled FASTA sequences can be done, for example with IgBLAST. The standardized output using version v.1.9.0 of IgBLAST can be delivered by wrapping IgBLASTn with default parameters in some instances. In other instances, the output from the IgBLAST service can be extracted using a purpose-built parser tool designed to extract the CDR1, CDR2, and CDR3 nucleotide and amino acid sequences.

### Exemplary SARS-CoV-2 Associated Antibodies or Antigen-Binding Fragments Thereof

The present disclosure provides SARS-CoV-2 associated antibodies or antigen-binding fragments comprising a consensus sequence. In some embodiments, the antibodies or antigen-binding fragment thereof neutralize SARS-CoV-2.

In one aspect, an antibody or antigen-binding fragment thereof is provided, wherein the antibody or antigen-binding fragment thereof comprises a heavy chain variable domain (V_{H}) having at least 90%, 91%, 92%, 93%, 94%, 95%, 96%, 97%, 98%, 99%, or 100% sequence identity to the amino acid sequence of any one of SEQ ID NOs: 17501-18750. In some embodiments, a V_{H} sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to same antigen as of the parent (e.g., SARS-CoV-2 associated antigen). In some embodiments, a total of 1 to 10 amino acids have been substituted, inserted and/or deleted in the amino acid sequence of any one of SEQ ID NOs: 17501-18750. In some embodiments, substitutions, insertions, or deletions occur in regions outside the CDRs (e.g., in the FRs). Optionally, the antibody or antigen-binding fragment thereof comprises the V_{H} sequence of any one of SEQ ID NOs: 17501-18750, including one or more post-translational modifications of that sequence.

In some embodiments, the VH comprises one, two or three CDRs selected from: (a) CDR-H1, comprising the amino acid sequence of any one of SEQ ID NOs: 10001-11250, (b) CDR-H2, comprising the amino acid sequence of any one of SEQ ID NOs: 12501-13750, and (c) CDR-H3, comprising the amino acid sequence of any one of SEQ ID NOs: 15001-16250.

In some embodiments, the VH comprises one, two or three CDRs selected from: (a) CDR-H1, comprising the amino acid sequence of any one of SEQ ID NOs: 10001-11250, (b) CDR-H2, comprising the amino acid sequence of any one of SEQ ID NOs: 12501-13750, and (c) CDR-H3, comprising the amino acid sequence of any one of SEQ ID NOs: 15001-16250, wherein the selected CDR-H1, CDR-H2, and CDR-H3 are paired according to Table 1.

In one aspect, an antibody or antigen-binding fragment thereof is provided, wherein the antibody or antigen-binding fragment thereof comprises a light chain variable domain (V_{L}) having at least 90%, 91%, 92%, 93%, 94%, 95%, 96%, 97%, 98%, 99%, or 100% sequence identity to the amino acid sequence of any one of SEQ ID NOs: 18751-20000. In some embodiments, a V_{L} sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to same antigen as the parent (e.g., SARS-CoV-2 associated antigen). In some embodiments, a total of 1 to 10 amino acids have been substituted, inserted and/or deleted in the amino acid sequence of any one of SEQ ID NOs: 18751-20000. In some embodiments, the substitutions, insertions, or deletions occur in regions outside the CDRs (e.g., in the FRs). Optionally, the antibody or antigen-binding fragment thereof comprises the V_{L} sequence of any one of SEQ ID NOs: 18751-20000, including one or more post-translational modifications of that sequence.

In some embodiments, the V_{L} comprises one, two or three CDRs selected from: (a) CDR-L1, comprising the amino acid sequence of any one of SEQ ID NOs: 11251-12500, (b) CDR-L2, comprising the amino acid sequence of any one of SEQ ID NOs: 13751-15000, and (c) CDR-L3, comprising the amino acid sequence of any one of SEQ ID NOs: 16251-17500.

In some embodiments, the VL comprises one, two or three CDRs selected from: (a) CDR-L1, comprising the amino acid sequence of any one of SEQ ID NOs: 11251-12500, (b) CDR-L2, comprising the amino acid sequence of any one of SEQ ID NOs: 13751-15000, and (c) CDR-L3, comprising the amino acid sequence of any one of SEQ ID NOs: 16251-17500, wherein the selected CDR-L1, CDR-L2, and CDR-L3 are paired according to Table 1.

In one aspect, an antibody or antigen-binding fragment thereof is provided, wherein the antibody or antigen-binding fragment thereof comprises: (a) a V_{H} comprising the amino acid sequence of any one of SEQ ID NOs: 17501-18750, and (b) a V_{L}, comprising the amino acid sequence of any one of SEQ ID Nos: 18751-20000, and optionally including post-translational modifications of those sequences.

In one aspect, an antibody or antigen-binding fragment thereof is provided, wherein the antibody or antigen-binding fragment thereof comprises: (a) a VH comprising the amino acid sequence of any one of SEQ ID NOs: 17501-18750, and (b) a V_{L}, comprising the amino acid sequence of any one of SEQ ID Nos: 18751-20000, wherein the selected V_{H} and V_{L} are paired according to Table 1.

In one aspect, an antibody or antigen-binding fragment thereof is provided, wherein the antibody or antigen-binding fragment thereof comprises: (a) a CDR-H1 selected from any one of SEQ ID NOs: 10001-11250, and (b) a CDR-L1 selected from any one of SEQ ID NOs: 11251-12500, wherein the selected CDR-H1 and CDR-L1 are paired according to Table 1.

In one aspect, an antibody or antigen-binding fragment thereof is provided, wherein the antibody or antigen-binding fragment thereof comprises: (a) a CDR-H2 selected from any one of SEQ ID NOs: 12501-13750, and (b) a CDR-L2 selected from any one of SEQ ID NOs: 13751-15000, wherein the selected CDR-H2 and CDR-L2 are paired according to Table 1.

In one aspect, an antibody or antigen-binding fragment thereof is provided, wherein the antibody or antigen-binding fragment thereof comprises: (a) a CDR-H3 selected from any one of SEQ ID NOs: 15001-16250, and (b) a CDR-L3 selected from any one of SEQ ID NOs: 16251-17500, wherein the selected CDR-H3 and CDR-L3 are paired according to Table 1.

In one aspect, an antibody or antigen-binding fragment thereof is provided, wherein the antibody or antigen-binding fragment thereof comprises: (a) a CDR-H1 selected from any one of SEQ ID NOs: 10001-11250, a CDR-H2 selected from any one of SEQ ID NOs: 12501-13750, and a CDR-H3 selected from any one of SEQ ID NOs: 15001-16250, and (b) a CDR-L1 selected from any one of SEQ ID NOs: 11251-12500, a CDR-L2 selected from any one of SEQ ID NOs: 13751-15000, and a CDR-L3 selected from any one of SEQ ID NOs: 16251-17500, wherein the selected CDR-H1, CDR-H2, CDR-H3, CDR-L1, CDR-L2, and CDR-L3 are paired according to Table 1.

### TOTCOVID00425

In one aspect, the present disclosure provides an antibody or antigen-binding fragment thereof comprises one or more variable regions selected from the group consisting of (a) VH comprising the amino acid sequence of SEQ ID NO: 17869, (b) VL comprising the amino acid sequence of SEQ ID NO: 19119, and (c) a combination thereof.

In one aspect, the present disclosure provides an antibody or antigen-binding fragment thereof comprising at least one, two, three, four, five, or six CDRs selected from (a) CDR-H1 comprising the amino acid sequence of SEQ ID NO: 10369; (b) CDR-H2 comprising the amino acid sequence of SEQ ID NO: 12869; (c) CDR-H3 comprising the amino acid sequence of SEQ ID NO: 15369; (d) CDR-L1 comprising the amino acid sequence of SEQ ID NO: 11619; (e) CDR-L2 comprising the amino acid sequence of SEQ ID NO: 14119; and (f) CDR-L3 comprising the amino acid sequence of SEQ ID NO: 16619.

In one aspect, the present disclosure provides an antibody or antigen-binding fragment thereof comprising at least one, at least two, or all three VH CDR sequences selected from (a) CDR-H1 comprising the amino acid sequence of SEQ ID NO: 10369; (b) CDR-H2 comprising the amino acid sequence of SEQ ID NO: 12869; and (c) CDR-H3 comprising the amino acid sequence of SEQ ID NO: 15369; and (d) a VL comprising the amino acid sequence of SEQ ID NO: 19119.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 11619; (b) CDR-L2 comprising the amino acid sequence of SEQ ID NO: 14119; and (c) CDR-L3 comprising the amino acid sequence of SEQ ID NO: 16619; and a VH comprising the amino acid sequence of SEQ ID NO: 17869.

In one aspect, the present disclosure provides an antibody or antigen-binding fragment thereof comprising the CDRs: CDR-H3 comprising the amino acid sequence of SEQ ID NO: 15369; and CDR-L3 comprising the amino acid sequence of SEQ ID NO: 16619.

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 11619; (b) CDR-L2 comprising the amino acid sequence of SEQ ID NO: 14119 and (c) CDR-L3 comprising the amino acid sequence of SEQ ID NO: 16619. In one aspect, the disclosure provides an antibody or antigen-binding fragment thereof comprising at least one, at least two, or all three VH CDR sequences selected from (a) CDR-H1 comprising the amino acid sequence of SEQ ID NO: 10369; (b) CDR-H2 comprising the amino acid sequence of SEQ ID NO: 12869 and (c) CDR-H3 comprising the amino acid sequence of SEQ ID NO: 15369.

In one aspect, the disclosure provides an antibody or antigen-binding fragment thereof comprising the CDRs: (a) CDR-H1 comprising the amino acid sequence of SEQ ID NO: 10369; (b) CDR-H2 comprising the amino acid sequence of SEQ ID NO: 12869; (c) CDR-H3 comprising the amino acid sequence of SEQ ID NO: 15369; (d) CDR-L1 comprising the amino acid sequence of SEQ ID NO: 11619; (e) CDR-L2 comprising the amino acid sequence of SEQ ID NO: 14119; and (f) CDR-L3 comprising the amino acid sequence of SEQ ID NO: 16619.

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 17869. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 17869 In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 17869, including post-translational modifications of that sequence. In a particular embodiment, the VH comprises one, two or three CDRs selected from: (a) CDR-H1 comprising the amino acid sequence of SEQ ID NO: 10369, (b) CDR-H2 comprising the amino acid sequence of SEQ ID NO: 12869, and (c) CDR-H3 comprising the amino acid sequence of SEQ ID NO: 15369.

In one aspect, an antibody or antigen-binding fragment thereof is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 19119. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 19119. In some embodiments, the substitutions, insertions, or deletions occur in regions outside the CDRs (e.g., in the FRs). Optionally, the antibody or antigen-binding fragment thereof comprises the VL sequence of SEQ ID NO: 19119, including post-translational modifications of that sequence. In a particular embodiment, the VL comprises one, two or three CDRs selected from (a) CDR-L1 comprising the amino acid sequence of SEQ ID NO: 11619; (b) CDR-L2 comprising the amino acid sequence of SEQ ID NO: 14119; and (c) CDR-L3 comprising the amino acid sequence of SEQ ID NO: 16619.

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody comprises a VH comprising the amino acid sequence of SEQ ID NO: 17869, and a VL sequence in SEQ ID NO: 19119, including post-translational modifications of those sequences.

### TOTCOVID00316

In one aspect, the present disclosure provides an antibody or antigen-binding fragment thereof comprises one or more variable regions selected from the group consisting of (a) VH comprising the amino acid sequence of SEQ ID NO: 17760, (b) VL comprising the amino acid sequence of SEQ ID NO: 19010, and (c) a combination thereof.

In one aspect, the present disclosure provides an antibody or antigen-binding fragment thereof comprising at least one, two, three, four, five, or six CDRs selected from (a) CDR-H1 comprising the amino acid sequence of SEQ ID NO: 10260; (b) CDR-H2 comprising the amino acid sequence of SEQ ID NO: 12760; (c) CDR-H3 comprising the amino acid sequence of SEQ ID NO: 15260; (d) CDR-L1 comprising the amino acid sequence of SEQ ID NO: 11510; (e) CDR-L2 comprising the amino acid sequence of SEQ ID NO: 14010; and (f) CDR-L3 comprising the amino acid sequence of SEQ ID NO: 16510.

In one aspect, the present disclosure provides an antibody or antigen-binding fragment thereof comprising at least one, at least two, or all three VH CDR sequences selected from (a) CDR-H1 comprising the amino acid sequence of SEQ ID NO: 10260; (b) CDR-H2 comprising the amino acid sequence of SEQ ID NO: 12760; and (c) CDR-H3 comprising the amino acid sequence of SEQ ID NO: 15260; and (d) a VL comprising the amino acid sequence of SEQ ID NO: 19010.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 11510; (b) CDR-L2 comprising the amino acid sequence of SEQ ID NO: 14010; and (c) CDR-L3 comprising the amino acid sequence of SEQ ID NO: 16510; and a VH comprising the amino acid sequence of SEQ ID NO: 17760.

In one aspect, the present disclosure provides an antibody or antigen-binding fragment thereof comprising the CDRs: CDR-H3 comprising the amino acid sequence of SEQ ID NO: 15260; and CDR-L3 comprising the amino acid sequence of SEQ ID NO: 16510.

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 11510; (b) CDR-L2 comprising the amino acid sequence of SEQ ID NO: 14010 and (c) CDR-L3 comprising the amino acid sequence of SEQ ID NO: 16510. In one aspect, the disclosure provides an antibody or antigen-binding fragment thereof comprising at least one, at least two, or all three VH CDR sequences selected from (a) CDR-H1 comprising the amino acid sequence of SEQ ID NO: 10260; (b) CDR-H2 comprising the amino acid sequence of SEQ ID NO: 12760 and (c) CDR-H3 comprising the amino acid sequence of SEQ ID NO: 15260.

In one aspect, the disclosure provides an antibody or antigen-binding fragment thereof comprising the CDRs: (a) CDR-H1 comprising the amino acid sequence of SEQ ID NO: 10260; (b) CDR-H2 comprising the amino acid sequence of SEQ ID NO: 12760; (c) CDR-H3 comprising the amino acid sequence of SEQ ID NO: 15260; (d) CDR-L1 comprising the amino acid sequence of SEQ ID NO: 11510; (e) CDR-L2 comprising the amino acid sequence of SEQ ID NO: 14010; and (f) CDR-L3 comprising the amino acid sequence of SEQ ID NO: 16510.

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 17760. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 17760. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 17760, including post-translational modifications of that sequence. In a particular embodiment, the VH comprises one, two or three CDRs selected from: (a) CDR-H1 comprising the amino acid sequence of SEQ ID NO: 10260, (b) CDR-H2 comprising the amino acid sequence of SEQ ID NO: 12760, and (c) CDR-H3 comprising the amino acid sequence of SEQ ID NO: 15260.

In one aspect, an antibody or antigen-binding fragment thereof is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 19010. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 19010. In some embodiments, the substitutions, insertions, or deletions occur in regions outside the CDRs (e.g., in the FRs). Optionally, the antibody or antigen-binding fragment thereof comprises the VL sequence of SEQ ID NO: 19010, including post-translational modifications of that sequence. In a particular embodiment, the VL comprises one, two or three CDRs selected from (a) CDR-L1 comprising the amino acid sequence of SEQ ID NO: 11510; (b) CDR-L2 comprising the amino acid sequence of SEQ ID NO: 14010; and (c) CDR-L3 comprising the amino acid sequence of SEQ ID NO: 16510.

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody comprises a VH comprising the amino acid sequence of SEQ ID NO: 17760, and a VL sequence in SEQ ID NO: 19010, including post-translational modifications of those sequences.

### TOTCOVID00761

In one aspect, the present disclosure provides an antibody or antigen-binding fragment thereof comprises one or more variable regions selected from the group consisting of (a) VH comprising the amino acid sequence of SEQ ID NO: 18205, (b) VL comprising the amino acid sequence of SEQ ID NO: 19455, and (c) a combination thereof.

In one aspect, the present disclosure provides an antibody or antigen-binding fragment thereof comprising at least one, two, three, four, five, or six CDRs selected from (a) CDR-H1 comprising the amino acid sequence of SEQ ID NO: 10705; (b) CDR-H2 comprising the amino acid sequence of SEQ ID NO: 13205; (c) CDR-H3 comprising the amino acid sequence of SEQ ID NO: 15705; (d) CDR-L1 comprising the amino acid sequence of SEQ ID NO: 11955; (e) CDR-L2 comprising the amino acid sequence of SEQ ID NO: 14455; and (f) CDR-L3 comprising the amino acid sequence of SEQ ID NO: 16955.

In one aspect, the present disclosure provides an antibody or antigen-binding fragment thereof comprising at least one, at least two, or all three VH CDR sequences selected from (a) CDR-H1 comprising the amino acid sequence of SEQ ID NO: 10705; (b) CDR-H2 comprising the amino acid sequence of SEQ ID NO: 13205; and (c) CDR-H3 comprising the amino acid sequence of SEQ ID NO: 15705; and (d) a VL comprising the amino acid sequence of SEQ ID NO: 19455.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 11955; (b) CDR-L2 comprising the amino acid sequence of SEQ ID NO: 14455; and (c) CDR-L3 comprising the amino acid sequence of SEQ ID NO: 16955; and a VH comprising the amino acid sequence of SEQ ID NO: 18205.

In one aspect, the present disclosure provides an antibody or antigen-binding fragment thereof comprising the CDRs: CDR-H3 comprising the amino acid sequence of SEQ ID NO: 15705; and CDR-L3 comprising the amino acid sequence of SEQ ID NO: 16955.

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 11955; (b) CDR-L2 comprising the amino acid sequence of SEQ ID NO: 14455 and (c) CDR-L3 comprising the amino acid sequence of SEQ ID NO: 16955. In one aspect, the disclosure provides an antibody or antigen-binding fragment thereof comprising at least one, at least two, or all three VH CDR sequences selected from (a) CDR-H1 comprising the amino acid sequence of SEQ ID NO: 10705; (b) CDR-H2 comprising the amino acid sequence of SEQ ID NO: 13205 and (c) CDR-H3 comprising the amino acid sequence of SEQ ID NO: 15705.

In one aspect, the disclosure provides an antibody or antigen-binding fragment thereof comprising the CDRs: (a) CDR-H1 comprising the amino acid sequence of SEQ ID NO: 10705; (b) CDR-H2 comprising the amino acid sequence of SEQ ID NO: 13205; (c) CDR-H3 comprising the amino acid sequence of SEQ ID NO: 15705; (d) CDR-L1 comprising the amino acid sequence of SEQ ID NO: 11955; (e) CDR-L2 comprising the amino acid sequence of SEQ ID NO: 14455; and (f) CDR-L3 comprising the amino acid sequence of SEQ ID NO: 16955.

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 18205. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 18205. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 18205, including post-translational modifications of that sequence. In a particular embodiment, the VH comprises one, two or three CDRs selected from: (a) CDR-H1 comprising the amino acid sequence of SEQ ID NO: 10705, (b) CDR-H2 comprising the amino acid sequence of SEQ ID NO: 13205, and (c) CDR-H3 comprising the amino acid sequence of SEQ ID NO: 15705.

In one aspect, an antibody or antigen-binding fragment thereof is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 19455. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 19455. In some embodiments, the substitutions, insertions, or deletions occur in regions outside the CDRs (e.g., in the FRs). Optionally, the antibody or antigen-binding fragment thereof comprises the VL sequence of SEQ ID NO: 19455, including post-translational modifications of that sequence. In a particular embodiment, the VL comprises one, two or three CDRs selected from (a) CDR-L1 comprising the amino acid sequence of SEQ ID NO: 11955; (b) CDR-L2 comprising the amino acid sequence of SEQ ID NO: 14455; and (c) CDR-L3 comprising the amino acid sequence of SEQ ID NO: 16955.

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody comprises a VH comprising the amino acid sequence of SEQ ID NO: 18205, and a VL sequence in SEQ ID NO: 19455, including post-translational modifications of those sequences.

### TOTCOVID00540

In one aspect, the present disclosure provides an antibody or antigen-binding fragment thereof comprises one or more variable regions selected from the group consisting of (a) VH comprising the amino acid sequence of SEQ ID NO: 17984, (b) VL comprising the amino acid sequence of SEQ ID NO: 19234, and (c) a combination thereof.

In one aspect, the present disclosure provides an antibody or antigen-binding fragment thereof comprising at least one, two, three, four, five, or six CDRs selected from (a) CDR-H1 comprising the amino acid sequence of SEQ ID NO: 10484; (b) CDR-H2 comprising the amino acid sequence of SEQ ID NO: 12984; (c) CDR-H3 comprising the amino acid sequence of SEQ ID NO: 15484; (d) CDR-L1 comprising the amino acid sequence of SEQ ID NO: 11734; (e) CDR-L2 comprising the amino acid sequence of SEQ ID NO: 14234; and (f) CDR-L3 comprising the amino acid sequence of SEQ ID NO: 16734.

In one aspect, the present disclosure provides an antibody or antigen-binding fragment thereof comprising at least one, at least two, or all three VH CDR sequences selected from (a) CDR-H1 comprising the amino acid sequence of SEQ ID NO: 10484; (b) CDR-H2 comprising the amino acid sequence of SEQ ID NO: 12984; and (c) CDR-H3 comprising the amino acid sequence of SEQ ID NO: 15484; and (d) a VL comprising the amino acid sequence of SEQ ID NO: 19234.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 11734; (b) CDR-L2 comprising the amino acid sequence of SEQ ID NO: 14234; and (c) CDR-L3 comprising the amino acid sequence of SEQ ID NO: 16734; and a VH comprising the amino acid sequence of SEQ ID NO: 17984.

In one aspect, the present disclosure provides an antibody or antigen-binding fragment thereof comprising the CDRs: CDR-H3 comprising the amino acid sequence of SEQ ID NO: 15484; and CDR-L3 comprising the amino acid sequence of SEQ ID NO: 16734.

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 11734; (b) CDR-L2 comprising the amino acid sequence of SEQ ID NO: 14234 and (c) CDR-L3 comprising the amino acid sequence of SEQ ID NO: 16734. In one aspect, the disclosure provides an antibody or antigen-binding fragment thereof comprising at least one, at least two, or all three VH CDR sequences selected from (a) CDR-H1 comprising the amino acid sequence of SEQ ID NO: 10484; (b) CDR-H2 comprising the amino acid sequence of SEQ ID NO: 12984 and (c) CDR-H3 comprising the amino acid sequence of SEQ ID NO: 15484.

In one aspect, the disclosure provides an antibody or antigen-binding fragment thereof comprising the CDRs: (a) CDR-H1 comprising the amino acid sequence of SEQ ID NO: 10484; (b) CDR-H2 comprising the amino acid sequence of SEQ ID NO: 12984; (c) CDR-H3 comprising the amino acid sequence of SEQ ID NO: 15484; (d) CDR-L1 comprising the amino acid sequence of SEQ ID NO: 11734; (e) CDR-L2 comprising the amino acid sequence of SEQ ID NO: 14234; and (f) CDR-L3 comprising the amino acid sequence of SEQ ID NO: 16734.

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 17984. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 17984. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 17984, including post-translational modifications of that sequence. In a particular embodiment, the VH comprises one, two or three CDRs selected from: (a) CDR-H1 comprising the amino acid sequence of SEQ ID NO: 10484, (b) CDR-H2 comprising the amino acid sequence of SEQ ID NO: 12984, and (c) CDR-H3 comprising the amino acid sequence of SEQ ID NO: 15484.

In one aspect, an antibody or antigen-binding fragment thereof is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 19234. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 19234. In some embodiments, the substitutions, insertions, or deletions occur in regions outside the CDRs (e.g., in the FRs). Optionally, the antibody or antigen-binding fragment thereof comprises the VL sequence of SEQ ID NO: 19234, including post-translational modifications of that sequence. In a particular embodiment, the VL comprises one, two or three CDRs selected from (a) CDR-L1 comprising the amino acid sequence of SEQ ID NO: 11734; (b) CDR-L2 comprising the amino acid sequence of SEQ ID NO: 14234; and (c) CDR-L3 comprising the amino acid sequence of SEQ ID NO: 16734.

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody comprises a VH comprising the amino acid sequence of SEQ ID NO: 17984, and a VL sequence in SEQ ID NO: 19234, including post-translational modifications of those sequences.

### TOTCOVID00347

In one aspect, the present disclosure provides an antibody or antigen-binding fragment thereof comprises one or more variable regions selected from the group consisting of (a) VH comprising the amino acid sequence of SEQ ID NO: 17791, (b) VL comprising the amino acid sequence of SEQ ID NO: 19041, and (c) a combination thereof.

In one aspect, the present disclosure provides an antibody or antigen-binding fragment thereof comprising at least one, two, three, four, five, or six CDRs selected from (a) CDR-H1 comprising the amino acid sequence of SEQ ID NO: 10291; (b) CDR-H2 comprising the amino acid sequence of SEQ ID NO: 12791; (c) CDR-H3 comprising the amino acid sequence of SEQ ID NO: 15291; (d) CDR-L1 comprising the amino acid sequence of SEQ ID NO: 11541; (e) CDR-L2 comprising the amino acid sequence of SEQ ID NO: 14041; and (f) CDR-L3 comprising the amino acid sequence of SEQ ID NO: 16541.

In one aspect, the present disclosure provides an antibody or antigen-binding fragment thereof comprising at least one, at least two, or all three VH CDR sequences selected from (a) CDR-H1 comprising the amino acid sequence of SEQ ID NO: 10291; (b) CDR-H2 comprising the amino acid sequence of SEQ ID NO: 12791; and (c) CDR-H3 comprising the amino acid sequence of SEQ ID NO: 15291; and (d) a VL comprising the amino acid sequence of SEQ ID NO: 19041.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 11541; (b) CDR-L2 comprising the amino acid sequence of SEQ ID NO: 14041; and (c) CDR-L3 comprising the amino acid sequence of SEQ ID NO: 16541; and a VH comprising the amino acid sequence of SEQ ID NO: 17791.

In one aspect, the present disclosure provides an antibody or antigen-binding fragment thereof comprising the CDRs: CDR-H3 comprising the amino acid sequence of SEQ ID NO: 15291; and CDR-L3 comprising the amino acid sequence of SEQ ID NO: 16541.

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 11541; (b) CDR-L2 comprising the amino acid sequence of SEQ ID NO: 14041 and (c) CDR-L3 comprising the amino acid sequence of SEQ ID NO: 16541. In one aspect, the disclosure provides an antibody or antigen-binding fragment thereof comprising at least one, at least two, or all three VH CDR sequences selected from (a) CDR-H1 comprising the amino acid sequence of SEQ ID NO: 10291; (b) CDR-H2 comprising the amino acid sequence of SEQ ID NO: 12791 and (c) CDR-H3 comprising the amino acid sequence of SEQ ID NO: 15291.

In one aspect, the disclosure provides an antibody or antigen-binding fragment thereof comprising the CDRs: (a) CDR-H1 comprising the amino acid sequence of SEQ ID NO: 10291; (b) CDR-H2 comprising the amino acid sequence of SEQ ID NO: 12791; (c) CDR-H3 comprising the amino acid sequence of SEQ ID NO: 15291; (d) CDR-L1 comprising the amino acid sequence of SEQ ID NO: 11541; (e) CDR-L2 comprising the amino acid sequence of SEQ ID NO: 14041; and (f) CDR-L3 comprising the amino acid sequence of SEQ ID NO: 16541.

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 17791. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 17791. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 17791, including post-translational modifications of that sequence. In a particular embodiment, the VH comprises one, two or three CDRs selected from: (a) CDR-H1 comprising the amino acid sequence of SEQ ID NO: 10291, (b) CDR-H2 comprising the amino acid sequence of SEQ ID NO: 12791, and (c) CDR-H3 comprising the amino acid sequence of SEQ ID NO: 15291.

In one aspect, an antibody or antigen-binding fragment thereof is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 19041. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 19041. In some embodiments, the substitutions, insertions, or deletions occur in regions outside the CDRs (e.g., in the FRs). Optionally, the antibody or antigen-binding fragment thereof comprises the VL sequence of SEQ ID NO: 19041, including post-translational modifications of that sequence. In a particular embodiment, the VL comprises one, two or three CDRs selected from (a) CDR-L1 comprising the amino acid sequence of SEQ ID NO: 11541; (b) CDR-L2 comprising the amino acid sequence of SEQ ID NO: 14041; and (c) CDR-L3 comprising the amino acid sequence of SEQ ID NO: 16541.

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody comprises a VH comprising the amino acid sequence of SEQ ID NO: 17791, and a VL sequence in SEQ ID NO: 19041, including post-translational modifications of those sequences.

### TOTCOVID00124

In one aspect, the present disclosure provides an antibody or antigen-binding fragment thereof comprises one or more variable regions selected from the group consisting of (a) VH comprising the amino acid sequence of SEQ ID NO: 17614, (b) VL comprising the amino acid sequence of SEQ ID NO: 18864, and (c) a combination thereof.

In one aspect, the present disclosure provides an antibody or antigen-binding fragment thereof comprising at least one, two, three, four, five, or six CDRs selected from (a) CDR-H1 comprising the amino acid sequence of SEQ ID NO: 10114; (b) CDR-H2 comprising the amino acid sequence of SEQ ID NO: 12614; (c) CDR-H3 comprising the amino acid sequence of SEQ ID NO: 15114; (d) CDR-L1 comprising the amino acid sequence of SEQ ID NO: 11364; (e) CDR-L2 comprising the amino acid sequence of SEQ ID NO: 13864; and (f) CDR-L3 comprising the amino acid sequence of SEQ ID NO: 16364.

In one aspect, the present disclosure provides an antibody or antigen-binding fragment thereof comprising at least one, at least two, or all three VH CDR sequences selected from (a) CDR-H1 comprising the amino acid sequence of SEQ ID NO: 10114; (b) CDR-H2 comprising the amino acid sequence of SEQ ID NO: 12614; and (c) CDR-H3 comprising the amino acid sequence of SEQ ID NO: 15114; and (d) a VL comprising the amino acid sequence of SEQ ID NO: 18864.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 11364; (b) CDR-L2 comprising the amino acid sequence of SEQ ID NO: 13864; and (c) CDR-L3 comprising the amino acid sequence of SEQ ID NO: 16364; and a VH comprising the amino acid sequence of SEQ ID NO: 17614.

In one aspect, the present disclosure provides an antibody or antigen-binding fragment thereof comprising the CDRs: CDR-H3 comprising the amino acid sequence of SEQ ID NO: 15114; and CDR-L3 comprising the amino acid sequence of SEQ ID NO: 16364.

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 11364; (b) CDR-L2 comprising the amino acid sequence of SEQ ID NO: 13864 and (c) CDR-L3 comprising the amino acid sequence of SEQ ID NO: 16364. In one aspect, the disclosure provides an antibody or antigen-binding fragment thereof comprising at least one, at least two, or all three VH CDR sequences selected from (a) CDR-H1 comprising the amino acid sequence of SEQ ID NO: 10114; (b) CDR-H2 comprising the amino acid sequence of SEQ ID NO: 12614 and (c) CDR-H3 comprising the amino acid sequence of SEQ ID NO: 15114.

In one aspect, the disclosure provides an antibody or antigen-binding fragment thereof comprising the CDRs: (a) CDR-H1 comprising the amino acid sequence of SEQ ID NO: 10114; (b) CDR-H2 comprising the amino acid sequence of SEQ ID NO: 12614; (c) CDR-H3 comprising the amino acid sequence of SEQ ID NO: 15114; (d) CDR-L1 comprising the amino acid sequence of SEQ ID NO: 11364; (e) CDR-L2 comprising the amino acid sequence of SEQ ID NO: 13864; and (f) CDR-L3 comprising the amino acid sequence of SEQ ID NO: 16364.

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 17614. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 17614. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 17614, including post-translational modifications of that sequence. In a particular embodiment, the VH comprises one, two or three CDRs selected from: (a) CDR-H1 comprising the amino acid sequence of SEQ ID NO: 10114, (b) CDR-H2 comprising the amino acid sequence of SEQ ID NO: 12614, and (c) CDR-H3 comprising the amino acid sequence of SEQ ID NO: 15114.

In one aspect, an antibody or antigen-binding fragment thereof is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 18864. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 18864. In some embodiments, the substitutions, insertions, or deletions occur in regions outside the CDRs (e.g., in the FRs). Optionally, the antibody or antigen-binding fragment thereof comprises the VL sequence of SEQ ID NO: 18864, including post-translational modifications of that sequence. In a particular embodiment, the VL comprises one, two or three CDRs selected from (a) CDR-L1 comprising the amino acid sequence of SEQ ID NO: 11364; (b) CDR-L2 comprising the amino acid sequence of SEQ ID NO: 13864; and (c) CDR-L3 comprising the amino acid sequence of SEQ ID NO: 16364.

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody comprises a VH comprising the amino acid sequence of SEQ ID NO: 17614, and a VL sequence in SEQ ID NO: 18864, including post-translational modifications of those sequences.

### TOTCOVID00450

In one aspect, the present disclosure provides an antibody or antigen-binding fragment thereof comprises one or more variable regions selected from the group consisting of (a) VH comprising the amino acid sequence of SEQ ID NO: 17894, (b) VL comprising the amino acid sequence of SEQ ID NO: 19144, and (c) a combination thereof.

In one aspect, the present disclosure provides an antibody or antigen-binding fragment thereof comprising at least one, two, three, four, five, or six CDRs selected from (a) CDR-H1 comprising the amino acid sequence of SEQ ID NO: 10394; (b) CDR-H2 comprising the amino acid sequence of SEQ ID NO: 12894; (c) CDR-H3 comprising the amino acid sequence of SEQ ID NO: 15394; (d) CDR-L1 comprising the amino acid sequence of SEQ ID NO: 11644; (e) CDR-L2 comprising the amino acid sequence of SEQ ID NO: 14144; and (f) CDR-L3 comprising the amino acid sequence of SEQ ID NO: 16644.

In one aspect, the present disclosure provides an antibody or antigen-binding fragment thereof comprising at least one, at least two, or all three VH CDR sequences selected from (a) CDR-H1 comprising the amino acid sequence of SEQ ID NO: 10394; (b) CDR-H2 comprising the amino acid sequence of SEQ ID NO: 12894; and (c) CDR-H3 comprising the amino acid sequence of SEQ ID NO: 15394; and (d) a VL comprising the amino acid sequence of SEQ ID NO: 19144.

In one aspect, the present disclosure provides an antibody or antigen-binding fragment thereof comprising at least one, at least two, or all three VL CDR sequences selected from (a) CDR-L1 comprising the amino acid sequence of SEQ ID NO: 11644; (b) CDR-L2 comprising the amino acid sequence of SEQ ID NO: 14144; and (c) CDR-L3 comprising the amino acid sequence of SEQ ID NO: 16644; and a VH comprising the amino acid sequence of SEQ ID NO: 17894.

In one aspect, the present disclosure provides an antibody or antigen-binding fragment thereof comprising the CDRs: CDR-H3 comprising the amino acid sequence of SEQ ID NO: 15394; and CDR-L3 comprising the amino acid sequence of SEQ ID NO: 16644.

In one aspect, the disclosure herein provides an antibody or antigen-binding fragment thereof comprising at least one, at least two, or all three VL CDR sequences selected from (a) CDR-L1 comprising the amino acid sequence of SEQ ID NO: 11644; (b) CDR-L2 comprising the amino acid sequence of SEQ ID NO: 14144 and (c) CDR-L3 comprising the amino acid sequence of SEQ ID NO: 16644. In one aspect, the disclosure provides an antibody or antigen-binding fragment thereof comprising at least one, at least two, or all three VH CDR sequences selected from (a) CDR-H1 comprising the amino acid sequence of SEQ ID NO: 10394; (b) CDR-H2 comprising the amino acid sequence of SEQ ID NO: 12894 and (c) CDR-H3 comprising the amino acid sequence of SEQ ID NO: 15394.

In one aspect, the disclosure provides an antibody or antigen-binding fragment thereof comprising the CDRs: (a) CDR-H1 comprising the amino acid sequence of SEQ ID NO: 10394; (b) CDR-H2 comprising the amino acid sequence of SEQ ID NO: 12894; (c) CDR-H3 comprising the amino acid sequence of SEQ ID NO: 15394; (d) CDR-L1 comprising the amino acid sequence of SEQ ID NO: 11644; (e) CDR-L2 comprising the amino acid sequence of SEQ ID NO: 14144; and (f) CDR-L3 comprising the amino acid sequence of SEQ ID NO: 16644.

In one aspect, an antibody or antigen-binding fragment thereof comprises a VH sequence having at least 90%, 91%, 92%, 93%, 94%, 95%, 96%, 97%, 98%, 99%, or 100% sequence identity to the amino acid sequence of SEQ ID NO: 17894. In some embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 17894. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 17894, including post-translational modifications of that sequence. In a particular embodiment, the VH comprises one, two or three CDRs selected from: (a) CDR-H1 comprising the amino acid sequence of SEQ ID NO: 10394, (b) CDR-H2 comprising the amino acid sequence of SEQ ID NO: 12894, and (c) CDR-H3 comprising the amino acid sequence of SEQ ID NO: 15394.

In one aspect, an antibody or antigen-binding fragment thereof is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO: 19144. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 19144. In some embodiments, the substitutions, insertions, or deletions occur in regions outside the CDRs (e.g., in the FRs). Optionally, the antibody or antigen-binding fragment thereof comprises the VL sequence of SEQ ID NO: 19144, including post-translational modifications of that sequence. In a particular embodiment, the VL comprises one, two or three CDRs selected from (a) CDR-L1 comprising the amino acid sequence of SEQ ID NO: 11644; (b) CDR-L2 comprising the amino acid sequence of SEQ ID NO: 14144; and (c) CDR-L3 comprising the amino acid sequence of SEQ ID NO: 16644.

In one aspect, an antibody or antigen-binding fragment thereof is provided, wherein the antibody or antigen-binding fragment thereof comprises a VH as in any of the embodiments provided above, and a VL as in any of the embodiments provided above. In some embodiments, the antibody comprises a VH comprising the amino acid sequence of SEQ ID NO: 17894, and a VL sequence in SEQ ID NO: 19144, including post-translational modifications of those sequences.

### Variants and Modifications

In another aspect, provided herein are variants of antibodies or antigen-binding fragments thereof.

### Substitution, Insertion, and Deletion Variants

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of techniques well known in the art. For example, it may be desirable to improve the binding affinity and/or other biological properties of the antibody amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, antibody variants or antigen-binding fragment thereof having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved ADCC or CDC function.

| Original Residue | Exemplary Conserved Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Hydrophobic amino acids include: Norleucine, Met, Ala, Val, Leu, and Ile. Neutral hydrophilic amino acids include: Cys, Ser, Thr, Asn, and Gln. Acidic amino acids include: Asp and Glu. Basic amino acids include: His, Lys, and Arg. Amino acids with residues that influence chain orientation include: Gly and Pro. Aromatic amino acids include: Trp, Tyr, and Phe.

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots” or SDRs. In some embodiments of the variant V_{H} and V_{L} sequences, each CDR either is unaltered, or contains no more than one, two or three amino acid substitutions.

Alterations (e.g., substitutions) may be made in CDRs, e.g., to improve antibody affinity. Such alterations may be made in CDR encoding codons with a high mutation rate during somatic maturation (see, e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (see, e.g., Hoogenboom et al. in Methods Mol. Biol. 178:1-37 (2001)). CDR residues involved in antigen-binding may be specifically identified, e.g., using alanine scanning mutagenesis or modeling (see, e.g., Cunningham and Wells, Science 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to a polypeptide which increases serum half-life of the antibody, for example, at the N-terminus or C-terminus. The term “epitope tagged” refers to the antibody fused to an epitope tag. The epitope tag polypeptide has enough residues to provide an epitope against which an antibody there against can be made, yet is short enough such that it does not interfere with activity of the antibody. The epitope tag preferably is sufficiently unique so that the antibody there against does not substantially cross-react with other epitopes. Suitable tag polypeptides generally have at least 6 amino acid residues and usually between about 8-50 amino acid residues (preferably between about 9-30 residues). Examples include the flu HA tag polypeptide and its antibody 12CA5 (Field et al., Mal. Cell. Biol. 8:2159-2165 (1988)); the c-myc tag and the 8F9, 3C7, 6E10, G4, B7 and 9E10 antibodies thereto (Evan et al., Mal. Cell. Biol. 5(12):3610-3616 (1985)); and the Herpes Simplex virus glycoprotein D (gD) tag and its antibody (Paborsky et al., Protein Engineering 3(6):547-553 (1990)). Other exemplary tags are a poly-histidine sequence, generally around six histidine residues, that permits isolation of a compound so labeled using nickel chelation. Other labels and tags, such as the FLAG® tag (Eastman Kodak, Rochester, N.Y.), well known and routinely used in the art, are embraced by the invention.

Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

### Glycosylation Variants

In some embodiments, the antibodies are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn297 of the C_{H}2 domain of the Fc region (see, e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennary oligosaccharide structure. Modifications of the oligosaccharide in an antibody can be made, for example, to create antibody variants with certain improved properties. Antibody glycosylation variants can have improved ADCC and/or CDC function.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (see, e.g., WO 08/077546). Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants can have improved ADCC function (see, e.g. Okazaki et al. J. Mol. Biol. 336:1239-1249 (2004); and Yamane-Ohnuki et al. Biotech. Bioeng. 87: 614 (2004)). Cell lines, e.g., knockout cell lines and methods of their use can be used to produce defucosylated antibodies, e.g., Lec13 CHO cells deficient in protein fucosylation and alpha-1,6-fucosyltransferase gene (FUT8) knockout CHO cells (see, e.g., Ripka et al. Arch. Biochem. Biophys. 249:533-545 (1986); Yamane-Ohnuki et al. Biotech. Bioeng. 87: 614 (2004); Kanda, Y. et al., Biotechnol. Bioeng. 94(4):680-688 (2006). Other antibody glycosylation variants are also contemplated.

In still another embodiment, the glycosylation of an antibody is modified. For example, an aglycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Conservative substitutions involve replacing an amino acid with another member of its class. Non-conservative substitutions involve replacing a member of one of these classes with a member of another class.

Accordingly, an antibody or antigen-binding fragment thereof of the present disclosure can be produced by a host cell with one or more of exogenous and/or high endogenous glycosyltransferase activities. Genes with glycosyltransferase activity include β(1,4)-N-acetylglucosaminyltransferase III (GnTII), α-mannosidase II (ManII), β(1,4)-galactosyltransferase (GalT), β(1,2)-N-acetylglucosaminyltransferase I (GnTI), and β(1,2)-N-acetylglucosaminyltransferase II (GnTII). The glycotranferases can comprise a fusion comprising a Golgi localization domain (see, e.g., Lifely et al., Glycobiology 318:813-22 (1995); Schachter, Biochem. Cell Biol. 64:163-81 (1986)). In some embodiments, an antibody can be expressed in a host cell comprising a disrupted or deactivated glycosyltransferase gene. Accordingly, in some embodiments, the present disclosure is directed to a host cell comprising (a) an isolated nucleic acid comprising a sequence encoding a polypeptide having a glycosyltransferase activity; and (b) an isolated polynucleotide encoding an antibody or antigen-binding fragment thereof of the present disclosure. In a particular embodiment, the modified antibody produced by the host cell has an IgG constant region or a fragment thereof comprising the Fc region. In another particular embodiment the antibody is a humanized antibody or a fragment thereof comprising an Fc region.

Antibodies with altered glycosylation produced by the host cells of the invention can exhibit increased Fc receptor binding affinity (e.g., increased binding to a Fcγ activating receptor, such as the FcγRIIIa receptor) and/or increased effector function. The increased effector function can be an increase in one or more of the following: increased antibody-dependent cellular cytotoxicity, increased antibody-dependent cellular phagocytosis (ADCP), increased cytokine secretion, increased immune-complex-mediated antigen uptake by antigen-presenting cells, increased Fc-mediated cellular cytotoxicity, increased binding to NK cells, increased binding to macrophages, increased binding to polymorphonuclear cells (PMNs), increased binding to monocytes, increased crosslinking of target-bound antibodies, increased direct signaling inducing apoptosis, increased dendritic cell maturation, and increased T cell priming. Accordingly, in one aspect, the present invention provides glycoforms of an antibody having increased effector function as compared to the antibody that has not been glycoengineered. (see, e.g., Tang et al., J. Immunol. 179: 2815-2823 (2007)).

The present disclosure is also directed to a method for producing an antibody or antigen-binding fragment thereof, described herein having modified oligosaccharides, comprising (a) culturing a host cell engineered to express at least one nucleic acid encoding a polypeptide having glycosyltransferase activity under conditions which permit the production of an antibody according to the present disclosure, wherein said polypeptide having glycosyltransferase activity is expressed in an amount sufficient to modify the oligosaccharides in the Fc region of said antibody produced by said host cell; and (b) isolating said antibody. In another embodiment, there are two polypeptides having glycosyltransferase activity. The antibodies or antigen-binding fragment thereof produced by the methods of the present invention can have increased Fc receptor binding affinity and/or increased effector function.

In some embodiments, the percentage of bisected N-linked oligosaccharides in the Fc region of the antibody is at least about 10% to about 100%, specifically at least about 50%, more specifically, at least about 60%, at least about 70%, at least about 80%, or at least about 90-95% of the total oligosaccharides. In yet another embodiment, the antibody produced by the methods of the invention has an increased proportion of nonfucosylated oligosaccharides in the Fc region as a result of the modification of its oligosaccharides by the methods of the present invention. In some embodiments, the percentage of nonfucosylated oligosaccharides is at least about 20% to about 100%, specifically at least about 50%, at least about 60% to about 70%, and more specifically, at least about 75%. The nonfucosylated oligosaccharides may be of the hybrid or complex type. In yet another embodiment, the antibody or antigen-binding fragment thereof produced by the methods of the invention has an increased proportion of bisected oligosaccharides in the Fc region as a result of the modification of its oligosaccharides by the methods of the present invention. In some embodiments, the percentage of bisected oligosaccharides is at least about 20% to about 100%, specifically at least about 50%, at least about 60% to about 70%, and more specifically, at least about 75%.

In another embodiment, the present invention is directed to an antibody or antigen-binding fragment thereof engineered to have increased effector function and/or increased Fc receptor binding affinity, produced by the methods of the disclosure. In some embodiments, the antibody is an intact antibody. In some embodiments, the antibody is an antibody fragment containing the Fc region, or a fusion protein that includes a region equivalent to the Fc region of an immunoglobulin.

In one aspect, the present disclosure provides host cell expression systems for the generation of the antibodies or antigen-binding fragment thereof of the present disclosure having modified glycosylation patterns. In particular, the present disclosure provides host cell systems for the generation of glycoforms of the antibodies or antigen-binding fragment thereof, disclosed herein, having an improved therapeutic value. Therefore, the present disclosure provides host cell expression systems selected or engineered to express a polypeptide having a glycosyltransferase activity. Generally, any type of cultured cell line, including the cell lines discussed above, can be used as a background to engineer the host cell lines of the present invention. In some embodiments, CHO cells, BHK cells, NSO cells, SP2/0 cells, YO myeloma cells, P3X63 mouse myeloma cells, PER cells, PER.C6 cells or hybridoma cells, other mammalian cells, yeast cells, insect cells, or plant cells are used as the background cell line to generate the engineered host cells of the invention.

The host cells which contain the coding sequence of an antibody or antigen-binding fragment thereof of the invention and which express the biologically active gene products may be identified by at least four general approaches: (a) DNA-DNA or DNA-RNA hybridization; (b) the presence or absence of “marker” gene functions; (c) assessing the level of transcription as measured by the expression of the respective mRNA transcripts in the host cell; and (d) detection of the gene product as measured by immunoassay or by its biological activity.

### Cysteine Engineered Antibody Variants

In some embodiments, it may be desirable to create cysteine engineered antibodies or antigen-binding fragments thereof, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker-drug moieties, to create an immunoconjugate. In some embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described.

Any cysteine residue not involved in maintaining the proper conformation of the monoclonal, human, humanized, or variant antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

### Fc Region Variants

Mutation of residues within Fc receptor binding sites can result in altered effector function, such as altered ADCC, CDC activity, and/or altered half-life. Mutations include, for example, insertion, deletion, and/or substitution of one or more residues as described in more detail above, including substitution with alanine, a conservative substitution, a non-conservative substitution, and/or replacement with a corresponding amino acid residue at the same position from a different IgG subclass (e.g., replacing an IgG1 residue with a corresponding IgG2 residue at that position).

An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

Previous studies mapped the binding site on human and murine IgG for FcγR primarily to the lower hinge region composed of IgG residues 233-239. Other studies proposed additional broad segments, e.g., Gly316-Lys338 for human Fc gamma receptor I, Lys274-Arg301 and Tyr407Arg416 for human Fc gamma receptor III, or found a few specific residues outside the lower hinge, e.g., Asn297 and Glu318 for murine IgG2b interacting with murine Fc gamma receptor II. The report of the 3.2-A crystal structure of the human IgG Fc fragment with human Fc gamma receptor IIIA delineated IgG1 residues Leu234-Ser239, Asp265-Glu269, Asn297-Thr299, and Ala327-Ile332 as involved in binding to Fc receptor γIIIA. It has been suggested based on crystal structure that in addition to the lower hinge (Leu234-Gly237), residues in IgG C_{H}2 domain loops FG (residues 326-330) and BC (residues 265-271) might play a role in binding to Fc gamma receptor IIA. See Shields et al., J. Biol. Chem. 276(9):6591-6604 (2001). Shields et al. reported that IgG1 residues involved in binding to all human Fc receptors are located in the C_{H}2 domain proximal to the hinge and fall into two categories as follows: 1) positions that may interact directly with all FcR include Leu234-Pro238, Ala327, and Pro329 (and possibly Asp265); 2) positions that influence carbohydrate nature or position include Asp265 and Asn297.

In some embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the effect of one or more Fc amino acid modifications on CDC and/or ADCC activities. For example, Fc receptor (FcR) binding assays can be conducted to ensure that the antibody lacks FcγR binding (hence likely lacking ADCC activity), but retains FcRn binding ability.

Fc Variants with Altered Binding to an Fc Gamma Receptor

In some instances, an Fc variant exhibits altered affinity for one or more Fc gamma receptors (FcγR). For example, an Fc variant exhibits increased affinity for one or more Fc gamma receptors (FcγR), decreased affinity for one or more Fc gamma receptors (FcγR), or a combination thereof. In one instance, an Fc variant exhibits increased ADCC activity. In yet another example, the Fc region is modified to increase the ability of the antibody to mediate antibody dependent cellular cytotoxicity (ADCC). The binding sites on human IgG1 for Fc gamma RI (FcγRI), Fc gamma RH (FcγRII), Fc gamma RIII (FcγRIIII), and FcRn have been mapped and variants with altered binding have been described. Non-limiting examples of such modifications are described in, for example, U.S. Pat. No. 6,737,056; PCT Publication WO 00/42072 by Presta; Shields, R. L. et al. (2001) J. Biol. Chem. 276:6591-6604; U.S. Pat. No. 7,332,581, etc. In some embodiments, the constant region of the antibodies disclosed herein is replaced with an IGHG1.

Armour et al. (Mol Immunol. 2003; 40(9):585-93) identified IgG1 variants which react with the activating receptor, FcγRIIa, at least 10-fold less efficiently than wildtype IgG1, but whose binding to the inhibitory receptor, FcγRIIb, is only four-fold reduced. Mutations were made in the region of amino acids 233-236 and/or at amino acid positions 327, 330 and 331. See also WO 99/58572.

Non-limiting examples of in vitro assays to assess ADCC activity of a molecule of interest are described, for example, in U.S. Pat. Nos. 5,500,362 and 5,821,337. Alternatively, non-radioactive assays methods may be employed (e.g., ACTI™ and CYTOTOX 96® non-radioactive cytotoxicity assays). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model (see, e.g., Clynes et al., Proc. Nat'l Acad. Sci. USA 95:652-656 (1998)).

Fc Variants with Decreased C1q Binding

In another instance, an Fc variant exhibits reduced C1q binding. C1q binding assays may also be carried out to confirm that the antibody is able or unable bind C1q and, hence, contains or lacks CDC activity (Idusogie et al., J. Immunol. 164: 4178-4184 (2000)). To assess complement activation, a CDC assay may be performed (see, e.g., Gazzano-Santoro et al., J. Immunol. Methods 202:163 (1996); Cragg, M. S. et al., Blood 101:1045-1052 (2003); and Cragg et al., Blood 103:2738-2743 (2004)).

In another example, one or more amino acids can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al. In another example, one or more amino acid residues are altered to thereby alter the ability of the antibody to fix complement. This approach is described further in PCT Publication WO 94/29351 by Bodmer et al. In one instance, an Fc variant provided herein can contain a mutation at amino acid position 329, 331, and/or 322 (using Kabat numbering), and exhibits reduced Clq binding and/or CDC activity. In some instances, Clq binding activity and/or CDC activity of an antibody can be reduced by mutating amino acid residue 318, 320, and/or 322 (using Kabat numbering) of a heavy chain; replacing residue 297 (Asn) may result in removal of lytic activity of an antibody.

Cytophilic activity of IgG1 is a property of its heavy chain C_{H}2 domain. In one instance, where an Fc variant is an IgG, amino acid residues 234-237 are maintained as wild type to preserve cytophilic activity of the molecule. An IgG2 antibody containing the entire ELLGGP sequence (residues 233-238) may, in some instances, be more active than wild-type IgG1.

In some instances, Clq binding activity and/or lytic activity of an IgG1 antibody can be reduced by mutating amino acid residue Pro331 to Ser. In other instances, Clq binding activity and/or lytic activity of an IgG4 antibody can be reduced by mutating amino acid residue Pro for Ser331 (Xu et al., J Biol Chem. 1994; 269(5):3469-74).

Fc Variants with Interchain Disulfide Bonds or Dual Fc Regions

In yet another embodiment, it may be desirable to modify the antibody of the invention with respect to effector function, so as to enhance the therapeutic effectiveness of the antibody. For example, one or more cysteine residue(s) may be introduced in the Fc region, thereby allowing interchain disulfide bond formation in this region. The homodimeric antibody thus generated may have improved internalization capability, increased complement-mediated cell killing, and/or antibody-dependent cellular cytotoxicity (ADCC). See Caron et al., J. Exp Med. 176:1191-1195 (1992) and Shapes, B. J. Immunol. 148:2918-2922 (1992). Alternatively, an antibody can be engineered which has dual Fc regions and may thereby have enhanced complement lysis and/or ADCC capabilities. See, Stevenson et al., Anti-Cancer Drug Design 3: 219-230 (1989).

Fc Variants with Increased FcRn Binding and In Vivo Half-Life

Fc region variants with altered binding affinity for the neonatal receptor (FcRn) are also contemplated herein. Fc region variants with improved affinity for FcRn are anticipated to have longer serum half-lives, and such variants are useful in methods of treating subjects where long half-life of the administered polypeptide is desired, e.g., to treat a chronic infection. Fc region variants with decreased FcRn binding affinity, on the contrary, are expected to have shorter half-lives, and such variants may be administered to a subject where a shortened circulation time may be preferred, e.g. for in vivo diagnostic imaging or for antibodies which have toxic side effects when left circulating in the blood stream for extended periods, etc. Determination of FcRn binding and in vivo clearance/half-life can be performed using methods known in the art (see, e.g., Petkova, S. B. et al., Int'l. Immunol. 18(12):1759-1769 (2006)).

Schuurman et al., Mol Immunol. 2001; 38(1):1-8, incorporated by reference herein in its entirety, report that mutating one of the hinge cysteines involved in the inter-heavy chain bond formation, Cys226, to serine resulted in a more stable inter-heavy chain linkage. Mutating the IgG4 hinge sequence Cys-Pro-Ser-Cys to the IgG1 hinge sequence Cys-Pro-Pro-Cys also markedly stabilizes the covalent interaction between the heavy chains. Angal et al., Mol Immunol. 1993; 30(1):105-8, incorporated by reference herein in its entirety, report that mutating the serine at amino acid position 241 in IgG4 to praline (found at that position in IgG1 and IgG2) led to the production of a homogeneous antibody, as well as extending serum half-life and improving tissue distribution compared to the original chimeric IgG4. Other such examples of Fc region variants are also contemplated (see, e.g., Duncan & Winter, Nature 322:738-40 (1988); Chan C A and Carter P J (2010) Nature Rev. Immunol. 10:301-316); and Shields et al., J. Biol. Chem. 9(2): 6591-6604 (2001).

### Antibody Composition and Structural Conformation

In various embodiments of the invention, the resulting antibody polypeptides may take on a range of compositions or structural conformations. Included herein are bispecific antibodies, multispecific antibodies, multivalent antibodies, chimeric antibodies, human antibodies, humanized antibodies, monoclonal antibodies, deimmunized antibodies, or a combination thereof.

### Bispecific and Multispecific Antibodies

In some embodiments, it may be desirable to generate multispecific (e.g. bispecific) monoclonal antibody including monoclonal, human, humanized, or variant antibodies having binding specificities for at least two different epitopes. In some embodiments, the antibodies disclosed herein are multispecific. Exemplary bispecific antibodies may bind to two different epitopes of an antigen (e.g., SARS-CoV-2 associated antigen). Alternatively, an antigen-binding region may be combined with a region which binds to a triggering molecule on a leukocyte such as a T-cell receptor molecule (e.g., CD2 or CD3), or Fe receptors for IgG (FcγR), such as FcγRI (CD64), FcγRII (CD32) and FcγRIII (CD16) so as to focus cellular defense mechanisms to the antigen-expressing cell. Bispecific antibodies can be prepared as full length antibodies or antibody fragments (e.g., F(ab′)2 bispecific antibodies).

According to another approach for making bispecific antibodies, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are contemplated, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes. In yet a further embodiment, Fab′-SH fragments directly recovered from E. coli can be chemically coupled in vitro to form bispecific antibodies. (Shalaby et al., J. Exp. Med. 175:217-225 (1992))

Exemplary techniques for making multispecific antibodies include recombinant co-expression of two immunoglobulin heavy chain-light chain pairs having different specificities, engineering electrostatic steering effects for making antibody Fc-heterodimeric molecules, cross-linking two or more antibodies or fragments, using leucine zippers to produce bi-specific antibodies, using “diabody” technology for making bispecific antibody fragments, using single-chain Fv (sFv) dimers, preparing trispecific antibodies, and “knob-in-hole” engineering (see, e.g., Milstein and Cuello, Nature 305: 537 (1983); Traunecker et al., EMBO J. 10: 3655 (1991); U.S. Pat. Nos. 4,676,980 and 5,731,168; Brennan et al., Science, 229: 81 (1985); Kostelny et al., J. Immunol. 148(5):1547-1553 (1992); Hollinger et al., Proc. Natl. Acad. Sci. USA 90:6444-6448 (1993); Gruber et al., J. Immunol. 152:5368 (1994)); and Tutt et al. J. Immunol. 147: 60 (1991)). Engineered antibodies with three or more functional antigen-binding sites are also contemplated.

### Chimeric Antibodies

In some embodiments, an antibody provided herein is a chimeric. A chimeric antibody is an antibody in which a portion of the heavy and/or light chain is derived from a particular source or species, while the remainder of the heavy and/or light chain is derived from a different source or species. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof. For details, see, for example, Jones et al., Nature 321: 522-525 (1986); Reichmann et al., Nature 332: 323-329 (1988); Presta, Curr. Op. Struct. Biol. 2: 593-596 (1992); and Morrison et al., Proc. Natl. Acad. Sci. USA 81:6851-6855 (1984).

### Human Antibodies

In some embodiments, an antibody provided herein is a human antibody. Human antibodies can be produced using various techniques known in the art (see, e.g., van Dijk and van de Winkel, Curr. Opin. Pharmacol. 5: 368-74 (2001); and Lonberg, Curr. Opin. Immunol. 20:450-459 (2008)). A human antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies may be prepared by administering an immunogen (e.g., a SARS-CoV-2 antigen) to a transgenic animal that has been modified to produce intact human antibodies or intact antibodies with human variable regions in response to antigenic challenge. (see, e.g., Lonberg, Nat. Biotech. 23:1117-1125 (2005)). Human variable regions from intact antibodies generated by such animals may be further modified, e.g., by combining with a different human constant region.

Human antibodies can also be made by hybridoma-based methods. For example, human antibodies can be produced from human myeloma and mouse-human heteromyeloma cell lines, using human B-cell hybridoma technology, and other methods (see, e.g., Kozbor J. Immunol. 133: 3001 (1984); Brodeur et al., Monoclonal Antibody Production Techniques and Applications, pp. 51-63 (1987); Boerner et al., J. Immunol. 147: 86 (1991); Li et al., Proc. Natl. Acad. USA 103:3557-3562 (2006); Ni, Xiandai Mianyixue, 26(4):265-268 (2006); Vollmers and Brandlein, Histology and Histopathology 20(3):927-937 (2005); and Vollmers and Brandlein, Methods and Findings in Experimental and Clinical Pharmacology 27(3):185-91 (2005)). Human antibodies may also be generated by isolating Fv clone variable domain sequences selected from human-derived phage display libraries. Such variable domain sequences may then be combined with a desired human constant domain.

### Recombinant Human Antibodies

The term “recombinant human antibody”, as used herein, includes all human antibodies that are prepared, expressed, created or isolated by recombinant means, such as: (a) antibodies isolated from an animal (e.g., a mouse) that is transgenic or transchromosomal for human immunoglobulin genes or a hybridoma prepared therefrom (described further below); (b) antibodies isolated from a host cell transformed to express the human antibody, e.g., from a transfectoma; (c) antibodies isolated from a recombinant, combinatorial human antibody library; and (d) antibodies prepared, expressed, created or isolated by any other means that involve splicing of human immunoglobulin gene sequences to other DNA sequences. Such recombinant human antibodies have variable regions in which the framework and CDR regions are derived from reconstructed immunoglobulin consensus sequences, disclosed herein. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human immunoglobulin V_{H} and V_{L} sequences, may not naturally exist within the human antibody germline repertoire in vivo.

### Humanized Antibodies

In some embodiments, an antibody provided herein is a humanized antibody. In one embodiment, a humanized antibody is an antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. See, e.g., Almagro and Fransson, Front. Biosci. 13:1619-1633 (2008); Riechmann et al., Nature 332:323-329 (1988); Queen et al., Proc. Nat'l Acad. Sci. USA 86:10029-10033 (1989); Kashmiri et al., Methods 36:25-34 (2005); Padlan, Mol. Immunol. 28:489-498 (1991); Dall'Acqua et al., Methods 36:43-60 (2005); Osbourn et al., Methods 36:61-68 (2005); and Klimka et al., Br. J. Cancer 83:252-260 (2000).

A non-human antibody can be humanized to reduce immunogenicity to humans, while retaining the specificity and affinity of the parental non-human antibody. A humanized antibody can comprise one or more variable domains comprising one or more CDRs, or portions thereof, derived from a non-human antibody. A humanized antibody can comprise one or more variable domains comprising one or more FRs, or portions thereof, derived from human antibody sequences. A humanized antibody can optionally comprise at least a portion of a human constant region. In some embodiments, one or more FR residues in a humanized antibody are substituted with corresponding residues from a non-human antibody (e.g., the antibody from which the CDR residues are derived), e.g., to restore or improve antibody specificity or affinity.

Human framework regions that may be used for humanization include but are not limited to: framework regions selected using a “best-fit” method; framework regions derived from the consensus sequence of human antibodies of a particular subgroup of light or heavy chain variable regions; human mature (somatically mutated) framework regions or human germline framework regions; and framework regions derived from screening FR libraries (see, e.g., Sims et al. J. Immunol. 151:2296 (1993); Carter et al. Proc. Natl. Acad. Sci. USA 89:4285 (1992); Presta et al. J. Immunol. 151:2623 (1993); Baca et al. J. Biol. Chem. 272:10678-10684 (1997); and Rosok et al. J. Biol. Chem. 271:22611-22618 (1996)).

### Monoclonal Antibodies

A monoclonal antibody is obtained from a population of substantially homogeneous antibodies, i.e., the individual antibodies comprising the population are identical except for possible naturally occurring mutations that may be present in minor amounts. In some embodiments, the antibodies of the present disclosure are monoclonal. In a preferred embodiment, monoclonal antibodies may be made using recombinant DNA methods, or in an alternative embodiment, by the hybridoma method first described by Kohler et al., Nature, 256:495 (1975).

### Deimmunized Antibodies

An antibody or an antigen-binding fragment thereof described herein can be optionally assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized, yet the antibody retains its binding activity. Analysis of immunogenicity and T-cell epitopes present in the antibodies and antigen-binding fragments described herein can be carried out via the use of software and specific databases known in the art. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7 and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

### Engineered and Modified Antibodies

An antibody according to at least some embodiments of the invention further can be prepared using an antibody having one or more of the V_{H} and/or V_{L} sequences derived from an antibody or antigen-binding fragment thereof, disclosed herein, starting material to engineer a modified antibody, which modified antibody may have altered properties from the starting antibody. Provided herein are complete reconstructed amino acid and nucleic acid consensus sequences of V_{H} and V_{L} chain regions of antibodies disclosed herein. Also provided herein, are the amino acid and nucleic acid sequences of the CDR3 regions of the V_{H} and V_{L} of the antibodies, described herein. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant regions, for example to alter the effector functions of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific antibodies by constructing expression vectors that include CDR sequences from the specific antibody (e.g., antibodies disclosed herein) grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. Sci. USA. 86:10029-10033; U.S. Pat. No. 5,225,539 to Winter, and U.S. Pat. Nos. 5,530,101; 5,585,089; 5,693,762 and 6,180,370 to Queen et al.)

Suitable framework sequences can be obtained from public DNA databases or published references that include germline antibody gene sequences. For example, germline DNA sequences for human heavy and light chain variable region genes can be found in the “VBase” human germline sequence database (available on the Internet), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference.

Another type of variable region modification is to mutate amino acid residues within the V_{H} and/or V_{L} CDR 1, CDR2 and/or CDR3 regions to thereby improve one or more binding properties (e.g., affinity) of the antibody of interest. Site-directed mutagenesis or PCR-mediated mutagenesis can be performed to introduce the mutations and the effect on antibody binding, or other functional property of interest, can be evaluated in appropriate in vitro or in vivo assays. Preferably conservative modifications (as discussed above) are introduced. The mutations may be amino acid substitutions, additions or deletions, but are preferably substitutions. Moreover, typically no more than one, two, three, four or five residues within a CDR region are altered.

Engineered antibodies according to at least some embodiments of the invention include those in which modifications have been made to framework residues within V_{H} and/or V_{L}, e.g. to improve the properties of the antibody. Typically, such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

In addition or alternative to modifications made within the framework or CDR regions, antibodies according to at least some embodiments of the disclosure may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described above. The numbering of residues in the Fc region is that of the EU index of Kabat.

In one embodiment, the hinge region of C_{H}1 is modified such that the number of cysteine residues in the hinge region is altered, e.g., increased or decreased. This approach is described further in U.S. Pat. No. 5,677,425 by Bodmer et al. The number of cysteine residues in the hinge region of C_{H}1 is altered to, for example, facilitate assembly of the light and heavy chains or to increase or decrease the stability of the antibody. In another embodiment, the Fc hinge region of an antibody is mutated to decrease the biological half-life of the antibody. More specifically, one or more amino acid mutations are introduced into the C_{H}2-C_{H}3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, to increase the biological half-life, the antibody can be altered within the C_{H}1 or C_{L} region to contain a salvage receptor binding epitope taken from two loops of a C_{H}2 domain of an Fc region of an IgG, as described in U.S. Pat. Nos. 5,869,046 and 6,121,022 by Presta et al.

### Antigen-Binding Fragments

### Antigen-Binding Fragment Terminology

The terms “antibody fragment,” “antigen-binding fragment,” or “antibody binding domain” refer to at least one portion of an antibody, or recombinant variants thereof, that contains the antigen-binding domain, i.e., an antigenic determining variable region of an intact antibody, that is sufficient to confer recognition and specific binding of the antibody fragment to a target, such as an antigen and its defined epitope. Examples of antigen-binding fragments include, but are not limited to, Fab, Fab′, F(ab′)₂, and Fv fragments, single-chain (sc)Fv (“scFv”) antibody fragments, linear antibodies, single domain antibodies such as sdAb (either V_{L} or V_{H}), camelid V_{H}H domains, and multi-specific antibodies formed from antibody fragments.

Antibody fragments can be made by various techniques, including but not limited to proteolytic digestion of an intact antibody as well as production by recombinant host cells (e.g., E. coli or phage), as described herein.

An Fv is the minimum antibody fragment that contains a complete antigen-recognition and antigen-binding site. This fragment contains a dimer of one heavy- and one light-chain variable region domain in tight, non-covalent association. From the folding of these two domains emanate six hypervariable loops (three loops each from the H and L chain) that contribute the amino acid residues for antigen-binding and confer antigen-binding specificity to the antibody. However, even a single variable region (or half of an Fv comprising only three CDRs specific for an antigen) has the ability to recognize and bind antigen, although at a lower affinity than the entire binding site.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived. Unless specified, as used herein a scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

A diabody is a small antibody fragment prepared by constructing a scFv fragment with a short linker (about 5-10 residues) between the V_{H} and V_{L} domains such that inter-chain but not intra-chain pairing of the V domains is achieved, resulting in a bivalent fragment. Bispecific diabodies are heterodimers of two crossover scFv fragments in which the V_{H} and V_{L} domains of the two antibodies are present on different polypeptide chains. (see, e.g., Hollinger et al. Proc. Natl. Acad. Sci. USA 90:6444-6448 (1993)).

Domain antibodies (dAbs), which can be produced in fully human form, are the smallest known antigen-binding fragments of antibodies, ranging from about 11 kDa to about 15 kDa. DAbs are the robust variable regions of the heavy and light chains of immunoglobulins (V_{H} and V_{L}, respectively). They are highly expressed in microbial cell culture, show favorable biophysical properties including, for example, but not limited to, solubility and temperature stability, and are well suited to selection and affinity maturation by in vitro selection systems such as, for example, phage display. DAbs are bioactive as monomers and, owing to their small size and inherent stability can be formatted into larger molecules to create drugs with prolonged serum half-lives or other pharmacological activities.

Fv and scFv are the only species with intact combining sites that are devoid of constant regions. Thus, they are suitable for reduced nonspecific binding during in vivo use. scFv fusion proteins can be constructed to yield fusion of an effector protein at either the amino or the carboxy terminus of an scFv. The antibody fragment also can be a “linear antibody”. Such linear antibody fragments can be monospecific or bispecific.

### Antigen-Binding Fragment SEQ ID NOs

In an alternative embodiment of the invention, an antigen-binding fragment may be produced in a variety of forms where the antigen-binding domain is expressed as part of a contiguous polypeptide chain including, for example, a single domain antibody fragment (sdAb), a single chain antibody (scFv) derived from a human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In such an embodiment, the antigen-binding domain comprises:
(a) one or more (e.g., one, two, or all three) of:
   (i) heavy chain complementary determining region 1 (CDR-H1), wherein the CDR-H1 comprises a sequence selected from any one of SEQ ID NOs: 10001-11250,
   (ii) heavy chain complementary determining region 2 (CDR-H2), wherein the CDR-H2 comprises a sequence selected from any one of SEQ ID NOs: 12501-13750,
   (iii) heavy chain complementary determining region 3 (CDR-H3), wherein the CDR-H3 comprises a sequence selected from any one of SEQ ID NOS: 15001-16250, and/or
(b) one or more (e.g., one, two, or all three) of:
   (i) light chain complementary determining region 1 (CDR-L1), wherein the CDR-L1 comprises a sequence selected from any one of SEQ ID NOs: 11251-12500,
   (ii) light chain complementary determining region 2 (CDR-L2), wherein the CDR-L2 comprises a sequence selected from any one of SEQ ID NOs: 13751-15000,
   (iii) light chain complementary determining region 3 (CDR-L3), wherein the CDR-L3 comprises a sequence selected from any one of SEQ ID NOs: 16251-17500.
      In one embodiment, the antigen-binding domain comprises a heavy chain variable region described herein and/or a light chain variable region described herein. In some embodiments:
(a) the heavy chain variable region comprises a sequence selected from any one of SEQ ID NOs: 17501-18750, and/or
(b) the light chain variable region comprises a sequence selected from any one of SEQ ID NOs: 18751-20000.
   In one embodiment, the antigen-binding domain is a scFv comprising a heavy chain variable region and a light chain variable region of an amino acid sequence, e.g., a heavy chain variable region and light chain variable region described herein. In an embodiment, the antigen-binding domain (e.g., an scFv) comprises:
(a) a heavy chain variable region comprising:
   (i) an amino acid sequence having at least one, two or three modifications (e.g., substitutions) but not more than 30, 20 or 10 modifications (e.g., substitutions) of an amino acid sequence of a heavy chain variable region provided herein, or
   (ii) a sequence with 85-99% (e.g., 90-99%, or 95-99%) identity to an amino acid sequence provided herein; and/or
(b) a light chain variable region comprising:
   (i) an amino acid sequence having at least one, two or three modifications (e.g., substitutions) but not more than 30, 20 or 10 modifications (e.g., substitutions) of an amino acid sequence of a light chain variable region provided herein, or
   (ii) a sequence with 85-99% (e.g., 90-99%, or 95-99%) identity to an amino acid sequence provided herein.

### Synthesis of Antigen Binding Fragments

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame. The isolated DNA encoding the V_{H} region can be converted to a full-length heavy chain gene by operatively linking the V_{H}-encoding DNA to another DNA molecule encoding heavy chain constant regions (C_{H}1, C_{H}2 and C_{H}3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H}1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the V_{H}- and V_{L}-encoding DNA fragments are operatively linked to another fragment encoding a flexible linker, e.g., encoding the amino acid sequence (Gly-Gly-Gly-Gly-Ser)₃, such that the V_{H} and V_{L} sequences can be expressed as a contiguous single-chain protein, with the V_{L} and V_{H} regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### Alternative Paths to Antibody Production

As an alternative to direct synthesis using recombinant DNA methods, the antibody or antigen binding fragments described in this disclosure may be produced via hybridoma. In the hybridoma method, a mouse or other appropriate host animal, such as a hamster or macaque monkey, is immunized as herein described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Preferred myeloma cells are those that fuse efficiently, support stable high-level production of antibody by the selected antibody-producing cells, and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Exemplary murine myeloma lines include those derived from MOP-21 and M.C.-11 mouse tumors available from the Salk Institute Cell Distribution Center, San Diego, Calif USA, and SP-2 or X63-Ag8-653 cells available from the American Type Culture Collection, Rockville, Md. USA. Culture medium in which hybridoma cells are growing is assayed for production of monoclonal antibodies directed against the antigen. Accordingly, in one aspect the present disclosure provides a hybridoma producing the antibody or antigen-binding fragment thereof, described herein. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by Scatchard analysis (Munson et al., Anal. Biochem. 107:220 (1980)).

After hybridoma cells are identified that produce antibodies of the desired specificity, affinity, and/or activity, the clones may be subcloned by limiting dilution procedures and grown by standard methods (Goding, Monoclonal Anti-bodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

### Screening Methods for Identification of Target Antigens

### Binding Affinity

Antibodies may be screened for binding affinity by methods known in the art. For example, gel-shift assays, Western blots, radiolabeled competition assay, co-fractionation by chromatography, co-precipitation, cross linking, ELISA, and the like may be used, which are described in, for example, Current Protocols in Molecular Biology (1999) John Wiley & Sons, NY, which is incorporated herein by reference in its entirety.

To initially screen for antibodies which bind to the desired epitope on an antigen (e.g., a SARS-CoV-2 associated antigen), a routine cross-blocking assay such as that described in Antibodies, A Laboratory Manual, Cold Spring Harbor Laboratory, Ed Harlow and David Lane (1988), can be performed. Routine competitive binding assays may also be used, in which the unknown antibody is characterized by its ability to inhibit binding of antigen to an antigen specific antibody of the invention. Intact antigen, fragments thereof, or linear epitopes can be used. Epitope mapping is described in Champe et al., J. Biol. Chem. 270: 1388-1394 (1995).

In one variation of an in vitro assay, the present disclosure provides a method comprising the steps of (a) contacting an immobilized antigen with a candidate antibody and (b) detecting binding of the candidate antibody to the antigen. In an alternative embodiment, the candidate antibody is immobilized and binding of antigen is detected. Immobilization is accomplished using any of the methods well known in the art, including covalent bonding to a support, a bead, or a chromatographic resin, as well as non-covalent, high affinity interaction such as antibody binding, or use of streptavidin/biotin binding wherein the immobilized compound includes a biotin moiety. Detection of binding can be accomplished (a) using a radioactive label on the compound that is not immobilized, (b) using a fluorescent label on the non-immobilized compound, (c) using an antibody immunospecific for the non-immobilized compound, (d) using a label on the non-immobilized compound that excites a fluorescent support to which the immobilized compound is attached, as well as other techniques well known and routinely practiced in the art.

### Modulator Activity

Another aspect of the present invention is directed to methods of identifying antibodies which modulate (i.e., decrease) activity of a target antigen comprising contacting a target antigen with an antibody, and determining whether the antibody modifies activity of the antigen. The activity in the presence of the test antibody is compared to the activity in the absence of the test antibody. Where the activity of the sample containing the test antibody is lower than the activity in the sample lacking the test antibody, the antibody will have inhibited activity.

Antibodies that modulate (i.e., increase, decrease, or block) the activity or expression of desired target may be identified by incubating a putative modulator with a cell expressing the desired target and determining the effect of the putative modulator on the activity or expression of the target. The selectivity of an antibody that modulates the activity of a target polypeptide or polynucleotide can be evaluated by comparing its effects on the target polypeptide or polynucleotide to its effect on other related compounds. Selective modulators may include, for example, antibodies and other proteins, peptides, or organic molecules which specifically bind to target polypeptides or to a nucleic acid encoding a target polypeptide. Modulators of target activity will be therapeutically useful in treatment of diseases and physiological conditions in which normal or aberrant activity of target polypeptide is involved. The target can be a for example, a SARS-CoV-2 associated antigen.

In one embodiment of the invention, methods of screening for antibodies which modulate the activity of target antigen comprise contacting antibodies with a target antigen polypeptide and assaying for the presence of a complex between the antibody and the target antigen. In such assays, the ligand is typically labeled. After suitable incubation, free ligand is separated from that present in bound form, and the amount of free or uncomplexed label is a measure of the ability of the particular antibody to bind to the target antigen.

### High Throughput Screening

The invention also contemplates high throughput screening (HTS) assays to identify antibodies that interact with or inhibit biological activity (i.e., inhibit enzymatic activity, binding activity, etc.) of an antigen. FITS assays permit screening of large numbers of compounds in an efficient manner. Cell-based HTS systems are contemplated to investigate the interaction between antibodies and their target antigen and their binding partners. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and target antigen.

An FITS array may consist of one or more protein arrays (e.g., antibody arrays, antibody microarrays, protein microarray). The array can comprise one or more antibodies or antigen-binding fragment thereof, disclosed herein, immobilized on a solid support. Methods of production and use of such arrays are known well known in art (e.g., (Buessow et al., Nucleic Acids Res. 1998, Lueking et al., Mol Cell Proteomics., 2003; Angenendt et al., Mol Cell Proteomics., 2006) In some embodiments, very small amounts (e.g., 1 to 500 μg) of antibody or antigen-binding fragment thereof is immobilized. In some embodiments, there will be from 1 μg to 100 from 1 μg to 50 μg, from 1 μg to 20 μg, from 3 μg to 100 μg, from 3 μg to 50 μg, from 3 μg to 20, from 5 μg to 100 from 5 μg to 50 from 5 to 20 μg of antibody present in a single sample. In one aspect, at least one of the samples in a plurality of samples will have from 1 μg to 100 from 1 μg to 50 from 1 μg to 20 from 3 μg to 100 from 3 μg to 50 from 3 μg to 20, from 5 μg to 100 from 5 μg to 50 from 5 μg to 20 μg of antibody present. A solid support refers to an insoluble, functionalized material to which the antibodies can be reversibly attached, either directly or indirectly, allowing them to be separated from unwanted materials, for example, excess reagents, contaminants, and solvents. Examples of solid supports include, for example, functionalized polymeric materials, e.g., agarose, or its bead form Sepharose®, dextran, polystyrene and polypropylene, or mixtures thereof; compact discs comprising microfluidic channel structures; protein array chips; pipet tips; membranes, e.g., nitrocellulose or PVDF membranes; and microparticles, e.g., paramagnetic or non-paramagnetic beads. In some embodiments, an affinity medium will be bound to the solid support and the antibody will be indirectly attached to solid support via the affinity medium. In one aspect, the solid support comprises a protein A affinity medium or protein G affinity medium. A “protein A affinity medium” and a “protein G affinity medium” each refer to a solid phase onto which is bound a natural or synthetic protein comprising an Fc-binding domain of protein A or protein G, respectively, or a mutated variant or fragment of an Fc-binding domain of protein A or protein G, respectively, which variant or fragment retains the affinity for an Fc-portion of an antibody. Antibody arrays can be fabricated by the transfer of antibodies onto the solid surface in an organized high-density format followed by chemical immobilization. Representative techniques for fabrication of an array include photolithography, ink jet and contact printing, liquid dispensing and piezoelectrics. The patterns and dimensions of antibody arrays are to be determined by each specific application. The sizes of each antibody spot may be easily controlled by the users. Antibodies may be attached to various kinds of surfaces via diffusion, adsorption/absorption, or covalent cross-linking and affinity. Antibodies may be directly spotted onto a plain glass surface. To keep antibodies in a wet environment during the printing process, high percent glycerol (e.g., 30-40%) may be used in sample buffer and the spotting is carried out in a humidity-controlled environment.

### Antibody Arrays

The surface of a substrate may be modified to achieve better binding capacity. For example, the glass surface may be coated with a thin nitrocellulose membrane or poly-L-lysine such that antibodies can be passively adsorbed to the modified surface through non-specific interactions. Antibodies may be immobilized onto a support surface either by chemical ligation through a covalent bond or non-covalent binding. There are many known methods for covalently immobilizing antibodies onto a solid support. For example, MacBeath et al., (1999) J Am. Chem. Soc. 121:7967-7968) use the Michael addition to link thiol-containing compounds to maleimide-derivatized glass slides to form a microarray of small molecules. See also, Lam & Renil (2002) Current Opin. Chemical Biol. 6:353-358. Representative examples of biomarkers include, TROP/TNFRSF19, IL-1 sRI, uPAR, IL-10, VCAM-1 (CD106), IL-10 receptor-β, VE-cadherin, IL-13 receptor-α1, VEGF, IL-13 receptor-α2, VEGF R2 (KDR), IL-17, VEGF R3

The arrays can employ single-antibody (label-base) detection or 2-antibody (sandwich-based) detection. In some embodiments, an ELISA (also known as an antibody sandwich assay) may be performed following standard techniques as follows. Antibodies used as the capture antibodies for an antigen disposed on (e.g., coated onto) a solid support, which may then be washed at least once (e.g., with water and/or a buffer such as PBS-t), followed by a standard blocking buffer, and then at least one more wash. The solid support may then be brought into contact with the sample/biosample under conditions to allow antibody-antigen complexes to form (e.g., incubating from 1 hour to about 24 hours at a temperature from about 4° C. to about room temperature). As used herein, “biosample” and “sample” are used interchangeably and embrace both fluids (also referred to herein as fluid samples and biofluids) and tissue obtained from the subject. The term “biofluid” as used herein refers to a biological fluid sample such as blood samples, cerebral spinal fluid (CSF), urine and other liquids obtained from the subject, or a solubilized preparation of such fluids wherein the cell components have been lysed to release intra-cellular contents into a buffer or other liquid medium. The definition also includes samples that have been manipulated in any way after their procurement, such as by treatment with reagents, or enrichment for certain components, such as proteins or polynucleotides. The term “blood sample” embraces whole blood, plasma, and serum. Solid tissue samples include biopsy specimens and tissue cultures or cells derived therefrom, and the progeny thereof. A sample may comprise a single cell or more than a single cell. The biosample may also be a cultured population of cells derived from the subject human or animal. However, whenever the biosample comprises a population of cells, the method will first require that the constituents of the cells be solubilized by lysing the cells, and removing solid cell debris, thereby providing a solution of the biomarkers. Samples can be prepared by methods known in the art such as lysing, fractionation, purification, including affinity purification, FACS, laser capture micro-dissection or iospycnic centrifugation. The support may then be washed at least once (e.g., with a buffer such as PBS-t). To detect the complexation between the capture antibodies and the antigen that may be present in the sample, secondary or “detection” antibodies are applied to the solid support (e.g., diluted in blocking buffer) under conditions to allow complexation between the secondary antibodies and the respective biomarkers (e.g., at room temperature for at least one hour). The secondary antibodies are selected so as to bind a different epitope on the antigen than the capture antibody. The optimum concentrations of capture and detection antibodies are determined using standard techniques such as the “criss-cross” method of dilutions. The detection antibody may be conjugated, directly or indirectly, to a detectable label.

The term “detectable label” as used herein refers to labeling moieties known in the art. Said moiety may be, for example, a radiolabel (e.g., ³H, ¹²⁵I, ³⁵S, ¹⁴C, ³²P, etc.), detectable enzyme (e.g., horse radish peroxidase (HRP), alkaline phosphatase etc.), a dye (e.g., a fluorescent dye), a colorimetric label such as colloidal gold or colored glass or plastic (e.g., polystyrene, polypropylene, latex, etc.), beads, or any other moiety capable of generating a detectable signal such as a colorimetric, fluorescent, chemiluminescent or electrochemiluminescent (ECL) signal. The term “dye” as used herein refers to any reporter group whose presence can be detected by its light absorbing or light emitting properties. For example, Cy5 is a reactive water-soluble fluorescent dye of the cyanine dye family. Cy5 is fluorescent in the red region (about 650 to about 670 nm). It may be synthesized with reactive groups on either one or both of the nitrogen side chains so that they can be chemically linked to either nucleic acids or protein molecules. Labeling is done for visualization and quantification purposes. Cy5 is excited maximally at about 649 nm and emits maximally at about 670 nm, in the far red part of the spectrum; quantum yield is 0.28 (FW=792). Suitable fluorophores (chromes) for the probes of the disclosure may be selected from, but not intended to be limited to, fluorescein isothiocyanate (FITC, green), cyanine dyes Cy2, Cy3, Cy3.5, Cy5, Cy5.5 Cy7, Cy7.5 (ranging from green to near-infrared), Texas Red, and the like. Derivatives of these dyes for use in the embodiments of the disclosure may be, but are not limited to, Cy dyes (Amersham Bioscience), Alexa Fluors (Molecular Probes Inc.), HILYTE™ Fluors (AnaSpec), and DYLITE™ Fluors (Pierce, Inc). In some embodiments, the detectable label is a chromogenic label such as biotin, in which case the detection antibody-biotin conjugate is detected using Streptavidin/Horseradish Peroxidase (HRP) or the equivalent. The streptavidin may be diluted in an appropriate block and incubated for 30 minutes at room temperature. Other detectable labels suitable for use in the present invention include fluorescent labels and chemiluminescent labels.

The support may then be washed and the label (e.g., HRP enzymatic conjugate on the streptavidin) is detected using the following standard protocols such as a chromogenic system (the SIGMA FAST™ OPD system), a fluorescent system or a chemiluminescent system. The amounts of antigen present in the sample may then be read on an ELISA plate reader (e.g., SpectraMax 384 or the equivalent). The concentration of each of the antigens may then be back-calculated (e.g., by using the standard curve generated from purified antigens and multiplied by the dilution factor following standard curve fitting methods), and then compared to a control (generated from tissue samples obtained from healthy subjects).

In one embodiment, a biosample, e.g., a biofluid, is contacted with a system of reagents, well-known in the art, that can attach biotin moieties to some or all of the constituent components of the sample, and especially to the protein or peptide constituents thereof, including the biomarkers. Following this biotinylation step, the biotinylated biosample may then be contacted with the antibody array that contains an array of antibodies specific to each of the antigens.

After an adequate incubation period, readily selected to allow the binding of any antigen in the sample to its corresponding antibody of the array, the fluid sample is washed from the array. The array is then contacted with a biotin-binding polypeptide such as avidin or streptavidin, that has been conjugated with a detectable label (as described above in connection with the ELISA). Detection of the label on the array (relative to a control) will indicate which of the biomarkers captured by the respective antibody is present in the sample.

Regardless of the specific assay format, the biotin-label-based array methods are relatively advantageous from several standpoints. Biotin-label can be used as signal amplification. Biotin is the most common method for labeling protein and the label process can be highly efficient. Furthermore, biotin can be detected using fluorescence-streptavidin and, therefore, visualized via laser scanner, or HRP-streptavidin using chemiluminescence. Using biotin-label-based antibody arrays, most targeted proteins can be detected at pg/ml levels. The detection sensitivity of the present methods can be further enhanced by using 3-DNA detection technology or rolling circle amplification (Schweitzer et al., (2000) Proc. Natl. Acad. Sci. U.S.A. 97:10113-10119; Horie et al., (1996) Int. J. Hematol. 63:303-309).

As it relates to the present disclosure, the sample can be obtained from a subject having disease (e.g., infection with SARS-CoV-2) and a healthy subject.

### Protein Arrays

In some embodiments, protein arrays can be used where protein antigens with known identities are immobilized on a solid support as capture molecules and one seeks to determine whether the known antigens binds to a candidate antibody. The antigen can be labeled with a tag that allows detection or immunoprecipitation after capture by an immobilized antibody. Protein antigens can be obtained, for example, from a patient infected with SARS-CoV-2. A number of commercial protein arrays are available e.g., PROTOARRAY®, KINEX™, RAYBIO® Human RTK Phosphorylation Antibody Array. The antibody-antigen complexes can be obtained by methods known in the art (e.g., immunoprecipitation or Western blot). For reviews on Protein array and antibody array that can be of interest in this study, see Reymond Sutandy, et al. 2013; Liu, B. C.-S., et al. 2012; Haab B B, 2005.

In an exemplary immunoprecipitation method, an antibody or antigen-binding fragment thereof, described herein is added first to a sample comprising an antigen, and incubated to allow antigen-antibody complexes to form. Subsequently, the antigen-antibody complexes are or with protein A/G-coated beads to allow them to absorb the complexes. In a modified approach, the antibody or antigen-binding fragment thereof is fused to a His tag or other tags (e.g., FLAG tag, Biotin Tag) by recombinant DNA techniques, and immunoprecipitated using an antibody to the tag (pull-down assay). The beads are then thoroughly washed, and the antigen is eluted from the beads by an acidic solution or SDS. The eluted sampled can be analyzed using Mass Spectrometry or SDS page to identify and confirm the antigen. Methods to analyze antibody-antigen complexes formed on a protein microarray and identify the antigen via mass spec are known.

### ADCC and CDC Assays

In one aspect, the antibodies or antigen-binding fragment thereof, disclosed herein, are contemplated as therapeutic antibodies for treatment of infection with SARS-CoV-2. Accordingly, the antibodies or antigen-binding fragment thereof, can be further screened in an antibody-dependent cell-mediated cytotoxicity (ADCC) assay and/or Complement-dependent cytotoxicity (CDC) assay. “ADCC activity” refers to the ability of an antibody to elicit an ADCC reaction. ADCC is a cell-mediated reaction in which antigen-nonspecific cytotoxic cells that express FcRs (e.g., natural killer (NK) cells, neutrophils, and macrophages) recognize antibody bound to the surface of a target cell and subsequently cause lysis of (i.e., “kill”) the target cell (e.g., a cell which has been infected by SARS-CoV-2). The primary mediator cells are natural killer (NK) cells. NK cells express FcγRIII only, with FcγRIIIA being an activating receptor and FcγRIIIB an inhibiting one; monocytes express FcγRI, FcγRII and FcγRIII (Ravetch et al. (1991) Annu. Rev. Immunol. 9:457-92). ADCC activity can be assessed directly using an in vitro assay, e.g., a ⁵¹Cr release assay using peripheral blood mononuclear cells (PBMC) and/or NK effector cells as described in the Examples and Shields et al. (2001) J. Biol. Chem. 276:6591-6604, or another suitable method known in the art. ADCC activity may be expressed as a concentration of antibody at which the lysis of target cells is half-maximal. Accordingly, in some embodiments, the concentration of an antibody or antigen-binding fragment thereof of the disclosure, at which the lysis level is the same as the half-maximal lysis level by the wild-type control, is at least 2-, 3-, 5-, 10-, 20-, 50-, 100-fold lower than the concentration of the wild-type control itself.

Additionally, in some embodiments, the antibody or antigen-binding fragment thereof of the present disclosure may exhibit a higher maximal target cell lysis as compared to the wild-type control. For example, the maximal target cell lysis of an antibody or Fc fusion protein of the invention may be 10%, 15%, 20%, 25% or more higher than that of the wild-type control. “Complement dependent cytotoxicity” or “CDC” refer to the ability of a molecule to lyse a target (e.g. a cell infected with SARS-CoV-2) in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al. J. Immunol. Methods 202:163 (1996), may be performed.

### Target Antigen

In some embodiments, an antibody or antigen binding fragment thereof disclosed herein binds a coronavirus or an antigen on the coronavirus. In some embodiments, an antibody or antigen binding fragment thereof disclosed herein binds a Severe acute respiratory syndrome—related coronavirus (SARSr-CoV or SARS-CoV), or an antigen on the SARS-CoV. In some embodiments, an antibody or antigen binding fragment thereof disclosed herein binds a Middle East respiratory syndrome (MERS), or an antigen thereof. In some embodiments, an antibody or antigen binding fragment thereof binds a SARS-CoV-2, or an antigen thereof. In some embodiments, an antibody or antigen biding fragment thereof, disclosed herein, binds to a SARS-Cov-2 antigen, or a homolog thereof, or a variant thereof. In some embodiments, an antibody or antigen binding fragment thereof disclosed herein binds a SARS-Cov-2 spike (S) protein, or a homolog thereof, or a variant thereof. The S protein is comprised of an N-terminal subunit (S1) that mediates receptor binding and a C-terminal 47 subunit (S2) responsible for virus-cell membrane fusion (Wrapp et al., 2020). During viral entry into cells, the receptor-binding domain (RBD) of 51 engages a human host cell receptor; human angiotensin converting enzyme 2 (hACE2) (Letko et al., 2020). Processing of S by host cell proteases, typically TMPRSS2, TMPRSS4, or endosomal cathepsins, facilitates the S2 dependent fusion of viral and host-cell membranes (Hoffmann et al., 2020; Zang et al., 2020). In some embodiments, an antibody or antigen binding fragment thereof of the present disclosure binds subunit 51 of the SARS-Cov-2 spike (S) protein, or a homolog thereof, or a variant thereof. In some embodiments, an antibody or antigen binding fragment thereof of the present disclosure binds subunit S2 of the SARS-Cov-2 spike (S) protein, or a homolog thereof, or a variant thereof. In some embodiments, an antibody or antigen binding fragment thereof of the present disclosure binds a receptor-binding domain of subunit S1 of the SARS-Cov-2 spike (S) protein, or a homolog thereof, or a variant thereof. In some embodiments, an antibody or the antigen binding thereof of the present disclosure inhibits binding of the receptor-binding domain of subunit S1 of SARS-Cov-2 spike (S) protein to a host cell receptor (e.g., human angiotensin converting enzyme 2 (hACE2)).

In some embodiments, an antibody or antigen binding fragment thereof of the present disclosure binds an epitope on the target antigen (e.g., SARS-Cov-2 spike (S) protein). In some embodiments, an antibody or antigen binding fragment thereof of the present disclosure binds multiple (e.g., 1, 2, 3, 4, 5, 6, 7, 8, 9, 10, or more epitopes on a target antigen (e.g., SARS-Cov-2 spike (S) protein). In some embodiments, an antibody or antigen binding fragment thereof disclosed herein binds an epitope comprising an amino acid sequence selected Table 3. In some embodiments, an antibody or antigen binding fragment thereof disclosed herein binds an epitope comprising an amino acid sequence set forth at SEQ NO: 20061. In some embodiments, an antibody or antigen binding fragment thereof disclosed herein binds an epitope comprising an amino acid sequence set forth at SEQ NO: 20045. In some embodiments, an antibody or antigen binding fragment thereof disclosed herein binds an epitope comprising an amino acid sequence set forth at SEQ NO: 20082. In some embodiments, an antibody or antigen binding fragment thereof disclosed herein binds an epitope comprising an amino acid sequence set forth at SEQ NO: 20023.

The binding affinity and dissociation rate of an antibody or an antigen binding fragment thereof disclosed herein to an epitope on a SARS-CoV2 antigen (e.g., SARS-Cov-2 spike (S) protein, subunit S1, subunit S2, or a receptor-binding domain of subunit S1) can be determined by methods known in the art. The binding affinity can be measured by ELISAs, RIAs, flow cytometry, surface plasmon resonance, such as BIACORE™. The dissociate rate can be measured by surface plasmon resonance. Preferably, the binding affinity and dissociation rate is measured by surface plasmon resonance. More preferably, the binding affinity and dissociation rate are measured using BIACORE™.

### Epitope Mapping

The term “epitope,” as used herein, refers to an antigenic determinant that interacts with a specific antigen-binding site in the variable region of an antibody molecule known as a paratope. A single antigen can have more than one epitope. Thus, different antibodies may bind to different areas on an antigen and can have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, 2004, Methods Mol. Biol. 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antigen-binding domain of an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues, which correspond to the specific amino acids with which the antibody interacts. See, e.g., Ehring (1999) Analytical Biochemistry 267(2):252-259; Engen and Smith (2001) Anal. Chem. 73:256A-265A. X-ray crystallography of the antigen/antibody complex may also be used for epitope mapping purposes.

The epitope on a target antigen to which the antibody or antigen-binding fragment, disclosed herein, bind may consist of a single contiguous sequence of 3 or more (e.g., 3, 4, 5, 6, 7, 8, 9, 10, 11, 12, 13, 14, 15, 16, 17, 18, 19, 20 or more) amino acids of the target antigen. Alternatively, the epitope may consist of a plurality of non-contiguous amino acids (or amino acid sequences) of antigen.

### Pharmaceutical Compositions and Medicaments

In one aspect, the present disclosure provides a composition comprising an antibody or antigen binding fragment thereof disclosed herein and/or a nucleic acid encoding the antibody or antigen binding fragment thereof disclosed herein. The nucleic acids encoding the antibodies or antigen binding fragment are described above including their sequences. For the clinical use of the methods described herein, administration of the antibodies or antigen binding fragments thereof, and/or nucleic acids encoding the antibodies or antigen binding fragment thereof of the present disclosure can include formulation into pharmaceutical compositions, pharmaceutical formulations, or medicaments, for administration, e.g., subcutaneous, intravenous, intradermal, intraperitoneal, oral, intramuscular, intracranial or other routs of administration. In some embodiments, the antibodies or antigen binding fragments thereof, described herein, or nucleic acids encoding the antibodies or antigen binding fragment thereof can be administered along with any pharmaceutically acceptable carrier, excipient, or diluent, which results in an effective treatment and/or effective prophylaxis in the subject. Thus, in one aspect, the present disclosure provides pharmaceutical compositions comprising one or more antibodies or antigen binding fragment thereof, and/or nucleic acids encoding the one or more antibodies or antigen binding fragment thereof described herein, in combination with one or more pharmaceutically acceptable carrier, excipient, or diluent.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an antibody or antigen binding fragment thereof of the present disclosure. Examples include, but are not limited to, any of a number of standard pharmaceutical carriers such as sterile phosphate buffered saline solutions, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like. Each carrier must be “acceptable” in the sense of being compatible with the other ingredients of the formulation and not injurious to the patient. The terms “excipient”, “carrier”, “pharmaceutically acceptable carrier”, or the like are used interchangeably herein. The compositions of the present disclosure may further comprise one or more pharmaceutically acceptable carriers, excipients, and other agents that are incorporated into formulations to provide improved transfer, delivery, tolerance, and the like (herein collectively referred to as “pharmaceutically acceptable carriers or diluents”). A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA, 1998, J. Pharm. Sci. Technol. 52:238-311.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

Optionally, the formulations comprising the compositions described herein contain a pharmaceutically acceptable salt, typically, e.g., sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. Optionally, the formulations of the invention can include a pharmaceutically acceptable surfactant at a concentration of 0.005 to 0.02%.

The compositions described herein can be specially formulated for administration of the antibody or antigen binding fragment thereof to a subject in solid, liquid or gel form, including those adapted for the following: (a) parenteral administration, for example, by subcutaneous, intramuscular, intravenous or epidural injection as, for example, a sterile solution or suspension, or sustained-release formulation; (b) topical application, for example, as a cream, ointment, or a controlled-release patch or spray applied to the skin; (c) intravaginally or intrarectally, for example, as a pessary, cream or foam; (d) ocularly; (e) transdermally; (f) transmucosally; or (g) nasally. Additionally, an antibody or antigen binding fragment thereof, or compositions of the present disclosure can be implanted into a patient or injected using a drug delivery system. See, e.g., Urquhart et al., 24 Ann. Rev. Pharmacol. Toxicol. 199 (1984); Controlled Release of Pesticides & Pharmaceuticals (Lewis, ed., Plenum Press, New York, 1981); U.S. Pat. Nos. 3,773,919, 3,270,960.

In some embodiments, sustained-release preparations can be used. Suitable examples of sustained-release preparations include semipermeable matrices of solid hydrophobic polymers containing an antibody or antigen binding fragment of the present disclosure, in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions. In certain situations, the pharmaceutical composition can be delivered in a controlled release system. In one embodiment, a pump may be used (see Langer R. Science 249(4976):1527-1533 (1990); Sefton 1987 CRC Crit. Ref. Biomed. Eng. 14:201). In another embodiment, polymeric materials can be used. In yet another embodiment, a controlled release system can be placed in proximity of the composition's target, thus requiring only a fraction of the systemic dose (see, e.g., Goodson, in R. S. Langer and D. L. Wise (eds.), Medical Applications of Controlled Release, vol. 2, pp. 115-138 (CRC Press, Boca Raton, 1984)).

A pharmaceutical composition of the present disclosure can be delivered, e.g., subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN70130™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition include, but certainly are not limited to the SOLOSTAR™ pen (Sanofi-Aventis), the FLEXPEN™ (Novo Nordisk), and the KWIKPEN™ (Eli Lilly).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Compositions of the present disclosure can be in the form of, for example, granules, powders, tablets, capsules, syrup, suppositories, injections, emulsions, elixirs, suspensions or solutions. The amount of the aforesaid antibody contained can be about 5 to about 500 mg per dosage form in a unit dose; especially in the form of injection, it is preferred that the aforesaid antibody is contained in about 5 to about 100 mg and in about 10 to about 250 mg for the other dosage forms.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or par-enteral administration. In some embodiments, pharmaceutical compositions can be prepared in a lyophilized form. The lyophilized preparations can comprise a cryoprotectant known in the art. The term “cryoprotectants” as used herein generally includes agents, which provide stability to the protein from freezing-induced stresses. Examples of cryoprotectants include polyols such as, for example, mannitol, and include saccharides such as, for example, sucrose, as well as including surfactants such as, for example, polysorbate, poloxamer or polyethylene glycol, and the like. Cryoprotectants also contribute to the tonicity of the formulations. Pharmaceutically suitable surfactants, suspending agents, emulsifying agents, may be added for oral or par-enteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, iso-propyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For nasal administration, the pharmaceutical formulations and medicaments may be a spray or aerosol containing an appropriate solvent(s) and optionally other compounds such as, but not limited to, stabilizers, antimicrobial agents, antioxidants, pH modifiers, surfactants, bio-availability modifiers and combinations of these. A propellant for an aerosol formulation may include compressed air, nitrogen, carbon dioxide, or a hydrocarbon based low boiling solvent.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di-, or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

The concentration of an antibody or an antigen binding fragment thereof in these compositions can vary widely, i.e., from less than about 10%, usually at least about 25% to as much as 75% or 90% by weight and will be selected primarily by fluid volumes, viscosities, etc., in accordance with the particular mode of administration selected. Actual methods for preparing orally, topically and parenterally administrable compositions will be known or apparent to those skilled in the art and are described in detail in, for example, Remington's Pharmaceutical Science, 19th ed., Mack Publishing Co., Easton, Pa. (1995), which is incorporated herein by reference.

In another embodiment of the invention, an article of manufacture containing materials useful for prophylaxis against or treatment of an infection with SARS-CoV-2. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the antibody of the invention. The label on or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user stand-point, including other buffers, diluents, filters, needles, syringes, and package inserts with instructions for use.

Methods of Treatment and prevention

The present disclosure provides methods for treating a subject infected with a coronavirus, is at risk of infection with a coronavirus, or suffering from or suspected to suffer from a coronavirus associated disease. The present disclosure provides methods for treating a subject infected with a severe acute respiratory syndrome associated coronavirus (SARS-CoV), is at risk of infection with a SARS-CoV, or suffering from or suspected to suffer from a SARS-CoV associated disease. The present disclosure provides methods for treating a subject infected with a Middle East Respiratory Syndrome (MERS), is at risk of infection with a MERS, or suffering from or suspected to suffer from a MERS associated disease. The methods comprise administering to the subject, an effective amount of an antibody or antigen binding fragment thereof disclosed herein.

In one aspect, the disclosure provides methods for treatment or prevention of infection with a SARS-CoV-2, by the administration of an antibody or antigen-binding fragment thereof disclosed herein, to a patient in an amount effective to treat the patient.

The disclosure provides methods for treatment or prevention of infection with a SARS-CoV-2, by the administration of an antibody or antigen-binding fragment thereof disclosed herein, to a patient in an amount effective to treat the patient. The present disclosure provides a method of preventing an infection with a SARS-CoV-2 in a subject, the method comprising administering to the subject, an effective amount of an antibody or an antigen binding fragment thereof disclosed herein or an effective amount of a composition comprising an antibody or an antigen binding fragment disclosed herein or a nucleic acid encoding the antibody or antigen binding fragment thereof disclosed herein. The present disclosure provides a method of treating a subject infected with a SARS-Cov-2 (COVID) or suspected of being infected with a SARS-Cov-2, comprising administering to the subject an effective amount of an antibody or antigen-binding fragment herein, or an effective amount of a composition comprising an antibody or antigen binding fragment thereof disclosed herein or a nucleic acid encoding the antibody or antigen binding fragment thereof disclosed herein. In one aspect, the present disclosure provides a method for treating, or preventing COVID-19, comprising administering a subject in need thereof, an effective amount of an effective amount of an antibody or antigen-binding fragment herein, or an effective amount of a composition comprising an antibody or antigen binding fragment thereof disclosed herein or a nucleic acid encoding the antibody or antigen binding fragment thereof disclosed herein.

In some embodiments, the subject has one or more co-morbidities or has an increased risk of infection. Non-limiting exemplary co-morbidities or an underlying condition that the subject can have include high blood pressure, cardiac disease, diabetes, lung disease, cancer, clots, thrombosis, autoimmune disease, an inflammatory disease, or a combination thereof. In some embodiments, the subject is immunocompromised. In some embodiments, the subject is pregnant. In some embodiments, the subject to be treated is symptomatic prior to the administration. In other embodiments, the subject to be treated is asymptomatic prior to the administration.

In some embodiments, the subject is exhibiting one or more symptoms associated with infection with SARS-Cov-2. Non-limiting exemplary symptoms include fever, chills, cough, sore throat, diarrhea, shortness of breath or difficulty breathing, fatigue, muscle aches, body aches, headache, loss of taste, loss of smell, sore throat, congestion, runny nose, nausea, vomiting, diarrhea, trouble breathing, persistent pain or pressure in the chest, new confusion, inability to wake or stay awake and pale, gray, blue-colored skin, lips, or nail beds, depending on skin tone, dyspnea, hypoxemia, pneumonia, severe acute respiratory syndrome, renal failure, or any combination thereof.

As used herein, a “subject”, “patient”, “individual” and like terms are used interchangeably and refers to a vertebrate, a mammal, a primate, or a human. A subject can be male or female. A subject can be one who has been previously diagnosed with or identified as suffering from an infection with SARS-CoV-2. A subject can be one who is currently being treated for, or seeking treatment, monitoring, adjustment or modification of an existing therapeutic treatment, or is at a risk of developing an infection with SARS-CoV-2. Mammals include, without limitation, humans, primates, rodents, wild or domesticated animals, including feral animals, farm animals, sport animals, and pets. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. Mammals other than humans can be advantageously used as subjects that represent animal models of conditions or disorders associated with infection with SARS-CoV-2. In addition, the compositions and methods described herein can be used to treat domesticated animals and/or pets. In some embodiments, the subject is a human.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

The term “in need thereof” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

As used herein, the term “administering,” refers to the placement of a compound (e.g., an antibody or antigen binding fragment thereof as disclosed herein) into a subject by a method or route that results in at least partial delivery of the agent at a desired site. Pharmaceutical compositions comprising an antibody or antigen binding fragment thereof, disclosed herein can be administered by any appropriate route which results in an effective treatment in the subject, including but not limited to intravenous, intraarterial, injection or infusion directly into a tissue parenchyma, etc. Where necessary or desired, administration can include, for example, intracerebroventricular (“icy”) administration, intranasal administration, intracranial administration, intracelial administration, intracerebellar administration, or intrathecal administration.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the antibody or antibody fragment other than directly into a target site, tissue, or organ, such as a tumor site, such that it enters the subject's circulatory system and, thus, is subject to metabolism and other like processes.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with infection by SARS-CoV-2. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The term “effective amount” as used herein refers to the amount of an antibody or antigen binding fragment thereof or composition comprising the same needed to alleviate at least one or more symptom of the disease or disorder, and relates to a sufficient amount of pharmacological composition to provide the desired effect. The term “therapeutically effective amount” therefore refers to an amount of an antibody or antigen binding fragment thereof using the methods as disclosed herein, that is sufficient to effect a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. For any given case, however, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀-Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of the antibody or antigen binding fragment thereof), which achieves a half-maximal inhibition of symptoms as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The terms “increased”, “increase”, or “enhance” are all used herein to generally mean an increase by a statically significant amount; for the avoidance of doubt, the terms “increased”, “increase”, or “enhance”, mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms, “decrease”, “reduce”, “reduction”, “lower” or “lowering,” or “inhibit” are all used herein generally to mean a decrease by a statistically significant amount. For example, “decrease”, “reduce”, “reduction”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., tumor size after treatment as compared to a reference level prior to the treatment), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. Reduce or inhibit can refer to, for example, the symptoms of the disorder being treated, or the viral titer measurable in a subject's blood or other bodily fluids.

The antibodies or antigen binding fragment thereof or the compositions described herein (e.g., comprising an antibody or antigen binding fragment thereof, or a nucleic acid encoding said antibody or antigen binding fragment thereof described herein) can be administered alone or in combination with an additional therapeutic agent or therapy. In some embodiments, the methods of the present disclosure further comprise administering an additional therapeutic agent or therapy (e.g., administering a combination of an antibody disclosed herein and an additional therapeutic agent or therapy. In some embodiments, a combination with an additional therapeutic agent or therapy induces a synergistic effect relative to an effect induced upon administering the antibody or antigen binding fragment thereof or the composition alone, or the additional therapeutic agent or therapy alone. In some embodiments, the synergistic effect is therapeutic or prophylactic. In some embodiments, a combination with an additional therapeutic agent or therapy induces an additive effect relative to an effect induced upon administering the antibody or antigen binding fragment thereof, or the composition alone, or the additional therapeutic agent or therapy alone. In some embodiments, the additive effect is therapeutic or prophylactic.

In some embodiments, an antibody or an antigen binding fragment thereof or a composition disclosed herein is administered, for the prevention or treatment of a SARS-CoV-2 infection or COVID-19 e.g., at least 1 minute, 15 minutes, 30 minutes, 45 minutes, 1 hour, 2 hours, 4 hours, 6 hours, 8 hours, 12 hours, 24 hours, 2 days, or 1 week before), subsequent to administering an additional therapeutic agent or therapy. In some embodiments, an antibody or antigen binding fragment thereof or a composition disclosed herein is administered, for the prevention or treatment of a SARS-CoV-2 infection or COVID-19 e.g., at least 1 minute, 15 minutes, 30 minutes, 45 minutes, 1 hour, 2 hours, 4 hours, 6 hours, 8 hours, 12 hours, 24 hours, 2 days, or 1 week before), prior to administering an additional therapeutic agent or therapy. In some embodiments, an antibody or an antigen binding fragment thereof, or a composition disclosed herein is administered, for the prevention or treatment of a SARS-CoV-2 infection concomitantly with an additional therapeutic agent or therapy.

In some embodiments, the additional therapeutic agent or therapy is useful for treating an infection of SARS-CoV-2, or COVID-19. In some embodiments, the additional therapy is convalescent plasma therapy. In some embodiments, an additional therapeutic agent, can be a small molecule, an mRNA vaccine, a peptide, a pepti-body, a cytotoxic agent, a cytostatic agent, immunological modifier, interferon, interleukin, immunostimulatory growth hormone, cytokine, vitamin, mineral, aromatase inhibitor, RNAi, Histone Deacetylase Inhibitor, proteasome inhibitor, another antibody (for example, a SAR-Cov-2 neutralizing antibody), immunostimulatory antibody, a NSAID, a corticosteroid, a dietary supplement such as an antioxidant, cisplatin, ifosfamide, paclitaxel, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, taxol, one or more antibiotics (e.g., doxycycline, Azithromycin, etc.); one or more decongestants (e.g., Mucinex, Sudafed, etc.); one or more anti-histamines and/or glucocorticoids (e.g., Zyrtec, Claritin, Allegra, fluticasone luroate, etc.); one or more pain relievers (e.g., acetominophen); one or more zinc-containing medications (e.g., Zycam, etc.); Azithromycin, hydroquinolone, or a combination thereof; one or more integrase inhibitors (e.g. Bictegravir, dolutegravir (Tivicay), elvitegravir, raltegravir, or a combination thereof); one or more nucleoside/nucleotide reverse transcriptase inhibitors (NRTIs; e.g., abacavir (Ziagen), emtricitabine (Emtriva), lamivudine (Epivir), tenofovir alafenamide fumarate (Vemlidy), tenofovir disoproxil fumarate (Viread), zidovudine (Retrovir), didanosine (Videx, Videx EC), stavudine (Zerit), or a combination thereof); a combination of NRTIs (e.g., (i) abacavir, lamivudine, and zidovudine (Trizivir), abacavir and lamivudine (Epzicom), (iii) emtricitabine and tenofovir alafenamide fumarate (Descovy), (iv) emtricitabine and tenofovir disoproxil fumarate (Truvada), (v) lamivudine and tenofovir disoproxil fumarate (Cimduo, Temixys), (vi) lamivudine and zidovudine (Combivir), etc.); a combination of Descovy and Truvada; one or more non-nucleoside reverse transcriptase inhibitors (NNRTIs; e.g., doravirine (Pifeltro), efavirenz (Sustiva), etravirine (Intelence), nevirapine (Viramune, Viramune rilpivirine (Edurant), delavirdine (Rescriptor), or a combination thereof); one or more Cytochrome P4503A (CYP3A) inhibitors (e.g., cobicistat (Tybost), ritonavir (Norvir), etc.); one or more protease inhibitors (PIs; e.g., atazanavir (Reyataz), darunavir (Prezista), fosamprenavir (Lexiva), lopinavir, ritonavir (Norvir), tipranavir (Aptivus), etc.); one or PIs in combination with cobicistat, ritonavir, Lopinavir, Tipranavir, Atazanavir, fosamprenavir, indinavir (Crixivan), nelfinavir (Viracept), saquinavir (Invirase), or a combination thereof; Atazanavir; fosamprenavir; a combination of Atazanavir, darunavir and cobicistat; one or more fusion inhibitors (e.g., enfuvirtide (Fuzeon); one or more post-attachment inhibitors (e.g., ibalizumab-uiyk (Trogarzo)); one or more Chemokine coreceptor antagonists (CCR5 antagonists; maraviroc (Selzentry)); and one or more viral entry inhibitors (e.g., enfuvirtide (Fuzeon), ibalizumab-uiyk (Trogarzo), maraviroc (Selzentry), etc.); or a combination thereof.

In some embodiments, the additional therapeutic agent can be an additional anti-SARS-CoV-2 antibody or an antigen binding fragment thereof. In some embodiments, the additional anti-SARS-CoV-2 antibody is 2B04, 1B07, and 2H04 mAbs (Alsoussi et al., 2020, which is incorporated herein by reference in its entirety), bamlanivimab, etesevimab, casirivimab, imdevimab, Sotrovimab, JMB2002, LY-CovMab, ABBV-47D11, ADM03820, DXP604, ZRC-3308, HLX70, COR-101, VIR-7832, LY-CoV1404, LY3853113, COVI-AMG (STI-2020), DXP593, JS016, LY3832479, LY-CoV016, MW33, MAD0004J08, C144-LS, C-135-LS, SCTA01, ADG20, BRII-196, BRII-198, TY027, AZD7442 (AZD8895+AZD1061), CT-P59, VIR-7831, GSK4182136, LY-CoV555 (LY3819253), a combination of LY-CoV555 with LY-CoV016 (LY3832479), REGN10933, REGN10987, REGN-COV2 (REGN10933+REGN10987), or a combination thereof. In some embodiments, the additional therapeutic agent is an adjuvant (e.g., AddaVax). In some embodiments, the additional therapeutic agent is an anti-SARS-CoV-2 antibody described in Robbiani et al., Nature, 2020; Baum et al., Science 2020; Cao et al., Cell 2020; Hansen et al., Science 2020; Ju et al., Nature 2020; Liu et al., Nature 2020; Pinto et al., Nature 2020; Wang et al., 2020,Preprint; Zost et al., 2020a; Li et al., Nature 2020, each of which is incorporated herein by reference in its entirety. In some embodiments, the additional therapeutic agent is Remdesvir. In some embodiments, the additional therapeutic agent is Favipiravir. In some embodiments, an additional therapy is a cell based therapy including for example, administering mesenchymal stem cells. In some embodiments, the additional therapeutic agent is an immunomodulator. Non-limiting examples of immunomodulators include Colchicine, Corticosteroids (e.g., Budesonide (Inhaled), Dexamethasone (Systemic)), Fluvoxamine, Granulocyte-Macrophage Colony-Stimulating Factor Inhibitors (e.g., Lenzilumab, Mavrilimumab, Otilimab), Interferons (e.g., Interferon Alfa, Interferon Beta), Interleukin-1 Inhibitor (e.g., Anakinra), Interleukin-6 Inhibitors (e.g., Sarilumab, Tocilizumab), Anti-Interleukin-6 Monoclonal Antibody (e.g., Siltuximab), Kinase Inhibitors (e.g., Acalabrutinib, Ibrutinib, Zanubrutinib), Janus Kinase Inhibitors (e.g., Baricitinib, Ruxolitinib, Tofacitinib. In some embodiments, the additional therapy is an antithrombotic therapy (e.g., administering an anticoagulant). In some embodiments, the additional therapeutic agent is an angiotensin-converting enzyme [ACE] inhibitors, angiotensin receptor blockers [ARBs], HMG-CoA Reductase Inhibitors (Statins), systemic or inhaled corticosteroids, nonsteroidal anti-inflammatory drugs, acid-suppressive therapy, or acetaminophen. In some embodiments, the additional therapeutic agent is a dietary supplement (e.g., Vitamin C, Vitamin D, and Zinc). In some embodiments, the additional therapeutic agent is a therapeutic vaccine selected from a group consisting of exogenous vaccines including proteins, peptides, DNA, or mRNA used to mount an immunogenic response to a SARS-Cov-2, recombinant virus and bacteria vectors encoding SARS-CoV-2 antigens, DNA-based vaccines encoding SARS-CoV-2 antigens. In some embodiments, the additional therapeutic agent is EpiVacCorona, mRNA-1273 (RNA), BNT162b2, Ad5-nCoV, Sputnik V, Ad26.COV2.S, AZD1222, Covishield, Covaxin, BBIBP-CorV, Inactivated (Vero Cells), or CoronaVac. In some embodiments, the additional therapeutic agent is an antiviral agent. Non-limiting examples of an antiviral agent that can be used include Remdesivir, Ivermectin, Nitazoxanide, Hydroxychloroquine or Chloroquine and/or Azithromycin, Lopinavir/Ritonavir and Other HIV Protease Inhibitors.

The methods and compositions of the present disclosure contemplate single antibody or antigen binding fragment thereof, disclosed herein, as well as combinations, or “cocktails”, of more than one antibody or antigen binding fragment thereof, disclosed herein. In some embodiments, more than one antibody comprises at least 2, at least 3, at least 4, at least 5 or more antibodies or antigen binding fragment thereof, disclosed herein. In some embodiments, the methods of the present disclosure comprising administering to a subject, a first antibody disclosed herein, or a nucleic acid encoding the first antibody, and subsequently administering an additional antibody disclosed herein, or a nucleic acid encoding the additional antibody, wherein the first antibody and the additional antibody are not the same. In some embodiments, a subject is administered one of the antibodies or antigen-binding fragments herein one or more times. In some embodiments, a subject is administered two of the antibodies or antigen-binding fragments herein one or more times. In some embodiments, a subject is administered three of the antibodies or antigen-binding fragments herein one or more times. In some embodiments, a subject is administered four of the antibodies or antigen-binding fragments herein one or more times. In some embodiments, a subject is administered four or more of the antibodies or antigen-binding fragments herein one or more times. In some embodiments, a subject is administered five of the antibodies or antigen-binding fragments herein one or more times.

In some embodiments, an antibody or an antigen binding fragment thereof disclosed herein, or a composition disclosed herein (e.g., comprising an antibody or antigen binding fragment thereof, or a nucleic acid encoding said antibody or antigen binding fragment thereof described herein) can be administered as a booster dose after an initial dose. The term “booster” refers to an extra administration of an antibody or an antigen binding fragment thereof disclosed herein, or a composition disclosed herein typically provided subsequent to an initial dose of the antibody or an antigen binding fragment thereof, or a composition disclosed herein. In some embodiments, the methods of the present disclosure further comprise administering at least one booster dose to a subject. In some embodiments, the methods disclosed herein comprises administering at least 1, 2, 3, 4, or 5 booster doses. In some embodiments a booster dose is administered at least about 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years after administering an initial dose of an antibody, or a composition disclosed herein. In some embodiments, the booster dose comprises a reduced amount of an antibody or antigen binding fragment disclosed herein, or a composition disclosed herein than the initial dose. For example, a booster or subsequent dose of an antibody or antigen binding fragment thereof, or a composition can comprise an amount that is about: 1%, 2%, 3%, 4%, 5%, 7.5%, 10%, 15%, 20%, 25%, 30%, 40%, 50%, 60%, or 75% or less than the initial or preceding dose of the antibody or the antigen binding fragment thereof, or the composition. In some case a therapeutic or prophylactic effect is achieved in absence of a booster dose.

The present disclosure provides methods of reducing the death rate of infection by SARS-CoV-2 by administering to a population of subjects in need thereof an antibody or antigen-binding fragment disclosed herein, or a composition disclosed herein. Reduction in death rate can be determined for example by comparing the rate of death of subjects infected by SARS-CoV-2 between the population of subjects that receives an antibody or antigen binding fragment thereof, or a composition and a corresponding population of subjects that does not receive the antibody or antigen binding fragment thereof, or the composition, or are untreated. Death rate can be determined, for example, by determining the number of infected subjects of a population wherein infection by SARS-CoV-2 results in death. In some cases, the death rate can be reduced by at least 5%, at least 10%, at least 20%, at least 30%, at least 40%, at least 50%, at least 60%, at least 70%, at least 80%, or at least 90%. The present disclosure also provides methods for reducing the infection rate of SARS-CoV-2 by administering to a population of subjects non infected with SARS-CoV-2, an antibody or antigen binding fragment thereof disclosed herein or a composition disclosed herein. Reduction in infection rate can be determined for example by comparing the rate of infection of subjects exposed to SARS-CoV-2 between a population of subjects that receive an antibody or antigen binding fragment thereof disclosed herein or a composition disclosed herein, and a population of subjects that does not receive the antibody or antigen binding fragment thereof disclosed herein or the composition disclosed herein. Infection of a subject can be determined by analyzing a sample from the subject for the presence or absence of SARS-CoV-2 after suspected or confirmed exposure to SARS-CoV-2, or after an elapsed time in which exposure to SARS-CoV-2 is likely. In some embodiments, the infection rate can be reduced by at least 5%, at least 10%, at least 20%, at least 30%, at least 40%, at least 50%, at least 60%, at least 70%, at least 80%, or at least 90%.

The present disclosure also provides methods for slowing or preventing reproduction or replication of SARS-CoV-2 in a subject by administering to a subject infected with SARS-CoV-2 an antibody or antigen binding fragment thereof disclosed herein, or a composition disclosed herein. In some embodiments, the methods described herein slow or prevent reproduction or replication of SARS-CoV-2 in a subject relative that in a corresponding untreated subject.

Slowing or preventing reproduction or replication of a SARS-CoV-2 can be determined for example by comparing the rate of reproduction of the virus in a subject infected with SARS-CoV-2 between a subject who receives an antibody or antigen binding fragment thereof disclosed herein, or a composition disclosed herein and a corresponding subject that does not receive the antibody or antigen binding fragment thereof disclosed herein, or the composition disclosed herein, or a corresponding untreated subject. Replication of SARS-CoV-2 can be determined, for example by determining (directly or indirectly) the amount of SARS-Cov-2 in a sample acquired from a subject at different time points. Assays that can be used to determine amount of SARS-CoV-2 in a sample can include a plaque assay, a focus forming assay, an endpoint dilution assay, a protein assay (e.g., a bicinchoninic acid assay or a single radial immunodiffusion assay), transmission electron microscopy, tunable resistive pulse sensing, flow cytometry, qPCR, ELISA, or another acceptable method. An assay can be performed on a whole sample or a fraction of a sample, or SARS-CoV-2 can be isolated from the sample prior to performing an assay. In some embodiments, the reproduction of SARS-CoV-2 can be slowed by at least 5%, at least 10%, at least 20%, at least 30%, at least 40%, at least 50%, at least 60%, at least 70%, at least 80%, or at least 90%, or a range between any two foregoing values.

In some embodiments of the methods herein, said administering to a subject of an antibody or antigen binding fragment thereof, or a composition disclosed herein comprising the antibody or antigen binding fragment thereof, or a nucleic acid encoding the antibody or antigen binding fragment thereof results in inhibition of binding of a SARS-CoV-2 with a receptor (angiotensin-converting enzyme 2 (ACE2)) on a cell in the subject. In some embodiments, the inhibition of binding is by at least about 1%, 2%, 3%, 4%, 5%, 6%, 7%, 8%, 9%, 10%, 15%, 20%, 30%, 40%, 50%, 60%, 70%, 80%, 90%, or more or more compared to that in a subject not treated with the antibody or antigen-binding fragment thereof disclosed herein, or the composition disclosed herein or compared to an untreated subject.

In some embodiments of the methods herein, said administering to a subject of an antibody or antigen binding fragment thereof, or a composition disclosed herein comprising the antibody or antigen binding fragment thereof, or a nucleic acid encoding the antibody or antigen binding fragment thereof results in inhibition of entry of a SARS-CoV-2 in a cell in the subject. In some embodiments, the inhibition of entry is by at least about 1%, 2%, 3%, 4%, 5%, 6%, 7%, 8%, 9%, 10%, 15%, 20%, 30%, 40%, 50%, 60%, 70%, 80%, 90%, or more or more compared to that in a subject not treated with the antibody or antigen-binding fragment thereof disclosed herein, or the composition disclosed herein or compared to an untreated subject.

In some embodiments of the methods herein, said administering to a subject of an antibody or antigen binding fragment thereof, or a composition disclosed herein comprising the antibody or antigen binding fragment thereof, or a nucleic acid encoding the antibody or antigen binding fragment thereof results in inhibition of fusion of a SARS-CoV-2 cell membrane and the subject's cell membrane in the subject. In some embodiments, the inhibition of fusion is by at least about 1%, 2%, 3%, 4%, 5%, 6%, 7%, 8%, 9%, 10%, 15%, 20%, 30%, 40%, 50%, 60%, 70%, 80%, 90%, or more or more compared to that in a subject not treated with the antibody or antigen-binding fragment thereof disclosed herein, or the composition disclosed herein or compared to an untreated subject.

In some embodiments of the methods herein, said administering to a subject of an antibody or antigen binding fragment thereof, or a composition disclosed herein comprising the antibody or antigen binding fragment thereof, or a nucleic acid encoding the antibody or antigen binding fragment thereof results in decrease in viral load in the subject. In some embodiments, the decrease in viral load is by at least about 1%, 2%, 3%, 4%, 5%, 6%, 7%, 8%, 9%, 10%, 15%, 20%, 30%, 40%, 50%, 60%, 70%, 80%, 90%, or more or more compared to that in a subject not treated with the antibody or antigen-binding fragment thereof disclosed herein, or the composition disclosed herein or compared to an untreated subject.

In some embodiments of the methods herein, said administering to a subject of an antibody or antigen binding fragment thereof, or a composition disclosed herein comprising the antibody or antigen binding fragment thereof, or a nucleic acid encoding the antibody or antigen binding fragment thereof results in a decrease in one or more symptoms or conditions resulting from a SARS-CoV-2 infection in the subject for any period of time (e.g., for a day, a week, a month, 6 months, a year, or for the remainder of the subject's life).

In some embodiments of the methods herein, said administering to a subject of an antibody or antigen binding fragment thereof, or a composition disclosed herein comprising the antibody or antigen binding fragment thereof, or a nucleic acid encoding the antibody or antigen binding fragment thereof results in a decrease in one or more symptoms or conditions resulting from a SARS-CoV-2 infection in the subject for any period of time (e.g., for a day, a week, a month, 6 months, a year, or for the remainder of the subject's life).

In some embodiments of the methods herein, said administering to a subject of an antibody or antigen binding fragment thereof, or a composition disclosed herein comprising the antibody or antigen binding fragment thereof, or a nucleic acid encoding the antibody or antigen binding fragment thereof results in neutralization of SARS-CoV-2 in the subject, (i.e., inhibition of the SARS-CoV-2 to infect and cause a disease in the subject).

### Inhibition of a SARS-CoV-2 Activity

In some embodiments, the disclosure provides antibodies or antigen binding fragment thereof disclosed herein that are neutralizing antibodies. As used herein a “neutralizing antibody” is an antibody or antigen binding fragment thereof that binds to a SARS-CoV-2 and inhibits the ability to infect a host cell and/or cause a disease (e.g., COVID-19) in the subject. A neutralizing antibody specifically binds a target antigen on a SARS-CoV-2 and inhibits the ability of SARS-CoV-2 to infect a host cell and/or cause disease (e.g., COVID-19). Neutralization can be induced by an antibody or antigen binding fragment thereof disclosed herein by any mechanism, such as by inhibiting binding of a target antigen on SARS-Cov-2 (e.g., a SARS-Cov-2 spike (S) protein, a subunit S1, a subunit S2, or a receptor binding domain of subunit S1) with a receptor on a host cell. Neutralization can also be induced by inhibiting fusion of cell membrane of a SARS-CoV-2 with that of a host cell membrane, inhibiting entry of SARS-CoV-2 in a host cell, or a combination thereof. Neutralization assays are capable of being performed and measured in different ways, including the use of techniques such as plaque reduction (which compares counts of virus plaques in control wells with those in inoculated cultures), microneutralization (which is performed in microtiter plates filled with small amounts of sera), and colorimetric assays (which depend on biomarkers indicating metabolic inhibition of the virus). In some embodiments, the antibodies or antigen binding fragment thereof exhibits increased neutralizing activity relative to that by a corresponding control antibody or an antigen binding fragment thereof. In some embodiments, the increased neutralization activity is by at least about 1%, 2%, 3%, 4%, 5%, 6%, 7%, 8%, 9%, 10%, 15%, 20%, 30%, 40%, 50%, 60%, 70%, 80%, 90%, or more compared a corresponding control antibody or an antigen binding fragment thereof.

In another embodiment, the disclosure provides antibodies or antigen binding fragment thereof disclosed herein that inhibit, block, or decrease a SARS-CoV-2 binding to a receptor on a host cell, in particular, to angiotensin-converting enzyme 2 (ACE2). Provided herein is a method to inhibit binding of a SARS-CoV-2 to a receptor on a host cell, comprising contacting the SARS-CoV-2 with an antibody or an antigen binding fragment thereof disclosed herein. In some embodiments, inhibition of a SARS-CoV-2 binding comprises inhibition of a target antigen on SARS-CoV-2 (e.g., a SARS-Cov-2 spike (S) protein, a subunit S1, a subunit S2, or a receptor binding domain of subunit S1) to a receptor on a receptor on a host cell.

In another embodiment, the disclosure provides an antibody or an antigen binding fragment thereof that inhibits, blocks, or decreases SARS-CoV-2 entry into a host cell. Provided herein is a method to inhibit entry of a SARS-CoV-2 in a host cell, comprising contacting the SARS-CoV-2 with an antibody or an antigen binding fragment thereof disclosed herein.

In another embodiment, the disclosure provides an antibody or an antigen binding fragment thereof that inhibits, blocks, or decreases fusion of a SARS-CoV-2 cell membrane and a host cell membrane. Provided herein is a method to inhibit fusion of a SARS-CoV-2 cell membrane and a host cell membrane, comprising contacting the SARS-CoV-2 with an antibody or an antigen binding fragment thereof disclosed herein.

In another embodiment, the disclosure provides an antibody or an antigen binding fragment thereof disclosed herein that decreases viral load. Provided herein is a method to decrease viral load, comprising contacting the SARS-CoV-2 with an antibody or an antigen binding fragment thereof disclosed herein.

In another embodiment, the disclosure provides an antibody or an antigen binding fragment thereof disclosed herein that inhibits, blocks, or decreases one or more symptoms or conditions resulting from a SARS-CoV-2 infection for any period of time. In certain embodiments, the one or more symptoms are decreased for a day, a week, a month, 6 months, a year, or for the remainder of the subject's life. In certain embodiments, the disclosure provides an antibody or an antigen binding fragment thereof disclosed herein that can perform any combination of the preceding embodiments.

### Dosages

The compositions are to be used for in vivo administration to a subject by any available means, such as parenteral administration. For administration to a subject, a composition or medicament described herein can be sterile, which can readily be accomplished by filtration through sterile filtration membranes, or other methods known to those of skill in the art. In one embodiment, a composition of medicament has been treated to be free of pyrogens or endotoxins. Testing pharmaceutical compositions or medicaments for pyrogens or endotoxins and preparing pharmaceutical compositions or medicaments free of pyrogens or endotoxins, or preparing pharmaceutical compositions or medicaments that have endotoxins at a clinically-acceptable level, are well understood to one of ordinary skill in the art. Commercial kits are available to test pharmaceutical compositions or medicaments for pyrogens or endotoxins.

The antibodies or antigen binding fragments thereof, describe herein, are formulated, dosed, and administered in a fashion consistent with good medical practice. Factors for consideration in this context include the particular disorder being treated, the particular subject being treated, the clinical condition of the individual subject, the cause of the disorder, the site of delivery of the agent, the method of administration, the scheduling of administration, and other factors known to medical practitioners. The “therapeutically effective amount” to be administered will be governed by such considerations, and refers to the minimum amount necessary to ameliorate, treat, or resolve, an infection with SARS-CoV-2; or to prevent or protect against an infection with SARS-CoV-2.

The dose of antibody may vary depending upon the age and the size of a subject to be administered, target disease, conditions, route of administration, and the like. The preferred dose is typically calculated according to body weight or body surface area. When an antibody or antigen binding fragment thereof disclosed herein is used for treating a condition or disease in an adult patient, it may be advantageous to intravenously administer the antibody of the present invention normally at a single dose of about 0.01 to about 20 mg/kg body weight, more preferably about 0.02 to about 7, about 0.03 to about 5, or about 0.05 to about 3 mg/kg, about 5 mg/kg, about 7.5 mg/kg, about 10 mg/kg, or about 15 mg/kg body weight. Depending on the severity of the condition, the frequency and the duration of the treatment can be adjusted. Effective dosages and schedules for administering may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

The administration can be, for example, by one or more separate administrations, or by continuous infusion. However, other dosage regimens can be useful. In one non-limiting example, an antibody or antigen binding fragment thereof, disclosed herein is administered once every week, every two weeks, or every three weeks, at a dose range from about 5 mg/kg to about 15 mg/kg, including but not limited to 5 mg/kg, 7.5 mg/kg, 10 mg/kg or 15 mg/kg. The duration of a therapy using the methods described herein will continue for as long as medically indicated or until a desired therapeutic effect (e.g., those described herein) is achieved.

### Efficacy of Treatment

The efficacy of treatment or prevention of infection with SARS-CoV-2, comprising administering the antibodies or antigen binding fragment thereof, or pharmaceutical compositions of the present disclosure, may be assessed using standard techniques, for example by measuring a patient's SARS-CoV-2 viral load via reverse transcriptase quantitative PCR (RT-qPCR) (see, e.g., To, K. K. et al., Lancet Infect. Dis. 20(5):565-574 (2020)), microscopy, or phage assays. Other measures may include duration of survival, progression free survival, overall response rate, duration of response, and quality of life.

In some embodiments, an antibody or antigen binding fragment thereof disclosed herein is a neutralizing antibody or an antigen binding fragment thereof. In some embodiments, an antibody or antigen binding fragment thereof disclosed herein inhibits entry of SARS-Cov-2 in a host cell, viral replication, fusion of viral membrane to host cell membrane, endocytosis of SARS-Cov-2 in a host cell, activity of SARS-CoV-2 3-chymotrypsin-like protease (3CLpro) or the RNA-dependent RNA polymerase.

A subject can be administered an antibody or antigen-binding fragment thereof disclosed herein, or a composition disclosed herein in an amount that achieves at least partially, a partial, or complete reduction of one or more symptoms (e.g., one or more symptoms associated with COVID-19. Reduction can be, for example, a decrease of one or more symptoms by at least about 1%, 2%, 3%, 4%, 5%, 6%, 7%, 8%, 9%, 10%, 15%, 20%, 30%, 40%, 50%, 60%, 70%, 80%, 90%, or more or more compared to that in a subject not treated with the antibody or antigen-binding fragment thereof disclosed herein, or the composition disclosed herein or compared to an untreated subject. The amount of an antibody necessary to bring about therapeutic treatment of COVID-19 is not fixed per se. The amount of an antibody administered can vary for example with the extensiveness of the disease, the size of the human suffering from COVID-19, and if the subject is suffering from, or is at risk of another comorbidity. Treatment, in one instance, lowers infection rates in a population of subjects for example by at least about 1%, 2%, 3%, 4%, 5%, 6%, 7%, 8%, 9%, 10%, 15%, 20%, 30%, 40%, 50%, 60%, 70%, 80%, 90%, or more or more compared to treatment of a corresponding population of subjects with another treatment for COVID-19, or compared to a corresponding untreated subject population. Treatment can also result in a shortened recovery time, in fewer symptoms, or in less severe symptoms, or a combination thereof compared to an untreated subject who has COVID-19.

The antibodies and antigen-binding fragments herein can be used to treat a COVID-19 infection (an infection caused by SARS-Cov-2) in a subject in need thereof, thereby reducing one or more symptoms of the infection. The one or more symptoms to be treated include, but are not limited to, fever of over 100.4.degree.F, chills, cough, sore throat, diarrhea, shortness of breath or difficulty breathing, fatigue, muscle aches, body aches, headache, loss of taste, loss of smell, sore throat, congestion, runny nose, lung disease, nausea, vomiting, diarrhea, trouble breathing, persistent pain or pressure in the chest, new confusion, inability to wake or stay awake and pale, gray, blue-colored skin, lips, or nail beds, depending on skin tone, dyspnea, hypoxemia, pneumonia, severe acute respiratory syndrome, or renal failure, or any combination thereof. In some embodiments, treatment of a subject includes a reduction by at least 1%, 2%, 3%, 4%, 5%, 6%, 7%, 8%, 9%, 10%, 15%, 20%, 30%, 40%, 50%, 60%, 70%, 80%, 90%, or more in 1 symptom, 2 symptoms, 3 symptoms, 4 symptoms, 5 symptoms, 6 symptoms, 7 symptoms, 8 symptoms, 9 symptoms, 10 symptoms, or 11 symptoms. During at least a portion of this time period the antibody or antigen-binding fragment can protect the subject from infection by SARS-Cov-2. Protecting can comprise for example reducing an infection rate of SARS-Cov-2 or reducing or preventing reproduction of SARS-Cov-2. Treatment can comprise for example reducing symptoms of COVID-19, reducing a death rate, or reducing or preventing reproduction of SARS-Cov-2.

In some embodiments, the antibodies or antigen binding fragment thereof disclosed herein or a composition disclosed herein induce an inhibition of a SARS-CoV-2 activity in vivo, and in vitro, for example, binding of a SARS-CoV-2 to a receptor on a host cell, entry of a SARS-CoV-2 in a host cell, fusion of a SARS-CoV-2 cell membrane and a host cell membrane, or viral load. Methods to measure SARS-CoV-2 activity are known in the art, for example, assays for viral entry or fusion, viral load, and viral attachment to a cell membrane are described in Tai et al. J Vis Exp. 2015; (104): 53124, Pohl et al. J Vis Exp. 2015; (105): 53372, and Berry et al. Bio-protocol, Vol 7, Iss 2, Jan. 20, 2017, Schmidt, F. et al. Measuring SARS-CoV-2 neutralizing antibody activity using pseudotyped and chimeric viruses. J. Exp. Med. 217, e20201181 (2020), Greaney, A. J. et al. Complete mapping of mutations to the SARS-CoV-2 spike receptor-binding domain that escape antibody recognition. Preprint at bioRxiv doi.org/10.1101/2020.09.10.292078, Starr, T. N. et al. Deep mutational scanning of SARS-CoV-2 receptor binding domain reveals constraints on folding and ACE2 binding. Cell 182, 1295-1310 (2020), Tan, C. W. et al. A SARS-CoV-2 surrogate virus neutralization test based on antibody-mediated blockage of ACE2-spike protein-protein interaction. Nat. Biotechnol. 38, 1073-1078 (2020), and Abe, K. T. et al. A simple protein-based surrogate neutralization assay for SARS-CoV-2. JCI Insight 5, e142362 (2020). A person of skill in the art would be easily able to perform these measurements.

In some embodiments, the antibodies or antigen binding fragment thereof disclosed herein are neutralizing antibodies. The neutralization capacity of an antibody can be demonstrated by measuring the ability of antibodies to inhibit the binding of the receptor binding domain (RBD) of the SARS-CoV-2 to the ACE2. The assays for these measurements are well known in the art, and described for example, in Tan, C. W. et al. A SARS-CoV-2 surrogate virus neutralization test based on antibody-mediated blockage of ACE2-spike protein-protein interaction. Nat. Biotechnol. 38, 1073-1078 (2020), and Abe, K. T. et al. A simple protein-based surrogate neutralization assay for SARS-CoV-2. JCI Insight 5, e142362 (2020). Neutralization activity is measured by comparing infection levels in antibody-treated and untreated samples (e.g., from a subject), and efficacy is reported, for example, as an IC50 (the concentration of antibody required to reduce infection to 50% of that seen in an untreated sample). The IC50 in these assays is typically interpreted as the concentration of an antibody or an antigen binding fragment thereof required to neutralize 50% of SARS-Cov-2 virions. Neutralization assays are well known in the art, and are described for example, in Khoury, D. S., Wheatley, A. K., Ramuta, M. D. et al. Measuring immunity to SARS-CoV-2 infection: comparing assays and animal models. Nat Rev Immunol 20, 727-738 (2020). The Examples described herein demonstrate IC50 of antibodies disclosed herein, and their neutralization capacity.

### Modes of Administration

The antibodies or antigen binding fragment thereof, described herein, can be administered to a subject in need thereof by any appropriate route which results in an effective treatment in the subject. In some embodiments, the antibodies or antigen binding fragment thereof, described herein, or compositions comprising the same is administered to a subject infected with SARS-CoV-2, or seeking to prevent infection with SARS-CoV-2, by any mode of administration that delivers the agent systemically or to a desired surface or target, and can include, but is not limited to, injection, infusion, instillation, inhalation, parenteral, subcutaneous, intraperitoneal, intrapulmonary, oral and intranasal administration. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intracranial, intraspinal, intracerebro spinal, and intrasternal injection and infusion. Parenteral infusions include intramuscular, intravenous, intraarterial, intraperitoneal, or subcutaneous administration.

### Diagnostic and Other Uses

Provided herein are methods of using the antibodies or the antigen binding fragment disclosed herein for detection, diagnosis and monitoring of a disease, disorder or condition associated with the target antigen expression (either increased or decreased relative to a normal sample, and/or inappropriate expression, such as presence of expression in tissues(s) and/or cell(s) that normally lack the epitope expression). Provided herein are methods of determining whether a patient will respond to an antibody therapy. Provided herein are methods of diagnosing a subject suspected of being infected with SARS-CoV-2 or suffering from COVID-19 by contacting a sample obtained from the subject with one or more antibodies or antigen-binding fragments herein. Provided herein are methods of detecting infection with SARS-CoV-2 in a subject suspected of being infected with SARS-CoV-2 or suffering from COVID-19 by contacting a sample obtained from the subject with one or more antibodies or antigen-binding fragments herein.

Provided herein are methods of monitoring progression of COVID-19 in a subject suspected of being infected with SARS-CoV-2 or suffering from COVID-19 by contacting a first sample obtained from the subject with one or more antibodies or antigen-binding fragments herein, measuring a first level of binding of the one or more antibodies or antigen-binding fragments herein, contacting a second sample obtained from the subject with the one or more antibodies or antigen-binding fragments herein, and measuring a second level of binding of the one or more antibodies or antigen-binding fragments herein, wherein an increase in the second level relative to the first level indicates an increase in COVID-19 in the subject, and a decrease in the second level relative to the first indicates a decrease in COVID-19 in the subject, thereby monitoring the disease. In some embodiments, the first sample is obtained prior to administering a select treatment. In some embodiments, the second sample is obtained after administering a select treatment. A decrease in COVID-19 indicates the select treatment to be effective. A “sample” from a subject to be tested utilizing one or more of the assays described herein includes, but is not limited to, a nasal swab, a tissue sample, saliva, blood, etc. In some instances, the sample is treated prior to use in a diagnostic assay. For example, a nasal swab may be flushed with phosphate buffered saline (PBS); a fluid sample may be centrifuged to concentrate the sample components; blood may be treated with heparin to prevent coagulation, etc. The sample is contacted with an antibody or antigen-binding fragment herein, and when the presence of the antibody bound to a SARS-CoV-2 target antigen is detected, the subject is diagnosed as being infected with SARS-CoV-2 and/or having a COVID-19 infection. In some embodiments, a sample obtained from a subject is contacted with an antibody or antigen-binding fragment herein that selectively binds to a SARS-CoV-2 target antigen and the presence or absence of the antibody or antigen-binding fragment is determined. The subject is diagnosed as being infected with SARS-CoV-2 when the presence of the antibody or antigen-binding fragment is detected.

In some embodiments, the method of detection comprises contacting a sample from a subject with an antibody or antigen binding fragment thereof of the disclosure, and determining whether the level of binding differs from that of a reference or comparison sample (such as a control). In some embodiments, the method may be useful to determine whether the antibodies or polypeptides described herein are an appropriate treatment for the subject. When the sample show binding activity as compared to a corresponding reference sample, it can indicate that the subject would benefit from treatment with an antibody.

Samples can be tested in any suitable assay including, but not limited to, an enzyme linked immunosorbent assay (ELISA), an immunospot assay, a lateral flow assay, immunohistochemistry (IHC), western blot, flow cytometry, etc.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures.

Appropriate labels include, without limitation, radionuclides (for example 125I, 131I, 35S, 3H, or 32P), enzymes (for example, alkaline phosphatase, horseradish peroxidase, luciferase, or β-glactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

For purposes of diagnosis, the antibodies or antigen binding fragment thereof can be labeled with a detectable moiety including but not limited to radioisotopes, fluorescent labels, and various enzyme-substrate labels know in the art. Methods of conjugating labels to an antibody are known in the art.

In some embodiments, the antibodies need not be labeled, and the presence thereof can be detected using a second labeled antibody which binds to the first antibody. The antibodies or antigen binding fragment thereof of the present invention can be used as affinity purification agents for a SARS-CoV-2 target antigen or in diagnostic assays for COVID-19, e.g., detecting its presence in a sample from a subject suffering from or suspected to suffer from COVID-19. The antibodies or antigen binding fragment thereof, disclosed herein, may also be used for in vivo diagnostic assays. Generally, for these purposes the antibody is labeled with a radionuclide (such as u1In, 99Tc, 14C, 131I, 12sI, 3H, 32p or 3sS) so that the virus can be localized using immunoscintiography.

The antibodies of the present invention can be employed in any known assay method, such as competitive binding assays, direct and indirect sandwich assays, such as ELISAs, and immunoprecipitation assays. Zola, Monoclonal Antibodies: A Manual of Techniques, pp. 147-158 (CRC Press, Inc. 1987). The antibodies can also be used for immunohistochemistry, to label tumor samples using methods known in the art. As a matter of convenience, the antibody of the present invention can be provided in a kit, i.e., a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives can be included such as stabilizers, buffers (e.g., a block buffer or lysis buffer) and the like. The relative amounts of the various reagents can be varied widely to provide for concentrations in solution of the reagents which substantially optimize the sensitivity of the assay. Particularly, the reagents can be provided as dry powders, usually lyophilized, including excipients which on dissolution will provide a reagent solution having the appropriate concentration.

### Fusion Proteins

In one aspect, provided herein is a fusion protein comprising an antibody or an antigen binding fragment, disclosed herein. In some embodiments, fusion protein comprises one or more antibody or antigen binding fragment thereof, disclosed herein, and an immunomodulator or toxin moiety. Methods of making antibody fusion proteins are known. Antibody fusion proteins comprising an interleukin-2 moiety are described by Boleti et al., Ann. Oneal. 6:945 (1995), Nicolet et al., Cancer Gene Ther. 2:161 (1995), Becker et al., Proc. Natl Acad. Sci. USA 93:7826 (1996), Hank et al., Clin. Cancer Res. 2:1951 (1996), and Hu et al., Cancer Res. 56:4998 (1996). In addition, Yang et al., Hum. Antibodies Hybridomas 6:129 (1995), describe a fusion protein that includes an F(ab′)2 fragment and a tumor necrosis factor alpha moiety.

Methods of making antibody-toxin fusion proteins in which a recombinant molecule comprises one or more antibody components and a toxin or a therapeutic agent also are known to those of skill in the art. For example, antibody-Pseudomonas exotoxin A fusion proteins have been described by Chaudhary et al., Nature 339:394 (1989), Brinkmann et al., Proc. Nat'l Acad. Sci. USA 88:8616 (1991), Batra et al., Proc. Natl Acad. Sci. USA 89:5867 (1992), Friedman et al., J. Immunol. 150:3054 (1993), Weis et al., Int. J. Can. 60:137 (1995), Fominaya et al., J. Biol. Chem. 271:10560 (1996), Kuan et al., Biochemistry 35:2872 (1996), and Schmidt et al., Int. J. Can. 65:538 (1996). Antibody-toxin fusion proteins containing a diphtheria toxin moiety have been described by Kreitman et al., Leukemia 7:553 (1993), Nicholls et al., J. Biol. Chem. 268:5302 (1993), Thompson et al., J. Biol. Chem. 270:28037 (1995), and Vallera et al., Blood 88:2342 (1996). Deonarain et al., Tumor Targeting 1:177 (1995), have described an antibody-toxin fusion protein having an RNase moiety, while Linardou et al., Cell Biophys. 24-25:243 (1994), produced an antibody-toxin fusion protein comprising a DNase I component. Gelonin was used as the toxin moiety in the antibody-toxin fusion protein of Wang et al., Abstracts of the 209th ACS National Meeting, Anaheim, Calif., Apr. 2-6, 1995, Part 1, BIOT005. As a further example, Dohlsten et al., Proc. Natl Acad. Sci. USA 91:8945 (1994), reported an antibody-toxin fusion protein comprising Staphylococcal enterotoxin-A.

Illustrative of toxins which are suitably employed in the preparation of such conjugates are ricin, abrin, ribonuclease, DNase I, Staphylococcal enterotoxin-A, pokeweed antiviral protein, gelonin, diphtherin toxin, Pseudomonas exotoxin, and Pseudomonas endotoxin. See, for example, Pastan et al., Cell 47:641 (1986), and Goldenberg, C A-A Cancer Journal for Clinicians 44:43 (1994). Other suitable toxins are known to those of skill in the art.

Antibodies or antigen binding fragment thereof, disclosed herein, may also be used in ADEPT by conjugating the antibody to a prodrug-activating enzyme which converts a prodrug (e.g., a peptidyl chemotherapeutic agent, See WO81/01145) to an active anti-cancer drug. See, for example, WO88/07378 and U.S. Pat. No. 4,975,278. The enzyme component of the immunoconjugate useful for ADEPT includes any enzyme capable of acting on a prodrug in such a way so as to convert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as galactosidase and neuraminidase useful for converting glycosylated prodrugs into free drugs; lactamase useful for converting drugs derivatized with lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as abzymes, can be used to convert the prodrugs of the invention into free active drugs (See, e.g., Massey, Nature 328: 457-458 (1987)). Antibody-abzyme conjugates can be prepared as described herein for delivery of the abzyme to a tumor cell population. The enzymes can be covalently bound to the antibodies by techniques well known in the art such as the use of the heterobifunctional crosslinking reagents discussed above. Alternatively, fusion proteins comprising at least the antigen binding region of an antibody of the invention linked to at least a functionally active portion of an enzyme can be constructed using recombinant DNA techniques well known in the art (See, e.g., Neuberger et al., Nature 312: 604-608 (1984)).

### Immunoconjugates

The antibodies or antigen binding fragment thereof, disclosed herein, may be administered in their “naked” or unconjugated form, or may have an additional therapeutic agent conjugated to them. For example, the antibodies or antigen binding fragment of the present disclosure can have a toxin, radioisotope, or a label conjugated to them. In one embodiment, antibodies or antigen binding fragment thereof are used as a radiosensitizer. In such embodiments, the antibodies or antigen binding fragment are conjugated to a radiosensitizing agent. The term “radiosensitizer,” as used herein, is defined as a molecule, preferably a low molecular weight molecule, administered to animals in therapeutically effective amounts to increase the sensitivity of the cells to be detected by radiation, or radiosensitized to electromagnetic radiation and/or to promote the treatment of diseases that are treatable with electromagnetic radiation.

The terms “electromagnetic radiation” and “radiation” as used herein include, but are not limited to, radiation having the wavelength of 10-20 to 100 meters. Preferred embodiments of the present disclosure can employ for example, the electro-magnetic radiation of: gamma-radiation c10-20 to 10-13 m), X-ray radiation (10-12 to 10-9 m), ultraviolet light (10 nm to 400 nm), visible light (400 nm to 700 nm), infrared radiation (700 nm to 1.0 mm), and microwave radiation (1 mm to 30 cm).

Examples of photodynamic radiosensitizers include the following, but are not limited to: hematoporphyrin derivatives, Photofrin®, benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same that can be conjugated to the antibodies or antigen binding fragment thereof disclosed herein.

In another embodiment, the antibody may be conjugated to a receptor (such streptavidin), wherein the antibody-receptor conjugate is administered to the patient, followed by removal of unbound conjugate from the circulation using a clearing agent and then administration of a ligand (e.g., avidin) which is conjugated to an additional therapeutic agent (e.g., an anti-viral agent).

The present disclosure further provides the above-described antibodies or antigen binding thereof in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent or luminescent or bioluminescent labels (such as FITC or rhodamine, etc.), paramagnetic atoms, and the like. Procedures for accomplishing such labeling are well known in the art; for example, see (Sternberger, L. A. et al., J. Histochem. Cytochem. 18:315 (1970); Bayer, E. A. et al., Meth. Enzym. 62:308 (1979); Engval, E. et al., Immunol. 109:129 (1972); Goding, J. W. J. Immunol. Meth. 13:215 (1976)).

“Label” refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable. Alternatively, the label may not be detectable on its own but may be an element that is bound by another agent that is detectable (e.g. an epitope tag or one of a binding partner pair such as biotin-avidin, etc.). Thus, the antibody may comprise a label or tag that facilitates its isolation, and methods of the invention to identify antibodies include a step of isolating the antigen/antibody through interaction with the label or tag.

Exemplary therapeutic immunoconjugates comprise the antibody described herein conjugated to an antiviral agent, or a radioactive isotope (i.e., a radioconjugate). Fusion proteins are described in further detail above.

In some embodiments, antibodies and antigen binding fragments thereof disclosed herein can be conjugated to an additional therapeutic agent described herein. In another embodiment, antibodies and antigen binding fragments thereof disclosed herein are conjugated to a detectable substrate such as, e.g., an enzyme, fluorescent marker, chemiluminescent marker, bioluminescent material, or radioactive material. In some embodiments of the aspects described herein, the antibody and antibody fragments thereof disclosed herein are conjugated to a toxin (e.g., an enzymatically active toxin of bacterial, fungal, plant or animal origin, or fragments thereof), a small molecule, an siRNA, a nanoparticle, a targeting agent (e.g., a microbubble), or a radioactive isotope (i.e., a radioconjugate). Such conjugates are referred to herein as “immunoconjugates”. Such immunoconjugates can be used, for example, in diagnostic, theranostic, or targeting methods.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. A variety of radioisotopes are available for the production of radioconjugate antibodies. Examples include, but are not limited to, 212 Bi, 131 I, 131 In, 90Y and 186Re.

Conjugates of the antibodies or antigen binding fragments thereof described herein and a therapeutic agent can be made using any of a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., 238 Science 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62: 119-58 (1982).

Production of immunoconjugates is described in U.S. Pat. No. 6,306,393. Immunoconjugates can be pre-pared by indirectly conjugating a therapeutic agent to an antibody component. General techniques are described in Shih et al., Int. J. Cancer 41:832-839 (1988); Shih et al., Int. J. Cancer 46:1101-1106 (1990); and Shih et al., U.S. Pat. No. 5,057,313. The general method involves reacting an antibody component having an oxidized carbohydrate portion with a carrier polymer that has at least one free amine function and that is loaded with a plurality of drug, toxin, chelator, boron addends, or other therapeutic agent. This reaction results in an initial Schiff base (imine) linkage, which can be stabilized by reduction to a secondary amine to form the final conjugate.

### Kits

Provided herein are also kits, medicines, compositions, and unit dosage forms for use in any of the methods described herein. Provided herein is a kit comprising an effective amount of at least one of the antibody or antigen binding fragment thereof disclosed herein, or a composition comprising the at least one antibody or antigen binding fragment thereof or a nucleic acid encoding the at least one antibody or antigen binding fragment thereof disclosed herein. In some embodiments, the kit further comprises an additional therapeutic agent described herein. In some embodiments, the antibody or antigen binding fragment thereof disclosed herein, or a composition disclosed herein is an aqueous form or a lyophilized form. In some embodiments, the kit further comprises a diluent or a reconstitution solution.

Kits can include one or more containers comprising an antibody or a composition described herein (or unit dosage forms and/or articles of manufacture). In some embodiments, a unit dosage is provided wherein the unit dosage contains a predetermined amount of an antibody or antigen binding fragment thereof or a composition disclosed herein, with or without one or more additional agents. In some embodiments, such a unit dosage is supplied in single-use prefilled syringe for injection. In some embodiments, the composition can comprise saline, sucrose, or the like; a buffer, such as phosphate, or the like; and/or be formulated within a stable and effective pH range. In some embodiments, an antibody or antigen binding fragment thereof, or a composition of the disclosure can be provided as a lyophilized powder that can be reconstituted upon addition of an appropriate liquid, for example, sterile water. In some embodiments, the composition further comprises one or more substances that inhibit protein aggregation, including, but not limited to, sucrose and arginine. In some embodiments, the composition further comprises heparin and/or a proteoglycan.

In some embodiments, kits further comprise instructions for use in the treatment of disease associated an infection with a coronavirus (e.g., COVID-19) in accordance with any of the methods described herein. The kit may further comprise a description of selection an individual suitable or treatment. Instructions supplied in the kits are typically written instructions on a label or package insert (for example, a paper sheet included in the kit), but machine-readable instructions (for example, instructions carried on a magnetic or optical storage disk) are also acceptable. In some embodiments, the kit further comprises an additional therapeutic agent described herein.

The kits are in suitable packaging. Suitable packaging includes, but is not limited to, vials, bottles, jars, flexible packaging (for example, sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### EXAMPLES

Provided below are exemplary methods for in silico reconstruction of consensus sequences of SARS-CoV-2 associated antibodies, and identification of the clonal rearranged immunoglobulin CDR sequences present in the immunoglobulin repertoire. The approaches are contemplated for the reconstruction of complete consensus sequences of the variable heavy chain, variable light chain and the respective CDRs of said immunoglobulins.

### Example 1: Extraction of RNA-Seq Reads from Immunoglobulin Genes

RNA-Seq sequencing reads were run through a three-stage filtering procedure to remove: a.) reads known to have originated from non-immunoglobulin genes, b.) reads that may have arisen from other sources of human DNA contamination, and c.) reads from non-human sources of contamination. To execute the first stage, a Kallisto index file (see, e.g., Bray, N. L. et al. Nat. Biotechnol. 34(5):525-527 (2016)) was created of the human transcriptome of the HG38 human reference genome, with the immunoglobulin gene sequences removed. RNA-Seq FASTQ input files obtained from fluid specimens of patients diagnosed with infection by SARS-CoV-2 were run against this index file using Kallisto. For paired-end FASTQ files, reads in which one of the mates failed to map were saved for further analysis; all other reads were discarded. For single-ended FASTQ files, reads which failed to map were saved for further analysis, and all other reads were similarly discarded. For stage two, the remaining reads were then further filtered to eliminate other possible sources of human DNA contamination. The reads were aligned to the complete HG38 human reference genome using BWA-MEM (Li, H. arXiv: Genomics arXiv:1303.3997v2), and they were discarded unless they fit one of the following criteria: a.) paired-end reads in which one mate was unmapped (or, for single-ended reads, in which the read was unmapped), or b.) reads which mapped to known human immunoglobulin gene locations. For the third and final stage, any read sequences still remaining were compared to a database of common sources of non-human contamination (see, e.g., Wood, D. E. et al. Genome Biol. 15(3):R46 (2014)). Reads which matched a known sequence in a database were removed.

### Example 2: Consensus Sequence Assembly

RNA-Seq reads which passed the filtration procedure were assembled into contigs using a De Brujin graph-based sequence assembly algorithm, optimized for RNA-Seq data (see, e.g., Grabherr, M. G. et al. Nat. Biotechnol. 29(7):644-652 (2011)). Assembled contigs were passed through IgBLAST (Jian, Y. et al. Nucleic Acids Res. 41(W1):W34-W40 (2013)), and only sequences which were marked as significant matches to an immunoglobulin chain were kept.

### Example 3: Chain Quantification and Selection of the Top Clone

Immunoglobulin chains identified in the previous exemplary method were then quantified using Kallisto. A Kallisto index file was generated for the assembled immunoglobulin chain sequences, and the original set of RNA-Seq sequencing reads (prior to any filtering) was run through Kallisto quantification using this index file. The most abundant heavy and light chain antibody sequences counted by the Kallisto quantification step were identified as the top clones.

### Example 4: Annotation of the Reference Chain Segments

For each individual patient specimen, the heavy and light chains of the top clone were further analyzed using IgBLAST to annotate the V, D, and J segments of that clone. Amino acid translation was also performed to produce the polypeptide consensus sequence for the heavy and light chains of the top clone.

TABLE 1
SEQ ID NOs of exemplary amino acid sequences of complementarity determining, heavy chain, and
light chain regions of the antibodies disclosed herein or antigen binding fragment thereof.
Corresponding amino acid sequences are provided in the sequence listing submitted herewith.

| | CDR-1 | | CDR-2 | | CDR-3 | | Variable Region | |
|---|---|---|---|---|---|---|---|---|
| | Heavy | Light | Heavy | Light | Heavy | Light | Heavy | Light |
| | Chain | Chain | Chain | Chain | Chain | Chain | Chain | Chain |
| Antibody ID | (CDR-H1) | (CDR-L1) | (CDR-H2) | (CDR-L2) | (CDR-H3) | (CDR-L3) | (V_{H}) | (V_{L}) |
| TOTCOVID1 | 10001 | 11251 | 12501 | 13751 | 15001 | 16251 | 17501 | 18751 |
| TOTCOVID2 | 10002 | 11252 | 12502 | 13752 | 15002 | 16252 | 17502 | 18752 |
| TOTCOVID3 | 10003 | 11253 | 12503 | 13753 | 15003 | 16253 | 17503 | 18753 |
| TOTCOVID4 | 10004 | 11254 | 12504 | 13754 | 15004 | 16254 | 17504 | 18754 |
| TOTCOVID5 | 10005 | 11255 | 12505 | 13755 | 15005 | 16255 | 17505 | 18755 |
| TOTCOVID6 | 10006 | 11256 | 12506 | 13756 | 15006 | 16256 | 17506 | 18756 |
| TOTCOVID7 | 10007 | 11257 | 12507 | 13757 | 15007 | 16257 | 17507 | 18757 |
| TOTCOVID8 | 10008 | 11258 | 12508 | 13758 | 15008 | 16258 | 17508 | 18758 |
| TOTCOVID9 | 10009 | 11259 | 12509 | 13759 | 15009 | 16259 | 17509 | 18759 |
| TOTCOVID282 | 10010 | 11260 | 12510 | 13760 | 15010 | 16260 | 17510 | 18760 |
| TOTCOVID10 | 10011 | 11261 | 12511 | 13761 | 15011 | 16261 | 17511 | 18761 |
| TOTCOVID11 | 10012 | 11262 | 12512 | 13762 | 15012 | 16262 | 17512 | 18762 |
| TOTCOVID12 | 10013 | 11263 | 12513 | 13763 | 15013 | 16263 | 17513 | 18763 |
| TOTCOVID13 | 10014 | 11264 | 12514 | 13764 | 15014 | 16264 | 17514 | 18764 |
| TOTCOVID14 | 10015 | 11265 | 12515 | 13765 | 15015 | 16265 | 17515 | 18765 |
| TOTCOVID15 | 10016 | 11266 | 12516 | 13766 | 15016 | 16266 | 17516 | 18766 |
| TOTCOVID16 | 10017 | 11267 | 12517 | 13767 | 15017 | 16267 | 17517 | 18767 |
| TOTCOVID17 | 10018 | 11268 | 12518 | 13768 | 15018 | 16268 | 17518 | 18768 |
| TOTCOVID18 | 10019 | 11269 | 12519 | 13769 | 15019 | 16269 | 17519 | 18769 |
| TOTCOVID25 | 10020 | 11270 | 12520 | 13770 | 15020 | 16270 | 17520 | 18770 |
| TOTCOVID26 | 10021 | 11271 | 12521 | 13771 | 15021 | 16271 | 17521 | 18771 |
| TOTCOVID27 | 10022 | 11272 | 12522 | 13772 | 15022 | 16272 | 17522 | 18772 |
| TOTCOVID28 | 10023 | 11273 | 12523 | 13773 | 15023 | 16273 | 17523 | 18773 |
| TOTCOVID29 | 10024 | 11274 | 12524 | 13774 | 15024 | 16274 | 17524 | 18774 |
| TOTCOVID30 | 10025 | 11275 | 12525 | 13775 | 15025 | 16275 | 17525 | 18775 |
| TOTCOVID31 | 10026 | 11276 | 12526 | 13776 | 15026 | 16276 | 17526 | 18776 |
| TOTCOVID32 | 10027 | 11277 | 12527 | 13777 | 15027 | 16277 | 17527 | 18777 |
| TOTCOVID33 | 10028 | 11278 | 12528 | 13778 | 15028 | 16278 | 17528 | 18778 |
| TOTCOVID34 | 10029 | 11279 | 12529 | 13779 | 15029 | 16279 | 17529 | 18779 |
| TOTCOVID35 | 10030 | 11280 | 12530 | 13780 | 15030 | 16280 | 17530 | 18780 |
| TOTCOVID36 | 10031 | 11281 | 12531 | 13781 | 15031 | 16281 | 17531 | 18781 |
| TOTCOVID37 | 10032 | 11282 | 12532 | 13782 | 15032 | 16282 | 17532 | 18782 |
| TOTCOVID38 | 10033 | 11283 | 12533 | 13783 | 15033 | 16283 | 17533 | 18783 |
| TOTCOVID39 | 10034 | 11284 | 12534 | 13784 | 15034 | 16284 | 17534 | 18784 |
| TOTCOVID40 | 10035 | 11285 | 12535 | 13785 | 15035 | 16285 | 17535 | 18785 |
| TOTCOVID41 | 10036 | 11286 | 12536 | 13786 | 15036 | 16286 | 17536 | 18786 |
| TOTCOVID42 | 10037 | 11287 | 12537 | 13787 | 15037 | 16287 | 17537 | 18787 |
| TOTCOVID43 | 10038 | 11288 | 12538 | 13788 | 15038 | 16288 | 17538 | 18788 |
| TOTCOVID44 | 10039 | 11289 | 12539 | 13789 | 15039 | 16289 | 17539 | 18789 |
| TOTCOVID45 | 10040 | 11290 | 12540 | 13790 | 15040 | 16290 | 17540 | 18790 |
| TOTCOVID46 | 10041 | 11291 | 12541 | 13791 | 15041 | 16291 | 17541 | 18791 |
| TOTCOVID47 | 10042 | 11292 | 12542 | 13792 | 15042 | 16292 | 17542 | 18792 |
| TOTCOVID48 | 10043 | 11293 | 12543 | 13793 | 15043 | 16293 | 17543 | 18793 |
| TOTCOVID49 | 10044 | 11294 | 12544 | 13794 | 15044 | 16294 | 17544 | 18794 |
| TOTCOVID50 | 10045 | 11295 | 12545 | 13795 | 15045 | 16295 | 17545 | 18795 |
| TOTCOVID51 | 10046 | 11296 | 12546 | 13796 | 15046 | 16296 | 17546 | 18796 |
| TOTCOVID53 | 10047 | 11297 | 12547 | 13797 | 15047 | 16297 | 17547 | 18797 |
| TOTCOVID54 | 10048 | 11298 | 12548 | 13798 | 15048 | 16298 | 17548 | 18798 |
| TOTCOVID55 | 10049 | 11299 | 12549 | 13799 | 15049 | 16299 | 17549 | 18799 |
| TOTCOVID56 | 10050 | 11300 | 12550 | 13800 | 15050 | 16300 | 17550 | 18800 |
| TOTCOVID57 | 10051 | 11301 | 12551 | 13801 | 15051 | 16301 | 17551 | 18801 |
| TOTCOVID58 | 10052 | 11302 | 12552 | 13802 | 15052 | 16302 | 17552 | 18802 |
| TOTCOVID59 | 10053 | 11303 | 12553 | 13803 | 15053 | 16303 | 17553 | 18803 |
| TOTCOVID60 | 10054 | 11304 | 12554 | 13804 | 15054 | 16304 | 17554 | 18804 |
| TOTCOVID61 | 10055 | 11305 | 12555 | 13805 | 15055 | 16305 | 17555 | 18805 |
| TOTCOVID62 | 10056 | 11306 | 12556 | 13806 | 15056 | 16306 | 17556 | 18806 |
| TOTCOVID63 | 10057 | 11307 | 12557 | 13807 | 15057 | 16307 | 17557 | 18807 |
| TOTCOVID64 | 10058 | 11308 | 12558 | 13808 | 15058 | 16308 | 17558 | 18808 |
| TOTCOVID65 | 10059 | 11309 | 12559 | 13809 | 15059 | 16309 | 17559 | 18809 |
| TOTCOVID66 | 10060 | 11310 | 12560 | 13810 | 15060 | 16310 | 17560 | 18810 |
| TOTCOVID67 | 10061 | 11311 | 12561 | 13811 | 15061 | 16311 | 17561 | 18811 |
| TOTCOVID68 | 10062 | 11312 | 12562 | 13812 | 15062 | 16312 | 17562 | 18812 |
| TOTCOVID69 | 10063 | 11313 | 12563 | 13813 | 15063 | 16313 | 17563 | 18813 |
| TOTCOVID70 | 10064 | 11314 | 12564 | 13814 | 15064 | 16314 | 17564 | 18814 |
| TOTCOVID71 | 10065 | 11315 | 12565 | 13815 | 15065 | 16315 | 17565 | 18815 |
| TOTCOVID72 | 10066 | 11316 | 12566 | 13816 | 15066 | 16316 | 17566 | 18816 |
| TOTCOVID73 | 10067 | 11317 | 12567 | 13817 | 15067 | 16317 | 17567 | 18817 |
| TOTCOVID74 | 10068 | 11318 | 12568 | 13818 | 15068 | 16318 | 17568 | 18818 |
| TOTCOVID75 | 10069 | 11319 | 12569 | 13819 | 15069 | 16319 | 17569 | 18819 |
| TOTCOVID76 | 10070 | 11320 | 12570 | 13820 | 15070 | 16320 | 17570 | 18820 |
| TOTCOVID77 | 10071 | 11321 | 12571 | 13821 | 15071 | 16321 | 17571 | 18821 |
| TOTCOVID78 | 10072 | 11322 | 12572 | 13822 | 15072 | 16322 | 17572 | 18822 |
| TOTCOVID79 | 10073 | 11323 | 12573 | 13823 | 15073 | 16323 | 17573 | 18823 |
| TOTCOVID80 | 10074 | 11324 | 12574 | 13824 | 15074 | 16324 | 17574 | 18824 |
| TOTCOVID81 | 10075 | 11325 | 12575 | 13825 | 15075 | 16325 | 17575 | 18825 |
| TOTCOVID82 | 10076 | 11326 | 12576 | 13826 | 15076 | 16326 | 17576 | 18826 |
| TOTCOVID83 | 10077 | 11327 | 12577 | 13827 | 15077 | 16327 | 17577 | 18827 |
| TOTCOVID84 | 10078 | 11328 | 12578 | 13828 | 15078 | 16328 | 17578 | 18828 |
| TOTCOVID85 | 10079 | 11329 | 12579 | 13829 | 15079 | 16329 | 17579 | 18829 |
| TOTCOVID86 | 10080 | 11330 | 12580 | 13830 | 15080 | 16330 | 17580 | 18830 |
| TOTCOVID87 | 10081 | 11331 | 12581 | 13831 | 15081 | 16331 | 17581 | 18831 |
| TOTCOVID88 | 10082 | 11332 | 12582 | 13832 | 15082 | 16332 | 17582 | 18832 |
| TOTCOVID90 | 10083 | 11333 | 12583 | 13833 | 15083 | 16333 | 17583 | 18833 |
| TOTCOVID91 | 10084 | 11334 | 12584 | 13834 | 15084 | 16334 | 17584 | 18834 |
| TOTCOVID92 | 10085 | 11335 | 12585 | 13835 | 15085 | 16335 | 17585 | 18835 |
| TOTCOVID93 | 10086 | 11336 | 12586 | 13836 | 15086 | 16336 | 17586 | 18836 |
| TOTCOVID94 | 10087 | 11337 | 12587 | 13837 | 15087 | 16337 | 17587 | 18837 |
| TOTCOVID95 | 10088 | 11338 | 12588 | 13838 | 15088 | 16338 | 17588 | 18838 |
| TOTCOVID96 | 10089 | 11339 | 12589 | 13839 | 15089 | 16339 | 17589 | 18839 |
| TOTCOVID97 | 10090 | 11340 | 12590 | 13840 | 15090 | 16340 | 17590 | 18840 |
| TOTCOVID98 | 10091 | 11341 | 12591 | 13841 | 15091 | 16341 | 17591 | 18841 |
| TOTCOVID99 | 10092 | 11342 | 12592 | 13842 | 15092 | 16342 | 17592 | 18842 |
| TOTCOVID100 | 10093 | 11343 | 12593 | 13843 | 15093 | 16343 | 17593 | 18843 |
| TOTCOVID101 | 10094 | 11344 | 12594 | 13844 | 15094 | 16344 | 17594 | 18844 |
| TOTCOVID102 | 10095 | 11345 | 12595 | 13845 | 15095 | 16345 | 17595 | 18845 |
| TOTCOVID103 | 10096 | 11346 | 12596 | 13846 | 15096 | 16346 | 17596 | 18846 |
| TOTCOVID105 | 10097 | 11347 | 12597 | 13847 | 15097 | 16347 | 17597 | 18847 |
| TOTCOVID107 | 10098 | 11348 | 12598 | 13848 | 15098 | 16348 | 17598 | 18848 |
| TOTCOVID108 | 10099 | 11349 | 12599 | 13849 | 15099 | 16349 | 17599 | 18849 |
| TOTCOVID109 | 10100 | 11350 | 12600 | 13850 | 15100 | 16350 | 17600 | 18850 |
| TOTCOVID110 | 10101 | 11351 | 12601 | 13851 | 15101 | 16351 | 17601 | 18851 |
| TOTCOVID111 | 10102 | 11352 | 12602 | 13852 | 15102 | 16352 | 17602 | 18852 |
| TOTCOVID112 | 10103 | 11353 | 12603 | 13853 | 15103 | 16353 | 17603 | 18853 |
| TOTCOVID113 | 10104 | 11354 | 12604 | 13854 | 15104 | 16354 | 17604 | 18854 |
| TOTCOVID114 | 10105 | 11355 | 12605 | 13855 | 15105 | 16355 | 17605 | 18855 |
| TOTCOVID115 | 10106 | 11356 | 12606 | 13856 | 15106 | 16356 | 17606 | 18856 |
| TOTCOVID116 | 10107 | 11357 | 12607 | 13857 | 15107 | 16357 | 17607 | 18857 |
| TOTCOVID117 | 10108 | 11358 | 12608 | 13858 | 15108 | 16358 | 17608 | 18858 |
| TOTCOVID118 | 10109 | 11359 | 12609 | 13859 | 15109 | 16359 | 17609 | 18859 |
| TOTCOVID119 | 10110 | 11360 | 12610 | 13860 | 15110 | 16360 | 17610 | 18860 |
| TOTCOVID120 | 10111 | 11361 | 12611 | 13861 | 15111 | 16361 | 17611 | 18861 |
| TOTCOVID121 | 10112 | 11362 | 12612 | 13862 | 15112 | 16362 | 17612 | 18862 |
| TOTCOVID122 | 10113 | 11363 | 12613 | 13863 | 15113 | 16363 | 17613 | 18863 |
| TOTCOVID124 | 10114 | 11364 | 12614 | 13864 | 15114 | 16364 | 17614 | 18864 |
| TOTCOVID125 | 10115 | 11365 | 12615 | 13865 | 15115 | 16365 | 17615 | 18865 |
| TOTCOVID126 | 10116 | 11366 | 12616 | 13866 | 15116 | 16366 | 17616 | 18866 |
| TOTCOVID127 | 10117 | 11367 | 12617 | 13867 | 15117 | 16367 | 17617 | 18867 |
| TOTCOVID128 | 10118 | 11368 | 12618 | 13868 | 15118 | 16368 | 17618 | 18868 |
| TOTCOVID129 | 10119 | 11369 | 12619 | 13869 | 15119 | 16369 | 17619 | 18869 |
| TOTCOVID130 | 10120 | 11370 | 12620 | 13870 | 15120 | 16370 | 17620 | 18870 |
| TOTCOVID131 | 10121 | 11371 | 12621 | 13871 | 15121 | 16371 | 17621 | 18871 |
| TOTCOVID132 | 10122 | 11372 | 12622 | 13872 | 15122 | 16372 | 17622 | 18872 |
| TOTCOVID133 | 10123 | 11373 | 12623 | 13873 | 15123 | 16373 | 17623 | 18873 |
| TOTCOVID134 | 10124 | 11374 | 12624 | 13874 | 15124 | 16374 | 17624 | 18874 |
| TOTCOVID135 | 10125 | 11375 | 12625 | 13875 | 15125 | 16375 | 17625 | 18875 |
| TOTCOVID136 | 10126 | 11376 | 12626 | 13876 | 15126 | 16376 | 17626 | 18876 |
| TOTCOVID137 | 10127 | 11377 | 12627 | 13877 | 15127 | 16377 | 17627 | 18877 |
| TOTCOVID138 | 10128 | 11378 | 12628 | 13878 | 15128 | 16378 | 17628 | 18878 |
| TOTCOVID139 | 10129 | 11379 | 12629 | 13879 | 15129 | 16379 | 17629 | 18879 |
| TOTCOVID140 | 10130 | 11380 | 12630 | 13880 | 15130 | 16380 | 17630 | 18880 |
| TOTCOVID141 | 10131 | 11381 | 12631 | 13881 | 15131 | 16381 | 17631 | 18881 |
| TOTCOVID142 | 10132 | 11382 | 12632 | 13882 | 15132 | 16382 | 17632 | 18882 |
| TOTCOVID143 | 10133 | 11383 | 12633 | 13883 | 15133 | 16383 | 17633 | 18883 |
| TOTCOVID144 | 10134 | 11384 | 12634 | 13884 | 15134 | 16384 | 17634 | 18884 |
| TOTCOVID150 | 10135 | 11385 | 12635 | 13885 | 15135 | 16385 | 17635 | 18885 |
| TOTCOVID152 | 10136 | 11386 | 12636 | 13886 | 15136 | 16386 | 17636 | 18886 |
| TOTCOVID154 | 10137 | 11387 | 12637 | 13887 | 15137 | 16387 | 17637 | 18887 |
| TOTCOVID155 | 10138 | 11388 | 12638 | 13888 | 15138 | 16388 | 17638 | 18888 |
| TOTCOVID156 | 10139 | 11389 | 12639 | 13889 | 15139 | 16389 | 17639 | 18889 |
| TOTCOVID158 | 10140 | 11390 | 12640 | 13890 | 15140 | 16390 | 17640 | 18890 |
| TOTCOVID159 | 10141 | 11391 | 12641 | 13891 | 15141 | 16391 | 17641 | 18891 |
| TOTCOVID160 | 10142 | 11392 | 12642 | 13892 | 15142 | 16392 | 17642 | 18892 |
| TOTCOVID161 | 10143 | 11393 | 12643 | 13893 | 15143 | 16393 | 17643 | 18893 |
| TOTCOVID162 | 10144 | 11394 | 12644 | 13894 | 15144 | 16394 | 17644 | 18894 |
| TOTCOVID163 | 10145 | 11395 | 12645 | 13895 | 15145 | 16395 | 17645 | 18895 |
| TOTCOVID164 | 10146 | 11396 | 12646 | 13896 | 15146 | 16396 | 17646 | 18896 |
| TOTCOVID165 | 10147 | 11397 | 12647 | 13897 | 15147 | 16397 | 17647 | 18897 |
| TOTCOVID166 | 10148 | 11398 | 12648 | 13898 | 15148 | 16398 | 17648 | 18898 |
| TOTCOVID168 | 10149 | 11399 | 12649 | 13899 | 15149 | 16399 | 17649 | 18899 |
| TOTCOVID170 | 10150 | 11400 | 12650 | 13900 | 15150 | 16400 | 17650 | 18900 |
| TOTCOVID173 | 10151 | 11401 | 12651 | 13901 | 15151 | 16401 | 17651 | 18901 |
| TOTCOVID174 | 10152 | 11402 | 12652 | 13902 | 15152 | 16402 | 17652 | 18902 |
| TOTCOVID176 | 10153 | 11403 | 12653 | 13903 | 15153 | 16403 | 17653 | 18903 |
| TOTCOVID177 | 10154 | 11404 | 12654 | 13904 | 15154 | 16404 | 17654 | 18904 |
| TOTCOVID178 | 10155 | 11405 | 12655 | 13905 | 15155 | 16405 | 17655 | 18905 |
| TOTCOVID179 | 10156 | 11406 | 12656 | 13906 | 15156 | 16406 | 17656 | 18906 |
| TOTCOVID182 | 10157 | 11407 | 12657 | 13907 | 15157 | 16407 | 17657 | 18907 |
| TOTCOVID186 | 10158 | 11408 | 12658 | 13908 | 15158 | 16408 | 17658 | 18908 |
| TOTCOVID189 | 10159 | 11409 | 12659 | 13909 | 15159 | 16409 | 17659 | 18909 |
| TOTCOVID191 | 10160 | 11410 | 12660 | 13910 | 15160 | 16410 | 17660 | 18910 |
| TOTCOVID193 | 10161 | 11411 | 12661 | 13911 | 15161 | 16411 | 17661 | 18911 |
| TOTCOVID194 | 10162 | 11412 | 12662 | 13912 | 15162 | 16412 | 17662 | 18912 |
| TOTCOVID195 | 10163 | 11413 | 12663 | 13913 | 15163 | 16413 | 17663 | 18913 |
| TOTCOVID196 | 10164 | 11414 | 12664 | 13914 | 15164 | 16414 | 17664 | 18914 |
| TOTCOVID197 | 10165 | 11415 | 12665 | 13915 | 15165 | 16415 | 17665 | 18915 |
| TOTCOVID198 | 10166 | 11416 | 12666 | 13916 | 15166 | 16416 | 17666 | 18916 |
| TOTCOVID199 | 10167 | 11417 | 12667 | 13917 | 15167 | 16417 | 17667 | 18917 |
| TOTCOVID200 | 10168 | 11418 | 12668 | 13918 | 15168 | 16418 | 17668 | 18918 |
| TOTCOVID201 | 10169 | 11419 | 12669 | 13919 | 15169 | 16419 | 17669 | 18919 |
| TOTCOVID203 | 10170 | 11420 | 12670 | 13920 | 15170 | 16420 | 17670 | 18920 |
| TOTCOVID204 | 10171 | 11421 | 12671 | 13921 | 15171 | 16421 | 17671 | 18921 |
| TOTCOVID207 | 10172 | 11422 | 12672 | 13922 | 15172 | 16422 | 17672 | 18922 |
| TOTCOVID208 | 10173 | 11423 | 12673 | 13923 | 15173 | 16423 | 17673 | 18923 |
| TOTCOVID209 | 10174 | 11424 | 12674 | 13924 | 15174 | 16424 | 17674 | 18924 |
| TOTCOVID210 | 10175 | 11425 | 12675 | 13925 | 15175 | 16425 | 17675 | 18925 |
| TOTCOVID211 | 10176 | 11426 | 12676 | 13926 | 15176 | 16426 | 17676 | 18926 |
| TOTCOVID212 | 10177 | 11427 | 12677 | 13927 | 15177 | 16427 | 17677 | 18927 |
| TOTCOVID213 | 10178 | 11428 | 12678 | 13928 | 15178 | 16428 | 17678 | 18928 |
| TOTCOVID214 | 10179 | 11429 | 12679 | 13929 | 15179 | 16429 | 17679 | 18929 |
| TOTCOVID215 | 10180 | 11430 | 12680 | 13930 | 15180 | 16430 | 17680 | 18930 |
| TOTCOVID217 | 10181 | 11431 | 12681 | 13931 | 15181 | 16431 | 17681 | 18931 |
| TOTCOVID218 | 10182 | 11432 | 12682 | 13932 | 15182 | 16432 | 17682 | 18932 |
| TOTCOVID219 | 10183 | 11433 | 12683 | 13933 | 15183 | 16433 | 17683 | 18933 |
| TOTCOVID222 | 10184 | 11434 | 12684 | 13934 | 15184 | 16434 | 17684 | 18934 |
| TOTCOVID223 | 10185 | 11435 | 12685 | 13935 | 15185 | 16435 | 17685 | 18935 |
| TOTCOVID225 | 10186 | 11436 | 12686 | 13936 | 15186 | 16436 | 17686 | 18936 |
| TOTCOVID226 | 10187 | 11437 | 12687 | 13937 | 15187 | 16437 | 17687 | 18937 |
| TOTCOVID227 | 10188 | 11438 | 12688 | 13938 | 15188 | 16438 | 17688 | 18938 |
| TOTCOVID230 | 10189 | 11439 | 12689 | 13939 | 15189 | 16439 | 17689 | 18939 |
| TOTCOVID232 | 10190 | 11440 | 12690 | 13940 | 15190 | 16440 | 17690 | 18940 |
| TOTCOVID233 | 10191 | 11441 | 12691 | 13941 | 15191 | 16441 | 17691 | 18941 |
| TOTCOVID236 | 10192 | 11442 | 12692 | 13942 | 15192 | 16442 | 17692 | 18942 |
| TOTCOVID237 | 10193 | 11443 | 12693 | 13943 | 15193 | 16443 | 17693 | 18943 |
| TOTCOVID240 | 10194 | 11444 | 12694 | 13944 | 15194 | 16444 | 17694 | 18944 |
| TOTCOVID241 | 10195 | 11445 | 12695 | 13945 | 15195 | 16445 | 17695 | 18945 |
| TOTCOVID242 | 10196 | 11446 | 12696 | 13946 | 15196 | 16446 | 17696 | 18946 |
| TOTCOVID244 | 10197 | 11447 | 12697 | 13947 | 15197 | 16447 | 17697 | 18947 |
| TOTCOVID245 | 10198 | 11448 | 12698 | 13948 | 15198 | 16448 | 17698 | 18948 |
| TOTCOVID247 | 10199 | 11449 | 12699 | 13949 | 15199 | 16449 | 17699 | 18949 |
| TOTCOVID249 | 10200 | 11450 | 12700 | 13950 | 15200 | 16450 | 17700 | 18950 |
| TOTCOVID250 | 10201 | 11451 | 12701 | 13951 | 15201 | 16451 | 17701 | 18951 |
| TOTCOVID251 | 10202 | 11452 | 12702 | 13952 | 15202 | 16452 | 17702 | 18952 |
| TOTCOVID253 | 10203 | 11453 | 12703 | 13953 | 15203 | 16453 | 17703 | 18953 |
| TOTCOVID255 | 10204 | 11454 | 12704 | 13954 | 15204 | 16454 | 17704 | 18954 |
| TOTCOVID256 | 10205 | 11455 | 12705 | 13955 | 15205 | 16455 | 17705 | 18955 |
| TOTCOVID257 | 10206 | 11456 | 12706 | 13956 | 15206 | 16456 | 17706 | 18956 |
| TOTCOVID259 | 10207 | 11457 | 12707 | 13957 | 15207 | 16457 | 17707 | 18957 |
| TOTCOVID260 | 10208 | 11458 | 12708 | 13958 | 15208 | 16458 | 17708 | 18958 |
| TOTCOVID261 | 10209 | 11459 | 12709 | 13959 | 15209 | 16459 | 17709 | 18959 |
| TOTCOVID263 | 10210 | 11460 | 12710 | 13960 | 15210 | 16460 | 17710 | 18960 |
| TOTCOVID266 | 10211 | 11461 | 12711 | 13961 | 15211 | 16461 | 17711 | 18961 |
| TOTCOVID267 | 10212 | 11462 | 12712 | 13962 | 15212 | 16462 | 17712 | 18962 |
| TOTCOVID268 | 10213 | 11463 | 12713 | 13963 | 15213 | 16463 | 17713 | 18963 |
| TOTCOVID269 | 10214 | 11464 | 12714 | 13964 | 15214 | 16464 | 17714 | 18964 |
| TOTCOVID270 | 10215 | 11465 | 12715 | 13965 | 15215 | 16465 | 17715 | 18965 |
| TOTCOVID271 | 10216 | 11466 | 12716 | 13966 | 15216 | 16466 | 17716 | 18966 |
| TOTCOVID272 | 10217 | 11467 | 12717 | 13967 | 15217 | 16467 | 17717 | 18967 |
| TOTCOVID273 | 10218 | 11468 | 12718 | 13968 | 15218 | 16468 | 17718 | 18968 |
| TOTCOVID274 | 10219 | 11469 | 12719 | 13969 | 15219 | 16469 | 17719 | 18969 |
| TOTCOVID275 | 10220 | 11470 | 12720 | 13970 | 15220 | 16470 | 17720 | 18970 |
| TOTCOVID276 | 10221 | 11471 | 12721 | 13971 | 15221 | 16471 | 17721 | 18971 |
| TOTCOVID277 | 10222 | 11472 | 12722 | 13972 | 15222 | 16472 | 17722 | 18972 |
| TOTCOVID278 | 10223 | 11473 | 12723 | 13973 | 15223 | 16473 | 17723 | 18973 |
| TOTCOVID279 | 10224 | 11474 | 12724 | 13974 | 15224 | 16474 | 17724 | 18974 |
| TOTCOVID280 | 10225 | 11475 | 12725 | 13975 | 15225 | 16475 | 17725 | 18975 |
| TOTCOVID281 | 10226 | 11476 | 12726 | 13976 | 15226 | 16476 | 17726 | 18976 |
| TOTCOVID00283 | 10227 | 11477 | 12727 | 13977 | 15227 | 16477 | 17727 | 18977 |
| TOTCOVID00284 | 10228 | 11478 | 12728 | 13978 | 15228 | 16478 | 17728 | 18978 |
| TOTCOVID00285 | 10229 | 11479 | 12729 | 13979 | 15229 | 16479 | 17729 | 18979 |
| TOTCOVID00286 | 10230 | 11480 | 12730 | 13980 | 15230 | 16480 | 17730 | 18980 |
| TOTCOVID00287 | 10231 | 11481 | 12731 | 13981 | 15231 | 16481 | 17731 | 18981 |
| TOTCOVID00288 | 10232 | 11482 | 12732 | 13982 | 15232 | 16482 | 17732 | 18982 |
| TOTCOVID00289 | 10233 | 11483 | 12733 | 13983 | 15233 | 16483 | 17733 | 18983 |
| TOTCOVID00290 | 10234 | 11484 | 12734 | 13984 | 15234 | 16484 | 17734 | 18984 |
| TOTCOVID00291 | 10235 | 11485 | 12735 | 13985 | 15235 | 16485 | 17735 | 18985 |
| TOTCOVID00292 | 10236 | 11486 | 12736 | 13986 | 15236 | 16486 | 17736 | 18986 |
| TOTCOVID00293 | 10237 | 11487 | 12737 | 13987 | 15237 | 16487 | 17737 | 18987 |
| TOTCOVID00294 | 10238 | 11488 | 12738 | 13988 | 15238 | 16488 | 17738 | 18988 |
| TOTCOVID00295 | 10239 | 11489 | 12739 | 13989 | 15239 | 16489 | 17739 | 18989 |
| TOTCOVID00296 | 10240 | 11490 | 12740 | 13990 | 15240 | 16490 | 17740 | 18990 |
| TOTCOVID00297 | 10241 | 11491 | 12741 | 13991 | 15241 | 16491 | 17741 | 18991 |
| TOTCOVID00298 | 10242 | 11492 | 12742 | 13992 | 15242 | 16492 | 17742 | 18992 |
| TOTCOVID00299 | 10243 | 11493 | 12743 | 13993 | 15243 | 16493 | 17743 | 18993 |
| TOTCOVID00300 | 10244 | 11494 | 12744 | 13994 | 15244 | 16494 | 17744 | 18994 |
| TOTCOVID00301 | 10245 | 11495 | 12745 | 13995 | 15245 | 16495 | 17745 | 18995 |
| TOTCOVID00302 | 10246 | 11496 | 12746 | 13996 | 15246 | 16496 | 17746 | 18996 |
| TOTCOVID00303 | 10247 | 11497 | 12747 | 13997 | 15247 | 16497 | 17747 | 18997 |
| TOTCOVID00304 | 10248 | 11498 | 12748 | 13998 | 15248 | 16498 | 17748 | 18998 |
| TOTCOVID00305 | 10249 | 11499 | 12749 | 13999 | 15249 | 16499 | 17749 | 18999 |
| TOTCOVID00306 | 10250 | 11500 | 12750 | 14000 | 15250 | 16500 | 17750 | 19000 |
| TOTCOVID00307 | 10251 | 11501 | 12751 | 14001 | 15251 | 16501 | 17751 | 19001 |
| TOTCOVID00308 | 10252 | 11502 | 12752 | 14002 | 15252 | 16502 | 17752 | 19002 |
| TOTCOVID00309 | 10253 | 11503 | 12753 | 14003 | 15253 | 16503 | 17753 | 19003 |
| TOTCOVID00310 | 10254 | 11504 | 12754 | 14004 | 15254 | 16504 | 17754 | 19004 |
| TOTCOVID00311 | 10255 | 11505 | 12755 | 14005 | 15255 | 16505 | 17755 | 19005 |
| TOTCOVID00312 | 10256 | 11506 | 12756 | 14006 | 15256 | 16506 | 17756 | 19006 |
| TOTCOVID00313 | 10257 | 11507 | 12757 | 14007 | 15257 | 16507 | 17757 | 19007 |
| TOTCOVID00314 | 10258 | 11508 | 12758 | 14008 | 15258 | 16508 | 17758 | 19008 |
| TOTCOVID00315 | 10259 | 11509 | 12759 | 14009 | 15259 | 16509 | 17759 | 19009 |
| TOTCOVID00316 | 10260 | 11510 | 12760 | 14010 | 15260 | 16510 | 17760 | 19010 |
| TOTCOVID00317 | 10261 | 11511 | 12761 | 14011 | 15261 | 16511 | 17761 | 19011 |
| TOTCOVID00318 | 10262 | 11512 | 12762 | 14012 | 15262 | 16512 | 17762 | 19012 |
| TOTCOVID00319 | 10263 | 11513 | 12763 | 14013 | 15263 | 16513 | 17763 | 19013 |
| TOTCOVID00320 | 10264 | 11514 | 12764 | 14014 | 15264 | 16514 | 17764 | 19014 |
| TOTCOVID00321 | 10265 | 11515 | 12765 | 14015 | 15265 | 16515 | 17765 | 19015 |
| TOTCOVID00322 | 10266 | 11516 | 12766 | 14016 | 15266 | 16516 | 17766 | 19016 |
| TOTCOVID00323 | 10267 | 11517 | 12767 | 14017 | 15267 | 16517 | 17767 | 19017 |
| TOTCOVID00324 | 10268 | 11518 | 12768 | 14018 | 15268 | 16518 | 17768 | 19018 |
| TOTCOVID00325 | 10269 | 11519 | 12769 | 14019 | 15269 | 16519 | 17769 | 19019 |
| TOTCOVID00326 | 10270 | 11520 | 12770 | 14020 | 15270 | 16520 | 17770 | 19020 |
| TOTCOVID00327 | 10271 | 11521 | 12771 | 14021 | 15271 | 16521 | 17771 | 19021 |
| TOTCOVID00328 | 10272 | 11522 | 12772 | 14022 | 15272 | 16522 | 17772 | 19022 |
| TOTCOVID00329 | 10273 | 11523 | 12773 | 14023 | 15273 | 16523 | 17773 | 19023 |
| TOTCOVID00330 | 10274 | 11524 | 12774 | 14024 | 15274 | 16524 | 17774 | 19024 |
| TOTCOVID00331 | 10275 | 11525 | 12775 | 14025 | 15275 | 16525 | 17775 | 19025 |
| TOTCOVID00332 | 10276 | 11526 | 12776 | 14026 | 15276 | 16526 | 17776 | 19026 |
| TOTCOVID00333 | 10277 | 11527 | 12777 | 14027 | 15277 | 16527 | 17777 | 19027 |
| TOTCOVID00334 | 10278 | 11528 | 12778 | 14028 | 15278 | 16528 | 17778 | 19028 |
| TOTCOVID00335 | 10279 | 11529 | 12779 | 14029 | 15279 | 16529 | 17779 | 19029 |
| TOTCOVID00336 | 10280 | 11530 | 12780 | 14030 | 15280 | 16530 | 17780 | 19030 |
| TOTCOVID00337 | 10281 | 11531 | 12781 | 14031 | 15281 | 16531 | 17781 | 19031 |
| TOTCOVID00338 | 10282 | 11532 | 12782 | 14032 | 15282 | 16532 | 17782 | 19032 |
| TOTCOVID00339 | 10283 | 11533 | 12783 | 14033 | 15283 | 16533 | 17783 | 19033 |
| TOTCOVID00340 | 10284 | 11534 | 12784 | 14034 | 15284 | 16534 | 17784 | 19034 |
| TOTCOVID00341 | 10285 | 11535 | 12785 | 14035 | 15285 | 16535 | 17785 | 19035 |
| TOTCOVID00342 | 10286 | 11536 | 12786 | 14036 | 15286 | 16536 | 17786 | 19036 |
| TOTCOVID00343 | 10287 | 11537 | 12787 | 14037 | 15287 | 16537 | 17787 | 19037 |
| TOTCOVID00344 | 10288 | 11538 | 12788 | 14038 | 15288 | 16538 | 17788 | 19038 |
| TOTCOVID00345 | 10289 | 11539 | 12789 | 14039 | 15289 | 16539 | 17789 | 19039 |
| TOTCOVID00346 | 10290 | 11540 | 12790 | 14040 | 15290 | 16540 | 17790 | 19040 |
| TOTCOVID00347 | 10291 | 11541 | 12791 | 14041 | 15291 | 16541 | 17791 | 19041 |
| TOTCOVID00348 | 10292 | 11542 | 12792 | 14042 | 15292 | 16542 | 17792 | 19042 |
| TOTCOVID00349 | 10293 | 11543 | 12793 | 14043 | 15293 | 16543 | 17793 | 19043 |
| TOTCOVID00350 | 10294 | 11544 | 12794 | 14044 | 15294 | 16544 | 17794 | 19044 |
| TOTCOVID00351 | 10295 | 11545 | 12795 | 14045 | 15295 | 16545 | 17795 | 19045 |
| TOTCOVID00352 | 10296 | 11546 | 12796 | 14046 | 15296 | 16546 | 17796 | 19046 |
| TOTCOVID00353 | 10297 | 11547 | 12797 | 14047 | 15297 | 16547 | 17797 | 19047 |
| TOTCOVID00354 | 10298 | 11548 | 12798 | 14048 | 15298 | 16548 | 17798 | 19048 |
| TOTCOVID00355 | 10299 | 11549 | 12799 | 14049 | 15299 | 16549 | 17799 | 19049 |
| TOTCOVID00356 | 10300 | 11550 | 12800 | 14050 | 15300 | 16550 | 17800 | 19050 |
| TOTCOVID00357 | 10301 | 11551 | 12801 | 14051 | 15301 | 16551 | 17801 | 19051 |
| TOTCOVID00358 | 10302 | 11552 | 12802 | 14052 | 15302 | 16552 | 17802 | 19052 |
| TOTCOVID00359 | 10303 | 11553 | 12803 | 14053 | 15303 | 16553 | 17803 | 19053 |
| TOTCOVID00360 | 10304 | 11554 | 12804 | 14054 | 15304 | 16554 | 17804 | 19054 |
| TOTCOVID00361 | 10305 | 11555 | 12805 | 14055 | 15305 | 16555 | 17805 | 19055 |
| TOTCOVID00362 | 10306 | 11556 | 12806 | 14056 | 15306 | 16556 | 17806 | 19056 |
| TOTCOVID00363 | 10307 | 11557 | 12807 | 14057 | 15307 | 16557 | 17807 | 19057 |
| TOTCOVID00364 | 10308 | 11558 | 12808 | 14058 | 15308 | 16558 | 17808 | 19058 |
| TOTCOVID00365 | 10309 | 11559 | 12809 | 14059 | 15309 | 16559 | 17809 | 19059 |
| TOTCOVID00366 | 10310 | 11560 | 12810 | 14060 | 15310 | 16560 | 17810 | 19060 |
| TOTCOVID00367 | 10311 | 11561 | 12811 | 14061 | 15311 | 16561 | 17811 | 19061 |
| TOTCOVID00368 | 10312 | 11562 | 12812 | 14062 | 15312 | 16562 | 17812 | 19062 |
| TOTCOVID00369 | 10313 | 11563 | 12813 | 14063 | 15313 | 16563 | 17813 | 19063 |
| TOTCOVID00370 | 10314 | 11564 | 12814 | 14064 | 15314 | 16564 | 17814 | 19064 |
| TOTCOVID00371 | 10315 | 11565 | 12815 | 14065 | 15315 | 16565 | 17815 | 19065 |
| TOTCOVID00372 | 10316 | 11566 | 12816 | 14066 | 15316 | 16566 | 17816 | 19066 |
| TOTCOVID00373 | 10317 | 11567 | 12817 | 14067 | 15317 | 16567 | 17817 | 19067 |
| TOTCOVID00374 | 10318 | 11568 | 12818 | 14068 | 15318 | 16568 | 17818 | 19068 |
| TOTCOVID00375 | 10319 | 11569 | 12819 | 14069 | 15319 | 16569 | 17819 | 19069 |
| TOTCOVID00376 | 10320 | 11570 | 12820 | 14070 | 15320 | 16570 | 17820 | 19070 |
| TOTCOVID00377 | 10321 | 11571 | 12821 | 14071 | 15321 | 16571 | 17821 | 19071 |
| TOTCOVID00378 | 10322 | 11572 | 12822 | 14072 | 15322 | 16572 | 17822 | 19072 |
| TOTCOVID00379 | 10323 | 11573 | 12823 | 14073 | 15323 | 16573 | 17823 | 19073 |
| TOTCOVID00380 | 10324 | 11574 | 12824 | 14074 | 15324 | 16574 | 17824 | 19074 |
| TOTCOVID00381 | 10325 | 11575 | 12825 | 14075 | 15325 | 16575 | 17825 | 19075 |
| TOTCOVID00382 | 10326 | 11576 | 12826 | 14076 | 15326 | 16576 | 17826 | 19076 |
| TOTCOVID00383 | 10327 | 11577 | 12827 | 14077 | 15327 | 16577 | 17827 | 19077 |
| TOTCOVID00384 | 10328 | 11578 | 12828 | 14078 | 15328 | 16578 | 17828 | 19078 |
| TOTCOVID00385 | 10329 | 11579 | 12829 | 14079 | 15329 | 16579 | 17829 | 19079 |
| TOTCOVID00386 | 10330 | 11580 | 12830 | 14080 | 15330 | 16580 | 17830 | 19080 |
| TOTCOVID00387 | 10331 | 11581 | 12831 | 14081 | 15331 | 16581 | 17831 | 19081 |
| TOTCOVID00388 | 10332 | 11582 | 12832 | 14082 | 15332 | 16582 | 17832 | 19082 |
| TOTCOVID00389 | 10333 | 11583 | 12833 | 14083 | 15333 | 16583 | 17833 | 19083 |
| TOTCOVID00390 | 10334 | 11584 | 12834 | 14084 | 15334 | 16584 | 17834 | 19084 |
| TOTCOVID00391 | 10335 | 11585 | 12835 | 14085 | 15335 | 16585 | 17835 | 19085 |
| TOTCOVID00392 | 10336 | 11586 | 12836 | 14086 | 15336 | 16586 | 17836 | 19086 |
| TOTCOVID00393 | 10337 | 11587 | 12837 | 14087 | 15337 | 16587 | 17837 | 19087 |
| TOTCOVID00394 | 10338 | 11588 | 12838 | 14088 | 15338 | 16588 | 17838 | 19088 |
| TOTCOVID00395 | 10339 | 11589 | 12839 | 14089 | 15339 | 16589 | 17839 | 19089 |
| TOTCOVID00396 | 10340 | 11590 | 12840 | 14090 | 15340 | 16590 | 17840 | 19090 |
| TOTCOVID00397 | 10341 | 11591 | 12841 | 14091 | 15341 | 16591 | 17841 | 19091 |
| TOTCOVID00398 | 10342 | 11592 | 12842 | 14092 | 15342 | 16592 | 17842 | 19092 |
| TOTCOVID00399 | 10343 | 11593 | 12843 | 14093 | 15343 | 16593 | 17843 | 19093 |
| TOTCOVID00400 | 10344 | 11594 | 12844 | 14094 | 15344 | 16594 | 17844 | 19094 |
| TOTCOVID00401 | 10345 | 11595 | 12845 | 14095 | 15345 | 16595 | 17845 | 19095 |
| TOTCOVID00402 | 10346 | 11596 | 12846 | 14096 | 15346 | 16596 | 17846 | 19096 |
| TOTCOVID00403 | 10347 | 11597 | 12847 | 14097 | 15347 | 16597 | 17847 | 19097 |
| TOTCOVID00404 | 10348 | 11598 | 12848 | 14098 | 15348 | 16598 | 17848 | 19098 |
| TOTCOVID00405 | 10349 | 11599 | 12849 | 14099 | 15349 | 16599 | 17849 | 19099 |
| TOTCOVID00406 | 10350 | 11600 | 12850 | 14100 | 15350 | 16600 | 17850 | 19100 |
| TOTCOVID00407 | 10351 | 11601 | 12851 | 14101 | 15351 | 16601 | 17851 | 19101 |
| TOTCOVID00408 | 10352 | 11602 | 12852 | 14102 | 15352 | 16602 | 17852 | 19102 |
| TOTCOVID00409 | 10353 | 11603 | 12853 | 14103 | 15353 | 16603 | 17853 | 19103 |
| TOTCOVID00410 | 10354 | 11604 | 12854 | 14104 | 15354 | 16604 | 17854 | 19104 |
| TOTCOVID00411 | 10355 | 11605 | 12855 | 14105 | 15355 | 16605 | 17855 | 19105 |
| TOTCOVID00412 | 10356 | 11606 | 12856 | 14106 | 15356 | 16606 | 17856 | 19106 |
| TOTCOVID00413 | 10357 | 11607 | 12857 | 14107 | 15357 | 16607 | 17857 | 19107 |
| TOTCOVID00414 | 10358 | 11608 | 12858 | 14108 | 15358 | 16608 | 17858 | 19108 |
| TOTCOVID00415 | 10359 | 11609 | 12859 | 14109 | 15359 | 16609 | 17859 | 19109 |
| TOTCOVID00416 | 10360 | 11610 | 12860 | 14110 | 15360 | 16610 | 17860 | 19110 |
| TOTCOVID00417 | 10361 | 11611 | 12861 | 14111 | 15361 | 16611 | 17861 | 19111 |
| TOTCOVID00418 | 10362 | 11612 | 12862 | 14112 | 15362 | 16612 | 17862 | 19112 |
| TOTCOVID00419 | 10363 | 11613 | 12863 | 14113 | 15363 | 16613 | 17863 | 19113 |
| TOTCOVID00420 | 10364 | 11614 | 12864 | 14114 | 15364 | 16614 | 17864 | 19114 |
| TOTCOVID00421 | 10365 | 11615 | 12865 | 14115 | 15365 | 16615 | 17865 | 19115 |
| TOTCOVID00422 | 10366 | 11616 | 12866 | 14116 | 15366 | 16616 | 17866 | 19116 |
| TOTCOVID00423 | 10367 | 11617 | 12867 | 14117 | 15367 | 16617 | 17867 | 19117 |
| TOTCOVID00424 | 10368 | 11618 | 12868 | 14118 | 15368 | 16618 | 17868 | 19118 |
| TOTCOVID00425 | 10369 | 11619 | 12869 | 14119 | 15369 | 16619 | 17869 | 19119 |
| TOTCOVID00426 | 10370 | 11620 | 12870 | 14120 | 15370 | 16620 | 17870 | 19120 |
| TOTCOVID00427 | 10371 | 11621 | 12871 | 14121 | 15371 | 16621 | 17871 | 19121 |
| TOTCOVID00428 | 10372 | 11622 | 12872 | 14122 | 15372 | 16622 | 17872 | 19122 |
| TOTCOVID00429 | 10373 | 11623 | 12873 | 14123 | 15373 | 16623 | 17873 | 19123 |
| TOTCOVID00430 | 10374 | 11624 | 12874 | 14124 | 15374 | 16624 | 17874 | 19124 |
| TOTCOVID00431 | 10375 | 11625 | 12875 | 14125 | 15375 | 16625 | 17875 | 19125 |
| TOTCOVID00432 | 10376 | 11626 | 12876 | 14126 | 15376 | 16626 | 17876 | 19126 |
| TOTCOVID00433 | 10377 | 11627 | 12877 | 14127 | 15377 | 16627 | 17877 | 19127 |
| TOTCOVID00434 | 10378 | 11628 | 12878 | 14128 | 15378 | 16628 | 17878 | 19128 |
| TOTCOVID00435 | 10379 | 11629 | 12879 | 14129 | 15379 | 16629 | 17879 | 19129 |
| TOTCOVID00436 | 10380 | 11630 | 12880 | 14130 | 15380 | 16630 | 17880 | 19130 |
| TOTCOVID00437 | 10381 | 11631 | 12881 | 14131 | 15381 | 16631 | 17881 | 19131 |
| TOTCOVID00438 | 10382 | 11632 | 12882 | 14132 | 15382 | 16632 | 17882 | 19132 |
| TOTCOVID00439 | 10383 | 11633 | 12883 | 14133 | 15383 | 16633 | 17883 | 19133 |
| TOTCOVID00440 | 10384 | 11634 | 12884 | 14134 | 15384 | 16634 | 17884 | 19134 |
| TOTCOVID00441 | 10385 | 11635 | 12885 | 14135 | 15385 | 16635 | 17885 | 19135 |
| TOTCOVID00442 | 10386 | 11636 | 12886 | 14136 | 15386 | 16636 | 17886 | 19136 |
| TOTCOVID00443 | 10387 | 11637 | 12887 | 14137 | 15387 | 16637 | 17887 | 19137 |
| TOTCOVID00444 | 10388 | 11638 | 12888 | 14138 | 15388 | 16638 | 17888 | 19138 |
| TOTCOVID00445 | 10389 | 11639 | 12889 | 14139 | 15389 | 16639 | 17889 | 19139 |
| TOTCOVID00446 | 10390 | 11640 | 12890 | 14140 | 15390 | 16640 | 17890 | 19140 |
| TOTCOVID00447 | 10391 | 11641 | 12891 | 14141 | 15391 | 16641 | 17891 | 19141 |
| TOTCOVID00448 | 10392 | 11642 | 12892 | 14142 | 15392 | 16642 | 17892 | 19142 |
| TOTCOVID00449 | 10393 | 11643 | 12893 | 14143 | 15393 | 16643 | 17893 | 19143 |
| TOTCOVID00450 | 10394 | 11644 | 12894 | 14144 | 15394 | 16644 | 17894 | 19144 |
| TOTCOVID00451 | 10395 | 11645 | 12895 | 14145 | 15395 | 16645 | 17895 | 19145 |
| TOTCOVID00452 | 10396 | 11646 | 12896 | 14146 | 15396 | 16646 | 17896 | 19146 |
| TOTCOVID00453 | 10397 | 11647 | 12897 | 14147 | 15397 | 16647 | 17897 | 19147 |
| TOTCOVID00454 | 10398 | 11648 | 12898 | 14148 | 15398 | 16648 | 17898 | 19148 |
| TOTCOVID00455 | 10399 | 11649 | 12899 | 14149 | 15399 | 16649 | 17899 | 19149 |
| TOTCOVID00456 | 10400 | 11650 | 12900 | 14150 | 15400 | 16650 | 17900 | 19150 |
| TOTCOVID00457 | 10401 | 11651 | 12901 | 14151 | 15401 | 16651 | 17901 | 19151 |
| TOTCOVID00458 | 10402 | 11652 | 12902 | 14152 | 15402 | 16652 | 17902 | 19152 |
| TOTCOVID00459 | 10403 | 11653 | 12903 | 14153 | 15403 | 16653 | 17903 | 19153 |
| TOTCOVID00460 | 10404 | 11654 | 12904 | 14154 | 15404 | 16654 | 17904 | 19154 |
| TOTCOVID00461 | 10405 | 11655 | 12905 | 14155 | 15405 | 16655 | 17905 | 19155 |
| TOTCOVID00462 | 10406 | 11656 | 12906 | 14156 | 15406 | 16656 | 17906 | 19156 |
| TOTCOVID00463 | 10407 | 11657 | 12907 | 14157 | 15407 | 16657 | 17907 | 19157 |
| TOTCOVID00464 | 10408 | 11658 | 12908 | 14158 | 15408 | 16658 | 17908 | 19158 |
| TOTCOVID00465 | 10409 | 11659 | 12909 | 14159 | 15409 | 16659 | 17909 | 19159 |
| TOTCOVID00466 | 10410 | 11660 | 12910 | 14160 | 15410 | 16660 | 17910 | 19160 |
| TOTCOVID00467 | 10411 | 11661 | 12911 | 14161 | 15411 | 16661 | 17911 | 19161 |
| TOTCOVID00468 | 10412 | 11662 | 12912 | 14162 | 15412 | 16662 | 17912 | 19162 |
| TOTCOVID00469 | 10413 | 11663 | 12913 | 14163 | 15413 | 16663 | 17913 | 19163 |
| TOTCOVID00470 | 10414 | 11664 | 12914 | 14164 | 15414 | 16664 | 17914 | 19164 |
| TOTCOVID00471 | 10415 | 11665 | 12915 | 14165 | 15415 | 16665 | 17915 | 19165 |
| TOTCOVID00472 | 10416 | 11666 | 12916 | 14166 | 15416 | 16666 | 17916 | 19166 |
| TOTCOVID00473 | 10417 | 11667 | 12917 | 14167 | 15417 | 16667 | 17917 | 19167 |
| TOTCOVID00474 | 10418 | 11668 | 12918 | 14168 | 15418 | 16668 | 17918 | 19168 |
| TOTCOVID00475 | 10419 | 11669 | 12919 | 14169 | 15419 | 16669 | 17919 | 19169 |
| TOTCOVID00476 | 10420 | 11670 | 12920 | 14170 | 15420 | 16670 | 17920 | 19170 |
| TOTCOVID00477 | 10421 | 11671 | 12921 | 14171 | 15421 | 16671 | 17921 | 19171 |
| TOTCOVID00478 | 10422 | 11672 | 12922 | 14172 | 15422 | 16672 | 17922 | 19172 |
| TOTCOVID00479 | 10423 | 11673 | 12923 | 14173 | 15423 | 16673 | 17923 | 19173 |
| TOTCOVID00480 | 10424 | 11674 | 12924 | 14174 | 15424 | 16674 | 17924 | 19174 |
| TOTCOVID00481 | 10425 | 11675 | 12925 | 14175 | 15425 | 16675 | 17925 | 19175 |
| TOTCOVID00482 | 10426 | 11676 | 12926 | 14176 | 15426 | 16676 | 17926 | 19176 |
| TOTCOVID00483 | 10427 | 11677 | 12927 | 14177 | 15427 | 16677 | 17927 | 19177 |
| TOTCOVID00484 | 10428 | 11678 | 12928 | 14178 | 15428 | 16678 | 17928 | 19178 |
| TOTCOVID00485 | 10429 | 11679 | 12929 | 14179 | 15429 | 16679 | 17929 | 19179 |
| TOTCOVID00486 | 10430 | 11680 | 12930 | 14180 | 15430 | 16680 | 17930 | 19180 |
| TOTCOVID00487 | 10431 | 11681 | 12931 | 14181 | 15431 | 16681 | 17931 | 19181 |
| TOTCOVID00488 | 10432 | 11682 | 12932 | 14182 | 15432 | 16682 | 17932 | 19182 |
| TOTCOVID00489 | 10433 | 11683 | 12933 | 14183 | 15433 | 16683 | 17933 | 19183 |
| TOTCOVID00490 | 10434 | 11684 | 12934 | 14184 | 15434 | 16684 | 17934 | 19184 |
| TOTCOVID00491 | 10435 | 11685 | 12935 | 14185 | 15435 | 16685 | 17935 | 19185 |
| TOTCOVID00492 | 10436 | 11686 | 12936 | 14186 | 15436 | 16686 | 17936 | 19186 |
| TOTCOVID00493 | 10437 | 11687 | 12937 | 14187 | 15437 | 16687 | 17937 | 19187 |
| TOTCOVID00494 | 10438 | 11688 | 12938 | 14188 | 15438 | 16688 | 17938 | 19188 |
| TOTCOVID00495 | 10439 | 11689 | 12939 | 14189 | 15439 | 16689 | 17939 | 19189 |
| TOTCOVID00496 | 10440 | 11690 | 12940 | 14190 | 15440 | 16690 | 17940 | 19190 |
| TOTCOVID00497 | 10441 | 11691 | 12941 | 14191 | 15441 | 16691 | 17941 | 19191 |
| TOTCOVID00498 | 10442 | 11692 | 12942 | 14192 | 15442 | 16692 | 17942 | 19192 |
| TOTCOVID00499 | 10443 | 11693 | 12943 | 14193 | 15443 | 16693 | 17943 | 19193 |
| TOTCOVID00500 | 10444 | 11694 | 12944 | 14194 | 15444 | 16694 | 17944 | 19194 |
| TOTCOVID00501 | 10445 | 11695 | 12945 | 14195 | 15445 | 16695 | 17945 | 19195 |
| TOTCOVID00502 | 10446 | 11696 | 12946 | 14196 | 15446 | 16696 | 17946 | 19196 |
| TOTCOVID00503 | 10447 | 11697 | 12947 | 14197 | 15447 | 16697 | 17947 | 19197 |
| TOTCOVID00504 | 10448 | 11698 | 12948 | 14198 | 15448 | 16698 | 17948 | 19198 |
| TOTCOVID00505 | 10449 | 11699 | 12949 | 14199 | 15449 | 16699 | 17949 | 19199 |
| TOTCOVID00506 | 10450 | 11700 | 12950 | 14200 | 15450 | 16700 | 17950 | 19200 |
| TOTCOVID00507 | 10451 | 11701 | 12951 | 14201 | 15451 | 16701 | 17951 | 19201 |
| TOTCOVID00508 | 10452 | 11702 | 12952 | 14202 | 15452 | 16702 | 17952 | 19202 |
| TOTCOVID00509 | 10453 | 11703 | 12953 | 14203 | 15453 | 16703 | 17953 | 19203 |
| TOTCOVID00510 | 10454 | 11704 | 12954 | 14204 | 15454 | 16704 | 17954 | 19204 |
| TOTCOVID00511 | 10455 | 11705 | 12955 | 14205 | 15455 | 16705 | 17955 | 19205 |
| TOTCOVID00512 | 10456 | 11706 | 12956 | 14206 | 15456 | 16706 | 17956 | 19206 |
| TOTCOVID00513 | 10457 | 11707 | 12957 | 14207 | 15457 | 16707 | 17957 | 19207 |
| TOTCOVID00514 | 10458 | 11708 | 12958 | 14208 | 15458 | 16708 | 17958 | 19208 |
| TOTCOVID00515 | 10459 | 11709 | 12959 | 14209 | 15459 | 16709 | 17959 | 19209 |
| TOTCOVID00516 | 10460 | 11710 | 12960 | 14210 | 15460 | 16710 | 17960 | 19210 |
| TOTCOVID00517 | 10461 | 11711 | 12961 | 14211 | 15461 | 16711 | 17961 | 19211 |
| TOTCOVID00518 | 10462 | 11712 | 12962 | 14212 | 15462 | 16712 | 17962 | 19212 |
| TOTCOVID00519 | 10463 | 11713 | 12963 | 14213 | 15463 | 16713 | 17963 | 19213 |
| TOTCOVID00520 | 10464 | 11714 | 12964 | 14214 | 15464 | 16714 | 17964 | 19214 |
| TOTCOVID00521 | 10465 | 11715 | 12965 | 14215 | 15465 | 16715 | 17965 | 19215 |
| TOTCOVID00522 | 10466 | 11716 | 12966 | 14216 | 15466 | 16716 | 17966 | 19216 |
| TOTCOVID00523 | 10467 | 11717 | 12967 | 14217 | 15467 | 16717 | 17967 | 19217 |
| TOTCOVID00524 | 10468 | 11718 | 12968 | 14218 | 15468 | 16718 | 17968 | 19218 |
| TOTCOVID00525 | 10469 | 11719 | 12969 | 14219 | 15469 | 16719 | 17969 | 19219 |
| TOTCOVID00526 | 10470 | 11720 | 12970 | 14220 | 15470 | 16720 | 17970 | 19220 |
| TOTCOVID00527 | 10471 | 11721 | 12971 | 14221 | 15471 | 16721 | 17971 | 19221 |
| TOTCOVID00528 | 10472 | 11722 | 12972 | 14222 | 15472 | 16722 | 17972 | 19222 |
| TOTCOVID00529 | 10473 | 11723 | 12973 | 14223 | 15473 | 16723 | 17973 | 19223 |
| TOTCOVID00530 | 10474 | 11724 | 12974 | 14224 | 15474 | 16724 | 17974 | 19224 |
| TOTCOVID00531 | 10475 | 11725 | 12975 | 14225 | 15475 | 16725 | 17975 | 19225 |
| TOTCOVID00532 | 10476 | 11726 | 12976 | 14226 | 15476 | 16726 | 17976 | 19226 |
| TOTCOVID00533 | 10477 | 11727 | 12977 | 14227 | 15477 | 16727 | 17977 | 19227 |
| TOTCOVID00534 | 10478 | 11728 | 12978 | 14228 | 15478 | 16728 | 17978 | 19228 |
| TOTCOVID00535 | 10479 | 11729 | 12979 | 14229 | 15479 | 16729 | 17979 | 19229 |
| TOTCOVID00536 | 10480 | 11730 | 12980 | 14230 | 15480 | 16730 | 17980 | 19230 |
| TOTCOVID00537 | 10481 | 11731 | 12981 | 14231 | 15481 | 16731 | 17981 | 19231 |
| TOTCOVID00538 | 10482 | 11732 | 12982 | 14232 | 15482 | 16732 | 17982 | 19232 |
| TOTCOVID00539 | 10483 | 11733 | 12983 | 14233 | 15483 | 16733 | 17983 | 19233 |
| TOTCOVID00540 | 10484 | 11734 | 12984 | 14234 | 15484 | 16734 | 17984 | 19234 |
| TOTCOVID00541 | 10485 | 11735 | 12985 | 14235 | 15485 | 16735 | 17985 | 19235 |
| TOTCOVID00542 | 10486 | 11736 | 12986 | 14236 | 15486 | 16736 | 17986 | 19236 |
| TOTCOVID00543 | 10487 | 11737 | 12987 | 14237 | 15487 | 16737 | 17987 | 19237 |
| TOTCOVID00544 | 10488 | 11738 | 12988 | 14238 | 15488 | 16738 | 17988 | 19238 |
| TOTCOVID00545 | 10489 | 11739 | 12989 | 14239 | 15489 | 16739 | 17989 | 19239 |
| TOTCOVID00546 | 10490 | 11740 | 12990 | 14240 | 15490 | 16740 | 17990 | 19240 |
| TOTCOVID00547 | 10491 | 11741 | 12991 | 14241 | 15491 | 16741 | 17991 | 19241 |
| TOTCOVID00548 | 10492 | 11742 | 12992 | 14242 | 15492 | 16742 | 17992 | 19242 |
| TOTCOVID00549 | 10493 | 11743 | 12993 | 14243 | 15493 | 16743 | 17993 | 19243 |
| TOTCOVID00550 | 10494 | 11744 | 12994 | 14244 | 15494 | 16744 | 17994 | 19244 |
| TOTCOVID00551 | 10495 | 11745 | 12995 | 14245 | 15495 | 16745 | 17995 | 19245 |
| TOTCOVID00552 | 10496 | 11746 | 12996 | 14246 | 15496 | 16746 | 17996 | 19246 |
| TOTCOVID00553 | 10497 | 11747 | 12997 | 14247 | 15497 | 16747 | 17997 | 19247 |
| TOTCOVID00554 | 10498 | 11748 | 12998 | 14248 | 15498 | 16748 | 17998 | 19248 |
| TOTCOVID00555 | 10499 | 11749 | 12999 | 14249 | 15499 | 16749 | 17999 | 19249 |
| TOTCOVID00556 | 10500 | 11750 | 13000 | 14250 | 15500 | 16750 | 18000 | 19250 |
| TOTCOVID00557 | 10501 | 11751 | 13001 | 14251 | 15501 | 16751 | 18001 | 19251 |
| TOTCOVID00558 | 10502 | 11752 | 13002 | 14252 | 15502 | 16752 | 18002 | 19252 |
| TOTCOVID00559 | 10503 | 11753 | 13003 | 14253 | 15503 | 16753 | 18003 | 19253 |
| TOTCOVID00560 | 10504 | 11754 | 13004 | 14254 | 15504 | 16754 | 18004 | 19254 |
| TOTCOVID00561 | 10505 | 11755 | 13005 | 14255 | 15505 | 16755 | 18005 | 19255 |
| TOTCOVID00562 | 10506 | 11756 | 13006 | 14256 | 15506 | 16756 | 18006 | 19256 |
| TOTCOVID00563 | 10507 | 11757 | 13007 | 14257 | 15507 | 16757 | 18007 | 19257 |
| TOTCOVID00564 | 10508 | 11758 | 13008 | 14258 | 15508 | 16758 | 18008 | 19258 |
| TOTCOVID00565 | 10509 | 11759 | 13009 | 14259 | 15509 | 16759 | 18009 | 19259 |
| TOTCOVID00566 | 10510 | 11760 | 13010 | 14260 | 15510 | 16760 | 18010 | 19260 |
| TOTCOVID00567 | 10511 | 11761 | 13011 | 14261 | 15511 | 16761 | 18011 | 19261 |
| TOTCOVID00568 | 10512 | 11762 | 13012 | 14262 | 15512 | 16762 | 18012 | 19262 |
| TOTCOVID00569 | 10513 | 11763 | 13013 | 14263 | 15513 | 16763 | 18013 | 19263 |
| TOTCOVID00570 | 10514 | 11764 | 13014 | 14264 | 15514 | 16764 | 18014 | 19264 |
| TOTCOVID00571 | 10515 | 11765 | 13015 | 14265 | 15515 | 16765 | 18015 | 19265 |
| TOTCOVID00572 | 10516 | 11766 | 13016 | 14266 | 15516 | 16766 | 18016 | 19266 |
| TOTCOVID00573 | 10517 | 11767 | 13017 | 14267 | 15517 | 16767 | 18017 | 19267 |
| TOTCOVID00574 | 10518 | 11768 | 13018 | 14268 | 15518 | 16768 | 18018 | 19268 |
| TOTCOVID00575 | 10519 | 11769 | 13019 | 14269 | 15519 | 16769 | 18019 | 19269 |
| TOTCOVID00576 | 10520 | 11770 | 13020 | 14270 | 15520 | 16770 | 18020 | 19270 |
| TOTCOVID00577 | 10521 | 11771 | 13021 | 14271 | 15521 | 16771 | 18021 | 19271 |
| TOTCOVID00578 | 10522 | 11772 | 13022 | 14272 | 15522 | 16772 | 18022 | 19272 |
| TOTCOVID00579 | 10523 | 11773 | 13023 | 14273 | 15523 | 16773 | 18023 | 19273 |
| TOTCOVID00580 | 10524 | 11774 | 13024 | 14274 | 15524 | 16774 | 18024 | 19274 |
| TOTCOVID00581 | 10525 | 11775 | 13025 | 14275 | 15525 | 16775 | 18025 | 19275 |
| TOTCOVID00582 | 10526 | 11776 | 13026 | 14276 | 15526 | 16776 | 18026 | 19276 |
| TOTCOVID00583 | 10527 | 11777 | 13027 | 14277 | 15527 | 16777 | 18027 | 19277 |
| TOTCOVID00584 | 10528 | 11778 | 13028 | 14278 | 15528 | 16778 | 18028 | 19278 |
| TOTCOVID00585 | 10529 | 11779 | 13029 | 14279 | 15529 | 16779 | 18029 | 19279 |
| TOTCOVID00586 | 10530 | 11780 | 13030 | 14280 | 15530 | 16780 | 18030 | 19280 |
| TOTCOVID00587 | 10531 | 11781 | 13031 | 14281 | 15531 | 16781 | 18031 | 19281 |
| TOTCOVID00588 | 10532 | 11782 | 13032 | 14282 | 15532 | 16782 | 18032 | 19282 |
| TOTCOVID00589 | 10533 | 11783 | 13033 | 14283 | 15533 | 16783 | 18033 | 19283 |
| TOTCOVID00590 | 10534 | 11784 | 13034 | 14284 | 15534 | 16784 | 18034 | 19284 |
| TOTCOVID00591 | 10535 | 11785 | 13035 | 14285 | 15535 | 16785 | 18035 | 19285 |
| TOTCOVID00592 | 10536 | 11786 | 13036 | 14286 | 15536 | 16786 | 18036 | 19286 |
| TOTCOVID00593 | 10537 | 11787 | 13037 | 14287 | 15537 | 16787 | 18037 | 19287 |
| TOTCOVID00594 | 10538 | 11788 | 13038 | 14288 | 15538 | 16788 | 18038 | 19288 |
| TOTCOVID00595 | 10539 | 11789 | 13039 | 14289 | 15539 | 16789 | 18039 | 19289 |
| TOTCOVID00596 | 10540 | 11790 | 13040 | 14290 | 15540 | 16790 | 18040 | 19290 |
| TOTCOVID00597 | 10541 | 11791 | 13041 | 14291 | 15541 | 16791 | 18041 | 19291 |
| TOTCOVID00598 | 10542 | 11792 | 13042 | 14292 | 15542 | 16792 | 18042 | 19292 |
| TOTCOVID00599 | 10543 | 11793 | 13043 | 14293 | 15543 | 16793 | 18043 | 19293 |
| TOTCOVID00600 | 10544 | 11794 | 13044 | 14294 | 15544 | 16794 | 18044 | 19294 |
| TOTCOVID00601 | 10545 | 11795 | 13045 | 14295 | 15545 | 16795 | 18045 | 19295 |
| TOTCOVID00602 | 10546 | 11796 | 13046 | 14296 | 15546 | 16796 | 18046 | 19296 |
| TOTCOVID00603 | 10547 | 11797 | 13047 | 14297 | 15547 | 16797 | 18047 | 19297 |
| TOTCOVID00604 | 10548 | 11798 | 13048 | 14298 | 15548 | 16798 | 18048 | 19298 |
| TOTCOVID00605 | 10549 | 11799 | 13049 | 14299 | 15549 | 16799 | 18049 | 19299 |
| TOTCOVID00606 | 10550 | 11800 | 13050 | 14300 | 15550 | 16800 | 18050 | 19300 |
| TOTCOVID00607 | 10551 | 11801 | 13051 | 14301 | 15551 | 16801 | 18051 | 19301 |
| TOTCOVID00608 | 10552 | 11802 | 13052 | 14302 | 15552 | 16802 | 18052 | 19302 |
| TOTCOVID00609 | 10553 | 11803 | 13053 | 14303 | 15553 | 16803 | 18053 | 19303 |
| TOTCOVID00610 | 10554 | 11804 | 13054 | 14304 | 15554 | 16804 | 18054 | 19304 |
| TOTCOVID00611 | 10555 | 11805 | 13055 | 14305 | 15555 | 16805 | 18055 | 19305 |
| TOTCOVID00612 | 10556 | 11806 | 13056 | 14306 | 15556 | 16806 | 18056 | 19306 |
| TOTCOVID00613 | 10557 | 11807 | 13057 | 14307 | 15557 | 16807 | 18057 | 19307 |
| TOTCOVID00614 | 10558 | 11808 | 13058 | 14308 | 15558 | 16808 | 18058 | 19308 |
| TOTCOVID00615 | 10559 | 11809 | 13059 | 14309 | 15559 | 16809 | 18059 | 19309 |
| TOTCOVID00616 | 10560 | 11810 | 13060 | 14310 | 15560 | 16810 | 18060 | 19310 |
| TOTCOVID00617 | 10561 | 11811 | 13061 | 14311 | 15561 | 16811 | 18061 | 19311 |
| TOTCOVID00618 | 10562 | 11812 | 13062 | 14312 | 15562 | 16812 | 18062 | 19312 |
| TOTCOVID00619 | 10563 | 11813 | 13063 | 14313 | 15563 | 16813 | 18063 | 19313 |
| TOTCOVID00620 | 10564 | 11814 | 13064 | 14314 | 15564 | 16814 | 18064 | 19314 |
| TOTCOVID00621 | 10565 | 11815 | 13065 | 14315 | 15565 | 16815 | 18065 | 19315 |
| TOTCOVID00622 | 10566 | 11816 | 13066 | 14316 | 15566 | 16816 | 18066 | 19316 |
| TOTCOVID00623 | 10567 | 11817 | 13067 | 14317 | 15567 | 16817 | 18067 | 19317 |
| TOTCOVID00624 | 10568 | 11818 | 13068 | 14318 | 15568 | 16818 | 18068 | 19318 |
| TOTCOVID00625 | 10569 | 11819 | 13069 | 14319 | 15569 | 16819 | 18069 | 19319 |
| TOTCOVID00626 | 10570 | 11820 | 13070 | 14320 | 15570 | 16820 | 18070 | 19320 |
| TOTCOVID00627 | 10571 | 11821 | 13071 | 14321 | 15571 | 16821 | 18071 | 19321 |
| TOTCOVID00628 | 10572 | 11822 | 13072 | 14322 | 15572 | 16822 | 18072 | 19322 |
| TOTCOVID00629 | 10573 | 11823 | 13073 | 14323 | 15573 | 16823 | 18073 | 19323 |
| TOTCOVID00630 | 10574 | 11824 | 13074 | 14324 | 15574 | 16824 | 18074 | 19324 |
| TOTCOVID00631 | 10575 | 11825 | 13075 | 14325 | 15575 | 16825 | 18075 | 19325 |
| TOTCOVID00632 | 10576 | 11826 | 13076 | 14326 | 15576 | 16826 | 18076 | 19326 |
| TOTCOVID00633 | 10577 | 11827 | 13077 | 14327 | 15577 | 16827 | 18077 | 19327 |
| TOTCOVID00634 | 10578 | 11828 | 13078 | 14328 | 15578 | 16828 | 18078 | 19328 |
| TOTCOVID00635 | 10579 | 11829 | 13079 | 14329 | 15579 | 16829 | 18079 | 19329 |
| TOTCOVID00636 | 10580 | 11830 | 13080 | 14330 | 15580 | 16830 | 18080 | 19330 |
| TOTCOVID00637 | 10581 | 11831 | 13081 | 14331 | 15581 | 16831 | 18081 | 19331 |
| TOTCOVID00638 | 10582 | 11832 | 13082 | 14332 | 15582 | 16832 | 18082 | 19332 |
| TOTCOVID00639 | 10583 | 11833 | 13083 | 14333 | 15583 | 16833 | 18083 | 19333 |
| TOTCOVID00640 | 10584 | 11834 | 13084 | 14334 | 15584 | 16834 | 18084 | 19334 |
| TOTCOVID00641 | 10585 | 11835 | 13085 | 14335 | 15585 | 16835 | 18085 | 19335 |
| TOTCOVID00642 | 10586 | 11836 | 13086 | 14336 | 15586 | 16836 | 18086 | 19336 |
| TOTCOVID00643 | 10587 | 11837 | 13087 | 14337 | 15587 | 16837 | 18087 | 19337 |
| TOTCOVID00644 | 10588 | 11838 | 13088 | 14338 | 15588 | 16838 | 18088 | 19338 |
| TOTCOVID00645 | 10589 | 11839 | 13089 | 14339 | 15589 | 16839 | 18089 | 19339 |
| TOTCOVID00646 | 10590 | 11840 | 13090 | 14340 | 15590 | 16840 | 18090 | 19340 |
| TOTCOVID00647 | 10591 | 11841 | 13091 | 14341 | 15591 | 16841 | 18091 | 19341 |
| TOTCOVID00648 | 10592 | 11842 | 13092 | 14342 | 15592 | 16842 | 18092 | 19342 |
| TOTCOVID00649 | 10593 | 11843 | 13093 | 14343 | 15593 | 16843 | 18093 | 19343 |
| TOTCOVID00650 | 10594 | 11844 | 13094 | 14344 | 15594 | 16844 | 18094 | 19344 |
| TOTCOVID00651 | 10595 | 11845 | 13095 | 14345 | 15595 | 16845 | 18095 | 19345 |
| TOTCOVID00652 | 10596 | 11846 | 13096 | 14346 | 15596 | 16846 | 18096 | 19346 |
| TOTCOVID00653 | 10597 | 11847 | 13097 | 14347 | 15597 | 16847 | 18097 | 19347 |
| TOTCOVID00654 | 10598 | 11848 | 13098 | 14348 | 15598 | 16848 | 18098 | 19348 |
| TOTCOVID00655 | 10599 | 11849 | 13099 | 14349 | 15599 | 16849 | 18099 | 19349 |
| TOTCOVID00656 | 10600 | 11850 | 13100 | 14350 | 15600 | 16850 | 18100 | 19350 |
| TOTCOVID00657 | 10601 | 11851 | 13101 | 14351 | 15601 | 16851 | 18101 | 19351 |
| TOTCOVID00658 | 10602 | 11852 | 13102 | 14352 | 15602 | 16852 | 18102 | 19352 |
| TOTCOVID00659 | 10603 | 11853 | 13103 | 14353 | 15603 | 16853 | 18103 | 19353 |
| TOTCOVID00660 | 10604 | 11854 | 13104 | 14354 | 15604 | 16854 | 18104 | 19354 |
| TOTCOVID00661 | 10605 | 11855 | 13105 | 14355 | 15605 | 16855 | 18105 | 19355 |
| TOTCOVID00662 | 10606 | 11856 | 13106 | 14356 | 15606 | 16856 | 18106 | 19356 |
| TOTCOVID00663 | 10607 | 11857 | 13107 | 14357 | 15607 | 16857 | 18107 | 19357 |
| TOTCOVID00664 | 10608 | 11858 | 13108 | 14358 | 15608 | 16858 | 18108 | 19358 |
| TOTCOVID00665 | 10609 | 11859 | 13109 | 14359 | 15609 | 16859 | 18109 | 19359 |
| TOTCOVID00666 | 10610 | 11860 | 13110 | 14360 | 15610 | 16860 | 18110 | 19360 |
| TOTCOVID00667 | 10611 | 11861 | 13111 | 14361 | 15611 | 16861 | 18111 | 19361 |
| TOTCOVID00668 | 10612 | 11862 | 13112 | 14362 | 15612 | 16862 | 18112 | 19362 |
| TOTCOVID00669 | 10613 | 11863 | 13113 | 14363 | 15613 | 16863 | 18113 | 19363 |
| TOTCOVID00670 | 10614 | 11864 | 13114 | 14364 | 15614 | 16864 | 18114 | 19364 |
| TOTCOVID00671 | 10615 | 11865 | 13115 | 14365 | 15615 | 16865 | 18115 | 19365 |
| TOTCOVID00672 | 10616 | 11866 | 13116 | 14366 | 15616 | 16866 | 18116 | 19366 |
| TOTCOVID00673 | 10617 | 11867 | 13117 | 14367 | 15617 | 16867 | 18117 | 19367 |
| TOTCOVID00674 | 10618 | 11868 | 13118 | 14368 | 15618 | 16868 | 18118 | 19368 |
| TOTCOVID00675 | 10619 | 11869 | 13119 | 14369 | 15619 | 16869 | 18119 | 19369 |
| TOTCOVID00676 | 10620 | 11870 | 13120 | 14370 | 15620 | 16870 | 18120 | 19370 |
| TOTCOVID00677 | 10621 | 11871 | 13121 | 14371 | 15621 | 16871 | 18121 | 19371 |
| TOTCOVID00678 | 10622 | 11872 | 13122 | 14372 | 15622 | 16872 | 18122 | 19372 |
| TOTCOVID00679 | 10623 | 11873 | 13123 | 14373 | 15623 | 16873 | 18123 | 19373 |
| TOTCOVID00680 | 10624 | 11874 | 13124 | 14374 | 15624 | 16874 | 18124 | 19374 |
| TOTCOVID00681 | 10625 | 11875 | 13125 | 14375 | 15625 | 16875 | 18125 | 19375 |
| TOTCOVID00682 | 10626 | 11876 | 13126 | 14376 | 15626 | 16876 | 18126 | 19376 |
| TOTCOVID00683 | 10627 | 11877 | 13127 | 14377 | 15627 | 16877 | 18127 | 19377 |
| TOTCOVID00684 | 10628 | 11878 | 13128 | 14378 | 15628 | 16878 | 18128 | 19378 |
| TOTCOVID00685 | 10629 | 11879 | 13129 | 14379 | 15629 | 16879 | 18129 | 19379 |
| TOTCOVID00686 | 10630 | 11880 | 13130 | 14380 | 15630 | 16880 | 18130 | 19380 |
| TOTCOVID00687 | 10631 | 11881 | 13131 | 14381 | 15631 | 16881 | 18131 | 19381 |
| TOTCOVID00688 | 10632 | 11882 | 13132 | 14382 | 15632 | 16882 | 18132 | 19382 |
| TOTCOVID00689 | 10633 | 11883 | 13133 | 14383 | 15633 | 16883 | 18133 | 19383 |
| TOTCOVID00690 | 10634 | 11884 | 13134 | 14384 | 15634 | 16884 | 18134 | 19384 |
| TOTCOVID00691 | 10635 | 11885 | 13135 | 14385 | 15635 | 16885 | 18135 | 19385 |
| TOTCOVID00692 | 10636 | 11886 | 13136 | 14386 | 15636 | 16886 | 18136 | 19386 |
| TOTCOVID00693 | 10637 | 11887 | 13137 | 14387 | 15637 | 16887 | 18137 | 19387 |
| TOTCOVID00694 | 10638 | 11888 | 13138 | 14388 | 15638 | 16888 | 18138 | 19388 |
| TOTCOVID00695 | 10639 | 11889 | 13139 | 14389 | 15639 | 16889 | 18139 | 19389 |
| TOTCOVID00696 | 10640 | 11890 | 13140 | 14390 | 15640 | 16890 | 18140 | 19390 |
| TOTCOVID00697 | 10641 | 11891 | 13141 | 14391 | 15641 | 16891 | 18141 | 19391 |
| TOTCOVID00698 | 10642 | 11892 | 13142 | 14392 | 15642 | 16892 | 18142 | 19392 |
| TOTCOVID00699 | 10643 | 11893 | 13143 | 14393 | 15643 | 16893 | 18143 | 19393 |
| TOTCOVID00700 | 10644 | 11894 | 13144 | 14394 | 15644 | 16894 | 18144 | 19394 |
| TOTCOVID00701 | 10645 | 11895 | 13145 | 14395 | 15645 | 16895 | 18145 | 19395 |
| TOTCOVID00702 | 10646 | 11896 | 13146 | 14396 | 15646 | 16896 | 18146 | 19396 |
| TOTCOVID00703 | 10647 | 11897 | 13147 | 14397 | 15647 | 16897 | 18147 | 19397 |
| TOTCOVID00704 | 10648 | 11898 | 13148 | 14398 | 15648 | 16898 | 18148 | 19398 |
| TOTCOVID00705 | 10649 | 11899 | 13149 | 14399 | 15649 | 16899 | 18149 | 19399 |
| TOTCOVID00706 | 10650 | 11900 | 13150 | 14400 | 15650 | 16900 | 18150 | 19400 |
| TOTCOVID00707 | 10651 | 11901 | 13151 | 14401 | 15651 | 16901 | 18151 | 19401 |
| TOTCOVID00708 | 10652 | 11902 | 13152 | 14402 | 15652 | 16902 | 18152 | 19402 |
| TOTCOVID00709 | 10653 | 11903 | 13153 | 14403 | 15653 | 16903 | 18153 | 19403 |
| TOTCOVID00710 | 10654 | 11904 | 13154 | 14404 | 15654 | 16904 | 18154 | 19404 |
| TOTCOVID00711 | 10655 | 11905 | 13155 | 14405 | 15655 | 16905 | 18155 | 19405 |
| TOTCOVID00712 | 10656 | 11906 | 13156 | 14406 | 15656 | 16906 | 18156 | 19406 |
| TOTCOVID00713 | 10657 | 11907 | 13157 | 14407 | 15657 | 16907 | 18157 | 19407 |
| TOTCOVID00714 | 10658 | 11908 | 13158 | 14408 | 15658 | 16908 | 18158 | 19408 |
| TOTCOVID00715 | 10659 | 11909 | 13159 | 14409 | 15659 | 16909 | 18159 | 19409 |
| TOTCOVID00716 | 10660 | 11910 | 13160 | 14410 | 15660 | 16910 | 18160 | 19410 |
| TOTCOVID00717 | 10661 | 11911 | 13161 | 14411 | 15661 | 16911 | 18161 | 19411 |
| TOTCOVID00718 | 10662 | 11912 | 13162 | 14412 | 15662 | 16912 | 18162 | 19412 |
| TOTCOVID00719 | 10663 | 11913 | 13163 | 14413 | 15663 | 16913 | 18163 | 19413 |
| TOTCOVID00720 | 10664 | 11914 | 13164 | 14414 | 15664 | 16914 | 18164 | 19414 |
| TOTCOVID00721 | 10665 | 11915 | 13165 | 14415 | 15665 | 16915 | 18165 | 19415 |
| TOTCOVID00722 | 10666 | 11916 | 13166 | 14416 | 15666 | 16916 | 18166 | 19416 |
| TOTCOVID00723 | 10667 | 11917 | 13167 | 14417 | 15667 | 16917 | 18167 | 19417 |
| TOTCOVID00724 | 10668 | 11918 | 13168 | 14418 | 15668 | 16918 | 18168 | 19418 |
| TOTCOVID00725 | 10669 | 11919 | 13169 | 14419 | 15669 | 16919 | 18169 | 19419 |
| TOTCOVID00726 | 10670 | 11920 | 13170 | 14420 | 15670 | 16920 | 18170 | 19420 |
| TOTCOVID00727 | 10671 | 11921 | 13171 | 14421 | 15671 | 16921 | 18171 | 19421 |
| TOTCOVID00728 | 10672 | 11922 | 13172 | 14422 | 15672 | 16922 | 18172 | 19422 |
| TOTCOVID00729 | 10673 | 11923 | 13173 | 14423 | 15673 | 16923 | 18173 | 19423 |
| TOTCOVID00730 | 10674 | 11924 | 13174 | 14424 | 15674 | 16924 | 18174 | 19424 |
| TOTCOVID00731 | 10675 | 11925 | 13175 | 14425 | 15675 | 16925 | 18175 | 19425 |
| TOTCOVID00732 | 10676 | 11926 | 13176 | 14426 | 15676 | 16926 | 18176 | 19426 |
| TOTCOVID00733 | 10677 | 11927 | 13177 | 14427 | 15677 | 16927 | 18177 | 19427 |
| TOTCOVID00734 | 10678 | 11928 | 13178 | 14428 | 15678 | 16928 | 18178 | 19428 |
| TOTCOVID00735 | 10679 | 11929 | 13179 | 14429 | 15679 | 16929 | 18179 | 19429 |
| TOTCOVID00736 | 10680 | 11930 | 13180 | 14430 | 15680 | 16930 | 18180 | 19430 |
| TOTCOVID00737 | 10681 | 11931 | 13181 | 14431 | 15681 | 16931 | 18181 | 19431 |
| TOTCOVID00738 | 10682 | 11932 | 13182 | 14432 | 15682 | 16932 | 18182 | 19432 |
| TOTCOVID00739 | 10683 | 11933 | 13183 | 14433 | 15683 | 16933 | 18183 | 19433 |
| TOTCOVID00740 | 10684 | 11934 | 13184 | 14434 | 15684 | 16934 | 18184 | 19434 |
| TOTCOVID00741 | 10685 | 11935 | 13185 | 14435 | 15685 | 16935 | 18185 | 19435 |
| TOTCOVID00742 | 10686 | 11936 | 13186 | 14436 | 15686 | 16936 | 18186 | 19436 |
| TOTCOVID00743 | 10687 | 11937 | 13187 | 14437 | 15687 | 16937 | 18187 | 19437 |
| TOTCOVID00744 | 10688 | 11938 | 13188 | 14438 | 15688 | 16938 | 18188 | 19438 |
| TOTCOVID00745 | 10689 | 11939 | 13189 | 14439 | 15689 | 16939 | 18189 | 19439 |
| TOTCOVID00746 | 10690 | 11940 | 13190 | 14440 | 15690 | 16940 | 18190 | 19440 |
| TOTCOVID00747 | 10691 | 11941 | 13191 | 14441 | 15691 | 16941 | 18191 | 19441 |
| TOTCOVID00748 | 10692 | 11942 | 13192 | 14442 | 15692 | 16942 | 18192 | 19442 |
| TOTCOVID00749 | 10693 | 11943 | 13193 | 14443 | 15693 | 16943 | 18193 | 19443 |
| TOTCOVID00750 | 10694 | 11944 | 13194 | 14444 | 15694 | 16944 | 18194 | 19444 |
| TOTCOVID00751 | 10695 | 11945 | 13195 | 14445 | 15695 | 16945 | 18195 | 19445 |
| TOTCOVID00752 | 10696 | 11946 | 13196 | 14446 | 15696 | 16946 | 18196 | 19446 |
| TOTCOVID00753 | 10697 | 11947 | 13197 | 14447 | 15697 | 16947 | 18197 | 19447 |
| TOTCOVID00754 | 10698 | 11948 | 13198 | 14448 | 15698 | 16948 | 18198 | 19448 |
| TOTCOVID00755 | 10699 | 11949 | 13199 | 14449 | 15699 | 16949 | 18199 | 19449 |
| TOTCOVID00756 | 10700 | 11950 | 13200 | 14450 | 15700 | 16950 | 18200 | 19450 |
| TOTCOVID00757 | 10701 | 11951 | 13201 | 14451 | 15701 | 16951 | 18201 | 19451 |
| TOTCOVID00758 | 10702 | 11952 | 13202 | 14452 | 15702 | 16952 | 18202 | 19452 |
| TOTCOVID00759 | 10703 | 11953 | 13203 | 14453 | 15703 | 16953 | 18203 | 19453 |
| TOTCOVID00760 | 10704 | 11954 | 13204 | 14454 | 15704 | 16954 | 18204 | 19454 |
| TOTCOVID00761 | 10705 | 11955 | 13205 | 14455 | 15705 | 16955 | 18205 | 19455 |
| TOTCOVID00762 | 10706 | 11956 | 13206 | 14456 | 15706 | 16956 | 18206 | 19456 |
| TOTCOVID00763 | 10707 | 11957 | 13207 | 14457 | 15707 | 16957 | 18207 | 19457 |
| TOTCOVID00764 | 10708 | 11958 | 13208 | 14458 | 15708 | 16958 | 18208 | 19458 |
| TOTCOVID00765 | 10709 | 11959 | 13209 | 14459 | 15709 | 16959 | 18209 | 19459 |
| TOTCOVID00766 | 10710 | 11960 | 13210 | 14460 | 15710 | 16960 | 18210 | 19460 |
| TOTCOVID00767 | 10711 | 11961 | 13211 | 14461 | 15711 | 16961 | 18211 | 19461 |
| TOTCOVID00768 | 10712 | 11962 | 13212 | 14462 | 15712 | 16962 | 18212 | 19462 |
| TOTCOVID00769 | 10713 | 11963 | 13213 | 14463 | 15713 | 16963 | 18213 | 19463 |
| TOTCOVID00770 | 10714 | 11964 | 13214 | 14464 | 15714 | 16964 | 18214 | 19464 |
| TOTCOVID00771 | 10715 | 11965 | 13215 | 14465 | 15715 | 16965 | 18215 | 19465 |
| TOTCOVID00772 | 10716 | 11966 | 13216 | 14466 | 15716 | 16966 | 18216 | 19466 |
| TOTCOVID00773 | 10717 | 11967 | 13217 | 14467 | 15717 | 16967 | 18217 | 19467 |
| TOTCOVID00774 | 10718 | 11968 | 13218 | 14468 | 15718 | 16968 | 18218 | 19468 |
| TOTCOVID00775 | 10719 | 11969 | 13219 | 14469 | 15719 | 16969 | 18219 | 19469 |
| TOTCOVID00776 | 10720 | 11970 | 13220 | 14470 | 15720 | 16970 | 18220 | 19470 |
| TOTCOVID00777 | 10721 | 11971 | 13221 | 14471 | 15721 | 16971 | 18221 | 19471 |
| TOTCOVID00778 | 10722 | 11972 | 13222 | 14472 | 15722 | 16972 | 18222 | 19472 |
| TOTCOVID00779 | 10723 | 11973 | 13223 | 14473 | 15723 | 16973 | 18223 | 19473 |
| TOTCOVID00780 | 10724 | 11974 | 13224 | 14474 | 15724 | 16974 | 18224 | 19474 |
| TOTCOVID00781 | 10725 | 11975 | 13225 | 14475 | 15725 | 16975 | 18225 | 19475 |
| TOTCOVID00782 | 10726 | 11976 | 13226 | 14476 | 15726 | 16976 | 18226 | 19476 |
| TOTCOVID00783 | 10727 | 11977 | 13227 | 14477 | 15727 | 16977 | 18227 | 19477 |
| TOTCOVID00784 | 10728 | 11978 | 13228 | 14478 | 15728 | 16978 | 18228 | 19478 |
| TOTCOVID00785 | 10729 | 11979 | 13229 | 14479 | 15729 | 16979 | 18229 | 19479 |
| TOTCOVID00786 | 10730 | 11980 | 13230 | 14480 | 15730 | 16980 | 18230 | 19480 |
| TOTCOVID00787 | 10731 | 11981 | 13231 | 14481 | 15731 | 16981 | 18231 | 19481 |
| TOTCOVID00788 | 10732 | 11982 | 13232 | 14482 | 15732 | 16982 | 18232 | 19482 |
| TOTCOVID00789 | 10733 | 11983 | 13233 | 14483 | 15733 | 16983 | 18233 | 19483 |
| TOTCOVID00790 | 10734 | 11984 | 13234 | 14484 | 15734 | 16984 | 18234 | 19484 |
| TOTCOVID00791 | 10735 | 11985 | 13235 | 14485 | 15735 | 16985 | 18235 | 19485 |
| TOTCOVID00792 | 10736 | 11986 | 13236 | 14486 | 15736 | 16986 | 18236 | 19486 |
| TOTCOVID00793 | 10737 | 11987 | 13237 | 14487 | 15737 | 16987 | 18237 | 19487 |
| TOTCOVID00794 | 10738 | 11988 | 13238 | 14488 | 15738 | 16988 | 18238 | 19488 |
| TOTCOVID00795 | 10739 | 11989 | 13239 | 14489 | 15739 | 16989 | 18239 | 19489 |
| TOTCOVID00796 | 10740 | 11990 | 13240 | 14490 | 15740 | 16990 | 18240 | 19490 |
| TOTCOVID00797 | 10741 | 11991 | 13241 | 14491 | 15741 | 16991 | 18241 | 19491 |
| TOTCOVID00798 | 10742 | 11992 | 13242 | 14492 | 15742 | 16992 | 18242 | 19492 |
| TOTCOVID00799 | 10743 | 11993 | 13243 | 14493 | 15743 | 16993 | 18243 | 19493 |
| TOTCOVID00800 | 10744 | 11994 | 13244 | 14494 | 15744 | 16994 | 18244 | 19494 |
| TOTCOVID00801 | 10745 | 11995 | 13245 | 14495 | 15745 | 16995 | 18245 | 19495 |
| TOTCOVID00802 | 10746 | 11996 | 13246 | 14496 | 15746 | 16996 | 18246 | 19496 |
| TOTCOVID00803 | 10747 | 11997 | 13247 | 14497 | 15747 | 16997 | 18247 | 19497 |
| TOTCOVID00804 | 10748 | 11998 | 13248 | 14498 | 15748 | 16998 | 18248 | 19498 |
| TOTCOVID00805 | 10749 | 11999 | 13249 | 14499 | 15749 | 16999 | 18249 | 19499 |
| TOTCOVID00806 | 10750 | 12000 | 13250 | 14500 | 15750 | 17000 | 18250 | 19500 |
| TOTCOVID00807 | 10751 | 12001 | 13251 | 14501 | 15751 | 17001 | 18251 | 19501 |
| TOTCOVID00808 | 10752 | 12002 | 13252 | 14502 | 15752 | 17002 | 18252 | 19502 |
| TOTCOVID00809 | 10753 | 12003 | 13253 | 14503 | 15753 | 17003 | 18253 | 19503 |
| TOTCOVID00810 | 10754 | 12004 | 13254 | 14504 | 15754 | 17004 | 18254 | 19504 |
| TOTCOVID00811 | 10755 | 12005 | 13255 | 14505 | 15755 | 17005 | 18255 | 19505 |
| TOTCOVID00812 | 10756 | 12006 | 13256 | 14506 | 15756 | 17006 | 18256 | 19506 |
| TOTCOVID00813 | 10757 | 12007 | 13257 | 14507 | 15757 | 17007 | 18257 | 19507 |
| TOTCOVID00814 | 10758 | 12008 | 13258 | 14508 | 15758 | 17008 | 18258 | 19508 |
| TOTCOVID00815 | 10759 | 12009 | 13259 | 14509 | 15759 | 17009 | 18259 | 19509 |
| TOTCOVID00816 | 10760 | 12010 | 13260 | 14510 | 15760 | 17010 | 18260 | 19510 |
| TOTCOVID00817 | 10761 | 12011 | 13261 | 14511 | 15761 | 17011 | 18261 | 19511 |
| TOTCOVID00818 | 10762 | 12012 | 13262 | 14512 | 15762 | 17012 | 18262 | 19512 |
| TOTCOVID00819 | 10763 | 12013 | 13263 | 14513 | 15763 | 17013 | 18263 | 19513 |
| TOTCOVID00820 | 10764 | 12014 | 13264 | 14514 | 15764 | 17014 | 18264 | 19514 |
| TOTCOVID00821 | 10765 | 12015 | 13265 | 14515 | 15765 | 17015 | 18265 | 19515 |
| TOTCOVID00822 | 10766 | 12016 | 13266 | 14516 | 15766 | 17016 | 18266 | 19516 |
| TOTCOVID00823 | 10767 | 12017 | 13267 | 14517 | 15767 | 17017 | 18267 | 19517 |
| TOTCOVID00824 | 10768 | 12018 | 13268 | 14518 | 15768 | 17018 | 18268 | 19518 |
| TOTCOVID00825 | 10769 | 12019 | 13269 | 14519 | 15769 | 17019 | 18269 | 19519 |
| TOTCOVID00826 | 10770 | 12020 | 13270 | 14520 | 15770 | 17020 | 18270 | 19520 |
| TOTCOVID00827 | 10771 | 12021 | 13271 | 14521 | 15771 | 17021 | 18271 | 19521 |
| TOTCOVID00828 | 10772 | 12022 | 13272 | 14522 | 15772 | 17022 | 18272 | 19522 |
| TOTCOVID00829 | 10773 | 12023 | 13273 | 14523 | 15773 | 17023 | 18273 | 19523 |
| TOTCOVID00830 | 10774 | 12024 | 13274 | 14524 | 15774 | 17024 | 18274 | 19524 |
| TOTCOVID00831 | 10775 | 12025 | 13275 | 14525 | 15775 | 17025 | 18275 | 19525 |
| TOTCOVID00832 | 10776 | 12026 | 13276 | 14526 | 15776 | 17026 | 18276 | 19526 |
| TOTCOVID00833 | 10777 | 12027 | 13277 | 14527 | 15777 | 17027 | 18277 | 19527 |
| TOTCOVID00834 | 10778 | 12028 | 13278 | 14528 | 15778 | 17028 | 18278 | 19528 |
| TOTCOVID00835 | 10779 | 12029 | 13279 | 14529 | 15779 | 17029 | 18279 | 19529 |
| TOTCOVID00836 | 10780 | 12030 | 13280 | 14530 | 15780 | 17030 | 18280 | 19530 |
| TOTCOVID00837 | 10781 | 12031 | 13281 | 14531 | 15781 | 17031 | 18281 | 19531 |
| TOTCOVID00838 | 10782 | 12032 | 13282 | 14532 | 15782 | 17032 | 18282 | 19532 |
| TOTCOVID00839 | 10783 | 12033 | 13283 | 14533 | 15783 | 17033 | 18283 | 19533 |
| TOTCOVID00840 | 10784 | 12034 | 13284 | 14534 | 15784 | 17034 | 18284 | 19534 |
| TOTCOVID00841 | 10785 | 12035 | 13285 | 14535 | 15785 | 17035 | 18285 | 19535 |
| TOTCOVID00842 | 10786 | 12036 | 13286 | 14536 | 15786 | 17036 | 18286 | 19536 |
| TOTCOVID00843 | 10787 | 12037 | 13287 | 14537 | 15787 | 17037 | 18287 | 19537 |
| TOTCOVID00844 | 10788 | 12038 | 13288 | 14538 | 15788 | 17038 | 18288 | 19538 |
| TOTCOVID00845 | 10789 | 12039 | 13289 | 14539 | 15789 | 17039 | 18289 | 19539 |
| TOTCOVID00846 | 10790 | 12040 | 13290 | 14540 | 15790 | 17040 | 18290 | 19540 |
| TOTCOVID00847 | 10791 | 12041 | 13291 | 14541 | 15791 | 17041 | 18291 | 19541 |
| TOTCOVID00848 | 10792 | 12042 | 13292 | 14542 | 15792 | 17042 | 18292 | 19542 |
| TOTCOVID00849 | 10793 | 12043 | 13293 | 14543 | 15793 | 17043 | 18293 | 19543 |
| TOTCOVID00850 | 10794 | 12044 | 13294 | 14544 | 15794 | 17044 | 18294 | 19544 |
| TOTCOVID00851 | 10795 | 12045 | 13295 | 14545 | 15795 | 17045 | 18295 | 19545 |
| TOTCOVID00852 | 10796 | 12046 | 13296 | 14546 | 15796 | 17046 | 18296 | 19546 |
| TOTCOVID00853 | 10797 | 12047 | 13297 | 14547 | 15797 | 17047 | 18297 | 19547 |
| TOTCOVID00854 | 10798 | 12048 | 13298 | 14548 | 15798 | 17048 | 18298 | 19548 |
| TOTCOVID00855 | 10799 | 12049 | 13299 | 14549 | 15799 | 17049 | 18299 | 19549 |
| TOTCOVID00856 | 10800 | 12050 | 13300 | 14550 | 15800 | 17050 | 18300 | 19550 |
| TOTCOVID00857 | 10801 | 12051 | 13301 | 14551 | 15801 | 17051 | 18301 | 19551 |
| TOTCOVID00858 | 10802 | 12052 | 13302 | 14552 | 15802 | 17052 | 18302 | 19552 |
| TOTCOVID00859 | 10803 | 12053 | 13303 | 14553 | 15803 | 17053 | 18303 | 19553 |
| TOTCOVID00860 | 10804 | 12054 | 13304 | 14554 | 15804 | 17054 | 18304 | 19554 |
| TOTCOVID00861 | 10805 | 12055 | 13305 | 14555 | 15805 | 17055 | 18305 | 19555 |
| TOTCOVID00862 | 10806 | 12056 | 13306 | 14556 | 15806 | 17056 | 18306 | 19556 |
| TOTCOVID00863 | 10807 | 12057 | 13307 | 14557 | 15807 | 17057 | 18307 | 19557 |
| TOTCOVID00864 | 10808 | 12058 | 13308 | 14558 | 15808 | 17058 | 18308 | 19558 |
| TOTCOVID00865 | 10809 | 12059 | 13309 | 14559 | 15809 | 17059 | 18309 | 19559 |
| TOTCOVID00866 | 10810 | 12060 | 13310 | 14560 | 15810 | 17060 | 18310 | 19560 |
| TOTCOVID00867 | 10811 | 12061 | 13311 | 14561 | 15811 | 17061 | 18311 | 19561 |
| TOTCOVID00868 | 10812 | 12062 | 13312 | 14562 | 15812 | 17062 | 18312 | 19562 |
| TOTCOVID00869 | 10813 | 12063 | 13313 | 14563 | 15813 | 17063 | 18313 | 19563 |
| TOTCOVID00870 | 10814 | 12064 | 13314 | 14564 | 15814 | 17064 | 18314 | 19564 |
| TOTCOVID00871 | 10815 | 12065 | 13315 | 14565 | 15815 | 17065 | 18315 | 19565 |
| TOTCOVID00872 | 10816 | 12066 | 13316 | 14566 | 15816 | 17066 | 18316 | 19566 |
| TOTCOVID00873 | 10817 | 12067 | 13317 | 14567 | 15817 | 17067 | 18317 | 19567 |
| TOTCOVID00874 | 10818 | 12068 | 13318 | 14568 | 15818 | 17068 | 18318 | 19568 |
| TOTCOVID00875 | 10819 | 12069 | 13319 | 14569 | 15819 | 17069 | 18319 | 19569 |
| TOTCOVID00876 | 10820 | 12070 | 13320 | 14570 | 15820 | 17070 | 18320 | 19570 |
| TOTCOVID00877 | 10821 | 12071 | 13321 | 14571 | 15821 | 17071 | 18321 | 19571 |
| TOTCOVID00878 | 10822 | 12072 | 13322 | 14572 | 15822 | 17072 | 18322 | 19572 |
| TOTCOVID00879 | 10823 | 12073 | 13323 | 14573 | 15823 | 17073 | 18323 | 19573 |
| TOTCOVID00880 | 10824 | 12074 | 13324 | 14574 | 15824 | 17074 | 18324 | 19574 |
| TOTCOVID00881 | 10825 | 12075 | 13325 | 14575 | 15825 | 17075 | 18325 | 19575 |
| TOTCOVID00882 | 10826 | 12076 | 13326 | 14576 | 15826 | 17076 | 18326 | 19576 |
| TOTCOVID00883 | 10827 | 12077 | 13327 | 14577 | 15827 | 17077 | 18327 | 19577 |
| TOTCOVID00884 | 10828 | 12078 | 13328 | 14578 | 15828 | 17078 | 18328 | 19578 |
| TOTCOVID00885 | 10829 | 12079 | 13329 | 14579 | 15829 | 17079 | 18329 | 19579 |
| TOTCOVID00886 | 10830 | 12080 | 13330 | 14580 | 15830 | 17080 | 18330 | 19580 |
| TOTCOVID00887 | 10831 | 12081 | 13331 | 14581 | 15831 | 17081 | 18331 | 19581 |
| TOTCOVID00888 | 10832 | 12082 | 13332 | 14582 | 15832 | 17082 | 18332 | 19582 |
| TOTCOVID00889 | 10833 | 12083 | 13333 | 14583 | 15833 | 17083 | 18333 | 19583 |
| TOTCOVID00890 | 10834 | 12084 | 13334 | 14584 | 15834 | 17084 | 18334 | 19584 |
| TOTCOVID00891 | 10835 | 12085 | 13335 | 14585 | 15835 | 17085 | 18335 | 19585 |
| TOTCOVID00892 | 10836 | 12086 | 13336 | 14586 | 15836 | 17086 | 18336 | 19586 |
| TOTCOVID00893 | 10837 | 12087 | 13337 | 14587 | 15837 | 17087 | 18337 | 19587 |
| TOTCOVID00894 | 10838 | 12088 | 13338 | 14588 | 15838 | 17088 | 18338 | 19588 |
| TOTCOVID00895 | 10839 | 12089 | 13339 | 14589 | 15839 | 17089 | 18339 | 19589 |
| TOTCOVID00896 | 10840 | 12090 | 13340 | 14590 | 15840 | 17090 | 18340 | 19590 |
| TOTCOVID00897 | 10841 | 12091 | 13341 | 14591 | 15841 | 17091 | 18341 | 19591 |
| TOTCOVID00898 | 10842 | 12092 | 13342 | 14592 | 15842 | 17092 | 18342 | 19592 |
| TOTCOVID00899 | 10843 | 12093 | 13343 | 14593 | 15843 | 17093 | 18343 | 19593 |
| TOTCOVID00900 | 10844 | 12094 | 13344 | 14594 | 15844 | 17094 | 18344 | 19594 |
| TOTCOVID00901 | 10845 | 12095 | 13345 | 14595 | 15845 | 17095 | 18345 | 19595 |
| TOTCOVID00902 | 10846 | 12096 | 13346 | 14596 | 15846 | 17096 | 18346 | 19596 |
| TOTCOVID00903 | 10847 | 12097 | 13347 | 14597 | 15847 | 17097 | 18347 | 19597 |
| TOTCOVID00904 | 10848 | 12098 | 13348 | 14598 | 15848 | 17098 | 18348 | 19598 |
| TOTCOVID00905 | 10849 | 12099 | 13349 | 14599 | 15849 | 17099 | 18349 | 19599 |
| TOTCOVID00906 | 10850 | 12100 | 13350 | 14600 | 15850 | 17100 | 18350 | 19600 |
| TOTCOVID00907 | 10851 | 12101 | 13351 | 14601 | 15851 | 17101 | 18351 | 19601 |
| TOTCOVID00908 | 10852 | 12102 | 13352 | 14602 | 15852 | 17102 | 18352 | 19602 |
| TOTCOVID00909 | 10853 | 12103 | 13353 | 14603 | 15853 | 17103 | 18353 | 19603 |
| TOTCOVID00910 | 10854 | 12104 | 13354 | 14604 | 15854 | 17104 | 18354 | 19604 |
| TOTCOVID00911 | 10855 | 12105 | 13355 | 14605 | 15855 | 17105 | 18355 | 19605 |
| TOTCOVID00912 | 10856 | 12106 | 13356 | 14606 | 15856 | 17106 | 18356 | 19606 |
| TOTCOVID00913 | 10857 | 12107 | 13357 | 14607 | 15857 | 17107 | 18357 | 19607 |
| TOTCOVID00914 | 10858 | 12108 | 13358 | 14608 | 15858 | 17108 | 18358 | 19608 |
| TOTCOVID00915 | 10859 | 12109 | 13359 | 14609 | 15859 | 17109 | 18359 | 19609 |
| TOTCOVID00916 | 10860 | 12110 | 13360 | 14610 | 15860 | 17110 | 18360 | 19610 |
| TOTCOVID00917 | 10861 | 12111 | 13361 | 14611 | 15861 | 17111 | 18361 | 19611 |
| TOTCOVID00918 | 10862 | 12112 | 13362 | 14612 | 15862 | 17112 | 18362 | 19612 |
| TOTCOVID00919 | 10863 | 12113 | 13363 | 14613 | 15863 | 17113 | 18363 | 19613 |
| TOTCOVID00920 | 10864 | 12114 | 13364 | 14614 | 15864 | 17114 | 18364 | 19614 |
| TOTCOVID00921 | 10865 | 12115 | 13365 | 14615 | 15865 | 17115 | 18365 | 19615 |
| TOTCOVID00922 | 10866 | 12116 | 13366 | 14616 | 15866 | 17116 | 18366 | 19616 |
| TOTCOVID00923 | 10867 | 12117 | 13367 | 14617 | 15867 | 17117 | 18367 | 19617 |
| TOTCOVID00924 | 10868 | 12118 | 13368 | 14618 | 15868 | 17118 | 18368 | 19618 |
| TOTCOVID00925 | 10869 | 12119 | 13369 | 14619 | 15869 | 17119 | 18369 | 19619 |
| TOTCOVID00926 | 10870 | 12120 | 13370 | 14620 | 15870 | 17120 | 18370 | 19620 |
| TOTCOVID00927 | 10871 | 12121 | 13371 | 14621 | 15871 | 17121 | 18371 | 19621 |
| TOTCOVID00928 | 10872 | 12122 | 13372 | 14622 | 15872 | 17122 | 18372 | 19622 |
| TOTCOVID00929 | 10873 | 12123 | 13373 | 14623 | 15873 | 17123 | 18373 | 19623 |
| TOTCOVID00930 | 10874 | 12124 | 13374 | 14624 | 15874 | 17124 | 18374 | 19624 |
| TOTCOVID00931 | 10875 | 12125 | 13375 | 14625 | 15875 | 17125 | 18375 | 19625 |
| TOTCOVID00932 | 10876 | 12126 | 13376 | 14626 | 15876 | 17126 | 18376 | 19626 |
| TOTCOVID00933 | 10877 | 12127 | 13377 | 14627 | 15877 | 17127 | 18377 | 19627 |
| TOTCOVID00934 | 10878 | 12128 | 13378 | 14628 | 15878 | 17128 | 18378 | 19628 |
| TOTCOVID00935 | 10879 | 12129 | 13379 | 14629 | 15879 | 17129 | 18379 | 19629 |
| TOTCOVID00936 | 10880 | 12130 | 13380 | 14630 | 15880 | 17130 | 18380 | 19630 |
| TOTCOVID00937 | 10881 | 12131 | 13381 | 14631 | 15881 | 17131 | 18381 | 19631 |
| TOTCOVID00938 | 10882 | 12132 | 13382 | 14632 | 15882 | 17132 | 18382 | 19632 |
| TOTCOVID00939 | 10883 | 12133 | 13383 | 14633 | 15883 | 17133 | 18383 | 19633 |
| TOTCOVID00940 | 10884 | 12134 | 13384 | 14634 | 15884 | 17134 | 18384 | 19634 |
| TOTCOVID00941 | 10885 | 12135 | 13385 | 14635 | 15885 | 17135 | 18385 | 19635 |
| TOTCOVID00942 | 10886 | 12136 | 13386 | 14636 | 15886 | 17136 | 18386 | 19636 |
| TOTCOVID00943 | 10887 | 12137 | 13387 | 14637 | 15887 | 17137 | 18387 | 19637 |
| TOTCOVID00944 | 10888 | 12138 | 13388 | 14638 | 15888 | 17138 | 18388 | 19638 |
| TOTCOVID00945 | 10889 | 12139 | 13389 | 14639 | 15889 | 17139 | 18389 | 19639 |
| TOTCOVID00946 | 10890 | 12140 | 13390 | 14640 | 15890 | 17140 | 18390 | 19640 |
| TOTCOVID00947 | 10891 | 12141 | 13391 | 14641 | 15891 | 17141 | 18391 | 19641 |
| TOTCOVID00948 | 10892 | 12142 | 13392 | 14642 | 15892 | 17142 | 18392 | 19642 |
| TOTCOVID00949 | 10893 | 12143 | 13393 | 14643 | 15893 | 17143 | 18393 | 19643 |
| TOTCOVID00950 | 10894 | 12144 | 13394 | 14644 | 15894 | 17144 | 18394 | 19644 |
| TOTCOVID00951 | 10895 | 12145 | 13395 | 14645 | 15895 | 17145 | 18395 | 19645 |
| TOTCOVID00952 | 10896 | 12146 | 13396 | 14646 | 15896 | 17146 | 18396 | 19646 |
| TOTCOVID00953 | 10897 | 12147 | 13397 | 14647 | 15897 | 17147 | 18397 | 19647 |
| TOTCOVID00954 | 10898 | 12148 | 13398 | 14648 | 15898 | 17148 | 18398 | 19648 |
| TOTCOVID00955 | 10899 | 12149 | 13399 | 14649 | 15899 | 17149 | 18399 | 19649 |
| TOTCOVID00956 | 10900 | 12150 | 13400 | 14650 | 15900 | 17150 | 18400 | 19650 |
| TOTCOVID00957 | 10901 | 12151 | 13401 | 14651 | 15901 | 17151 | 18401 | 19651 |
| TOTCOVID00958 | 10902 | 12152 | 13402 | 14652 | 15902 | 17152 | 18402 | 19652 |
| TOTCOVID00959 | 10903 | 12153 | 13403 | 14653 | 15903 | 17153 | 18403 | 19653 |
| TOTCOVID00960 | 10904 | 12154 | 13404 | 14654 | 15904 | 17154 | 18404 | 19654 |
| TOTCOVID00961 | 10905 | 12155 | 13405 | 14655 | 15905 | 17155 | 18405 | 19655 |
| TOTCOVID00962 | 10906 | 12156 | 13406 | 14656 | 15906 | 17156 | 18406 | 19656 |
| TOTCOVID00963 | 10907 | 12157 | 13407 | 14657 | 15907 | 17157 | 18407 | 19657 |
| TOTCOVID00964 | 10908 | 12158 | 13408 | 14658 | 15908 | 17158 | 18408 | 19658 |
| TOTCOVID00965 | 10909 | 12159 | 13409 | 14659 | 15909 | 17159 | 18409 | 19659 |
| TOTCOVID00966 | 10910 | 12160 | 13410 | 14660 | 15910 | 17160 | 18410 | 19660 |
| TOTCOVID00967 | 10911 | 12161 | 13411 | 14661 | 15911 | 17161 | 18411 | 19661 |
| TOTCOVID00968 | 10912 | 12162 | 13412 | 14662 | 15912 | 17162 | 18412 | 19662 |
| TOTCOVID00969 | 10913 | 12163 | 13413 | 14663 | 15913 | 17163 | 18413 | 19663 |
| TOTCOVID00970 | 10914 | 12164 | 13414 | 14664 | 15914 | 17164 | 18414 | 19664 |
| TOTCOVID00971 | 10915 | 12165 | 13415 | 14665 | 15915 | 17165 | 18415 | 19665 |
| TOTCOVID00972 | 10916 | 12166 | 13416 | 14666 | 15916 | 17166 | 18416 | 19666 |
| TOTCOVID00973 | 10917 | 12167 | 13417 | 14667 | 15917 | 17167 | 18417 | 19667 |
| TOTCOVID00974 | 10918 | 12168 | 13418 | 14668 | 15918 | 17168 | 18418 | 19668 |
| TOTCOVID00975 | 10919 | 12169 | 13419 | 14669 | 15919 | 17169 | 18419 | 19669 |
| TOTCOVID00976 | 10920 | 12170 | 13420 | 14670 | 15920 | 17170 | 18420 | 19670 |
| TOTCOVID00977 | 10921 | 12171 | 13421 | 14671 | 15921 | 17171 | 18421 | 19671 |
| TOTCOVID00978 | 10922 | 12172 | 13422 | 14672 | 15922 | 17172 | 18422 | 19672 |
| TOTCOVID00979 | 10923 | 12173 | 13423 | 14673 | 15923 | 17173 | 18423 | 19673 |
| TOTCOVID00980 | 10924 | 12174 | 13424 | 14674 | 15924 | 17174 | 18424 | 19674 |
| TOTCOVID00981 | 10925 | 12175 | 13425 | 14675 | 15925 | 17175 | 18425 | 19675 |
| TOTCOVID00982 | 10926 | 12176 | 13426 | 14676 | 15926 | 17176 | 18426 | 19676 |
| TOTCOVID00983 | 10927 | 12177 | 13427 | 14677 | 15927 | 17177 | 18427 | 19677 |
| TOTCOVID00984 | 10928 | 12178 | 13428 | 14678 | 15928 | 17178 | 18428 | 19678 |
| TOTCOVID00985 | 10929 | 12179 | 13429 | 14679 | 15929 | 17179 | 18429 | 19679 |
| TOTCOVID00986 | 10930 | 12180 | 13430 | 14680 | 15930 | 17180 | 18430 | 19680 |
| TOTCOVID00987 | 10931 | 12181 | 13431 | 14681 | 15931 | 17181 | 18431 | 19681 |
| TOTCOVID00988 | 10932 | 12182 | 13432 | 14682 | 15932 | 17182 | 18432 | 19682 |
| TOTCOVID00989 | 10933 | 12183 | 13433 | 14683 | 15933 | 17183 | 18433 | 19683 |
| TOTCOVID00990 | 10934 | 12184 | 13434 | 14684 | 15934 | 17184 | 18434 | 19684 |
| TOTCOVID00991 | 10935 | 12185 | 13435 | 14685 | 15935 | 17185 | 18435 | 19685 |
| TOTCOVID00992 | 10936 | 12186 | 13436 | 14686 | 15936 | 17186 | 18436 | 19686 |
| TOTCOVID00993 | 10937 | 12187 | 13437 | 14687 | 15937 | 17187 | 18437 | 19687 |
| TOTCOVID00994 | 10938 | 12188 | 13438 | 14688 | 15938 | 17188 | 18438 | 19688 |
| TOTCOVID00995 | 10939 | 12189 | 13439 | 14689 | 15939 | 17189 | 18439 | 19689 |
| TOTCOVID00996 | 10940 | 12190 | 13440 | 14690 | 15940 | 17190 | 18440 | 19690 |
| TOTCOVID00997 | 10941 | 12191 | 13441 | 14691 | 15941 | 17191 | 18441 | 19691 |
| TOTCOVID00998 | 10942 | 12192 | 13442 | 14692 | 15942 | 17192 | 18442 | 19692 |
| TOTCOVID00999 | 10943 | 12193 | 13443 | 14693 | 15943 | 17193 | 18443 | 19693 |
| TOTCOVID01000 | 10944 | 12194 | 13444 | 14694 | 15944 | 17194 | 18444 | 19694 |
| TOTCOVID01001 | 10945 | 12195 | 13445 | 14695 | 15945 | 17195 | 18445 | 19695 |
| TOTCOVID01002 | 10946 | 12196 | 13446 | 14696 | 15946 | 17196 | 18446 | 19696 |
| TOTCOVID01003 | 10947 | 12197 | 13447 | 14697 | 15947 | 17197 | 18447 | 19697 |
| TOTCOVID01004 | 10948 | 12198 | 13448 | 14698 | 15948 | 17198 | 18448 | 19698 |
| TOTCOVID01005 | 10949 | 12199 | 13449 | 14699 | 15949 | 17199 | 18449 | 19699 |
| TOTCOVID01006 | 10950 | 12200 | 13450 | 14700 | 15950 | 17200 | 18450 | 19700 |
| TOTCOVID01007 | 10951 | 12201 | 13451 | 14701 | 15951 | 17201 | 18451 | 19701 |
| TOTCOVID01008 | 10952 | 12202 | 13452 | 14702 | 15952 | 17202 | 18452 | 19702 |
| TOTCOVID01009 | 10953 | 12203 | 13453 | 14703 | 15953 | 17203 | 18453 | 19703 |
| TOTCOVID01010 | 10954 | 12204 | 13454 | 14704 | 15954 | 17204 | 18454 | 19704 |
| TOTCOVID01011 | 10955 | 12205 | 13455 | 14705 | 15955 | 17205 | 18455 | 19705 |
| TOTCOVID01012 | 10956 | 12206 | 13456 | 14706 | 15956 | 17206 | 18456 | 19706 |
| TOTCOVID01013 | 10957 | 12207 | 13457 | 14707 | 15957 | 17207 | 18457 | 19707 |
| TOTCOVID01014 | 10958 | 12208 | 13458 | 14708 | 15958 | 17208 | 18458 | 19708 |
| TOTCOVID01015 | 10959 | 12209 | 13459 | 14709 | 15959 | 17209 | 18459 | 19709 |
| TOTCOVID01016 | 10960 | 12210 | 13460 | 14710 | 15960 | 17210 | 18460 | 19710 |
| TOTCOVID01017 | 10961 | 12211 | 13461 | 14711 | 15961 | 17211 | 18461 | 19711 |
| TOTCOVID01018 | 10962 | 12212 | 13462 | 14712 | 15962 | 17212 | 18462 | 19712 |
| TOTCOVID01019 | 10963 | 12213 | 13463 | 14713 | 15963 | 17213 | 18463 | 19713 |
| TOTCOVID01020 | 10964 | 12214 | 13464 | 14714 | 15964 | 17214 | 18464 | 19714 |
| TOTCOVID01021 | 10965 | 12215 | 13465 | 14715 | 15965 | 17215 | 18465 | 19715 |
| TOTCOVID01022 | 10966 | 12216 | 13466 | 14716 | 15966 | 17216 | 18466 | 19716 |
| TOTCOVID01023 | 10967 | 12217 | 13467 | 14717 | 15967 | 17217 | 18467 | 19717 |
| TOTCOVID01024 | 10968 | 12218 | 13468 | 14718 | 15968 | 17218 | 18468 | 19718 |
| TOTCOVID01025 | 10969 | 12219 | 13469 | 14719 | 15969 | 17219 | 18469 | 19719 |
| TOTCOVID01026 | 10970 | 12220 | 13470 | 14720 | 15970 | 17220 | 18470 | 19720 |
| TOTCOVID01027 | 10971 | 12221 | 13471 | 14721 | 15971 | 17221 | 18471 | 19721 |
| TOTCOVID01028 | 10972 | 12222 | 13472 | 14722 | 15972 | 17222 | 18472 | 19722 |
| TOTCOVID01029 | 10973 | 12223 | 13473 | 14723 | 15973 | 17223 | 18473 | 19723 |
| TOTCOVID01030 | 10974 | 12224 | 13474 | 14724 | 15974 | 17224 | 18474 | 19724 |
| TOTCOVID01031 | 10975 | 12225 | 13475 | 14725 | 15975 | 17225 | 18475 | 19725 |
| TOTCOVID01032 | 10976 | 12226 | 13476 | 14726 | 15976 | 17226 | 18476 | 19726 |
| TOTCOVID01033 | 10977 | 12227 | 13477 | 14727 | 15977 | 17227 | 18477 | 19727 |
| TOTCOVID01034 | 10978 | 12228 | 13478 | 14728 | 15978 | 17228 | 18478 | 19728 |
| TOTCOVID01035 | 10979 | 12229 | 13479 | 14729 | 15979 | 17229 | 18479 | 19729 |
| TOTCOVID01036 | 10980 | 12230 | 13480 | 14730 | 15980 | 17230 | 18480 | 19730 |
| TOTCOVID01037 | 10981 | 12231 | 13481 | 14731 | 15981 | 17231 | 18481 | 19731 |
| TOTCOVID01038 | 10982 | 12232 | 13482 | 14732 | 15982 | 17232 | 18482 | 19732 |
| TOTCOVID01039 | 10983 | 12233 | 13483 | 14733 | 15983 | 17233 | 18483 | 19733 |
| TOTCOVID01040 | 10984 | 12234 | 13484 | 14734 | 15984 | 17234 | 18484 | 19734 |
| TOTCOVID01041 | 10985 | 12235 | 13485 | 14735 | 15985 | 17235 | 18485 | 19735 |
| TOTCOVID01042 | 10986 | 12236 | 13486 | 14736 | 15986 | 17236 | 18486 | 19736 |
| TOTCOVID01043 | 10987 | 12237 | 13487 | 14737 | 15987 | 17237 | 18487 | 19737 |
| TOTCOVID01044 | 10988 | 12238 | 13488 | 14738 | 15988 | 17238 | 18488 | 19738 |
| TOTCOVID01045 | 10989 | 12239 | 13489 | 14739 | 15989 | 17239 | 18489 | 19739 |
| TOTCOVID01046 | 10990 | 12240 | 13490 | 14740 | 15990 | 17240 | 18490 | 19740 |
| TOTCOVID01047 | 10991 | 12241 | 13491 | 14741 | 15991 | 17241 | 18491 | 19741 |
| TOTCOVID01048 | 10992 | 12242 | 13492 | 14742 | 15992 | 17242 | 18492 | 19742 |
| TOTCOVID01049 | 10993 | 12243 | 13493 | 14743 | 15993 | 17243 | 18493 | 19743 |
| TOTCOVID01050 | 10994 | 12244 | 13494 | 14744 | 15994 | 17244 | 18494 | 19744 |
| TOTCOVID01051 | 10995 | 12245 | 13495 | 14745 | 15995 | 17245 | 18495 | 19745 |
| TOTCOVID01052 | 10996 | 12246 | 13496 | 14746 | 15996 | 17246 | 18496 | 19746 |
| TOTCOVID01053 | 10997 | 12247 | 13497 | 14747 | 15997 | 17247 | 18497 | 19747 |
| TOTCOVID01054 | 10998 | 12248 | 13498 | 14748 | 15998 | 17248 | 18498 | 19748 |
| TOTCOVID01055 | 10999 | 12249 | 13499 | 14749 | 15999 | 17249 | 18499 | 19749 |
| TOTCOVID01056 | 11000 | 12250 | 13500 | 14750 | 16000 | 17250 | 18500 | 19750 |
| TOTCOVID01057 | 11001 | 12251 | 13501 | 14751 | 16001 | 17251 | 18501 | 19751 |
| TOTCOVID01058 | 11002 | 12252 | 13502 | 14752 | 16002 | 17252 | 18502 | 19752 |
| TOTCOVID01059 | 11003 | 12253 | 13503 | 14753 | 16003 | 17253 | 18503 | 19753 |
| TOTCOVID01060 | 11004 | 12254 | 13504 | 14754 | 16004 | 17254 | 18504 | 19754 |
| TOTCOVID01061 | 11005 | 12255 | 13505 | 14755 | 16005 | 17255 | 18505 | 19755 |
| TOTCOVID01062 | 11006 | 12256 | 13506 | 14756 | 16006 | 17256 | 18506 | 19756 |
| TOTCOVID01063 | 11007 | 12257 | 13507 | 14757 | 16007 | 17257 | 18507 | 19757 |
| TOTCOVID01064 | 11008 | 12258 | 13508 | 14758 | 16008 | 17258 | 18508 | 19758 |
| TOTCOVID01065 | 11009 | 12259 | 13509 | 14759 | 16009 | 17259 | 18509 | 19759 |
| TOTCOVID01066 | 11010 | 12260 | 13510 | 14760 | 16010 | 17260 | 18510 | 19760 |
| TOTCOVID01067 | 11011 | 12261 | 13511 | 14761 | 16011 | 17261 | 18511 | 19761 |
| TOTCOVID01068 | 11012 | 12262 | 13512 | 14762 | 16012 | 17262 | 18512 | 19762 |
| TOTCOVID01069 | 11013 | 12263 | 13513 | 14763 | 16013 | 17263 | 18513 | 19763 |
| TOTCOVID01070 | 11014 | 12264 | 13514 | 14764 | 16014 | 17264 | 18514 | 19764 |
| TOTCOVID01071 | 11015 | 12265 | 13515 | 14765 | 16015 | 17265 | 18515 | 19765 |
| TOTCOVID01072 | 11016 | 12266 | 13516 | 14766 | 16016 | 17266 | 18516 | 19766 |
| TOTCOVID01073 | 11017 | 12267 | 13517 | 14767 | 16017 | 17267 | 18517 | 19767 |
| TOTCOVID01074 | 11018 | 12268 | 13518 | 14768 | 16018 | 17268 | 18518 | 19768 |
| TOTCOVID01075 | 11019 | 12269 | 13519 | 14769 | 16019 | 17269 | 18519 | 19769 |
| TOTCOVID01076 | 11020 | 12270 | 13520 | 14770 | 16020 | 17270 | 18520 | 19770 |
| TOTCOVID01077 | 11021 | 12271 | 13521 | 14771 | 16021 | 17271 | 18521 | 19771 |
| TOTCOVID01078 | 11022 | 12272 | 13522 | 14772 | 16022 | 17272 | 18522 | 19772 |
| TOTCOVID01079 | 11023 | 12273 | 13523 | 14773 | 16023 | 17273 | 18523 | 19773 |
| TOTCOVID01080 | 11024 | 12274 | 13524 | 14774 | 16024 | 17274 | 18524 | 19774 |
| TOTCOVID01081 | 11025 | 12275 | 13525 | 14775 | 16025 | 17275 | 18525 | 19775 |
| TOTCOVID01082 | 11026 | 12276 | 13526 | 14776 | 16026 | 17276 | 18526 | 19776 |
| TOTCOVID01083 | 11027 | 12277 | 13527 | 14777 | 16027 | 17277 | 18527 | 19777 |
| TOTCOVID01084 | 11028 | 12278 | 13528 | 14778 | 16028 | 17278 | 18528 | 19778 |
| TOTCOVID01085 | 11029 | 12279 | 13529 | 14779 | 16029 | 17279 | 18529 | 19779 |
| TOTCOVID01086 | 11030 | 12280 | 13530 | 14780 | 16030 | 17280 | 18530 | 19780 |
| TOTCOVID01087 | 11031 | 12281 | 13531 | 14781 | 16031 | 17281 | 18531 | 19781 |
| TOTCOVID01088 | 11032 | 12282 | 13532 | 14782 | 16032 | 17282 | 18532 | 19782 |
| TOTCOVID01089 | 11033 | 12283 | 13533 | 14783 | 16033 | 17283 | 18533 | 19783 |
| TOTCOVID01090 | 11034 | 12284 | 13534 | 14784 | 16034 | 17284 | 18534 | 19784 |
| TOTCOVID01091 | 11035 | 12285 | 13535 | 14785 | 16035 | 17285 | 18535 | 19785 |
| TOTCOVID01092 | 11036 | 12286 | 13536 | 14786 | 16036 | 17286 | 18536 | 19786 |
| TOTCOVID01093 | 11037 | 12287 | 13537 | 14787 | 16037 | 17287 | 18537 | 19787 |
| TOTCOVID01094 | 11038 | 12288 | 13538 | 14788 | 16038 | 17288 | 18538 | 19788 |
| TOTCOVID01095 | 11039 | 12289 | 13539 | 14789 | 16039 | 17289 | 18539 | 19789 |
| TOTCOVID01096 | 11040 | 12290 | 13540 | 14790 | 16040 | 17290 | 18540 | 19790 |
| TOTCOVID01097 | 11041 | 12291 | 13541 | 14791 | 16041 | 17291 | 18541 | 19791 |
| TOTCOVID01098 | 11042 | 12292 | 13542 | 14792 | 16042 | 17292 | 18542 | 19792 |
| TOTCOVID01099 | 11043 | 12293 | 13543 | 14793 | 16043 | 17293 | 18543 | 19793 |
| TOTCOVID01100 | 11044 | 12294 | 13544 | 14794 | 16044 | 17294 | 18544 | 19794 |
| TOTCOVID01101 | 11045 | 12295 | 13545 | 14795 | 16045 | 17295 | 18545 | 19795 |
| TOTCOVID01102 | 11046 | 12296 | 13546 | 14796 | 16046 | 17296 | 18546 | 19796 |
| TOTCOVID01103 | 11047 | 12297 | 13547 | 14797 | 16047 | 17297 | 18547 | 19797 |
| TOTCOVID01104 | 11048 | 12298 | 13548 | 14798 | 16048 | 17298 | 18548 | 19798 |
| TOTCOVID01105 | 11049 | 12299 | 13549 | 14799 | 16049 | 17299 | 18549 | 19799 |
| TOTCOVID01106 | 11050 | 12300 | 13550 | 14800 | 16050 | 17300 | 18550 | 19800 |
| TOTCOVID01107 | 11051 | 12301 | 13551 | 14801 | 16051 | 17301 | 18551 | 19801 |
| TOTCOVID01108 | 11052 | 12302 | 13552 | 14802 | 16052 | 17302 | 18552 | 19802 |
| TOTCOVID01109 | 11053 | 12303 | 13553 | 14803 | 16053 | 17303 | 18553 | 19803 |
| TOTCOVID01110 | 11054 | 12304 | 13554 | 14804 | 16054 | 17304 | 18554 | 19804 |
| TOTCOVID01111 | 11055 | 12305 | 13555 | 14805 | 16055 | 17305 | 18555 | 19805 |
| TOTCOVID01112 | 11056 | 12306 | 13556 | 14806 | 16056 | 17306 | 18556 | 19806 |
| TOTCOVID01113 | 11057 | 12307 | 13557 | 14807 | 16057 | 17307 | 18557 | 19807 |
| TOTCOVID01114 | 11058 | 12308 | 13558 | 14808 | 16058 | 17308 | 18558 | 19808 |
| TOTCOVID01115 | 11059 | 12309 | 13559 | 14809 | 16059 | 17309 | 18559 | 19809 |
| TOTCOVID01116 | 11060 | 12310 | 13560 | 14810 | 16060 | 17310 | 18560 | 19810 |
| TOTCOVID01117 | 11061 | 12311 | 13561 | 14811 | 16061 | 17311 | 18561 | 19811 |
| TOTCOVID01118 | 11062 | 12312 | 13562 | 14812 | 16062 | 17312 | 18562 | 19812 |
| TOTCOVID01119 | 11063 | 12313 | 13563 | 14813 | 16063 | 17313 | 18563 | 19813 |
| TOTCOVID01120 | 11064 | 12314 | 13564 | 14814 | 16064 | 17314 | 18564 | 19814 |
| TOTCOVID01121 | 11065 | 12315 | 13565 | 14815 | 16065 | 17315 | 18565 | 19815 |
| TOTCOVID01122 | 11066 | 12316 | 13566 | 14816 | 16066 | 17316 | 18566 | 19816 |
| TOTCOVID01123 | 11067 | 12317 | 13567 | 14817 | 16067 | 17317 | 18567 | 19817 |
| TOTCOVID01124 | 11068 | 12318 | 13568 | 14818 | 16068 | 17318 | 18568 | 19818 |
| TOTCOVID01125 | 11069 | 12319 | 13569 | 14819 | 16069 | 17319 | 18569 | 19819 |
| TOTCOVID01126 | 11070 | 12320 | 13570 | 14820 | 16070 | 17320 | 18570 | 19820 |
| TOTCOVID01127 | 11071 | 12321 | 13571 | 14821 | 16071 | 17321 | 18571 | 19821 |
| TOTCOVID01128 | 11072 | 12322 | 13572 | 14822 | 16072 | 17322 | 18572 | 19822 |
| TOTCOVID01129 | 11073 | 12323 | 13573 | 14823 | 16073 | 17323 | 18573 | 19823 |
| TOTCOVID01130 | 11074 | 12324 | 13574 | 14824 | 16074 | 17324 | 18574 | 19824 |
| TOTCOVID01131 | 11075 | 12325 | 13575 | 14825 | 16075 | 17325 | 18575 | 19825 |
| TOTCOVID01132 | 11076 | 12326 | 13576 | 14826 | 16076 | 17326 | 18576 | 19826 |
| TOTCOVID01133 | 11077 | 12327 | 13577 | 14827 | 16077 | 17327 | 18577 | 19827 |
| TOTCOVID01134 | 11078 | 12328 | 13578 | 14828 | 16078 | 17328 | 18578 | 19828 |
| TOTCOVID01135 | 11079 | 12329 | 13579 | 14829 | 16079 | 17329 | 18579 | 19829 |
| TOTCOVID01136 | 11080 | 12330 | 13580 | 14830 | 16080 | 17330 | 18580 | 19830 |
| TOTCOVID01137 | 11081 | 12331 | 13581 | 14831 | 16081 | 17331 | 18581 | 19831 |
| TOTCOVID01138 | 11082 | 12332 | 13582 | 14832 | 16082 | 17332 | 18582 | 19832 |
| TOTCOVID01139 | 11083 | 12333 | 13583 | 14833 | 16083 | 17333 | 18583 | 19833 |
| TOTCOVID01140 | 11084 | 12334 | 13584 | 14834 | 16084 | 17334 | 18584 | 19834 |
| TOTCOVID01141 | 11085 | 12335 | 13585 | 14835 | 16085 | 17335 | 18585 | 19835 |
| TOTCOVID01142 | 11086 | 12336 | 13586 | 14836 | 16086 | 17336 | 18586 | 19836 |
| TOTCOVID01143 | 11087 | 12337 | 13587 | 14837 | 16087 | 17337 | 18587 | 19837 |
| TOTCOVID01144 | 11088 | 12338 | 13588 | 14838 | 16088 | 17338 | 18588 | 19838 |
| TOTCOVID01145 | 11089 | 12339 | 13589 | 14839 | 16089 | 17339 | 18589 | 19839 |
| TOTCOVID01146 | 11090 | 12340 | 13590 | 14840 | 16090 | 17340 | 18590 | 19840 |
| TOTCOVID01147 | 11091 | 12341 | 13591 | 14841 | 16091 | 17341 | 18591 | 19841 |
| TOTCOVID01148 | 11092 | 12342 | 13592 | 14842 | 16092 | 17342 | 18592 | 19842 |
| TOTCOVID01149 | 11093 | 12343 | 13593 | 14843 | 16093 | 17343 | 18593 | 19843 |
| TOTCOVID01150 | 11094 | 12344 | 13594 | 14844 | 16094 | 17344 | 18594 | 19844 |
| TOTCOVID01151 | 11095 | 12345 | 13595 | 14845 | 16095 | 17345 | 18595 | 19845 |
| TOTCOVID01152 | 11096 | 12346 | 13596 | 14846 | 16096 | 17346 | 18596 | 19846 |
| TOTCOVID01153 | 11097 | 12347 | 13597 | 14847 | 16097 | 17347 | 18597 | 19847 |
| TOTCOVID01154 | 11098 | 12348 | 13598 | 14848 | 16098 | 17348 | 18598 | 19848 |
| TOTCOVID01155 | 11099 | 12349 | 13599 | 14849 | 16099 | 17349 | 18599 | 19849 |
| TOTCOVID01156 | 11100 | 12350 | 13600 | 14850 | 16100 | 17350 | 18600 | 19850 |
| TOTCOVID01157 | 11101 | 12351 | 13601 | 14851 | 16101 | 17351 | 18601 | 19851 |
| TOTCOVID01158 | 11102 | 12352 | 13602 | 14852 | 16102 | 17352 | 18602 | 19852 |
| TOTCOVID01159 | 11103 | 12353 | 13603 | 14853 | 16103 | 17353 | 18603 | 19853 |
| TOTCOVID01160 | 11104 | 12354 | 13604 | 14854 | 16104 | 17354 | 18604 | 19854 |
| TOTCOVID01161 | 11105 | 12355 | 13605 | 14855 | 16105 | 17355 | 18605 | 19855 |
| TOTCOVID01162 | 11106 | 12356 | 13606 | 14856 | 16106 | 17356 | 18606 | 19856 |
| TOTCOVID01163 | 11107 | 12357 | 13607 | 14857 | 16107 | 17357 | 18607 | 19857 |
| TOTCOVID01164 | 11108 | 12358 | 13608 | 14858 | 16108 | 17358 | 18608 | 19858 |
| TOTCOVID01165 | 11109 | 12359 | 13609 | 14859 | 16109 | 17359 | 18609 | 19859 |
| TOTCOVID01166 | 11110 | 12360 | 13610 | 14860 | 16110 | 17360 | 18610 | 19860 |
| TOTCOVID01167 | 11111 | 12361 | 13611 | 14861 | 16111 | 17361 | 18611 | 19861 |
| TOTCOVID01168 | 11112 | 12362 | 13612 | 14862 | 16112 | 17362 | 18612 | 19862 |
| TOTCOVID01169 | 11113 | 12363 | 13613 | 14863 | 16113 | 17363 | 18613 | 19863 |
| TOTCOVID01170 | 11114 | 12364 | 13614 | 14864 | 16114 | 17364 | 18614 | 19864 |
| TOTCOVID01171 | 11115 | 12365 | 13615 | 14865 | 16115 | 17365 | 18615 | 19865 |
| TOTCOVID01172 | 11116 | 12366 | 13616 | 14866 | 16116 | 17366 | 18616 | 19866 |
| TOTCOVID01173 | 11117 | 12367 | 13617 | 14867 | 16117 | 17367 | 18617 | 19867 |
| TOTCOVID01174 | 11118 | 12368 | 13618 | 14868 | 16118 | 17368 | 18618 | 19868 |
| TOTCOVID01175 | 11119 | 12369 | 13619 | 14869 | 16119 | 17369 | 18619 | 19869 |
| TOTCOVID01176 | 11120 | 12370 | 13620 | 14870 | 16120 | 17370 | 18620 | 19870 |
| TOTCOVID01177 | 11121 | 12371 | 13621 | 14871 | 16121 | 17371 | 18621 | 19871 |
| TOTCOVID01178 | 11122 | 12372 | 13622 | 14872 | 16122 | 17372 | 18622 | 19872 |
| TOTCOVID01179 | 11123 | 12373 | 13623 | 14873 | 16123 | 17373 | 18623 | 19873 |
| TOTCOVID01180 | 11124 | 12374 | 13624 | 14874 | 16124 | 17374 | 18624 | 19874 |
| TOTCOVID01181 | 11125 | 12375 | 13625 | 14875 | 16125 | 17375 | 18625 | 19875 |
| TOTCOVID01182 | 11126 | 12376 | 13626 | 14876 | 16126 | 17376 | 18626 | 19876 |
| TOTCOVID01183 | 11127 | 12377 | 13627 | 14877 | 16127 | 17377 | 18627 | 19877 |
| TOTCOVID01184 | 11128 | 12378 | 13628 | 14878 | 16128 | 17378 | 18628 | 19878 |
| TOTCOVID01185 | 11129 | 12379 | 13629 | 14879 | 16129 | 17379 | 18629 | 19879 |
| TOTCOVID01186 | 11130 | 12380 | 13630 | 14880 | 16130 | 17380 | 18630 | 19880 |
| TOTCOVID01187 | 11131 | 12381 | 13631 | 14881 | 16131 | 17381 | 18631 | 19881 |
| TOTCOVID01188 | 11132 | 12382 | 13632 | 14882 | 16132 | 17382 | 18632 | 19882 |
| TOTCOVID01189 | 11133 | 12383 | 13633 | 14883 | 16133 | 17383 | 18633 | 19883 |
| TOTCOVID01190 | 11134 | 12384 | 13634 | 14884 | 16134 | 17384 | 18634 | 19884 |
| TOTCOVID01191 | 11135 | 12385 | 13635 | 14885 | 16135 | 17385 | 18635 | 19885 |
| TOTCOVID01192 | 11136 | 12386 | 13636 | 14886 | 16136 | 17386 | 18636 | 19886 |
| TOTCOVID01193 | 11137 | 12387 | 13637 | 14887 | 16137 | 17387 | 18637 | 19887 |
| TOTCOVID01194 | 11138 | 12388 | 13638 | 14888 | 16138 | 17388 | 18638 | 19888 |
| TOTCOVID01195 | 11139 | 12389 | 13639 | 14889 | 16139 | 17389 | 18639 | 19889 |
| TOTCOVID01196 | 11140 | 12390 | 13640 | 14890 | 16140 | 17390 | 18640 | 19890 |
| TOTCOVID01197 | 11141 | 12391 | 13641 | 14891 | 16141 | 17391 | 18641 | 19891 |
| TOTCOVID01198 | 11142 | 12392 | 13642 | 14892 | 16142 | 17392 | 18642 | 19892 |
| TOTCOVID01199 | 11143 | 12393 | 13643 | 14893 | 16143 | 17393 | 18643 | 19893 |
| TOTCOVID01200 | 11144 | 12394 | 13644 | 14894 | 16144 | 17394 | 18644 | 19894 |
| TOTCOVID01201 | 11145 | 12395 | 13645 | 14895 | 16145 | 17395 | 18645 | 19895 |
| TOTCOVID01202 | 11146 | 12396 | 13646 | 14896 | 16146 | 17396 | 18646 | 19896 |
| TOTCOVID01203 | 11147 | 12397 | 13647 | 14897 | 16147 | 17397 | 18647 | 19897 |
| TOTCOVID01204 | 11148 | 12398 | 13648 | 14898 | 16148 | 17398 | 18648 | 19898 |
| TOTCOVID01205 | 11149 | 12399 | 13649 | 14899 | 16149 | 17399 | 18649 | 19899 |
| TOTCOVID01206 | 11150 | 12400 | 13650 | 14900 | 16150 | 17400 | 18650 | 19900 |
| TOTCOVID01207 | 11151 | 12401 | 13651 | 14901 | 16151 | 17401 | 18651 | 19901 |
| TOTCOVID01208 | 11152 | 12402 | 13652 | 14902 | 16152 | 17402 | 18652 | 19902 |
| TOTCOVID01209 | 11153 | 12403 | 13653 | 14903 | 16153 | 17403 | 18653 | 19903 |
| TOTCOVID01210 | 11154 | 12404 | 13654 | 14904 | 16154 | 17404 | 18654 | 19904 |
| TOTCOVID01211 | 11155 | 12405 | 13655 | 14905 | 16155 | 17405 | 18655 | 19905 |
| TOTCOVID01212 | 11156 | 12406 | 13656 | 14906 | 16156 | 17406 | 18656 | 19906 |
| TOTCOVID01213 | 11157 | 12407 | 13657 | 14907 | 16157 | 17407 | 18657 | 19907 |
| TOTCOVID01214 | 11158 | 12408 | 13658 | 14908 | 16158 | 17408 | 18658 | 19908 |
| TOTCOVID01215 | 11159 | 12409 | 13659 | 14909 | 16159 | 17409 | 18659 | 19909 |
| TOTCOVID01216 | 11160 | 12410 | 13660 | 14910 | 16160 | 17410 | 18660 | 19910 |
| TOTCOVID01217 | 11161 | 12411 | 13661 | 14911 | 16161 | 17411 | 18661 | 19911 |
| TOTCOVID01218 | 11162 | 12412 | 13662 | 14912 | 16162 | 17412 | 18662 | 19912 |
| TOTCOVID01219 | 11163 | 12413 | 13663 | 14913 | 16163 | 17413 | 18663 | 19913 |
| TOTCOVID01220 | 11164 | 12414 | 13664 | 14914 | 16164 | 17414 | 18664 | 19914 |
| TOTCOVID01221 | 11165 | 12415 | 13665 | 14915 | 16165 | 17415 | 18665 | 19915 |
| TOTCOVID01222 | 11166 | 12416 | 13666 | 14916 | 16166 | 17416 | 18666 | 19916 |
| TOTCOVID01223 | 11167 | 12417 | 13667 | 14917 | 16167 | 17417 | 18667 | 19917 |
| TOTCOVID01224 | 11168 | 12418 | 13668 | 14918 | 16168 | 17418 | 18668 | 19918 |
| TOTCOVID01225 | 11169 | 12419 | 13669 | 14919 | 16169 | 17419 | 18669 | 19919 |
| TOTCOVID01226 | 11170 | 12420 | 13670 | 14920 | 16170 | 17420 | 18670 | 19920 |
| TOTCOVID01227 | 11171 | 12421 | 13671 | 14921 | 16171 | 17421 | 18671 | 19921 |
| TOTCOVID01228 | 11172 | 12422 | 13672 | 14922 | 16172 | 17422 | 18672 | 19922 |
| TOTCOVID01229 | 11173 | 12423 | 13673 | 14923 | 16173 | 17423 | 18673 | 19923 |
| TOTCOVID01230 | 11174 | 12424 | 13674 | 14924 | 16174 | 17424 | 18674 | 19924 |
| TOTCOVID01231 | 11175 | 12425 | 13675 | 14925 | 16175 | 17425 | 18675 | 19925 |
| TOTCOVID01232 | 11176 | 12426 | 13676 | 14926 | 16176 | 17426 | 18676 | 19926 |
| TOTCOVID01233 | 11177 | 12427 | 13677 | 14927 | 16177 | 17427 | 18677 | 19927 |
| TOTCOVID01234 | 11178 | 12428 | 13678 | 14928 | 16178 | 17428 | 18678 | 19928 |
| TOTCOVID01235 | 11179 | 12429 | 13679 | 14929 | 16179 | 17429 | 18679 | 19929 |
| TOTCOVID01236 | 11180 | 12430 | 13680 | 14930 | 16180 | 17430 | 18680 | 19930 |
| TOTCOVID01237 | 11181 | 12431 | 13681 | 14931 | 16181 | 17431 | 18681 | 19931 |
| TOTCOVID01238 | 11182 | 12432 | 13682 | 14932 | 16182 | 17432 | 18682 | 19932 |
| TOTCOVID01239 | 11183 | 12433 | 13683 | 14933 | 16183 | 17433 | 18683 | 19933 |
| TOTCOVID01240 | 11184 | 12434 | 13684 | 14934 | 16184 | 17434 | 18684 | 19934 |
| TOTCOVID01241 | 11185 | 12435 | 13685 | 14935 | 16185 | 17435 | 18685 | 19935 |
| TOTCOVID01242 | 11186 | 12436 | 13686 | 14936 | 16186 | 17436 | 18686 | 19936 |
| TOTCOVID01243 | 11187 | 12437 | 13687 | 14937 | 16187 | 17437 | 18687 | 19937 |
| TOTCOVID01244 | 11188 | 12438 | 13688 | 14938 | 16188 | 17438 | 18688 | 19938 |
| TOTCOVID01245 | 11189 | 12439 | 13689 | 14939 | 16189 | 17439 | 18689 | 19939 |
| TOTCOVID01246 | 11190 | 12440 | 13690 | 14940 | 16190 | 17440 | 18690 | 19940 |
| TOTCOVID01247 | 11191 | 12441 | 13691 | 14941 | 16191 | 17441 | 18691 | 19941 |
| TOTCOVID01248 | 11192 | 12442 | 13692 | 14942 | 16192 | 17442 | 18692 | 19942 |
| TOTCOVID01249 | 11193 | 12443 | 13693 | 14943 | 16193 | 17443 | 18693 | 19943 |
| TOTCOVID01250 | 11194 | 12444 | 13694 | 14944 | 16194 | 17444 | 18694 | 19944 |
| TOTCOVID01251 | 11195 | 12445 | 13695 | 14945 | 16195 | 17445 | 18695 | 19945 |
| TOTCOVID01252 | 11196 | 12446 | 13696 | 14946 | 16196 | 17446 | 18696 | 19946 |
| TOTCOVID01253 | 11197 | 12447 | 13697 | 14947 | 16197 | 17447 | 18697 | 19947 |
| TOTCOVID01254 | 11198 | 12448 | 13698 | 14948 | 16198 | 17448 | 18698 | 19948 |
| TOTCOVID01255 | 11199 | 12449 | 13699 | 14949 | 16199 | 17449 | 18699 | 19949 |
| TOTCOVID01256 | 11200 | 12450 | 13700 | 14950 | 16200 | 17450 | 18700 | 19950 |
| TOTCOVID01257 | 11201 | 12451 | 13701 | 14951 | 16201 | 17451 | 18701 | 19951 |
| TOTCOVID01258 | 11202 | 12452 | 13702 | 14952 | 16202 | 17452 | 18702 | 19952 |
| TOTCOVID01259 | 11203 | 12453 | 13703 | 14953 | 16203 | 17453 | 18703 | 19953 |
| TOTCOVID01260 | 11204 | 12454 | 13704 | 14954 | 16204 | 17454 | 18704 | 19954 |
| TOTCOVID01261 | 11205 | 12455 | 13705 | 14955 | 16205 | 17455 | 18705 | 19955 |
| TOTCOVID01262 | 11206 | 12456 | 13706 | 14956 | 16206 | 17456 | 18706 | 19956 |
| TOTCOVID01263 | 11207 | 12457 | 13707 | 14957 | 16207 | 17457 | 18707 | 19957 |
| TOTCOVID01264 | 11208 | 12458 | 13708 | 14958 | 16208 | 17458 | 18708 | 19958 |
| TOTCOVID01265 | 11209 | 12459 | 13709 | 14959 | 16209 | 17459 | 18709 | 19959 |
| TOTCOVID01266 | 11210 | 12460 | 13710 | 14960 | 16210 | 17460 | 18710 | 19960 |
| TOTCOVID01267 | 11211 | 12461 | 13711 | 14961 | 16211 | 17461 | 18711 | 19961 |
| TOTCOVID01268 | 11212 | 12462 | 13712 | 14962 | 16212 | 17462 | 18712 | 19962 |
| TOTCOVID01269 | 11213 | 12463 | 13713 | 14963 | 16213 | 17463 | 18713 | 19963 |
| TOTCOVID01270 | 11214 | 12464 | 13714 | 14964 | 16214 | 17464 | 18714 | 19964 |
| TOTCOVID01271 | 11215 | 12465 | 13715 | 14965 | 16215 | 17465 | 18715 | 19965 |
| TOTCOVID01272 | 11216 | 12466 | 13716 | 14966 | 16216 | 17466 | 18716 | 19966 |
| TOTCOVID01273 | 11217 | 12467 | 13717 | 14967 | 16217 | 17467 | 18717 | 19967 |
| TOTCOVID01274 | 11218 | 12468 | 13718 | 14968 | 16218 | 17468 | 18718 | 19968 |
| TOTCOVID01275 | 11219 | 12469 | 13719 | 14969 | 16219 | 17469 | 18719 | 19969 |
| TOTCOVID01276 | 11220 | 12470 | 13720 | 14970 | 16220 | 17470 | 18720 | 19970 |
| TOTCOVID01277 | 11221 | 12471 | 13721 | 14971 | 16221 | 17471 | 18721 | 19971 |
| TOTCOVID01278 | 11222 | 12472 | 13722 | 14972 | 16222 | 17472 | 18722 | 19972 |
| TOTCOVID01279 | 11223 | 12473 | 13723 | 14973 | 16223 | 17473 | 18723 | 19973 |
| TOTCOVID01280 | 11224 | 12474 | 13724 | 14974 | 16224 | 17474 | 18724 | 19974 |
| TOTCOVID01281 | 11225 | 12475 | 13725 | 14975 | 16225 | 17475 | 18725 | 19975 |
| TOTCOVID01282 | 11226 | 12476 | 13726 | 14976 | 16226 | 17476 | 18726 | 19976 |
| TOTCOVID01283 | 11227 | 12477 | 13727 | 14977 | 16227 | 17477 | 18727 | 19977 |
| TOTCOVID01284 | 11228 | 12478 | 13728 | 14978 | 16228 | 17478 | 18728 | 19978 |
| TOTCOVID01285 | 11229 | 12479 | 13729 | 14979 | 16229 | 17479 | 18729 | 19979 |
| TOTCOVID01286 | 11230 | 12480 | 13730 | 14980 | 16230 | 17480 | 18730 | 19980 |
| TOTCOVID01287 | 11231 | 12481 | 13731 | 14981 | 16231 | 17481 | 18731 | 19981 |
| TOTCOVID01288 | 11232 | 12482 | 13732 | 14982 | 16232 | 17482 | 18732 | 19982 |
| TOTCOVID01289 | 11233 | 12483 | 13733 | 14983 | 16233 | 17483 | 18733 | 19983 |
| TOTCOVID01290 | 11234 | 12484 | 13734 | 14984 | 16234 | 17484 | 18734 | 19984 |
| TOTCOVID01291 | 11235 | 12485 | 13735 | 14985 | 16235 | 17485 | 18735 | 19985 |
| TOTCOVID01292 | 11236 | 12486 | 13736 | 14986 | 16236 | 17486 | 18736 | 19986 |
| TOTCOVID01293 | 11237 | 12487 | 13737 | 14987 | 16237 | 17487 | 18737 | 19987 |
| TOTCOVID01294 | 11238 | 12488 | 13738 | 14988 | 16238 | 17488 | 18738 | 19988 |
| TOTCOVID01295 | 11239 | 12489 | 13739 | 14989 | 16239 | 17489 | 18739 | 19989 |
| TOTCOVID01296 | 11240 | 12490 | 13740 | 14990 | 16240 | 17490 | 18740 | 19990 |
| TOTCOVID01297 | 11241 | 12491 | 13741 | 14991 | 16241 | 17491 | 18741 | 19991 |
| TOTCOVID01298 | 11242 | 12492 | 13742 | 14992 | 16242 | 17492 | 18742 | 19992 |
| TOTCOVID01299 | 11243 | 12493 | 13743 | 14993 | 16243 | 17493 | 18743 | 19993 |
| TOTCOVID01300 | 11244 | 12494 | 13744 | 14994 | 16244 | 17494 | 18744 | 19994 |
| TOTCOVID01301 | 11245 | 12495 | 13745 | 14995 | 16245 | 17495 | 18745 | 19995 |
| TOTCOVID01302 | 11246 | 12496 | 13746 | 14996 | 16246 | 17496 | 18746 | 19996 |
| TOTCOVID01303 | 11247 | 12497 | 13747 | 14997 | 16247 | 17497 | 18747 | 19997 |
| TOTCOVID01304 | 11248 | 12498 | 13748 | 14998 | 16248 | 17498 | 18748 | 19998 |
| TOTCOVID01305 | 11249 | 12499 | 13749 | 14999 | 16249 | 17499 | 18749 | 19999 |
| TOTCOVID01306 | 11250 | 12500 | 13750 | 15000 | 16250 | 17500 | 18750 | 20000 |

### Example 5: Antibody Neutralization Experiment

Neutralizing antibodies against SARS-CoV-2 can block the interaction between the SARS-CoV-2 receptor binding domain (RBD) and the ACE2 receptor on target cells, and so have potential to reduce viral replication and lung damage. The following experiment was performed to evaluate the effectiveness of various antibodies of the disclosure for SARS-CoV-2 neutralization. A SARS-CoV-2 pseudovirus neutralization assay kit (Genscript, SC2087A) was used with a known monoclonal antibody directed against SARS-CoV-2 (Regeneron, REGN10933) selected as a positive control. Serial dilutions of positive control and antibody samples were prepared with Opti-MEM® reduced serum medium and 254 of each dilution was transferred to dedicated wells in a 96-well assay plate. 254 of pseudovirus solution was added to each well, mixed thoroughly, and incubated at room temperature for 1 hour to allow for neutralization. During pseudovirus incubation, Opti-HEK293/ACE2 cells were prepared with an adjusted cell density of 6×10⁵ cells/mL. 504 of this suspension was added to each of the wells and the plate was incubated at 37° C. in a 5% CO2 environment for 24 hours. 50 μL of prewarmed fresh DMEM with 10% FBS was then added for another 24-hour period. After 48 hours of infection, the supernatant in the 96-well plate was carefully aspirated and discarded, and 50 μL of fresh-made luciferase detection agent (L00877C) was added. After about 5 minutes, the bioluminescent signal from each well was read using a microplate reader at 560 nm.

Results for an exemplary antibody of the disclosure (TOTCOVID00425) are displayed in Table 2, below. The units provided indicate relative fluorescence observed for two replicate samples. Lower fluorescence indicates SARS-CoV-2 pseudovirus neutralization. As shown in Table 2, antibody TOTCOVID00425 has neutralizing properties like that of the positive control and therefore is a suitable candidate therapy for the treatment of COVID-19.

TABLE 2
Results of neutralization assay comparing antibody of the disclosure TOTCOVID00425 and positive control REGN-10933.

| | | | | | | | | | | |
|---|---|---|---|---|---|---|---|---|---|---|
| Conc. (mg/mL) | 0.5 | 0.1667 | 0.0556 | 0.0185 | 0.0062 | 0.0021 | 0.0007 | 0.0002 | 8E−05 | 3E−05 |
| TOTCOVID00425 | 1920 | 3120 | 7920 | 2.86E+04 | 1.49E+05 | 4.12E+05 | 6.56E+05 | 6.87E+05 | 7.97E+05 | 1.77E+06 |
| | 7520 | 3280 | 8680 | 3.32E+04 | 1.65E+05 | 4.34E+05 | 8.01E+05 | 1.14E+06 | 1.21E+06 | 1.65E+06 |
| Conc. (mg/mL) | 0.17 | 0.0567 | 0.0189 | 0.0063 | 0.0021 | 0.0007 | 0.0002 | 8E−05 | 3E−05 | 9E−09 |
| REGN 10933 | 440 | 360 | 600 | 360 | 1560 | 2.64E+04 | 2.98E+05 | 9.33E+05 | 1.14E+06 | 1.93E+06 |
| | 720 | 720 | 600 | 400 | 4560 | 4.94E+04 | 2.67E+05 | 1.02E+06 | 1.90E+06 | 2.11E+06 |

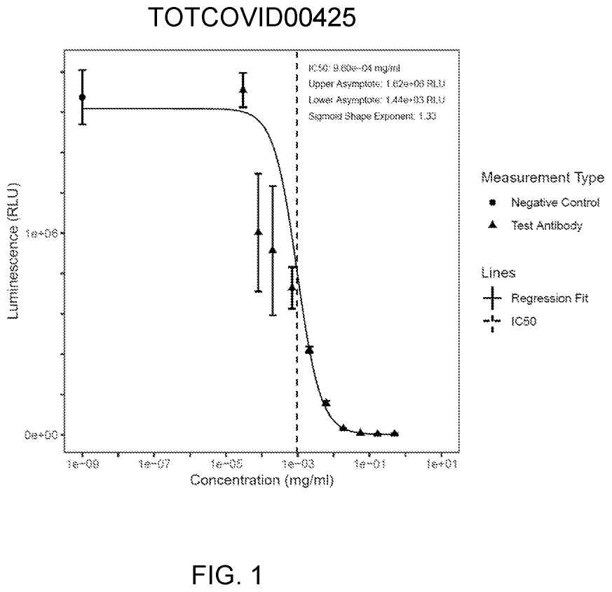

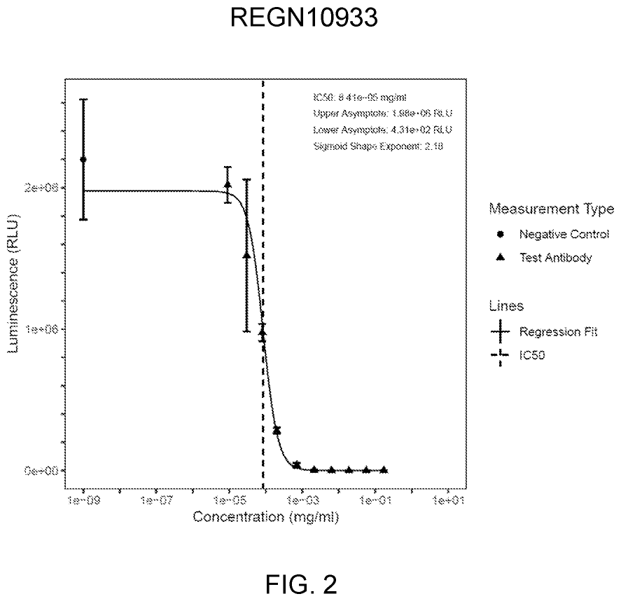

FIGS. 1-8 are charts depicting dose response curves generated from the concentration values and estimated fraction infectivity from the above experiment. The charts include calculated half maximal inhibitory concentrations (IC₅₀) values for various antibodies according to the disclosure, indicating the therapeutic effectiveness of each antibody for the treatment of COVID-19. As shown in FIGS. 1-2, antibody TOTCOVID00425 has an IC₅₀ of 9.60e-04 mg/mL, comparable to the positive control antibody REGN-10933 which has an IC₅₀ of 8.41e-05 mg/mL.

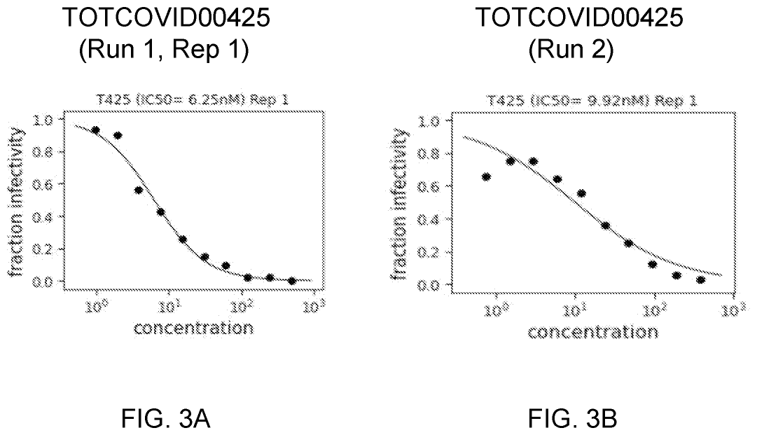

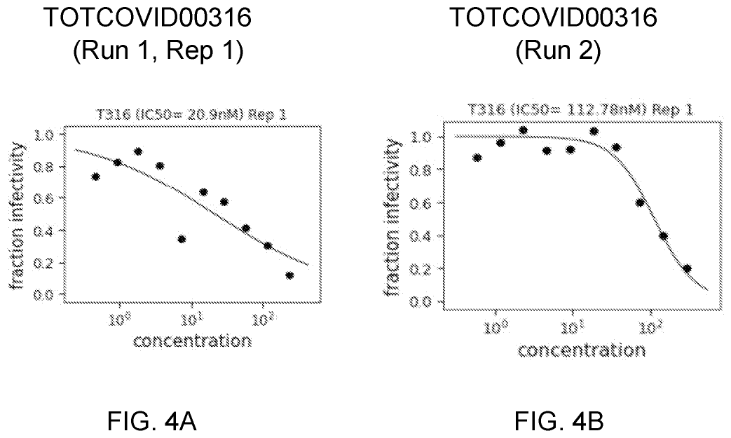

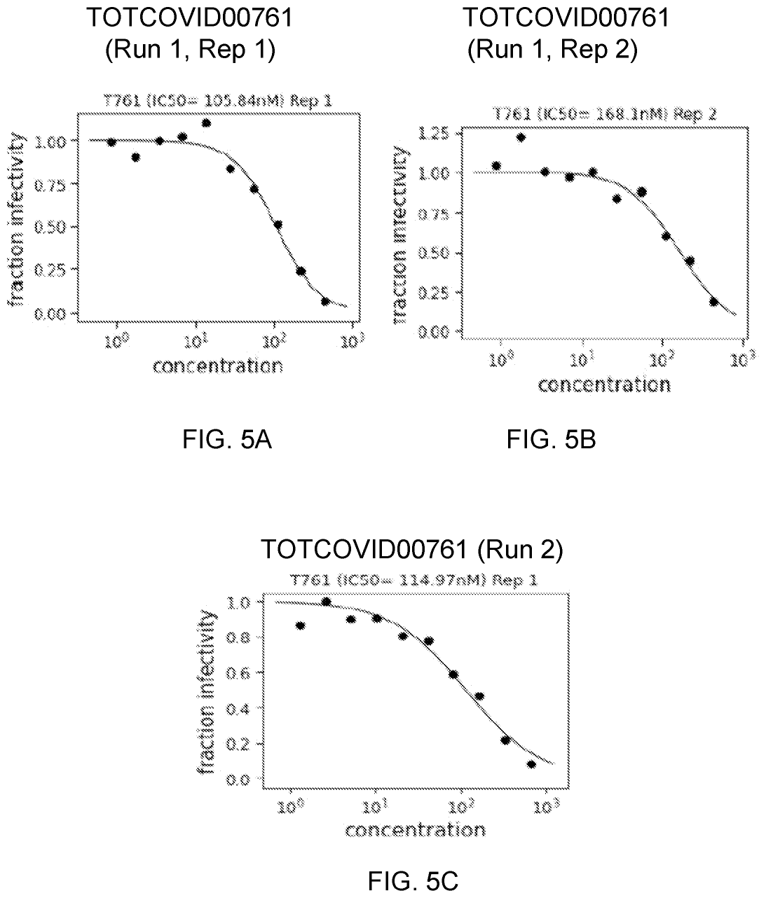

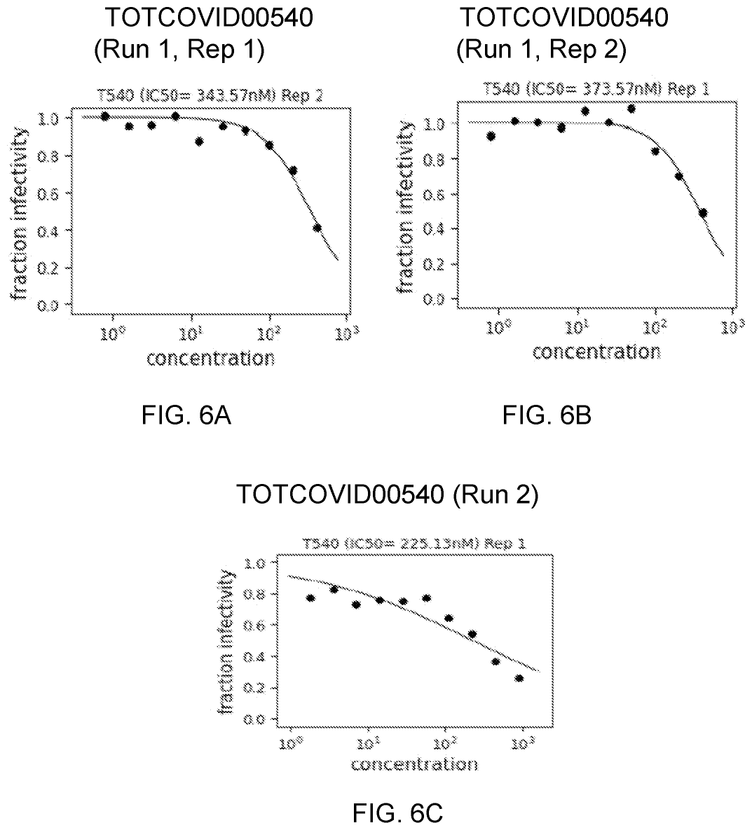

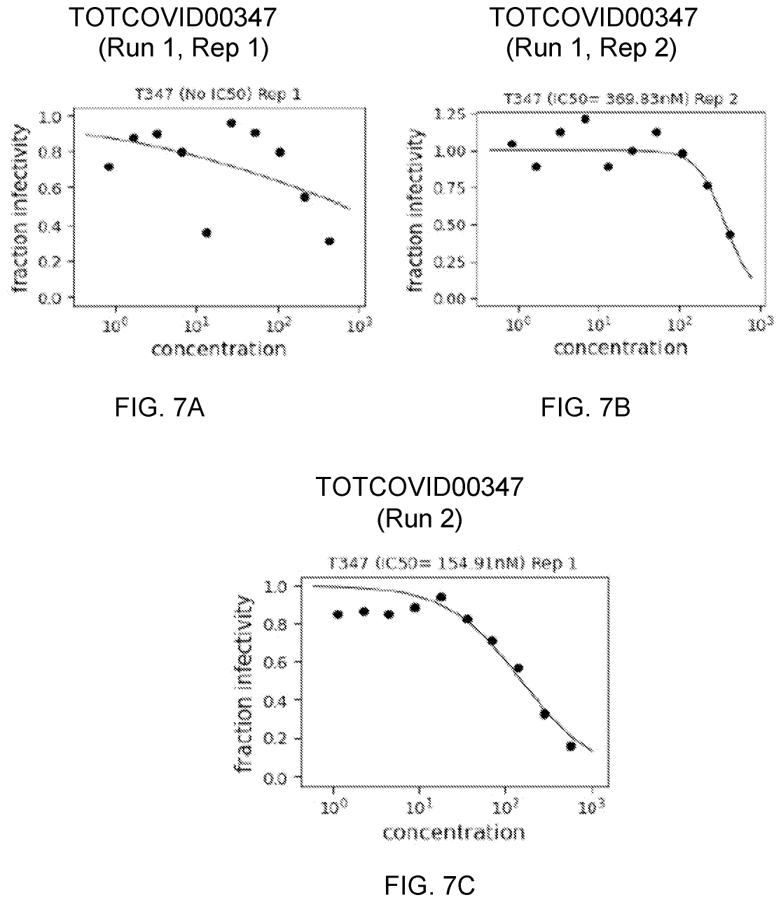

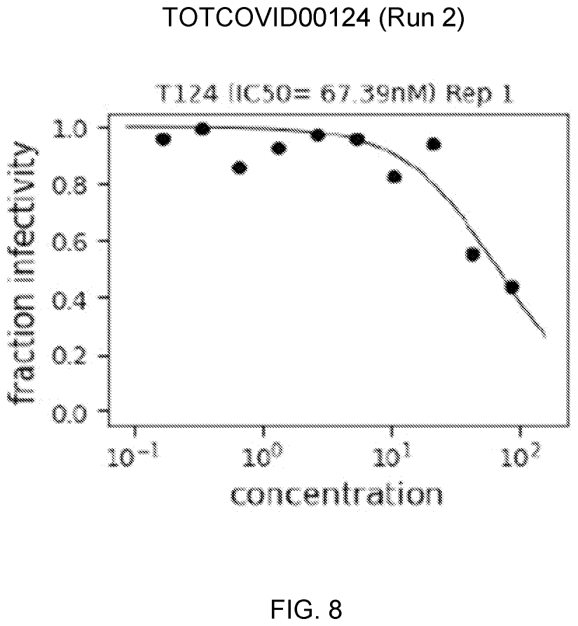

FIGS. 3A-3B depict dose response curves using additional data for antibody TOTCOVID00425, showing IC₅₀ values of 6.25 nM and 9.92 nM. FIGS. 4A-4B depict dose response curves for antibody TOTCOVID00316, showing IC₅₀ values of 20.9 nM and 112.78 nM. FIGS. 5A-5C depict dose response curves for antibody TOTCOVID00761, showing IC₅₀ values of 105.84 nM, 168.1 nM, and 114.97 nM. FIGS. 6A-6C depict dose response curves for antibody TOTCOVID00540, showing IC₅₀ values of 343 0.57 nM, 373 0.57 nM, and 225.13 nM. FIGS. 7A-7C depict dose response curves for antibody TOTCOVID00347, showing IC₅₀ values of 343.57 nM, 373.57 nM, and 225.13 nM. FIG. 8 depicts a dose response curve for antibody TOTCOVID00124, showing an IC₅₀ value of 67.39 nM.

The results demonstrate that the antibodies of the disclosure are functionally equivalent to the positive control, indicating their utility for the treatment of a SARS-CoV-2 infection and COVID-19.

### Example 6: Epitope Mapping

Epitope mapping was performed on various antibodies according to the disclosure to demonstrate their therapeutic utility. Antibody samples were processed using a microarray-based antibody detection assay including full-length proteins and peptides spanning the 51 and S2 subunits of the SARS-CoV-2 spike protein (CDI Labs SARS-CoV-2 Protein Microarray, CDICOV2-001.0).

Representative spike protein peptides on the array are listed in Table 3, below.

TABLE 3
Representative amino acid sequences, start and
end positions, and SEQ ID NOs for peptide
fragments from the S1 and S2 subunits of SARS-
COV-2 spike protein on a microarray-based
antibody detection assay.

| SEQ | | | | |
|---|---|---|---|---|
| ID | Peptide | Start | Amino acid | End |
| NO. | ID | Position | sequence | Position |
| 20001 | S1-1 | 1 | MFVFLVLLPLVS | 12 |
| 20002 | S1-2 | 7 | LLPLVSSQCVNL | 18 |
| 20003 | S1-3 | 13 | SQCVNLTTRTQL | 24 |
| 20004 | S1-4 | 19 | TTRTQLPPAYTN | 30 |
| 20005 | S1-5 | 25 | PPAYTNSFTRGV | 36 |
| 20006 | S1-6 | 31 | SFTRGVYYPDKV | 42 |
| 20007 | S1-7 | 37 | YYPDKVFRSSVL | 48 |
| 20008 | S1-8 | 43 | FRSSVLHSTQDL | 54 |
| 20009 | S1-9 | 49 | HSTQDLFLPFFS | 60 |
| 20010 | S1-10 | 55 | FLPFFSNVTWFH | 66 |
| 20011 | S1-11 | 61 | NVTWFHAIHVSG | 72 |
| 20012 | S1-12 | 67 | AIHVSGTNGTKR | 78 |
| 20013 | S1-13 | 73 | TNGTKRFDNPVL | 84 |
| 20014 | S1-14 | 7 | FDNPVLPFNDGV | 90 |
| 20015 | S1-15 | 85 | PFNDGVYFASTE | 96 |
| 20016 | S1-16 | 91 | YFASTEKSNIIR | 102 |
| 20017 | S1-17 | 97 | KSNIIRGWIFGT | 108 |
| 20018 | S1-18 | 103 | GWIFGTTLDSKT | 114 |
| 20019 | S1-19 | 109 | TLDSKTQSLLIV | 120 |
| 20020 | S1-20 | 115 | QSLLIVNNATNV | 126 |
| 20021 | S1-21 | 121 | NNATNVVIKVCE | 132 |
| 20022 | S1-22 | 127 | VIKVCEFQFCND | 138 |
| 20023 | S1-23 | 133 | FQFCNDPFLGVY | 144 |
| 20024 | S1-24 | 139 | PFLGVYYHKNNK | 150 |
| 20025 | S1-25 | 145 | YHKNNKSWMESE | 156 |
| 20026 | S1-26 | 151 | SWMESEFRVYSS | 162 |
| 20027 | S1-27 | 157 | FRVYSSANNCTF | 168 |
| 20028 | S1-28 | 163 | ANNCTFEYVSQP | 174 |
| 20029 | S1-29 | 169 | EYVSQPFLMDLE | 180 |
| 20030 | S1-30 | 175 | FLMDLEGKQGNF | 186 |
| 20031 | S1-31 | 181 | GKQGNFKNLREF | 192 |
| 20032 | S1-32 | 187 | KNLREFVFKNID | 198 |
| 20033 | S1-33 | 193 | VFKNIDGYFKIY | 204 |
| 20034 | S1-34 | 199 | GYFKIYSKHTPI | 210 |
| 20035 | S1-35 | 205 | SKHTPINLVRDL | 216 |
| 20036 | S1-36 | 211 | NLVRDLPQGFSA | 222 |
| 20037 | S1-37 | 217 | PQGFSALEPLVD | 228 |
| 20038 | S1-38 | 223 | LEPLVDLPIGIN | 234 |
| 20039 | S1-39 | 229 | LPIGINITRFQT | 240 |
| 20040 | S1-40 | 235 | ITRFQTLLALHR | 246 |
| 20041 | S1-41 | 241 | LLALHRSYLTPG | 252 |
| 20042 | S1-42 | 247 | SYLTPGDSSSGW | 258 |
| 20043 | S1-43 | 253 | DSSSGWTAGAAA | 264 |
| 20044 | S1-44 | 259 | TAGAAAYYVGYL | 270 |
| 20045 | S1-45 | 265 | YYVGYLQPRTFL | 276 |
| 20046 | S1-46 | 271 | QPRTFLLKYNEN | 282 |
| 20047 | S1-47 | 277 | LKYNENGTITDA | 288 |
| 20048 | S1-48 | 283 | GTITDAVDCALD | 294 |
| 20049 | S1-49 | 289 | VDCALDPLSETK | 300 |
| 20050 | S1-50 | 295 | PLSETKCTLKSF | 306 |
| 20051 | S1-51 | 301 | CTLKSFTVEKGI | 312 |
| 20052 | S1-52 | 307 | TVEKGIYQTSNF | 318 |
| 20053 | S1-53 | 313 | YQTSNFRVQPTE | 324 |
| 20054 | S1-54 | 319 | RVQPTESIVRFP | 330 |
| 20055 | S1-55 | 325 | SIVRFPNITNLC | 336 |
| 20056 | S1-56 | 331 | NITNLCPFGEVF | 342 |
| 20057 | S1-57 | 337 | PFGEVFNATRFA | 348 |
| 20058 | S1-58 | 343 | NATRFASVYAWN | 354 |
| 20059 | S1-59 | 349 | SVYAWNRKRISN | 360 |
| 20060 | S1-60 | 355 | RKRISNCVADYS | 366 |
| 20061 | S1-61 | 361 | CVADYSVLYNSA | 372 |
| 20062 | S1-62 | 367 | VLYNSASFSTFK | 378 |
| 20063 | S1-63 | 373 | SFSTFKCYGVSP | 384 |
| 20064 | S1-64 | 379 | CYGVSPTKLNDL | 390 |
| 20065 | S1-65 | 385 | TKLNDLCFTNVY | 396 |
| 20066 | S1-66 | 391 | CFTNVYADSFVI | 402 |
| 20067 | S1-67 | 397 | ADSFVIRGDEVR | 408 |
| 20068 | S1-68 | 403 | RGDEVRQIAPGQ | 414 |
| 20069 | S1-69 | 409 | QIAPGQTGKIAD | 420 |
| 20070 | S1-70 | 415 | TGKIADYNYKLP | 426 |
| 20071 | S1-71 | 421 | YNYKLPDDFTGC | 432 |
| 20072 | S1-72 | 427 | DDFTGCVIAWNS | 438 |
| 20073 | S1-73 | 433 | VIAWNSNNLDSK | 444 |
| 20074 | S1-74 | 439 | NNLDSKVGGNYN | 450 |
| 20075 | S1-75 | 445 | VGGNYNYLYRLF | 456 |
| 20076 | S1-76 | 451 | YLYRLFRKSNLK | 462 |
| 20077 | S1-77 | 457 | RKSNLKPFERDI | 468 |
| 20078 | S1-78 | 463 | PFERDISTEIYQ | 474 |
| 20079 | S1-79 | 469 | STEIYQAGSTPC | 480 |
| 20080 | S1-80 | 475 | AGSTPCNGVEGF | 486 |
| 20081 | S1-81 | 481 | NGVEGFNCYFPL | 492 |
| 20082 | S1-82 | 487 | NCYFPLQSYGFQ | 498 |
| 20083 | S1-83 | 493 | QSYGFQPTNGVG | 504 |
| 20084 | S1-84 | 499 | PTNGVGYQPYRV | 510 |
| 20085 | S1-85 | 505 | YQPYRVVVLSFE | 516 |
| 20086 | S1-86 | 511 | VVLSFELLHAPA | 522 |
| 20087 | S1-87 | 517 | LLHAPATVCGPK | 528 |
| 20088 | S1-88 | 523 | TVCGPKKSTNLV | 534 |
| 20089 | S1-89 | 529 | KSTNLVKNKCVN | 540 |
| 20090 | S1-90 | 535 | KNKCVNFNFNGL | 546 |
| 20091 | S1-91 | 541 | FNFNGLTGTGVL | 552 |
| 20092 | S1-92 | 547 | TGTGVLTESNKK | 558 |
| 20093 | S1-93 | 553 | TESNKKFLPFQQ | 564 |
| 20094 | S1-94 | 559 | FLPFQQFGRDIA | 570 |
| 20095 | S1-95 | 565 | FGRDIADTTDAV | 576 |
| 20096 | S1-96 | 571 | DTTDAVRDPQTL | 582 |
| 20097 | S1-97 | 577 | RDPQTLEILDIT | 588 |
| 20098 | S1-98 | 583 | EILDITPCSFGG | 594 |
| 20099 | S1-99 | 589 | PCSFGGVSVITP | 600 |
| 20100 | S1-100 | 595 | VSVITPGTNTSN | 606 |
| 20101 | S1-101 | 601 | GTNTSNQVAVLY | 612 |
| 20102 | S1-102 | 607 | QVAVLYQDVNCT | 618 |
| 20103 | S1-103 | 613 | QDVNCTEVPVAI | 624 |
| 20104 | S1-104 | 619 | EVPVAIHADQLT | 630 |
| 20105 | S1-105 | 625 | HADQLTPTWRVY | 636 |
| 20106 | S1-106 | 631 | PTWRVYSTGSNV | 642 |
| 20107 | S1-107 | 637 | STGSNVFQTRAG | 648 |
| 20108 | S1-108 | 643 | FQTRAGCLIGAE | 654 |
| 20109 | S1-109 | 649 | CLIGAEHVNNSY | 660 |
| 20110 | S1-110 | 655 | HVNNSYECDIPI | 666 |
| 20111 | S1-111 | 661 | ECDIPIGAGICA | 672 |
| 20112 | S1-112 | 667 | GAGICASYQTQT | 678 |
| 20113 | S1-113 | 673 | SYQTQTNSPRRA | 684 |
| 20114 | S1-114 | 679 | NSPRRARGGGGS | 685 |
| 20115 | S2-1 | 686 | SVASQSIIAYTM | 697 |
| 20116 | S2-2 | 692 | IIAYTMSLGAEN | 703 |
| 20117 | S2-3 | 698 | SLGAENSVAYSN | 709 |
| 20118 | S2-4 | 704 | SVAYSNNSIAIP | 715 |
| 20119 | S2-5 | 710 | NSIAIPTNFTIS | 721 |
| 20120 | S2-6 | 716 | TNFTISVTTEIL | 727 |
| 20121 | S2-7 | 722 | VTTEILPVSMTK | 733 |
| 20122 | S2-8 | 728 | PVSMTKTSVDCT | 739 |
| 20123 | S2-9 | 734 | TSVDCTMYICGD | 745 |
| 20124 | S2-10 | 740 | MYICGDSTECSN | 751 |
| 20125 | S2-11 | 746 | STECSNLLLQYG | 757 |
| 20126 | S2-12 | 752 | LLLQYGSFCTQL | 763 |
| 20127 | S2-13 | 758 | SFCTQLNRALTG | 769 |
| 20128 | S2-14 | 764 | NRALTGIAVEQD | 775 |
| 20129 | S2-15 | 770 | IAVEQDKNTQEV | 781 |
| 20130 | S2-16 | 776 | KNTQEVFAQVKQ | 787 |
| 20131 | S2-17 | 782 | FAQVKQIYKTPP | 793 |
| 20132 | S2-18 | 788 | TYKTPPIKDFGG | 799 |
| 20133 | S2-19 | 794 | IKDFGGFNFSQI | 805 |
| 20134 | S2-20 | 800 | FNFSQILPDPSK | 811 |
| 20135 | S2-21 | 806 | LPDPSKPSKRSF | 817 |
| 20136 | S2-22 | 812 | PSKRSFIEDLLF | 823 |
| 20137 | S2-23 | 818 | IEDLLFNKVTLA | 829 |
| 20138 | S2-24 | 824 | NKVTLADAGFIK | 835 |
| 20139 | S2-25 | 830 | DAGFIKQYGDCL | 841 |
| 20140 | S2-26 | 836 | QYGDCLGDIAAR | 847 |
| 20141 | S2-27 | 842 | GDIAARDLICAQ | 853 |
| 20142 | S2-28 | 848 | DLICAQKFNGLT | 859 |
| 20143 | S2-29 | 854 | KFNGLTVLPPLL | 865 |
| 20144 | S2-30 | 860 | VLPPLLTDEMIA | 871 |
| 20145 | S2-31 | 866 | TDEMIAQYTSAL | 877 |
| 20146 | S2-32 | 872 | QYTSALLAGTIT | 883 |
| 20147 | S2-33 | 878 | LAGTITSGWTFG | 889 |
| 20148 | S2-34 | 884 | SGWTFGAGAALQ | 895 |
| 20149 | S2-35 | 890 | AGAALQIPFAMQ | 901 |
| 20150 | S2-36 | 896 | IPFAMQMAYRFN | 907 |
| 20151 | S2-37 | 902 | MAYRFNGIGVTQ | 913 |
| 20152 | S2-38 | 908 | GIGVTQNVLYEN | 919 |
| 20153 | S2-39 | 914 | NVLYENQKLIAN | 925 |
| 20154 | S2-40 | 920 | QKLIANQFNSAI | 931 |
| 20155 | S2-41 | 926 | QFNSAIGKIQDS | 937 |
| 20156 | S2-42 | 932 | GKIQDSLSSTAS | 943 |
| 20157 | S2-43 | 938 | LSSTASALGKLQ | 949 |
| 20158 | S2-44 | 944 | ALGKLQDVVNQN | 955 |
| 20159 | S2-45 | 950 | DVVNQNAQALNT | 961 |
| 20160 | S2-46 | 956 | AQALNTLVKQLS | 967 |
| 20161 | S2-47 | 962 | LVKQLSSNFGAI | 973 |
| 20162 | S2-48 | 968 | SNFGAISSVLND | 979 |
| 20163 | S2-49 | 974 | SSVLNDILSRLD | 985 |
| 20164 | S2-50 | 980 | ILSRLDKVEAEV | 991 |
| 20165 | S2-51 | 986 | KVEAEVQIDRLI | 997 |
| 20166 | S2-52 | 992 | QIDRLITGRLQS | 1003 |
| 20167 | S2-53 | 998 | TGRLQSLQTYVT | 1009 |
| 20168 | S2-54 | 1004 | LQTYVTQQLIRA | 1015 |
| 20169 | S2-55 | 1010 | QQLIRAAEIRAS | 1021 |
| 20170 | S2-56 | 1016 | AEIRASANLAAT | 1027 |
| 20171 | S2-57 | 1022 | ANLAATKMSECV | 1033 |
| 20172 | S2-58 | 1028 | KMSECVLGQSKR | 1039 |
| 20173 | S2-59 | 1034 | LGQSKRVDFCGK | 1045 |
| 20174 | S2-60 | 1040 | VDFCGKGYHLMS | 1051 |
| 20175 | S2-61 | 1046 | GYHLMSFPQSAP | 1057 |
| 20176 | S2-62 | 1052 | FPQSAPHGVVFL | 1063 |
| 20177 | S2-63 | 1058 | HGVVFLHVTYVP | 1069 |
| 20178 | S2-64 | 1064 | HVTYVPAQEKNF | 1075 |
| 20179 | S2-65 | 1070 | AQEKNFTTAPAI | 1081 |
| 20180 | S2-66 | 1076 | TTAPAICHDGKA | 1087 |
| 20181 | S2-67 | 1082 | CHDGKAHFPREG | 1093 |
| 20182 | S2-68 | 1088 | HFPREGVFVSNG | 1099 |
| 20183 | S2-69 | 1094 | VFVSNGTHWFVT | 1105 |
| 20184 | S2-70 | 1100 | THWFVTQRNFYE | 1111 |
| 20185 | S2-71 | 1106 | QRNFYEPQIITT | 1117 |
| 20186 | S2-72 | 1112 | PQIITTDNTFVS | 1123 |
| 20187 | S2-73 | 1118 | DNTFVSGNCDVV | 1129 |
| 20188 | S2-74 | 1124 | GNCDVVIGIVNN | 1135 |
| 20189 | S2-75 | 1130 | IGIVNNTVYDPL | 1141 |
| 20190 | S2-76 | 1136 | TVYDPLQPELDS | 1147 |
| 20191 | S2-77 | 1142 | QPELDSFKEELD | 1153 |
| 20192 | S2-78 | 1148 | FKEELDKYFKNH | 1159 |
| 20193 | S2-79 | 1154 | KYFKNHTSPDVD | 1165 |
| 20194 | S2-80 | 1160 | TSPDVDLGDISG | 1171 |
| 20195 | S2-81 | 1166 | LGDISGINASVV | 1177 |
| 20196 | S2-82 | 1172 | INASVVNIQKEI | 1183 |
| 20197 | S2-83 | 1178 | NIQKEIDRLNEV | 1189 |
| 20198 | S2-84 | 1184 | DRLNEVAKNLNE | 1195 |
| 20199 | S2-85 | 1190 | AKNLNESLIDLQ | 1201 |
| 20200 | S2-86 | 1196 | SLIDLQELGKYE | 1207 |
| 20201 | S2-87 | 1202 | ELGKYEQYIKWP | 1213 |
| 20202 | S2-88 | 1208 | QYIKWPWYIWLG | 1219 |
| 20203 | S2-89 | 1214 | WYIWLGFIAGLI | 1225 |
| 20204 | S2-90 | 1220 | FIAGLIAIVMVT | 1231 |
| 20205 | S2-91 | 1226 | AIVMVTIMLCCM | 1237 |
| 20206 | S2-92 | 1232 | IMLCCMTSCCSC | 1243 |
| 20207 | S2-93 | 1238 | TSCCSCLKGCCS | 1249 |
| 20208 | S2-94 | 1244 | LKGCCSCGSCCK | 1255 |
| 20209 | S2-95 | 1250 | CGSCCKFDEDDS | 1261 |
| 20210 | S2-96 | 1256 | FDEDDSEPVLKG | 1267 |
| 20211 | S2-97 | 1262 | EPVLKGVKLHYT | 1273 |

Antibody samples were diluted (1:200) in PBS buffer containing 0.1% Tween 20, 1% BSA, and 0.5 mg/ml total E. coli lysate. 200 uL of diluted samples were then added to each of the wells and incubated at room temperature for 2 hours. Each well was then washed according to manufacturer's instructions to avoid contamination among samples. Secondary antibodies were diluted (according to manufacturer's recommend dilution) in PBS buffer containing 0.1% Tween 20 and 1% BSA and incubated at room temperature for 1 hour. This solution was then added to the wells and incubated for 1 hour with gentle shaking. The array was then washed according to manufacturer's instructions, dried, and scanned using a microarray scanner. Results were then analyzed using GenePix® Pro 7 Microarray Acquisitions and Analysis Software.

Results of the analysis for antibodies TOTCOVID00316, TOTCOVID00347, TOTCOVID00425, TOTCOVID00450, TOTCOVID00761, and a control (Anti-Human IgG) are provided in Tables 4-10, below. In each of Tables 4-10, the top 15 most significant hits are provided. As shown in Tables 4-10, each of the antibodies bound well to the S1 subunit, with peptide S1-61 representing the most likely epitope for antibodies TOTCOVID00316, TOTCOVID00347, TOTCOVID00425, TOTCOVID00450, and TOTCOVID00761. However, antibody TOTCOVID00761 also bound well to S1-45, S1-82, and S1-23, particularly when diluted to a higher concentration (see Tables 8, 9). This suggests that TOTCOVID00761 can be a multi-specific antibody and bind to separate epitopes from TOTCOVID00316, TOTCOVID00347, TOTCOVID00425, and TOTCOVID00450. Accordingly, this indicates that in one embodiment of the compositions, and methods of the present disclosure, antibody TOTCOVID00761 can be combined in a solution with one or more antibodies of the disclosure to target multiple regions of the SARS-CoV-2 spike protein, thereby increasing the efficacy of a therapeutic.

In conclusion, the inventors have analyzed the binding properties of the antibodies of the disclosure to the SARS-CoV-2 spike protein using the above-described experiment. As shown, the antibodies binds to at least one epitope, with at least one antibody having multi-specific properties.

Tables 4-10: Statistical analysis from microarray-based assay showing binding effectiveness of various antibodies of the disclosure to peptides from the S1 and S2 subunits of the SARS-CoV-2 spike unit protein. Human IgG, ACE2_Fc_0.5, and ACE2_Fc_0.17 are included as positive controls. F635 represents foreground fluorescence and F532 represents the frequency at which light is detected. Relative signal strength is provided as a Z-score.

TABLE 4
Results demonstrating target antigen binding by antibody TOTCOVID00316 (1 ug/mL)
Table 4: Antibody TOTCOVID00316 (1 ug/mL)

| Replicate 1 | | | | | Replicate 2 | | | | |
|---|---|---|---|---|---|---|---|---|---|
| Name | ID | F635 | F532 | Z-Score | Name | ID | F635 | F532 | Z-Score |
| Human IgG | 1G4 | 65535 | 211.7 | 7.52 | Human IgG | 1G4 | 65535 | 163.7 | 7.97 |
| ACE2_Fc_0.5 | 1.00E+07 | 65535 | 233.7 | 7.52 | ACE2_Fc_0.5 | 1.00E+07 | 65452.7 | 167 | 7.96 |
| ACE2_Fc_0.17 | 1H7 | 65535 | 197.7 | 7.52 | ACE2_Fc_0.17 | 1H7 | 60233.7 | 151 | 7.32 |
| S1_0.5 | 1B8 | 64421.7 | 241 | 7.39 | S1_0.5 | 1B8 | 54394 | 155.7 | 6.59 |
| S1_0.17 | 1C8 | 39154.3 | 197 | 4.43 | S1_0.17 | 1C8 | 35055 | 144 | 4.19 |
| S1 | 1H23 | 20538 | 150 | 2.24 | Anti-H-IgG | 1G20 | 23323 | 116 | 2.73 |
| Anti-H-IgG | 1G20 | 19796 | 134.7 | 2.16 | S1 | 1H23 | 16880.7 | 120 | 1.93 |
| S1-61 | 1O4 | 9907.7 | 161.3 | 1 | S1-61 | 1O4 | 9280 | 117 | 0.99 |

TABLE 5
Results demonstrating target antigen binding by antibody TOTCOVID00347 (1 ug/mL)
Table 5: Antibody TOTCOVID00347 (1 ug/mL)

| Replicate 1 | | | | | Replicate 2 | | | | |
|---|---|---|---|---|---|---|---|---|---|
| Name | ID | F635 | F532 | Z-Score | Name | ID | F635 | F532 | Z-Score |
| Human IgG | 1G4 | 65535 | 196.7 | 8.93 | Human IgG | 1G4 | 65535 | 179 | 8.89 |
| ACE2_Fc_0.5 | 1.00E+07 | 65535 | 219.3 | 8.93 | ACE2_Fc_0.5 | 1.00E+07 | 65535 | 160 | 8.89 |
| ACE2_Fc_0.17 | 1H7 | 65535 | 191.7 | 8.93 | ACE2_Fc_0.17 | 1H7 | 65535 | 138 | 8.89 |
| Anti-H-IgG | 1G20 | 22181.7 | 168.7 | 2.93 | Anti-H-IgG | 1G20 | 25767 | 166.3 | 3.41 |
| S1-61 | 1O4 | 16045 | 141 | 2.08 | S1-61 | 1O4 | 16238 | 123 | 2.09 |
| S1_0.5 | 1B8 | 10111.7 | 150.7 | 1.26 | S1_0.5 | 1B8 | 11066.7 | 134 | 1.38 |
| S1_0.17 | 1C8 | 8920.7 | 144.7 | 1.09 | S1_0.17 | 1C8 | 9034.7 | 120.3 | 1.1 |
| S1 | 1H23 | 4108.7 | 162.3 | 0.43 | S1 | 1H23 | 3235.3 | 156.3 | 0.3 |

TABLE 6
Results demonstrating target antigen binding by antibody TOTCOVID00425 (1 ug/mL)
Table 6: Antibody TOTCOVID00425 (1 ug/mL)

| Replicate 1 | | | | | Replicate 2 | | | | |
|---|---|---|---|---|---|---|---|---|---|
| Name | ID | F635 | F532 | Z-Score | Name | ID | F635 | F532 | Z-Score |
| Human IgG | 1G4 | 65535 | 215 | 8.38 | Human IgG | 1G4 | 65535 | 173.3 | 7.95 |
| ACE2_Fc_0.5 | 1.00E+07 | 65535 | 232.7 | 8.38 | ACE2_Fc_0.5 | 1.00E+07 | 65535 | 190.7 | 7.95 |
| ACE2_Fc_0.17 | 1H7 | 65535 | 192.3 | 8.38 | ACE2_Fc_0.17 | 1H7 | 65535 | 162 | 7.95 |
| S1_0.5 | 1B8 | 37755.3 | 168 | 4.76 | S1_0.5 | 1B8 | 48730.3 | 144.7 | 5.87 |
| S1_0.17 | 1C8 | 27634 | 156.3 | 3.45 | S1_0.17 | 1C8 | 35553.7 | 126 | 4.24 |
| Anti-H-IgG | 1G20 | 24947.3 | 162 | 3.1 | Anti-H-IgG | 1G20 | 28519.7 | 135.7 | 3.37 |
| S1 | 1H23 | 6928 | 159.7 | 0.75 | S1 | 1H23 | 9764 | 130 | 1.05 |
| S1-61 | 1O4 | 2761.7 | 144.3 | 0.21 | S1-61 | 1O4 | 2077.7 | 119.7 | 0.1 |

TABLE 7
Results demonstrating target antigen binding by antibody TOTCOVID00450 (1 ug/mL)
Table 7: Antibody TOTCOVID00450 (1 ug/mL)

| Replicate 1 | | | | | Replicate 2 | | | | |
|---|---|---|---|---|---|---|---|---|---|
| Name | ID | F635 | F532 | Z-Score | Name | ID | F635 | F532 | Z-Score |
| Human IgG | 1G4 | 65535 | 225.7 | 8.84 | Human IgG | 1G4 | 65535 | 173.3 | 8.73 |
| ACE2_Fc_0.5 | 1.00E+07 | 65535 | 256.3 | 8.84 | ACE2_Fc_0.5 | 1.00E+07 | 65535 | 206.7 | 8.73 |
| ACE2_Fc_0.17 | 1H7 | 65535 | 218.3 | 8.84 | ACE2_Fc_0.17 | 1H7 | 65535 | 160.3 | 8.73 |
| Anti-H-IgG | 1G20 | 29446.3 | 176.3 | 3.89 | Anti-H-IgG | 1G20 | 28447 | 127.7 | 3.7 |
| S1_0.17 | 1C8 | 14007.3 | 166 | 1.78 | S1_0.5 | 1B8 | 20314 | 136 | 2.6 |
| S1_0.5 | 1B8 | 13808.3 | 184.7 | 1.75 | S1_0.17 | 1C8 | 18819 | 119.7 | 2.4 |
| S1 | 1H23 | 4852.3 | 170 | 0.52 | S1 | 1H23 | 5411 | 127 | 0.58 |
| S1-61 | 1O4 | 959.3 | 160.3 | −0.01 | S1-61 | 1O4 | 3701.3 | 121 | 0.35 |

TABLE 8
Results demonstrating target antigen binding by antibody TOTCOVID00761 (1 ug/mL)
Table 8: Antibody TOTCOVID00761 (1 ug/mL)

| Replicate 1 | | | | | Replicate 2 | | | | |
|---|---|---|---|---|---|---|---|---|---|
| Name | ID | F635 | F532 | Z-Score | Name | ID | F635 | F532 | Z-Score |
| Human IgG | 1G4 | 65535 | 219.7 | 8.75 | Human IgG | 1G4 | 65535 | 169.7 | 8.79 |
| ACE2_Fc_0.5 | 1.00E+07 | 65535 | 248.3 | 8.75 | ACE2_Fc_0.5 | 1.00E+07 | 65535 | 192.3 | 8.79 |
| ACE2_Fc_0.17 | 1H7 | 65535 | 207 | 8.75 | ACE2_Fc_0.17 | 1H7 | 65535 | 155.7 | 8.79 |
| Anti-H-IgG | 1G20 | 39391.3 | 182.7 | 5.2 | Anti-H-IgG | 1G20 | 37940.7 | 143.3 | 5.03 |
| S1-61 | 1O4 | 2243.7 | 173 | 0.16 | S1-45 | 1P4 | 1804.7 | 169.3 | 0.11 |
| S1-45 | 1P4 | 1843.7 | 206 | 0.1 | S1-61 | 1O4 | 1669.7 | 133.7 | 0.09 |
| S1_0.5 | 1B8 | 1238.7 | 172.7 | 0.02 | S1_0.5 | 1B8 | 1251 | 134 | 0.03 |

TABLE 9
Results demonstrate target antigen binding by antibody TOTCOVID00761 (10 ug/mL)
Table 9: Antibody TOTCOVID00761 (10 ug/mL)

| Replicate 1 | | | | | Replicate 2 | | | | |
|---|---|---|---|---|---|---|---|---|---|
| Name | ID | F635 | F532 | Z-Score | Name | ID | F635 | F532 | Z-Score |
| Human IgG | 1G4 | 65535 | 236.3 | 7.28 | Human IgG | 1G4 | 65535 | 189.7 | 7.67 |
| ACE2_Fc_0.5 | 1.00E+07 | 65535 | 260.3 | 7.28 | ACE2_Fc_0.5 | 1.00E+07 | 65535 | 204 | 7.67 |
| ACE2_Fc_0.17 | 1H7 | 65535 | 210.7 | 7.28 | ACE2_Fc_0.17 | 1H7 | 65535 | 172.3 | 7.67 |
| Anti-H-IgG | 1G20 | 64244.7 | 172.3 | 7.13 | Anti-H-IgG | 1G20 | 55913.3 | 130.7 | 6.49 |
| S1-45 | 1P4 | 36227.7 | 196.7 | 3.87 | S1-45 | 1P4 | 31916.3 | 163.7 | 3.57 |
| RdRp_0.5 | 1A5 | 18134 | 190.3 | 1.76 | UBE2D3-His- | 1.00E+06 | 15571.7 | 192 | 1.58 |
| | | | | | biotin | | | | |
| UBE2D3-His- | 1.00E+06 | 18127.3 | 254.7 | 1.76 | NSP7_0.13 | 1A4 | 14432.7 | 211.3 | 1.44 |
| biotin | | | | | | | | | |
| S1-82 | 1J14 | 15226.7 | 171.3 | 1.42 | NSP9_0.25 | 1B3 | 13957.7 | 349.7 | 1.39 |
| NSP7_0.13 | 1A4 | 14377.7 | 264.7 | 1.32 | RdRp_0.5 | 1A5 | 12903.7 | 151.3 | 1.26 |
| NSP1_0.13 | 1B1 | 13128 | 238 | 1.17 | S1-82 | 1J14 | 10363.3 | 127.3 | 0.95 |
| ORF-3b_0.05 | 1H3 | 12776.7 | 209.3 | 1.13 | RdRp_0.17 | 1D5 | 10242.3 | 146.3 | 0.93 |
| NSP9_0.25 | 1B3 | 12688 | 401 | 1.12 | NSP4_0.1 | 1C2 | 9527 | 327.3 | 0.85 |
| NSP4_0.1 | 1C2 | 11066.7 | 405 | 0.93 | NSP14_0.5 | 1A6 | 8726 | 228.3 | 0.75 |
| RdRp_0.17 | 1D5 | 11058.7 | 178 | 0.93 | NSP1_0.13 | 1B1 | 8635 | 184 | 0.74 |
| S1-23 | 1C15 | 9728.7 | 172.3 | 0.78 | NSP8_0.25 | 1C3 | 8482 | 260.7 | 0.72 |

TABLE 10
Results demonstrate target antigen binding by Anti-Hu IgG (Control)
Table 10: Anti-Hu IgG (Control)

| Replicate 1 | | | | | Replicate 2 | | | | |
|---|---|---|---|---|---|---|---|---|---|
| Name | ID | F635 | F532 | Z-Score | Name | ID | F635 | F532 | Z-Score |
| Human IgG | 1G4 | 65535 | 228.7 | 9.27 | Human IgG | 1G4 | 65535 | 187.7 | 9.27 |
| ACE2_Fc_0.5 | 1.00E+07 | 65535 | 267.3 | 9.27 | ACE2_Fc_0.5 | 1.00E+07 | 65535 | 198.3 | 9.27 |
| ACE2_Fc_0.17 | 1H7 | 65535 | 209 | 9.27 | ACE2_Fc_0.17 | 1H7 | 65535 | 163 | 9.27 |
| Anti-H-IgG | 1G20 | 1830 | 151.7 | 0.14 | Anti-H-IgG | 1G20 | 1350 | 132.3 | 0.08 |
| S1_0.5 | 1B8 | 1559 | 150.7 | 0.1 | S1-61 | 1O4 | 1251.7 | 110 | 0.06 |
| S1_0.17 | 1C8 | 1413.7 | 151.7 | 0.08 | S1_0.5 | 1B8 | 1225.3 | 132.3 | 0.06 |
| S1-61 | 1O4 | 859.3 | 144.3 | 0 | S1_0.17 | 1C8 | 752.7 | 124.3 | −0.01 |

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. An antibody or antigen-binding fragment thereof comprising at least one of:
(a) a variable heavy chain complementarity-determining region CDR-H1, CDR-H2 and CDR-H3, wherein:
(i) CDR-H1 comprises a reconstructed polypeptide consensus sequence selected from any one of SEQ ID NOs: 10001-11250,
(ii) CDR-H2 comprises a reconstructed polypeptide consensus sequence selected from any one of SEQ ID NOs: 12501-13750, and
(iii) CDR-H3 comprises a reconstructed polypeptide consensus sequence selected from any one of SEQ ID NOs: 15001-16250; and
(b) a variable light chain complementarity-determining region CDR-L1, CDR-L2 and CDR-L3, wherein:
(i) CDR-L1 comprises a reconstructed polypeptide consensus sequence selected from any one of SEQ ID NOs: 11251-12500,
(ii) CDR-L2 comprises a reconstructed polypeptide consensus sequence selected from any one of SEQ ID NOs: 13751-15000, and
(iii) CDR-L3 comprises a reconstructed polypeptide consensus sequence selected from any one of SEQ ID NOs: 16251-17500.

2. An antibody or antigen-binding fragment thereof comprising at least one of:
(a) a variable heavy chain, wherein the variable heavy chain comprises a reconstructed polypeptide consensus sequence having at least 90% sequence identity to an amino acid sequence selected from any one of SEQ ID NOs: 17501-18750; and
(b) a variable light chain, wherein the variable light chain comprises a reconstructed polypeptide consensus sequence having at least 90% sequence identity to an amino acid sequence selected from any one of SEQ ID NOs: 18751-20000.

3. An antibody or antigen-binding fragment thereof that comprises:
(a) a variable heavy chain complementarity-determining region CDR-H1, CDR-H2, and CDR-H3, wherein CDR-H1 comprises a reconstructed polypeptide consensus sequence selected from any one of SEQ ID NOs: 10001-11250, CDR-H2 comprises a reconstructed polypeptide consensus sequence selected from any one of SEQ ID NOs: 12501-13750, and CDR-H3 comprises a reconstructed polypeptide sequence selected from any one of SEQ ID NOs: 15001-16250; and
(b) a variable light chain complementarity-determining region CDR-L1, CDR-L2, and CDR-L3, wherein CDR-L1 comprises a reconstructed polypeptide consensus sequence selected from any one of SEQ ID NOs: 11251-12500, CDR-L2 comprises a reconstructed polypeptide consensus sequence selected from any one of SEQ ID NOs: 13751-15000, and CDR-L3 comprises a reconstructed polypeptide sequence selected from any one of SEQ ID NOs: 16251-17500.

4. An antibody or antigen-binding fragment thereof that comprises a variable heavy chain, wherein the variable heavy chain comprises a reconstructed polypeptide consensus sequence selected from any one of SEQ ID NOs: 17501-18750; and a variable light chain, wherein the variable light chain comprises a reconstructed polypeptide consensus sequence selected from any one of SEQ ID NOs: 18751-20000.

5. An antibody or antigen-binding fragment thereof that comprises a variable heavy chain complementarity-determining region CDR-H1, CDR-H2, and CDR-H3, wherein CDR-H1 comprises a reconstructed polypeptide consensus sequence selected from any one of SEQ ID NOs: 10001-11250, CDR-H2 comprises a reconstructed polypeptide consensus sequence selected from any one of SEQ ID NOs: 12501-13750, and CDR-H3 comprises a reconstructed polypeptide sequence selected from any one of SEQ ID NOs: 15001-16250.

6. An antibody or antigen-binding fragment thereof that comprises a variable light chain complementarity-determining region CDR-L1, CDR-L2, and CDR-L3, wherein CDR-L1 comprises a reconstructed polypeptide consensus sequence selected from any one of SEQ ID NOs: 11251-12500, CDR-L2 comprises a reconstructed polypeptide consensus sequence selected from any one of SEQ ID NOs: 13751-15000, and CDR-L3 comprises a reconstructed polypeptide sequence selected from any one of SEQ ID NOs: 16251-17500.

7. An antibody or antigen-binding fragment thereof that comprises:
a variable heavy chain complementarity-determining region CDR-H1, CDR-H2, and CDR-H3, wherein CDR-H1 comprises a reconstructed polypeptide consensus sequence selected from any one of SEQ ID NOs: 10001-11250, CDR-H2 comprises a reconstructed polypeptide consensus sequence selected from any one of SEQ ID NOs: 12501-13750, and CDR-H3 comprises a reconstructed polypeptide sequence selected from any one of SEQ ID NOs: 15001-16250; and
a variable light chain complementarity-determining region CDR-L1, CDR-L2, and CDR-L3, wherein CDR-L1 comprises a reconstructed polypeptide consensus sequence selected from any one of SEQ ID NOs: 11251-12500, CDR-L2 comprises a reconstructed polypeptide consensus sequence selected from any one of SEQ ID NOs: 13751-15000, and CDR-L3 comprises a reconstructed polypeptide sequence selected from any one of SEQ ID NOs: 16251-17500.

8. An antibody or antigen-binding fragment thereof that comprises a variable heavy chain, wherein the variable heavy chain comprises a reconstructed polypeptide consensus sequence selected from any one of SEQ ID NOS: 17501-18750.

9. An antibody or antigen-binding fragment thereof that comprises a variable light chain, wherein the variable light chain comprises a reconstructed polypeptide consensus sequence selected from any of SEQ ID NOS: 18751-20000.

10. An antibody or antigen-binding fragment thereof that comprises:
(a) a variable heavy chain, wherein the variable heavy chain comprises a reconstructed polypeptide consensus sequence selected from any one of SEQ ID NOS: 17501-18750; and
(b) an antibody or antigen-binding fragment thereof that comprises a variable light chain, wherein the variable light chain comprises a reconstructed polypeptide consensus sequence selected from any of SEQ ID NOS: 18751-20000.

11. An antibody or antigen-binding fragment thereof that comprises:
(i) a variable heavy chain complementarity-determining region CDR-H1, CDR-H2 and CDR-H3, wherein
(a) the CDR-H1 comprises the amino acid sequence of SEQ ID NO: 10369,
(b) the CDR-H2 comprises the amino acid sequence of SEQ ID NO: 12869, and
(c) the CDR-H3 comprises the amino acid sequence of SEQ ID NO: 15369;
(ii) a variable light chain complementarity-determining region CDR-L1, CDR-L2 and CDR-L3, wherein:
(a) CDR-L1 comprises the amino acid sequence of SEQ ID NO: 11619,
(b) CDR-L2 comprises the amino acid sequence of SEQ ID NO: 14119, and
(c) CDR-L3 comprises the amino acid sequence of SEQ ID NO: 16619; or
(iii) the variable heavy chain complementarity-determining region CDR-H1, CDR-H2 and CDR-H3 of (i), and the variable light chain complementarity-determining region CDR-L1, CDR-L2 and CDR-L3 of (ii).

12. An antibody or antigen binding fragment thereof that comprises:
(a) a variable heavy chain that comprises a polypeptide sequence having at least 90% sequence identity to an amino acid sequence of SEQ ID NO: 17869;
(b) a variable light chain that comprises a polypeptide sequence having at least 90% sequence identity to an amino acid sequence of SEQ ID NO: 19119; or
(c) the variable heavy chain of (a), and the variable light chain of (b).

13. An antibody or antigen-binding fragment thereof that comprises:
(i) a variable heavy chain complementarity-determining region CDR-H1, CDR-H2 and CDR-H3, wherein
(a) the CDR-H1 comprises the amino acid sequence of SEQ ID NO: 10260,
(b) the CDR-H2 comprises the amino acid sequence of SEQ ID NO: 12760, and
(c) the CDR-H3 comprises the amino acid sequence of SEQ ID NO: 15260;
(ii) a variable light chain complementarity-determining region CDR-L1, CDR-L2 and CDR-L3, wherein:
(a) the CDR-L1 comprises the amino acid sequence of SEQ ID NO: 11510,
(b) the CDR-L2 comprises the amino acid sequence of SEQ ID NO: 14010, and
(c) the CDR-L3 comprises the amino acid sequence of SEQ ID NO: 16510; or
(iii) the variable heavy chain complementarity-determining region CDR-H1, CDR-H2 and CDR-H3 of (i), and the variable light chain complementarity-determining region CDR-L1, CDR-L2 and CDR-L3 of (ii).

14. An antibody or antigen binding fragment thereof that comprises:
(a) a variable heavy chain that comprises a polypeptide sequence having at least 90% sequence identity to an amino acid sequence of SEQ ID NO: 17760;
(b) a variable light chain that comprises a polypeptide sequence having at least 90% sequence identity to an amino acid sequence of SEQ ID NO: 19010; or
(c) the variable heavy chain of (a), and the variable light chain of (b).

15. An antibody or antigen-binding fragment thereof that comprises:
(i) a variable heavy chain complementarity-determining region CDR-H1, CDR-H2 and CDR-H3, wherein
(a) the CDR-H1 comprises the amino acid sequence of SEQ ID NO: 10705,
(b) the CDR-H2 comprises the amino acid sequence of SEQ ID NO: 13205, and
(c) the CDR-H3 comprises the amino acid sequence of SEQ ID NO: 15705;
(ii) a variable light chain complementarity-determining region CDR-L1, CDR-L2 and CDR-L3, wherein:
(a) the CDR-L1 comprises the amino acid sequence of SEQ ID NO: 11955,
(b) the CDR-L2 comprises the amino acid sequence of SEQ ID NO: 14455, and
(c) the CDR-L3 comprises the amino acid sequence of SEQ ID NO: 16955; or
(iii) the variable heavy chain complementarity-determining region CDR-H1, CDR-H2 and CDR-H3 of (i), and the variable light chain complementarity-determining region CDR-L1, CDR-L2 and CDR-L3 of (ii).

16. An antibody or antigen binding fragment thereof that comprises:
(a) a variable heavy chain that comprises a polypeptide sequence having at least 90% sequence identity to an amino acid sequence of SEQ ID NO: 18205;
(b) a variable light chain that comprises a polypeptide sequence having at least 90% sequence identity to an amino acid sequence of SEQ ID NO: 19455; or
(c) the variable heavy chain of (a), and the variable light chain of (b).

17. An antibody or antigen-binding fragment thereof that comprises:
(i) a variable heavy chain complementarity-determining region CDR-H1, CDR-H2 and CDR-H3, wherein
(a) the CDR-H1 comprises the amino acid sequence of SEQ ID NO: 10484,
(b) the CDR-H2 comprises the amino acid sequence of SEQ ID NO: 12984, and
(c) the CDR-H3 comprises the amino acid sequence of SEQ ID NO: 15484;
(ii) a variable light chain complementarity-determining region CDR-L1, CDR-L2 and CDR-L3, wherein:
(a) the CDR-L1 comprises the amino acid sequence of SEQ ID NO: 11734,
(b) the CDR-L2 comprises the amino acid sequence of SEQ ID NO: 14234, and
(c) the CDR-L3 comprises the amino acid sequence of SEQ ID NO: 16734; or
(iii) the variable heavy chain complementarity-determining region CDR-H1, CDR-H2 and CDR-H3 of (i), and the variable light chain complementarity-determining region CDR-L1, CDR-L2 and CDR-L3 of (ii).

18. An antibody or antigen binding fragment thereof that comprises:
(a) a variable heavy chain that comprises a polypeptide sequence having at least 90% sequence identity to an amino acid sequence of SEQ ID NO: 17984;
(b) a variable light chain that comprises a polypeptide sequence having at least 90% sequence identity to an amino acid sequence of SEQ ID NO: 19234; or
(c) the variable heavy chain of (a), and the variable light chain of (b).

19. An antibody or antigen-binding fragment thereof that comprises:
(i) a variable heavy chain complementarity-determining region CDR-H1, CDR-H2 and CDR-H3, wherein
(a) the CDR-H1 comprises the amino acid sequence of SEQ ID NO: 10291,
(b) the CDR-H2 comprises the amino acid sequence of SEQ ID NO: 12791, and
(c) the CDR-H3 comprises the amino acid sequence of SEQ ID NO: 15291;
(ii) a variable light chain complementarity-determining region CDR-L1, CDR-L2 and CDR-L3, wherein:
(a) the CDR-L1 comprises the amino acid sequence of SEQ ID NO: 11541,
(b) the CDR-L2 comprises the amino acid sequence of SEQ ID NO: 14041, and
(c) the CDR-L3 comprises the amino acid sequence of SEQ ID NO: 16541; or
(iii) the variable heavy chain complementarity-determining region CDR-H1, CDR-H2 and CDR-H3 of (i), and the variable light chain complementarity-determining region CDR-L1, CDR-L2 and CDR-L3 of (ii).

20. An antibody or antigen binding fragment thereof that comprises:
(a) a variable heavy chain that comprises a polypeptide sequence having at least 90% sequence identity to an amino acid sequence of SEQ ID NO: 17791;
(b) a variable light chain that comprises a polypeptide sequence having at least 90% sequence identity to an amino acid sequence of SEQ ID NO: 19041; or
(c) the variable heavy chain of (a), and the variable light chain of (b).

21. An antibody or antigen-binding fragment thereof that comprises:
(i) a variable heavy chain complementarity-determining region CDR-H1, CDR-H2 and CDR-H3, wherein
(a) the CDR-H1 comprises the amino acid sequence of SEQ ID NO: 10114,
(b) the CDR-H2 comprises the amino acid sequence of SEQ ID NO: 12614, and
(c) the CDR-H3 comprises the amino acid sequence of SEQ ID NO: 15114;
(ii) a variable light chain complementarity-determining region CDR-L1, CDR-L2 and CDR-L3, wherein:
(a) the CDR-L1 comprises the amino acid sequence of SEQ ID NO: 11364,
(b) the CDR-L2 comprises the amino acid sequence of SEQ ID NO: 13864, and
(c) the CDR-L3 comprises the amino acid sequence of SEQ ID NO: 16364; or
(iii) the variable heavy chain complementarity-determining region CDR-H1, CDR-H2 and CDR-H3 of (i), and the variable light chain complementarity-determining region CDR-L1, CDR-L2 and CDR-L3 of (ii).

22. An antibody or antigen binding fragment thereof that comprises:
(a) a variable heavy chain that comprises a polypeptide sequence having at least 90% sequence identity to an amino acid sequence of SEQ ID NO: 17614;
(b) a variable light chain that comprises a polypeptide sequence having at least 90% sequence identity to an amino acid sequence of SEQ ID NO: 18864; or
(c) the variable heavy chain of (a), and the variable light chain of (b).

23. An antibody or antigen-binding fragment thereof that comprises:
(i) a variable heavy chain complementarity-determining region CDR-H1, CDR-H2 and CDR-H3, wherein
(a) the CDR-H1 comprises the amino acid sequence of SEQ ID NO: 10394,
(b) the CDR-H2 comprises the amino acid sequence of SEQ ID NO: 12894, and
(c) the CDR-H3 comprises the amino acid sequence of SEQ ID NO: 15394;
(ii) a variable light chain complementarity-determining region CDR-L1, CDR-L2 and CDR-L3, wherein:
(a) the CDR-L1 comprises the amino acid sequence of SEQ ID NO: 11644,
(b) the CDR-L2 comprises the amino acid sequence of SEQ ID NO: 14144, and
(c) the CDR-L3 comprises the amino acid sequence of SEQ ID NO: 16644; or
(iii) the variable heavy chain complementarity-determining region CDR-H1, CDR-H2 and CDR-H3 of (i), and the variable light chain complementarity-determining region CDR-L1, CDR-L2 and CDR-L3 of (ii).

24. An antibody or antigen binding fragment thereof that comprises:
(a) a variable heavy chain that comprises a polypeptide sequence having at least 90% sequence identity to an amino acid sequence of SEQ ID NO: 17894;
(b) a variable light chain that comprises a polypeptide sequence having at least 90% sequence identity to an amino acid sequence of SEQ ID NO: 19144; or
(c) the variable heavy chain of (a), and the variable light chain of (b).

25. The antibody or antigen-binding fragment thereof of any one of claims 1-24, wherein the antibody comprises an IgG, IgA, IgM, or IgE antibody.

26. The antibody or antigen-binding fragment thereof of claim 25, wherein the IgG comprises IgG1, IgG2, IgG3, IgG4, IgGA1, or IgGA2.

27. The antibody or antigen-binding fragment thereof of any one of claims 1-26, wherein the antibody comprises a bispecific antibody, a multispecific antibody, a multivalent antibody, a chimeric antibody, a human antibody, humanized antibody, a monoclonal antibody, a deimmunized antibody, or a combination thereof.

28. The antibody or antigen-binding fragment thereof of any one of claims 1-27, wherein the antigen-binding fragment comprises a Fab, Fab′, Fab′-SH, Fv, scFv, F(ab′)₂, a diabody, a linear antibody, a single domain antibody (sdAb), a camelid V_{H}H domain, or a multi-specific antibody formed from antibody fragments.

29. The antibody or antigen-binding fragment thereof of any one of claims 1-28 wherein the antibody or antigen-binding fragment thereof is recombinant or synthetic.

30. The antibody or antigen-binding fragment thereof of any of claims 1-29, wherein the antibody or antigen-binding fragment thereof further comprise an enzyme, a substrate, cofactor, a fluorescent marker, a chemiluminescent marker, a peptide tag, a magnetic particle, a drug, a toxin, or a combination thereof.

31. The antibody or antigen-binding fragment thereof of any one of claims 1-30, wherein the antibody or antigen-binding fragment thereof binds to a SARS-CoV-2.

32. The antibody or antigen-binding fragment thereof of any one of claims 1-31, wherein the antibody or antigen binding fragment thereof binds a SARS-Cov-2 spike (S) protein, or a homolog thereof, or a variant thereof.

33. The antibody or antigen-binding fragment thereof of claim 32, wherein the antibody or antigen binding fragment thereof binds subunit S1, or a subunit S2 of the SARS-Cov-2 spike (S) protein.

34. The antibody or antigen-binding fragment thereof of claim 33, wherein the antibody or antigen binding fragment thereof binds a receptor binding domain of the subunit S1.

35. The antibody or antigen-binding fragment thereof of any one of claims 1-34, wherein the antibody or antigen-binding fragment thereof inhibits infection from SARS-CoV-2.

36. The antibody or antigen-binding fragment thereof of any one of claims 1-35, wherein the antibody or antigen-binding fragment thereof inhibits binding of a receptor binding domain of a subunit S1 of a SARS-CoV-2 with a receptor on a host cell.

37. The antibody or antigen-binding fragment thereof of any one of claims 1-36, wherein the antibody or antigen-binding fragment thereof inhibits entry of a SARS-CoV-2 in a host cell.

38. The antibody or antigen-binding fragment thereof of any one of claims 1-37, wherein the antibody or antigen-binding fragment is useful for treating COVID-19.

39. A pharmaceutical composition or a medicament that comprises the antibody or antigen-binding fragment thereof of any one of claims 1-38 and a pharmaceutically acceptable carrier, excipient or diluent.

40. The pharmaceutical composition or medicament of claim 39 formulated for administration via a subcutaneous, intravenous, intradermal, intraperitoneal, intramuscular, intracerebroventricular, intracranial, intracelial, or intracerebellar administration route.

41. The pharmaceutical composition or medicament of any one of claims 39-40, in an aqueous or in a lyophilized form.

42. The pharmaceutical composition or medicament of any one of claims 39-41, contained in a delivery device selected from the group consisting of a syringe, a blunt tip syringe, a catheter, and an implantable pump.

43. The pharmaceutical composition or medicament of any one of claims 39-42, comprising an additional therapeutic agent.

44. The pharmaceutical composition or medicament of claim 43, wherein the additional therapeutic agent is a nonsteroidal anti-inflammatory drug, a corticosteroid, a dietary supplement such as an antioxidant, a small molecule, a therapeutic vaccine, an immunomodulator, an angiotensin-converting enzyme [ACE] inhibitor, an angiotensin receptor blockers [ARBs], a HMG-CoA Reductase Inhibitors (Statins), an anti-viral agent, acetaminophen, or an additional anti-SARS-CoV-2 antibody.

45. A method for preventing a SARS-CoV-2 infection or COVID-19 in a subject in need thereof, the method comprising administering to the subject a therapeutically effective amount of either the antibody or antigen binding fragment of any one of claims 1-38, or the pharmaceutical composition of any one of claims 39-44.

46. A method for treating a SARS-CoV-2 infection or COVID-19 in a subject in need thereof, the method comprising administering to the subject,
(a) the antibody or antigen-binding fragment thereof of any one of claims 1-38; or
(b) the pharmaceutical composition or medicament of any one of claims 39-44.

47. The method of any one of claims 45-46, wherein the antibody or antigen-binding fragment thereof binds to the SARS-CoV-2.

48. The method of any one of claims 45-47, wherein the antibody or antigen binding fragment thereof binds a SARS-Cov-2 spike (S) protein, or a homolog thereof, or a variant thereof.

49. The method of claim 48, wherein the antibody or antigen binding fragment thereof binds subunit S1, or a subunit S2 of the SARS-Cov-2 spike (S) protein.

50. The method of claim 49, wherein the antibody or antigen binding fragment thereof binds a receptor binding domain of the subunit S1.

51. The method of any one of claims 45-50, wherein the antibody or antigen-binding fragment thereof inhibits binding of a receptor binding domain of a subunit S1 of the SARS-CoV-2 with a receptor on a host cell.

52. The method of any one of claims 45-51, wherein the antibody or antigen-binding fragment thereof inhibits entry of the SARS-CoV-2 in a host cell.

53. The method of any one of claims 45-52, wherein the antibody or antigen-binding fragment thereof inhibits fusion of the SARS-CoV-2 membrane with a host cell membrane.

54. The method of any one of claims 45-53, wherein the antibody or antigen binding fragment thereof neutralizes the SARS-CoV-2.

55. The method of any one of claims 46-54, wherein administering reduces one or more symptoms associated with a SARS-CoV-2 infection.

56. The method of any one of claims 46-55, wherein the administering reduces viral load in the subject.

57. The method of any one of claims 45-56, wherein the antibody or antigen binding fragment thereof is administered to the subject with an additional therapeutic agent.

58. The method of claim 57, wherein the additional therapeutic agent is a nonsteroidal anti-inflammatory drug, a corticosteroid, a dietary supplement such as an antioxidant, a small molecule, a therapeutic vaccine, an immunomodulator, an angiotensin-converting enzyme [ACE] inhibitor, an angiotensin receptor blockers [ARBs], a HMG-CoA Reductase Inhibitors (Statins), an anti-viral agent, acetaminophen, or an additional anti-SARS-CoV-2 antibody.

59. A hybridoma that produces the antibody or antigen-binding fragment thereof of any one of claims 1-38.

60. A fusion protein that comprises the antibody or antigen-binding fragment thereof of any one of claims 1-38.

61. An immunoconjugate comprising the antibody or the antigen binding fragment thereof of any one of claims 1-38, and a therapeutic agent.

62. An isolated nucleic acid comprising at least one of:
(a) a nucleic acid sequence encoding CDR-H1, wherein the nucleic acid sequence is selected from SEQ ID NOs: 1-1250;
(b) a nucleic acid sequence encoding CDR-L1, wherein the nucleic acid sequence is selected from SEQ ID NOs: 1251-2500;
(c) a nucleic acid sequence encoding CDR-H2, wherein the nucleic acid sequence is selected from SEQ ID NOs: 2501-3750;
(d) a nucleic acid sequence encoding CDR-L2, wherein the nucleic acid sequence is selected from SEQ ID NOs: 3751-5000;
(e) a nucleic acid sequence encoding CDR-H3, wherein the nucleic acid sequence is selected from SEQ ID NOs: 5001-6250; or
(f) a nucleic acid sequence encoding CDR-L3, wherein the nucleic acid sequence is selected from SEQ ID NOs: 6251-7500.

63. An isolated nucleic acid comprising at least one of:
(a) a nucleic acid sequence encoding a heavy chain polypeptide of an antibody, wherein the nucleic acid sequence is selected from any one of SEQ ID NOs: 7501-8750, and
(b) a nucleic acid sequence encoding a light chain polypeptide of an antibody, wherein the nucleic acid sequence is selected from any one of SEQ ID NOs: 8751-10000.

64. An isolated nucleic acid that comprises a reconstructed nucleic acid consensus sequence encoding a heavy chain polypeptide of an antibody, wherein the nucleic acid consensus sequence is selected from any of SEQ ID NOS: 7501-8750.

65. An isolated nucleic acid that comprises a reconstructed nucleic acid consensus sequence encoding a light chain polypeptide of an antibody, wherein the nucleic acid consensus sequence is selected from any of SEQ ID NOS: 8751-10000.

66. An expression vector comprising the isolated nucleic acid molecule of any one of claims 62-65.

67. The expression vector of claim 66, wherein the isolated nucleic acid is operably linked to a regulatory control sequence.

68. A host cell comprising the expression vector of any one of claims 66-67, or the isolated nucleic acid molecule of any one of claims 62-65.

69. The host cell of claim 68, wherein said host cell is a mammalian cell, or a bacterial cell.

70. The host cell of claim 68 or claim 69, wherein said bacterial cell is an Escherichia. coli cell.

71. The host cell of any one of claims 68-70, wherein the expression of the nucleic acid is under control of one or more inducible promoters.

72. A method of diagnosing a subject as being infected with a SARS-Cov-2 virus or suspected of being infected with a SARS-Cov-2 virus, the method comprising contacting a sample obtained from the subject with the antibody or the antigen-binding fragment of any one of claims 1-38; detecting the presence or absence of the antibody or the antigen-binding fragment; and diagnosing the subject as being infected with a SARS-CoV-2 virus when the presence of the antibody or the antigen-binding fragment is detected.

73. The method of claim 72, wherein the sample comprises a nasal swab, a tissue sample, saliva, or blood.

74. The method of any one of claims 72-73, wherein detecting the presence or absence of the antibody or the antigen-binding fragment comprises an enzyme linked immunosorbent assay (ELISA), an immunospot assay, a lateral flow assay, flow cytometry, immunohistochemistry, or a western blot.

75. An immunohistochemical assay comprising;
(a) contacting a sample with the antibody or antigen binding fragment thereof of any one of claims 1-38 under conditions permitting selective binding of the antibody or antigen binding fragment thereof with a SARS-CoV-2, to form an antibody-antigen complex; and
(b) detecting the presence or absence of the antibody-antigen complex by an immunodetection method.

76. The immunohistochemical assay of claim 75, wherein the sample is a nasal swab, a tissue sample, saliva, or blood.

77. The immunohistochemical assay of claim 75 or claim 76, wherein the sample is from a subject suspected to be suffering from a SARS-CoV-2 infection or COVID-19.

78. A method of inhibiting binding of a SARS-CoV-2 with a host cell, or inhibiting entry of a SARS-CoV2 in a host cell, the method comprising contacting the SARS-CoV-2 with the antibody or antigen binding fragment thereof of any one of claims 1-38.

79. The method of claim 78, wherein the antibody or antigen binding fragment thereof binds a SARS-Cov-2 spike (S) protein, or a homolog thereof, or a variant thereof.

80. The method of claim 79, wherein the antibody or antigen binding fragment thereof binds subunit S1, or a subunit S2 of the SARS-Cov-2 spike (S) protein.

81. The method of claim 79, wherein the antibody or antigen binding fragment thereof binds a receptor binding domain of the subunit S1.

82. The method of any one of claims 78-81, wherein the antibody or antigen-binding fragment thereof inhibits binding of a receptor binding domain of a subunit S1 of the SARS-CoV-2 with a receptor on the host cell.

83. The method of any one of claims 78-82, wherein the antibody or antigen-binding fragment thereof inhibits fusion of the SARS-CoV-2 membrane with the host cell membrane.

84. The method of any one of claims 78-83, wherein the antibody or antigen binding fragment thereof neutralizes the SARS-CoV-2.

85. A method of producing an antibody or an antigen binding fragment thereof, the method comprising:
(a) culturing the host cell of any one of claims 68-71, in a medium under conditions permitting expression of a polypeptide encoded by the isolated nucleic acid, and assembling of the antibody or an antigen binding fragment thereof; and
(b) purifying the antibody or antigen binding fragment thereof from the cultured cell or the cell culturing medium.

86. An antibody or antigen-binding fragment thereof comprising at least one of:
(a) a variable heavy chain complementarity-determining region CDR-H1, CDR-H2 and CDR-H3, wherein:
(i) CDR-H1 comprises a polypeptide sequence selected from any one of SEQ ID NOs: 10001-11250,
(ii) CDR-H2 comprises a polypeptide sequence selected from any one of SEQ ID NOs: 12501-13750, and
(iii) CDR-H3 comprises a polypeptide sequence selected from any one of SEQ ID NOs: 15001-16250; and
(b) a variable light chain complementarity-determining region CDR-L1, CDR-L2 and CDR-L3, wherein:
(i) CDR-L1 comprises a polypeptide sequence selected from any one of SEQ ID NOs: 11251-12500,
(ii) CDR-L2 comprises a polypeptide sequence selected from any one of SEQ ID NOs: 13751-15000, and
(iii) CDR-L3 comprises a polypeptide sequence selected from any one of SEQ ID NOs: 16251-17500.

87. An antibody or antigen-binding fragment thereof comprising at least one of:
(c) a variable heavy chain, wherein the variable heavy chain comprises a polypeptide sequence having at least 90% sequence identity to an amino acid sequence selected from any one of SEQ ID NOs: 17501-18750; and
(d) a variable light chain, wherein the variable light chain comprises a polypeptide sequence having at least 90% sequence identity to an amino acid sequence selected from any one of SEQ ID NOs: 18751-20000.

88. The antibody or antigen-binding fragment thereof of any one of claims 86-87, wherein the antibody comprises an IgG, IgA, IgM, or IgE antibody.

89. The antibody or antigen-binding fragment thereof of claim 88, wherein the IgG comprises IgG1, IgG2, IgG3, IgG4, IgGA1, or IgGA2.

90. The antibody or antigen-binding fragment thereof of any of claims 86-89, wherein the antibody comprises a bispecific antibody, a multispecific antibody, a multivalent antibody, a chimeric antibody, a human antibody, humanized antibody, a monoclonal antibody, a deimmunized antibody, or a combination thereof.

91. The antibody or antigen-binding fragment thereof of any one of claims 86-90, wherein the antigen-binding fragment comprises a Fab, Fab′, Fab′-SH, Fv, scFv, F(ab′)2, a diabody, a linear antibody, a single domain antibody (sdAb), a camelid V_{H}H domain, or a multi-specific antibody formed from antibody fragments.

92. The antibody or antigen-binding fragment thereof of any one of claims 86-91, wherein the antibody or antigen-binding fragment thereof is recombinant or synthetic.

93. The antibody or antigen-binding fragment thereof of any one of claims 86-92, wherein the antibody or antigen-binding fragment binds SARS-CoV-2, the virus that causes COVID-19.

94. A hybridoma that produces the antibody or antigen-binding fragment thereof of any one of claims 86-93.

95. A pharmaceutical composition or a medicament that comprises the antibody or antigen-binding fragment thereof of any one of claims 86-94, and a pharmaceutically acceptable carrier, excipient or diluent.

96. The pharmaceutical composition or medicament of claim 95, formulated for administration via a subcutaneous, intravenous, intradermal, intraperitoneal, intramuscular, intracerebroventricular, intracranial, intracelial, or intracerebellar administration route.

97. The pharmaceutical composition or medicament of any one of claims 95-96, in an aqueous or in a lyophilized form.

98. The pharmaceutical composition or medicament of any one of claims 95-97, contained in a delivery device selected from the group consisting of a syringe, a blunt tip syringe, a catheter, and an implantable pump.

99. A method for treating or preventing a SARS-CoV2 infection or a COVID-19 in a subject tin need thereof, the method comprising administering to the subject the antibody or antigen-binding fragment thereof of any one of claims 86-93, or the pharmaceutical composition or medicament of any one of claims 95-98.

100. Use of the antibody or antigen binding fragment of any one of claims 1-38, and claims 86-93 for treatment or prevention of a SARS-CoV-2 infection or COVID-19.
